(19) United States
(12) Patent Application Publication
(10) Pub. No.: US 2024/0182518 A1
(43) Pub. Date: Jun. 6, 2024

(54) COMPOSITIONS AND METHODS COMPRISING SPLICING-DERIVED ANTIGENS FOR TREATING CANCER

(71) Applicant: THE REGENTS OF THE UNIVERSITY OF CALIFORNIA, Oakland, CA (US)

(72) Inventors: Yi XING, Los Angeles, CA (US); Owen WITTE, Los Angeles, CA (US); John PHILLIPS, Los Angeles, CA (US); Yang PAN, Los Angeles, CA (US)

(73) Assignee: THE REGENTS OF THE UNIVERSITY OF CALIFORNIA, Oakland, CA (US)

(21) Appl. No.: 17/760,479

(22) PCT Filed: Feb. 12, 2021
(86) PCT No.: PCT/US2021/017958

§ 371 Date: Aug. 10, 2022

Related U.S. Application Data
(60) Provisional application No. 62/976,654, filed on Feb. 14, 2020.

(51) Int. Cl. C07K 7/02; A61K 39/00

(52) U.S. Cl. CPC C07K 7/02; A61K 39/00; C07K 2319/40; C07K 2319/92

(57) ABSTRACT

Methods and processes to identify neoplastic tissue antigens derived from alternative splicing (AS) are described, in accordance with various embodiments of the invention. Also described are novel tumor antigens that are useful as targets in various immunotherapeutic approaches to treating cancer as well as novel engineered T cell Receptors (TCRs) and chimeric antigen receptors (CARs) that target these antigenic peptides.

This application is claims benefit of priority of U.S. Provisional Application No. 62/976,654, filed Feb. 14, 2020 which is hereby incorporated by reference in its entirety.

This invention was made with government support under Grant Numbers CA092131. CA220238 and CA232979 awarded by the National Institutes of Health. The government has certain rights in the invention.

### BACKGROUND OF THE INVENTION

### I. Field of the Invention

This invention relates to the field of cancer therapies.

### II. Background

Cancer immunotherapy has gained tremendous momentum in the past decade. The clinical effectiveness of checkpoint inhibitors, such as neutralizing antibodies against PD-1 and CTLA-4, is thought to result from their ability to reactivate tumor-specific T cells. Meanwhile, adoptive cell therapies use genetically modified T-cell receptors (TCRs) or synthetic chimeric antigen receptor T cells (CAR-T) for tumor-specific antigen recognition. The finding that cancer cells express specific T-cell-reactive antigens has galvanized epitope discovery in recent years. Nevertheless, the identification of tumor antigens remains a major challenge. Although somatic mutation-derived antigens have been successfully targeted by cancer therapies, this approach remains largely ineffective for tumors with low or moderate mutation loads. Thus, there is a need for the identification and characterization of novel tumor antigens that are useful targets in cancer immunotherapies.

### SUMMARY OF THE INVENTION

Described herein are novel tumor antigens that are useful as targets in various immunotherapeutic approaches to treating cancer as well as novel engineered T cell Receptors (TCRs) and chimeric antigen receptors (CARs) that target these antigenic peptides.

Aspects of the disclosure relate to a peptide comprising at least 70% sequence identity to a peptide of SEQ ID NO:1-19. In some aspects, the peptide comprises or consists of a sequence that is, is at least, or is at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical to the sequence of one of SEQ ID NOS:1-19. Further aspects of the disclosure relate to a peptide comprising at least 6 contiguous amino acids from an alternatively spliced polypeptide, wherein the at least 6 contiguous amino acids comprises an alternative splice site junction of the polypeptide or wherein the peptide comprises at least 6 contiguous amino acids from an alternatively spliced exon; and wherein the alternatively spliced peptide, exon, or junction is one that is derived from an alternative splice event (AS event) identified in Table 1a, 1b, 1c, or 1d. The disclosure also describes a molecular complex comprising a peptide of the disclosure and a major histocompatibility complex (MHC) molecule. Further aspects relate to compositions comprising the peptide, nucleic acids encoding the peptide, and vectors comprising nucleic acids encoding the peptide. Also disclosed are cells comprising the peptide and methods of making and using the peptide.

Further aspects relate to an in vitro isolated dendritic cell comprising a peptide, nucleic acid, or vector of the disclosure. Further aspects relate to a method of making a cell comprising transferring a nucleic acid or expression vector of the disclosure into a cell. In some aspects, the disclosure relates to an in vitro method for making a dendritic cell vaccine comprising contacting a mature dendritic cell in vitro with a peptide of the disclosure. Further aspects relate to an in vitro composition comprising a dendritic cell and a peptide of the disclosure. Further aspects relate to an engineered T-cell Receptor (TCR) or chimeric antigen receptor (CAR) that specifically recognizes a peptide of the disclosure. Also provided are cells comprising the TCR or CARs of the disclosure. Further aspects relate to an antibody or antigen binding fragment thereof that specifically recognizes a peptide or molecular complex of the disclosure. Further aspects relate to a method of treating or preventing cancer in a subject comprising administering a peptide, molecular complex, composition, dendritic cell, nucleic acid, expression vector, peptide-specific binding molecule, TCR, or an antibody or antigen binding fragment of the disclosure. Further aspects relate to a method of stimulating an immune response in a subject, the method comprising administering an effective amount of a peptide, molecular complex, composition, nucleic acid or expression vector, cell, peptide-specific binding molecule, antibody, antigen binding fragment, or TCR of the disclosure.

The disclosure also describes a peptide-specific binding molecule, wherein the molecule specifically binds to a peptide or molecular complex of the disclosure. In some embodiments, the binding molecule is an antibody, a T cell receptor (TCR), TCR mimc antibody, scFV, camellid, aptamer, or DARPIN.

Further method aspects of the disclosure relate to a method of producing cancer-specific immune effector cells comprising: (a) obtaining a starting population of immune effector cells; and (b) contacting the starting population of immune effector cells with a peptide or molecular complex of the disclosure, thereby generating peptide-specific immune effector cells. Also provided are immune effector cells produced according to methods of the disclosure.

Further aspects relate to a method for prognosing a patient or for detecting T cell responses in a patient, the method comprising: contacting a biological sample from the patient with the peptide or molecular complex of the disclosure. Also disclosed is a method comprising contacting a composition of the disclosure with a composition comprising T cells and detecting T cells with bound peptide and/or MHC polypeptide by detecting a detection tag.

The peptide may be a peptide of one of SEQ ID NOS: 1-19. In some embodiments, the peptide comprises SEQ ID NO:1. In some embodiments, the peptide comprises SEQ ID NO:2. In some embodiments, the peptide comprises SEQ ID NO:3. In some embodiments, the peptide comprises SEQ ID NO:4. In some embodiments, the peptide comprises SEQ ID NO:5. In some embodiments, the peptide comprises SEQ ID NO:6. In some embodiments, the peptide comprises SEQ ID NO:7. In some embodiments, the peptide comprises SEQ ID NO:8. In some embodiments, the peptide comprises SEQ ID NO:9. In some embodiments, the peptide comprises SEQ ID NO: 10. In some embodiments, the peptide comprises SEQ ID NO:11. In some embodiments, the peptide comprises SEQ ID NO: 12. In some embodiments, the peptide comprises SEQ ID NO:13. In some embodiments, the peptide comprises SEQ ID NO:14. In some embodiments, the peptide comprises SEQ ID NO:15. In some embodiments, the peptide comprises SEQ ID NO: 16. In some embodiments, the peptide comprises SEQ ID NO:17. In some embodiments, the peptide comprises SEQ ID NO:18. In some embodiments, the peptide comprises SEQ ID NO:19. The peptide may be at least 6 contiguous amino acids of one of SEQ ID NOS: 1-19. In some embodiments, the peptide is, is at least, or is at most 5, 6, 7, 8, 9, 10, or 11 (or any derivable range therein) contiguous amino acids of one of SEQ ID NOS:1-19. In some embodiments, the peptide is 13 amino acids or fewer in length. In some embodiments, the peptide is, is more than, or is less than 15, 14, 13, 12, 11, 10, or 9 amino acids in length (or any derivable range therein). In some embodiments, the peptide consists of 9 amino acids. In some embodiments, the peptide consists of 6, 7, 8, 9, 10, 11, 12, 13, 14, or 15 amino acids. The peptide may be further defined as an immunogenic peptide. The term “immunogenic” in reference to a peptide refers to a peptide that can induce an immune response in vivo. In some embodiments, the peptide is modified. The modification may be, for example, conjugation to a molecule. Molecules include antibodies, lipids, adjuvants, and/or detection moieties. The peptide may have 1, 2, 3, 4, 5, or 6 substitutions relative to a peptide of one of SEQ ID NOS:1-19.

In some embodiments, the AS event is selected from an AS event in Table 1a. In some embodiments, the AS event is selected from an AS event in Table 1b. In some embodiments, the AS event is selected from an AS event in Table 1c. In some embodiments, the AS event is selected from an AS event in Table 1d. In some embodiments, the disclosure relates to a CAR that targets a peptide of the disclosure, wherein the peptide comprises an AS event from table 1a. In some embodiments, the disclosure relates to a CAR that targets a peptide of the disclosure, wherein the peptide comprises an AS event from table 1b. In some embodiments, the disclosure relates to a CAR that targets a peptide of the disclosure, wherein the peptide comprises an AS event from table 1c. In some embodiments, the disclosure relates to a CAR that targets a peptide of the disclosure, wherein the peptide comprises an AS event from table 1d. In some embodiments, the disclosure relates to a TCR that targets a peptide of the disclosure, wherein the peptide comprises an AS event from table 1a. In some embodiments, the disclosure relates to a TCR that targets a peptide of the disclosure, wherein the peptide comprises an AS event from table 1b. In some embodiments, the disclosure relates to a TCR that targets a peptide of the disclosure, wherein the peptide comprises an AS event from table 1c. In some embodiments, the disclosure relates to a TCR that targets a peptide of the disclosure, wherein the peptide comprises an AS event from table 1d.

In some embodiments, the peptide comprises at least 10 amino acids. In some embodiments, the peptide consists of 10 amino acids. In some embodiments, the peptide is less than 20 amino acids in length. In some embodiments, the peptide comprises at least, at most, exactly, or about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids (or any derivable range therein). In some embodiments, the peptide consists of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In some embodiments, the peptide is modified. Modifications include conjutation to a molecule, such as an antibody, lipid, adjuvant, or a detection moiety.

In some embodiments, the disclosure relates to a composition comprising a peptide of the disclosure, wherein the composition is formulated as a vaccine. In some embodiments, the composition further comprises an adjuvant. The composition may be formulated for parenteral administration, intravenous injection, intramuscular injection, inhalation, or subcutaneous injection.

In some embodiments, the TCR comprises a modification or is chimeric. In some embodiments, the variable region of the TCR is fused to a TCR constant region that is different from the constant region of the cloned TCR that specifically binds to a peptide of the disclosure.

In some embodiments, the nucleic acid of the disclosure comprises a cDNA encoding the TCR. In some embodiments, the TCR alpha and beta genes are on the same nucleic acid and/or on the same vector.

In some embodiments, a cell of the disclosure comprises a stem cell, a progenitor cell, or a T cell. In some embodiments, the cell comprises a hematopoietic stem or progenitor cell, a T cell, or an induced pluripotent stem cell (iPSC). In some embodiments, the cell is isolated from a cancer patient. In some embodiments, is a HLA-A type. In some embodiments, the cell is a HLA-A*03:01 type. In some embodiments, the cell comprises at least one TCR and at least one CAR and wherein the TCR and CAR each recognize a different peptide. For example, embodiments of the disclosure relate to a cell that comprises a TCR that targets one peptide of the disclosure and a CAR that targets a different peptide of the disclosure. In some embodiments, the dendritic cell comprises a monocyte-derived dendritic cell.

In some embodiments, the composition of the disclosure has been determined to be serum-free, mycoplasma-free, endotoxin-free, and/or sterile.

In some embodiments, the method further comprises culturing the cell in media, incubating the cell at conditions that allow for the division of the cell, screening the cell, and/or freezing the cell. In some embodiments, the method further comprises isolating the expressed peptide or polypeptide from a cell of the disclosure.

In some embodiments, the cancer comprises prostate cancer. In some embodiments, the cancer comprises breast cancer. In some embodiments, the cancer comprises lung cancer. In some embodiments, the subject has previously been treated for the cancer. In some embodiments, the subject has been determined to be resistant to the previous treatment. In some embodiments, the method further comprises the administration of an additional therapy. In some embodiments, the additional therapy comprises an immunotherapy, chemotherapy, or an additional therapy described herein. In some embodiments, the cancer comprises stage I, II, III, or IV cancer. In some embodiments, the cancer comprises metastatic and/or recurrent cancer.

Treating in the methods of the disclosure may comprise one or more of reducing tumor size; increasing the overall survival rate; reducing the risk of recurrence of the cancer; reducing the risk of progression; and/or increasing the chance of progression-free survival, relapse-free survival, and/or recurrence-free survival.

Also described are compositions comprising at least one MHC polypeptide and the peptides described herein and above.

In some embodiments, the compositions of the disclosure are formulated as a vaccine. In some embodiments, the compositions and methods of the disclosure provide for prophylactic therapies to prevent cancer. In some embodiments, the compositions and methods of the disclosure provide for therapeutic therapies to treat existing cancers, such as for the treatment of patients with a cancerous tumor. In some embodiments, the composition further comprises an adjuvant. Adjuvants are known in the art and include, for example, TLR agonists and aluminum salts.

In some embodiments, the dendritic cell comprises a mature dendritic cell. In some embodiments, the cell is a cell with an HLA type selected from HLA-A, HLA-B, or HLA-C.

In some embodiments the methods of the disclosure further comprise screening the dendritic cell for one or more cellular properties. In some embodiments, the method further comprises contacting the cell with one or more cytokines or growth factors. In some embodiments, the one or more cytokines or growth factors comprises GM-CSF. In some embodiments, the cellular property comprises cell surface expression of one or more of CD86, HLA, and CD14. In some embodiments, the dendritic cell is derived from a CD34+ hematopoietic stem or progenitor cell.

In some embodiments, the dendritic cell is derived from a peripheral blood monocyte (PBMC). In some embodiments, the dendritic cells is isolated from PBMCs. In some embodiments, the dendritic cells are cells in which the DCs are derived from are isolated by leukaphereses.

In some embodiments, the composition further comprises one or more cytokines, growth factors, or adjuvants. In some embodiments, the composition comprises GM-CSF. In some embodiments, the peptide and GM-CSF are linked. In some embodiments, the composition is determined to be serum-free, mycoplasma-free, endotoxin-free, and sterile. In some embodiments, the peptide is on the surface of the dendritic cell. In some embodiments, the peptide is bound to a MHC molecule on the surface of the dendritic cell. In some embodiments, the composition is enriched for dendritic cells expressing CD86 on the surface of the cell. In some embodiments, the dendritic cell is derived from a CD34+ hematopoietic stem or progenitor cell. In some embodiments, the dendritic cell is derived from a peripheral blood monocyte (PBMC). In some embodiments, the dendritic cells or cells in which the DCs are derived are isolated by leukaphereses.

In some embodiments of the disclosure, the cell comprises a stem cell, a progenitor cell, or a T cell. In some embodiments, the cell comprises a hematopoietic stem or progenitor cell, a T cell, or an induced pluripotent stem cell (iPSC).

In some embodiments, the method comprises administering a cell or a composition comprising a cell and wherein the cell comprises an autologous or allogenic cell. In some embodiments, the cell comprises a non-autologous cell. In some embodiments, contacting in the methods of the disclosure is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface. In some embodiments, the APCs are dendritic cells. The immune effector cells may be T cells, peripheral blood lymphocytes, NK cells, invariant NK cells, and/or NKT cells. In some embodiments, the immune effector cells have been differentiated from mesenchymal stem cell (MSC) or induced pluripotent stem (iPS) cells. The T cells may be CD8+ T cells, CD4+ T cells, or γδ T cells. The T cells may be cytotoxic T lymphocytes (CTLs). Obtaining in the method embodiments may comprise isolating the starting population of immune effector cells from peripheral blood mononuclear cells (PBMCs). In some embodiments, the starting population of immune effector cells is obtained from a subject. In some embodiments, the subject is a human. The subject may also be a non-human primate, a laboratory research animal, a rat, a mouse, a pig, a monkey, a guinea pig, a rabbit, or a horse, for example. In some embodiments, the subject is a mammal. The subject may be one that has a cancer and/or has been diagnosed with a cancer. In some embodiments, the cancer comprises tumor cells that are positive for expression of the peptide. In some embodiments, the cancer comprises prostate cancer. In some embodiments, the cancer comprises a cancer that is positive for expression of the peptide. In some embodiments, the subject has been determined to have a cancer that is positive for expression of the peptide. In some embodiments, the method further comprises introducing the peptide or a nucleic acid encoding the peptide into the dendritic cells prior to the co-culturing. The peptide or nucleic acids encoding the peptide may be introduced by electroporation. In some embodiments, the peptide or nucleic acids encoding the peptide are introduced by adding the peptide or nucleic acid encoding the peptide to the dendritic cell culture media. The immune effector cells may be co-cultured with a second population of dendritic cells into which the peptide or the nucleic acid encoding the peptide has been introduced. In some embodiments, a population of CD8 or CD4-positive and peptide MHC tetramer-positive T cells are purified from the immune effector cells following the co-culturing. In some embodiments, a clonal population of peptide-specific immune effector cells are generated by limiting or serial dilution followed by expansion of individual clones by a rapid expansion protocol. In some embodiments, the method further comprises cloning of a T cell receptor (TCR) from the clonal population of peptide-specific immune effector cells. Cloning of the TCR may comprise cloning of a TCR alpha and a beta chain. The TCR may be cloned using a 5′-Rapid amplification of cDNA ends (RACE) method. The cloned TCR may be is subcloned into an expression vector. The expression vector may be a retroviral or lentiviral vector. In some embodiments, a host cell is transduced with the expression vector to generate an engineered cell that expresses the TCR. The host cell may be an immune cell. In some embodiments, the immune cell is a T cell and the engineered cell is an engineered T cell. In some embodiments, the T cell is a CD8+ T cell, CD4+ T cell, or γδ T cell and the engineered cell is an engineered T cell. In some embodiments, the starting population of immune effector cells is obtained from a subject with cancer and the host cell is allogeneic or autologous to the subject.

The cancer may be one that is positive for expression of the peptide and/or the subject may be one that has been determined to have a biological sample that is positive for the peptide. In some embodiments, a population of CD8 or CD4-positive and peptide MHC tetramer-positive engineered T cells are purified from the transduced host cells. In some embodiments, a clonal population of peptide-specific engineered T cells are generated by limiting or serial dilution followed by expansion of individual clones by a rapid expansion protocol.

In embodiments of the disclosure, the biological sample may comprise a blood sample, a fraction of a blood sample, a tissue sample, a biopsy, or a tumor sample. In some embodiments, the biological sample comprises lymphocytes. In some embodiments, the biological sample comprises a fractionated sample comprising lymphocytes. In some embodiments, the peptide is linked to a solid support. In some embodiments, the peptide is conjugated to the solid support or is bound to an antibody that is conjugated to the solid support. In some embodiments, the solid support comprises a microplate, a bead, a glass surface, a slide, or a cell culture dish. In some embodiments, detecting T cell responses comprises detecting the binding of the peptide to the T cell or TCR. In some embodiments, detecting T cell responses comprises an ELISA, ELISPOT, or a tetramer assay.

In some embodiments, the MHC polypeptide is and/or peptide is conjugated to a detection tag. In some embodiments, the MHC polypeptide and peptide are operatively linked to form a peptide-MHC complex. In some embodiments, the MHC polypeptide and peptide are operatively linked through a peptide bond. In some embodiments, the MHC polypeptide and peptide are operatively linked through van der Waals forces. In some embodiments, at least two peptide-MHC complexes are operatively linked to each other. In some embodiments, at least 3 or 4 peptide-MHC complexes are operatively linked to each other. In some embodiments, the average ratio of MHC polypeptides to peptides is 1:1 to 4:1.

Method embodiments of the disclosure may further comprise counting the number of T cells bound with peptide and/or MHC. The composition comprising T cells may be isolated from a patient having or suspected of having a cancer. The cancer may comprise a peptide-specific cancer. The cancer may be prostate, breast, or lung cancer. In some embodiments, the peptide is selected from a peptide of one of SEQ ID NOS:1-19. In some embodiments, the method further comprises sorting the number of T cells bound with peptide and/or MHC. In some embodiments, the method further comprises sequencing one or more TCR genes from T cells bound with peptide and/or MHC. In some embodiments, the method further comprises grouping of lymphocyte interactions by paratope hotspots (GLIPH) analysis.

The disclosure also describes kits comprising a peptide of the disclosure in a container. The peptide may be comprised in a pharmaceutical preparation. The pharmaceutical preparation may be formulated for parenteral administration or inhalation. The peptide may be comprised in a cell culture media.

Further embodiments are disclosed in U.S. Patent Application Nos.: 62/934,914 and 62/932,751, which are incorporated by reference, and may be combined with the embodiments described herein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more.” “at least one.” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z.” “(x and y) or z.” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B. A global, exon-level analysis of alternative pre-mRNA splicing in normal prostate and prostate cancers identifies patterns of exon usage in RNA-binding proteins. (A) Schematic with alluvial plot depicting the data processing workflow combining RNA-Seq data from various prostate tissue disease states (left panel) and summary table depicting various exon events detected by rMATS-turbo before and after filtering for splice junction reads coverage, PSI range and commonality (right panel). The alluvial plot depicts the sorting of patient RNA-Seq datasets from individual studies on the left into prostate phenotypes on the right. (B) Scatter plot depiction of an unsupervised principle component analysis of exon usage matrices from eight different prostate datasets representing healthy tissue, tumor-adjacent benign tissue, primary prostate cancer, metastatic castration-resistant prostate cancer (mCRPC), and treatment-associated neuroendocrine prostate cancer (NEPC).

FIG. 2A-D. Pathway Enrichment-Guided Activity Study of Alternative Splicing (PEGASAS) analysis identifies exon correlates of oncogenic signaling in prostate cancers. (A) Workflow diagram describing PEGASAS correlation of gene signature score with exon usage. Each sample is scored for a gene expression signature of interest. Gene signature scores are correlated with exon usage matrices to identify pathway-correlated exon incorporation changes. (B) Heatmap of the correlation coefficients of the exon changes correlated with gene signatures in the GSEA Hallmark sets as generated by PEGASAS. The ten signatures that returned the highest number of exon correlates are shown here. Each row depicts the results of the correlation to a single Hallmark signature. Each column represents a single exon. The color represents the strength and direction of the correlation (red positive, blue negative) of a single exon with each pathway. Columns are sorted by hierarchical clustering. Rows are ranked by total number of exon correlates passing statistical metrics for each pathway (# Events, bar chart). The gene ontology term with the highest enrichment for the genes containing pathway correlated exons and the corresponding p-value is also depicted. The p-values correspond to the gene ontology enrichment and are not a measure of significance of pathway-exon correlation. (C) Hive plot depiction of exons correlated with selected prostate cancer-related gene signatures and the biological processes associated with genes containing those exons. All pathway-correlated exons are displayed on the left axis. Seven well-known prostate cancer driver pathways are represented as nodes on the middle axis. The area of these nodes reflects the number of exons correlated with each pathway. The right axis depicts four summary gene ontology terms. The width of the edges connecting the nodes on the middle axis to the nodes on the right axis is proportional to the enrichment of each pathway for each biological process. The size of the nodes on the right axis is proportional to the total number of exons associated with each biological process. (D) Area-proportional Venn diagram depicting the intersection of Myc-, E2F-, and MTOR-correlated exons in prostate cancer. Exons must share the same correlation direction (positive or negative) to appear in the intersection.

FIG. 3A-F. Exon incorporation events correlated with Myc activity are strongly enriched in RNA-binding proteins and are conserved in prostate and breast cancers. (A) Heatmap depiction of exon usage of 1,039 Myc-correlated exons across prostate cancer datasets in healthy tissue, primary, metastatic adenocarcinoma, and neuroendocrine prostate cancers. Columns represent samples ordered by disease phenotype and sorted by Myc Targets V2 signature score within each group. The Myc score annotation is colored from white (low) to black (high) based on the rank-transformed signature score of patient samples across the data sets. Rows represent exon inclusion events ordered by hierarchical clustering. (B) Scatterplots depicting examples of cassette exons in SRSF3 and HRAS transcripts whose incorporation is negatively correlated with Myc gene signature score. (C) Sashimi plots depicting average cassette exon incorporation levels of exons in SRSF3 and HRAS in prostate cancer datasets separated by cancer phenotype. Sashimi plots depict density of exon-including and exon-skipping reads as determined by rMATS-turbo analysis. (D) Workflow diagram for performing pathway-guided alternative splicing analysis on normal and cancerous breast and lung tissues. Each sample is scored for the Myc Targets V2 signature and correlated with the exon usage matrix to identify pathway-correlated exon incorporation changes. (E) Venn diagram indicating the intersection between Myc-correlated exon sets in prostate cancers with breast and lung adenocarcinomas. Exons must share the same correlation direction (positive or negative) to appear in the intersection. (F) REVIGO chart depicting the gene ontology of genes containing the 492 Myc-correlated exons from the triple intersection described above.

FIG. 4A-E. Enforced expression of activated AKT1 and doxycycline-regulated c-Myc initiates AR-negative prostate adenocarcinoma in human prostate cells. (A) Workflow diagram for derivation of Myc/myrAKT1 transformed human prostate cells from benign epithelium. (B) Depiction of lentiviral vectors used to enforce doxycycline-regulated expression of Myc and constitutive expression of myrAKT1. Histologic sections of transduced organoids. (C) Photomicrographs and fluorescent overlay of recovered grafts and tumor outgrowth after lentiviral transduction and subcutaneous implantation in NSG mice. “UT”=untreated, “C”=c-Myc transduction (GFP), “A”=myrAKT1 transduction (RFP), “CA”=dual transduction with c-Myc and myrAKT1 (GFP & RFP merge depicted as yellow). (D) H&E stain of histologic sections of recovered grafts and tumor outgrowths. (E) Photomicrographs of cell lines ICA-1, ICA-2, and ICA-3 derived from tumor outgrowths growing as suspended rafts in tissue culture.

FIG. 5A-D. Myc loss in the engineered cell lines produces a senescent-like phenotype and strongly affects the expression of RNA binding proteins. (A) Western blot of lysates from ICA1 cells withdrawn from doxycycline in a time course examining Myc expression and changes in proteins related to cell cycle state. Each of the three cell lines was examined in this manner and the data shown are representative of all three. (B) Volcano plot of gene-level expression changes after Myc withdrawal. Genes downregulated upon Myc loss appear on the left-hand side of the plot. Gene expression changes with the Cuffdiff q-value<0.05 appear red. (C) Selected top gene ontology terms from the gene ontology analysis of Myc-dependent gene expression changes displaying strong enrichment for RNA binding. CC: Cellular Component, MF: Molecular Function, BP: Biological Process. (D) Comparison of Myc Targets V2 signature score levels in engineered cell lines in the presence and absence of doxycycline.

FIG. 6A-F. Exon-level splicing analysis of c-Myc/myrAKT1 transformed human prostate cells identifies Myc-dependent exon incorporation events in splicing regulatory proteins. Summary table of exon incorporation changes occurring after Myc withdrawal. (B) Heatmap depicting changes in exon incorporation of 1,970 Myc-dependent cassette exons in three independent engineered cell lines. (C) Sashimi plots depicting the change in splice junction RNA-Seq reads in SRSF3 and HRAS exons in the engineered cell lines following Myc withdrawal. Sashimi plots depict density of exon-including and exon-skipping reads as determined by rMATS-turbo analysis. (D) REVIGO scatter plot depicting gene ontology terms enriched among genes containing exons whose incorporation is responsive to Myc withdrawal. Semantic distance is a measurement of relatedness between gene ontology terms calculated by REVIGO. Representative gene ontology terms have been selected to describe each cluster. Dashed line indicates adjusted p=0.05. (E) Venn diagram depicting the overlap between Myc-dependent exons (purple) and Myc-correlated exons identified in patient tissues (green). Exons must change incorporation level with Myc in the same direction as the correlation (positive or negative) in order to appear in the intersection of the two sets. (F) Heatmap depicting the annotated outcome of exon changes in validated Myc-dependent exons. The annotation identifies exons likely to produce premature termination codons (orange) or frameshifts (green).

FIG. 7A-B. Comparison of count-based and ratio-based isoform-level analyses of prostate RNA-Seq datasets. (A) Unsupervised analysis of count-based isoform expression from a combined prostate cancer dataset (left panel). The same methodology applied to the ratio-based alternative splicing approach from FIG. 1B in the main text is shown for comparison (right panel). (B) Silhouette width-based comparison of clustering fitness for each of the principle component analyses shown above. Mets, metastatic.

FIG. 8. Gene signature analysis identifies a common set of exons correlated with Myc. E2F, or mTOR pathways. Violin plot depiction of gene signature scores of AR, Myc Targets V2, and mTOR sets across prostate cancer datasets. Dashed lines indicate averages across datasets profiling a disease phenotype (normal prostate, benign prostate, primary prostate cancer, mCRPC, and NEPC).

FIG. 9A-E. Validation of Myc signature score and exon conservation across phylogeny and tumor type. (A) Box-and-whisker plot depiction of Myc signature scores in benign prostate tissues and primary prostate cancers stratified by Myc status. Samples with genomic amplifications of the Myc locus or single-gene overexpression are compared to samples without these alterations and adjacent benign tissues. (B) Kaplan-Meier disease-free survival plots of prostate cancers stratified by Myc signature score (first panel), Myc amplification status (second panel), or single-gene Myc expression (third panel). (C) Unsupervised two-way hierarchical clustering heatmap depiction of exon usage of 1.039 Myc-correlated exons across prostate cancer datasets in healthy tissue, and in primary, metastatic, and neuroendocrine prostate cancers. Columns depict patient samples. The Myc score annotation is colored from white (low) to black (high) based on the rank-transformed signature score of patient samples across the data sets. Rows represent exon inclusion events. Both are ordered by hierarchical clustering. (D) UCSC Genome Browser tracks depicting ultraconservation of Myc-regulated exons in SRSF3 (top panel) and HRAS (bottom panel) from humans to lamprey. (E) Heatmap of Myc-correlated exons in the prostate meta-dataset alongside tissues from normal breast and lung as well as breast and lung adenocarcinomas. Dashed line indicates separation between two cancer types. The Myc score annotation is colored from white (low) to black (high) based on the rank-transformed signature of patient samples across the datasets.

FIG. 10A-D. Establishment of engineered human tumor model with regulated Myc expression. (A) Representative scatterplot from florescence-activated cell sorting isolation of CD49f-high/Trop2-high basal cells from total dissociated benign human prostate. (B) Florescent photomicrograph of doubly transduced prostate organoids as well as single and untransduced controls. “UT”=untreated, “C”=c-Myc transduction (GFP), “A”=myrAKT1 (RFP), “CA”=c-Myc and myrAKT1 (merge=yellow). (C) Photomicrograph of fixed organoids to show histology. Hematoxylin and eosin staining. (D) Immunohistochemical staining of transformed xenograft outgrowth compared to normal prostate tissue controls.

FIG. 11A-C. Characterization of the response to Myc withdrawal in vitro. (A) Immunoblot of Myc expression levels in engineered cell line ICA1 in response to doxycycline titration. Data are representative of all three cell lines. (B) Growth response of ICA1 cell line in response to doxycycline titration as measured in a luciferase-based assay. (C) Stacked column chart depicting the change in cell cycle distribution over time after doxycycline withdrawal as measured by flow cytometry.

FIG. 12. Individual exon incorporation changes in response to Myc withdrawal. Semi-quantitative immunoblot of SRSF3 protein levels in response to Myc withdrawal for 24 h. Quantitation is the average reduction in SRSF3 levels measured in each cell line over the three independent replicates shown.

### DETAILED DESCRIPTION OF THE INVENTION

The inventors sought to define the landscape of alternative pre-mRNA splicing in prostate cancers and the relationship of exon choice to known cancer driver alterations. To do so, the inventors compiled a meta-dataset comprised of 876 RNA-Seq samples from five publicly available sources representing a range of prostate phenotypes from normal tissue to drug resistant metastases. The inventors subjected these samples to exon-level analysis with rMATS-turbo, purpose-built software designed for large-scale analyses of splicing, and identified 13,149 high-confidence cassette exon events with variable incorporation across samples. The inventors discovered that Myc signaling was correlated with incorporation of a set of 1,039 cassette exons enriched in genes encoding RNA binding proteins. Using a human prostate epithelial transformation assay, the inventors confirmed the Myc regulation of 147 of these exons, many of which introduced frameshifts or encoded premature stop codons. Our results connect changes in alternative pre-mRNA splicing to oncogenic alterations common in prostate and many other cancers. The inventors also establish a role for Myc in regulating RNA splicing by controlling the incorporation of nonsense mediated decay-determinant exons in genes encoding RNA binding proteins.

### I. Applications of Antigenic Peptides

Various embodiments are directed to development of and use of antigenic peptides that have been identified from neoplastic tissue. In many embodiments, antigenic peptides are produced by chemical synthesis or by molecular expression in a host cell. Peptides can be purified and utilized in a variety of applications including (but not limited to) assays to determine peptide immunogenicity, assays to determine recognition by T cells, peptide vaccines for treatment of cancer, development of modified TCRs of T cells, development of antibodies, and development of CAR-T cells to recognize extracellular peptides.

Peptides can be synthesized chemically by a number of methods. One common method is to use solid-phase peptide synthesis (SPPS). Generally, SPPS is performed by repeating cycles of alternate N-terminal deprotection and coupling reactions, building peptides from the c-terminus to the n-terminus. The c-terminus of the first amino acid is coupled the resin, wherein then the amine is deprecated and then coupled with the free acid of the second amino acid. This cycle repeats until the peptide is synthesized.

Peptides can also be synthesized utilizing molecular tools and a host cell. Nucleic acid sequences corresponding with antigenic peptides can be synthesized. In some embodiments, synthetic nucleic acids synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

Embodiments are also directed to expression vectors and expression systems that produce antigenic peptides or proteins. These expression systems can incorporate an expression vector to express transcripts and proteins in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or protein molecules can be purified from these systems using standard biotechnology production procedures.

Assays to determine immunogenicity and/or TCR binding can be performed. One such as is the dextramer flow cytometery assay. Generally, custom-made HLA-matched MHC Class I dextramer:peptide (pMHC) complexes are developed or purchased (Immudex, Copenhagen, Denmark). T cells from peripheral blood mononuclear cells (PBMCs) or tumor-infiltrating lymphocytes (TILs) are incubated the pMHC complexes and stained, which are then run through a flow cytometer to determine if the peptide is capable of binding a TCR of a T cell.

Peptide embodiments include those in the following table:

| | SEQ |
|---|---|
| | ID |
| Sequence | NO: |
| SLAIGGVTEA | 1 |
| LLLGIAKLLKV | 2 |
| HLMEENMIVYV | 3 |
| LLAEQPDQV | 4 |
| LVLISIVICV | 5 |
| LLAEQPDQV | 6 |
| ALVANLPLL | 7 |
| FTVTVTEPPV | 8 |
| ALAGKPLSL | 9 |
| FILTYWTPS | 10 |
| STMYYLWML | 11 |
| VSTMYYLWML | 12 |
| FLSELEPPAP | 13 |
| TLVLALVANL | 14 |
| STMYYLWML | 15 |
| FLSELEPPA | 16 |
| FLAIVFFASIV | 17 |
| STMYYLWML | 18 |
| KVWTETLIEA | 19 |

A peptide as described herein (e.g., a peptide of SEQ ID NOS: 1-19) may be used for immunotherapy of a cancer. For example, a peptide of the disclosure may be contacted with or used to stimulate a population of T cells to induce proliferation of the T cells that recognize or bind said peptide. In other embodiments, a peptide of the disclosure may be administered to a subject, such as a human patient, to enhance the immune response of the subject against a cancer.

A peptide of the disclosure may be included in an active immunotherapy (e.g., a cancer vaccine) or a passive immunotherapy (e.g., an adoptive immunotherapy). Active immunotherapies include immunizing a subject with a purified peptide antigen or an immunodominant peptide (native or modified); alternatively, antigen presenting cells pulsed with a peptide of the disclosure (or transfected with genes encoding an antigen comprising the peptide) may be administered to a subject. The peptide may be modified or contain one or more mutations such as, e.g., a substitution mutation. Passive immunotherapies include adoptive immunotherapies. Adoptive immunotherapies generally involve administering cells to a subject, wherein the cells (e.g., cytotoxic T cells) have been sensitized in vitro to a peptide of the disclosure (sec, e.g., U.S. Pat. No. 7,910,109).

In some embodiments, flow cytometry may be used in the adoptive immunotherapy for rapid isolation of human tumor antigen-specific T-cell clones by using, e.g., T-cell receptor (TCR) Vβ antibodies in combination with carboxyfluorescein succinimidyl ester (CFSE)-based proliferation assay. Sec, e.g., Lee et al., J. Immunol. Methods, 331:13-26, 2008, which is incorporated by reference for all purposes. In some embodiments, tetramer-guided cell sorting may be used such as, e.g., the methods described in Pollack, et al., J Immunother Cancer. 2014; 2: 36, which is herein incorporated by reference for all purposes. Various culture protocols are also known for adoptive immunotherapy and may be used in embodiments of the disclosure. In some embodiments, cells may be cultured in conditions which do not require the use of antigen presenting cells (e.g., Hida et al., Cancer Immunol. Immunotherapy, 51:219-228, 2002, which is incorporated by reference). In other embodiments, T cells may be expanded under culture conditions that utilize antigen presenting cells, such as dendritic cells (Nestle et al., 1998, incorporated by reference), and in some embodiments artificial antigen presenting cells may be used for this purpose (Maus et al., 2002 incorporated by reference). Additional methods for adoptive immunotherapy are disclosed in Dudley et al. (2003), which is incorporated by reference, that may be used with embodiments of the current disclosure. Various methods are known and may be used for cloning and expanding human antigen-specific T cells (see, e.g., Riddell et al., 1990, which is herein incorporated by reference).

In certain embodiments, the following protocol may be used to generate T cells that selectively recognize peptides of the disclosure. Peptide-specific T-cell lines may be generated from normal donors or HLA-restricted normal donors and patients using methods previously reported (Hida et al., 2002). Briefly, PBMCs (1×105 cells/well) can be stimulated with about 10 μg/ml of each peptide in quadruplicate in a 96-well, U-bottom-microculture plate (Corning Incorporated, Lowell, MA) in about 200 μl of culture medium. The culture medium may consist of 50% AIM-V medium (Invitrogen), 50% RPMI1640 medium (Invitrogen), 10% human AB serum (Valley Biomedical, Winchester, VA), and 100 IU/ml of interleukin-2 (IL-2). Cells may be restimulated with the corresponding peptide about every 3 days. After 5 stimulations, T cells from each well may be washed and incubated with T2 cells in the presence or absence of the corresponding peptide. After about 18 hours, the production of interferon (IFN)-γ may be determined in the supernatants by ELISA. T cells that secret large amounts of IFN-γ may be further expanded by a rapid expansion protocol (Riddell et al., 1990; Yee et al., 2002b).

In some embodiments, an immunotherapy may utilize a peptide of the disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail below. In some embodiments, an immunotherapy may utilize a nucleic acid encoding a peptide of the disclosure, wherein the nucleic acid is delivered, e.g., in a viral vector or non-viral vector.

In some embodiments, a peptide of the disclosure may be used in an immunotherapy to treat cancer in a mammalian subject, such as a human patient.

### III. Engineered T Cell Receptors

T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Ca domain, where the fold is unlike that of any other immunoglobulin-like domain. The half of the domain that is juxtaposed with the CB domain forms a β sheet similar to that found in other immunoglobulin-like domains, but the other half of the domain is formed of loosely packed strands and a short segment of a helix. The intramolecular disulfide bond, which in immunoglobulin-like domains normally joins two β strands, in a Cα domain joins a β strand to this segment of a helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the B strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

Embodiments of the disclosure relate to engineered T cell receptors. The term “engineered” refers to T cell receptors that have TCR variable regions grafted onto TCR constant regions to make a chimeric polypeptide that binds to peptides and antigens of the disclosure. In certain embodiments, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some embodiments, the TCR comprises non-TCR sequences. Accordingly, certain embodiments relate to TCRs with sequences that are not from a TCR gene. In some embodiments, the TCR is chimeric, in that it contains sequences normally found in a TCR gene, but contains sequences from at least two TCR genes that are not necessarily found together in nature.

### IV. Antibodies

Aspects of the disclosure relate to antibodies that target the peptides of the disclosure, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018)

Humana Press, New York, N.Y, incorporated herein by reference in its entirety. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986), each of which are specifically incorporated herein by reference in their entirety. Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013), incorporated herein by reference in its entirety.

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2. Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bispecific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)), each of which are specifically incorporated herein by reference in their entirety.

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804, which is incorporated herein by reference in its entirety) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1. CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1. H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970, which is incorporated herein by reference in its entirety); Chothia (as described in C. Chothia et al., “Conformations of immunoglobulin hypervariable regions,” Nature, vol. 342, no. 6252, pp. 877-883, December 1989, which is incorporated herein by reference in its entirety); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003, which is incorporated herein by reference in its entirety). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope. 2) Hydrogen-deuterium exchange and mass spectroscopy 3) Polypeptide fragmentation and peptide mapping approaches in which one generates multiple overlapping peptide fragments from the full length of the polypeptide and evaluates the binding affinity of these peptides for the epitope. 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992), which is incorporated herein by reference in its entirety, describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017), each of which are specifically incorporated herein by reference in their entirety.

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988), each of which are specifically incorporated herein by reference in their entirety.

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to a peptide of the disclosure. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003, each incorporated by reference in its entirety) constituted with a single VH or VL domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day. E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)₂ means bivalent or bispecific sFv polypeptide chains that include oligomerization domains at their C-termini, separated from the sFv by a hinge region. The oligomerization domain comprises self-associating α-helices, e.g., leucine zippers, which can be further stabilized by additional disulfide bonds. (scFv)₂ fragments are also known as “miniantibodies” or “minibodies.”

single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with Antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000), which is incorporated herein by reference in its entirety.

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 1. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 2. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012), which is incorporated herein by reference in its entirety. Several algorithms for predicting protein structures and the gene sequences that encode these have been developed, and many of these algorithms can be found at the National Center for Biotechnology Information the (on World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### V. Proteinaceous Compositions

As used herein, a “protein” “peptide” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a peptide, protein, or polypeptide (wild-type or modified), such as a peptide or protein of the disclosure comprising a peptide of one of SEQ ID NOS: 1-19 may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions and/or be at least, at most, or be 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous in sequence to at least, or at most 3, 4, 5, 6, 7, 8, or 9 contiguous amino acids of a peptide of one of SEQ ID NOS: 1-19 or nucleic acids encoding a peptide of one of SEQ ID NO:1-19. In certain embodiments, the peptide or polypeptide is not naturally occurring and/or is in a combination of peptides or polypeptides.

In some embodiments, the protein or polypeptide may comprise amino acids 1 to 2, 3, 4, 5, 6, 7, 8, 9, 10, 11, 12, 13, 14, 15, 16, 17, 18, 19, or 20 (or any derivable range therein) of a peptide of one of SEQ ID NOS: 1-19. In some embodiments, the peptides of the disclosure comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) amino acids flanking the carboxy and/or flanking the amino end of a peptide comprising or consisting of 3, 4, 5, 6, 7, 8, 9, 10, 11, 12, 13, 14, 15, 16, 17, 18, 19, or 20 contiguous amino acids of a peptide of one of SEQ ID NOS: 1-19.

In some embodiments, the protein may comprise, polypeptide may comprise, or nucleic acid may encode for a protein or polypeptide that comprises 1, 2, 3, 44, 5, 6, 7, 8, 9, 10, 11, 12, 13, 14, 15, 16, 17, 18, 19, or 20 (or any derivable range therein) contiguous amino acids of a peptide of one of SEQ ID NOS: 1-19.

In some embodiments, the protein may comprise, polypeptide may comprise, or nucleic acid may encode for a protein or polypeptide that comprises at least, at most, or exactly 1, 2, 3, 4, 5, 6, 7, 8, 9, 10, 11, 12, 13, 14, 15, 16, 17, 18, 19, or 20 (or any derivable range therein) contiguous amino acids of a peptide of one of SEQ ID NOS: 1-19 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to a peptide of one of SEQ ID NOS:1-19.

In some aspects there is a polypeptide (or a nucleic acid molecule encoding such a polypeptide) starting at position 1, 2, 3, 4, 5, 6, 7, 8, 9, 10, 11, 12, 13, 14, 15, 16, 17, 18, 19, or 20 of a peptide of one of SEQ ID NOS: 1-19 and comprising at least, at most, or exactly 2, 3, 4, 5, 6, 7, 8, 9, 10, 11, 12, 13, 14, 15, 16, 17, 18, or 19 (or any derivable range therein) contiguous amino acids of a peptide of one of SEQ ID NOS: 1-19.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); scrine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within +2 is included. In some aspects of the invention, those that are within +1 are included, and in other aspects of the invention, those within +0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0+1); glutamate (+3.0+1); serine (+0.3); asparaginc (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 are included, in other embodiments, those which are within +1 are included, and in still other embodiments, those within +0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of a polypeptide with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VI. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described hercin, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), incorporated by reference, 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68º C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VII. Antibody Production

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745, each of which are specifically incorporated herein by reference in their entirety. (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al. (1983); Merrifield. (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988, which is incorporated herein by reference in its entirety).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), which is incorporated herein by reference in its entirety, the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001, which is incorporated herein by reference in its entirety).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m²) (Johnson/Mead, NJ), cytokines such as β-interferon, IL-2, or IL-12, or genes encoding proteins involved in immune helper functions, such as B-7. A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991), which is incorporated herein by reference in its entirety.

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of humanized antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993), each of which are specifically incorporated herein by reference in their entirety. In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (u and y) and k light chain immunoglobulin sequences, together with targeted mutations that inactivate the endogenous u and K chain loci (Lonberg et al., Nature 368:856-859 (1994)). Accordingly, the mice exhibit reduced expression of mouse IgM or k chains and in response to immunization, the introduced human heavy and light chain transgenes undergo class switching and somatic mutation to generate high affinity human IgG K monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)), each of which are specifically incorporated herein by reference in their entirety. The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)), each of which are specifically incorporated herein by reference in their entirety. One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980), each of which are specifically incorporated herein by reference in their entirety.

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001), each of which are specifically incorporated herein by reference in their entirety). By combining different VL- and VH-comprising polypeptides, one can form multimeric scFvs that bind to different epitopes (Kriangkum et al., Biomol. Eng. 18:31-40 (2001), each of which are specifically incorporated herein by reference in their entirety). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988), each of which are specifically incorporated herein by reference in their entirety. Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003), each of which are specifically incorporated herein by reference in their entirety, also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH═CH— (cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. The coding sequences for the heavy and light chain portions of the Fab molecules selected from the phage display library can be isolated or synthesized and cloned into any suitable vector or replicon for expression. Any suitable expression system can be used.

### VIII. Polypeptide Expression

In some aspects, there are nucleic acid molecule encoding polypeptides or peptides of the disclosure (e.g antibodies, TCR genes, and immunogenic peptides). These may be generated by methods known in the art, e.g., isolated from B cells of mice that have been immunized and isolated, phage display, expressed in any suitable recombinant expression system and allowed to assemble to form antibody molecules or by recombinant methods.

### A. Expression

The nucleic acid molecules may be used to express large quantities of polypeptides. If the nucleic acid molecules are derived from a non-human, non-transgenic animal, the nucleic acid molecules may be used for humanization of the antibody or TCR genes.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 1. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 2. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624,5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990, which is incorporated herein by reference in its entirety); by using DEAE dextran followed by polyethylene glycol (Gopal, 1985, which is incorporated herein by reference in its entirety); by direct sonic loading (Fechheimer et al., 1987, which is incorporated herein by reference in its entirety); by liposome mediated transfection (Nicolau and Sene, 1982; Fraley et al., 1979; Nicolau et al., 1987; Wong et al., 1980; Kaneda et al., 1989; Kato et al., 1991, each of which are specifically incorporated herein by reference in their entirety); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985, which is incorporated herein by reference in its entirety). Other methods include viral transduction, such as gene transfer by lentiviral or retroviral transduction.

### 3. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. Sec, e.g., Kabat et al., 1991, supra. Nucleic acid molecules encoding the full-length heavy and/or light chains may then be expressed in a cell into which they have been introduced and the antibody isolated.

### IX. Additional Therapies

### A. Immunotherapy

In some embodiments, the methods comprise administration of an additional therapy. In some embodiments, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Checkpoint Inhibitors and Combination Treatment

Embodiments of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168, incorporated herein by reference in its entirety. Pembrolizumab, also known as MK-3475, Merck 3475, lambrolizumab, KEYTRUDA®, and SCH-900475, is an anti-PD-1 antibody described in WO2009/114335, incorporated herein by reference in its entirety. Pidilizumab, also known as CT-011, hBAT, or hBAT-1, is an anti-PD-1 antibody described in WO2009/101611, incorporated herein by reference in its entirety. AMP-224, also known as B7-DCIg, is a PDL2-Fc fusion soluble receptor described in WO2010/027827 and WO2011/066342, each of which are specifically incorporated herein by reference in their entirety. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

a. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101. and Yervoy®) or antigen binding fragments and variants thereof (sec, e.g., WO0 1/14424, which is incorporated herein by reference in its entirety).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### 2. Activation of Co-Stimulatory Molecules

In some embodiments, the immunotherapy comprises an activator of a co-stimulatory molecule. In some embodiments, the activator comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include activating antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.[60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### B. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adrenocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### C. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

### D. Surgery

In some embodiments, the additional therapy comprises surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months (or any range derivable therein). These treatments may be of varying dosages as well.

### X. Formulations and Culture of the Cells

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V. X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolglycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In some embodiments, the method further comprises priming the T cells. In some embodiments, the T cells are primed with antigen presenting cells. In some embodiments, the antigen presenting cells present tumor antigens or peptides, such as those disclosed herein.

In particular embodiments, the cells of the disclosure comprise an exogenous TCR, which may be of a defined antigen specificity. In some embodiments, the TCR can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the cells of the disclosure further comprise one or more chimeric antigen receptors (CARs). Examples of tumor cell antigens to which a CAR may be directed include at least 5T4, 8H9, αᵥβ₆ integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-α, FAP, FBP. fetal AchR. FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MCIR, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### XI. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 M; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 M to 50 M; or about 25 μM to 150 μM; or about 25 μM to 100 μM; or about 25 μM to 50 M; or about 50 μM to 150 μM; or about 50 M to 100 M (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

“Tumor.” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), lung cancer, non-small cell lung cancer, small cell lung cancer, lymphomas, AIDS-related lymphoma, brain cancer, glioblastoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic mycloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### XII. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from ovarian or endometrial tissue by any of the biopsy methods previously mentioned. In other embodiments the sample may be obtained from any of the tissues provided herein that include but are not limited to non-cancerous or cancerous tissue and non-cancerous or cancerous tissue from the ovarian epithelium, fallopian epithelium, ovaries, cervix, fallopian tube, or uterus. Alternatively, the sample may be obtained from any other source including but not limited to blood, serum, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XIII. Detection and Vaccination Kits

A peptide or antibody of the disclosure may be included in a kit. The peptide or antibody in the kit may be detectably labeled or immobilized on a surface of a support substrate also comprised in the kit. The peptide(s) or antibody may, for example, be provided in the kit in a suitable form, such as sterile, lyophilized, or both.

The support substrate comprised in a kit of the invention may be selected based on the method to be performed. By way of nonlimiting example, a support substrate may be a multi-well plate or microplate, a membrane, a filter, a paper, an emulsion, a bead, a microbead, a microsphere, a nanobead, a nanosphere, a nanoparticle, an ethosome, a liposome, a niosome, a transferosome, a dipstick, a card, a celluloid strip, a glass slide, a microslide, a biosensor, a lateral flow apparatus, a microchip, a comb, a silica particle, a magnetic particle, or a self-assembling monolayer.

As appropriate to the method being performed, a kit may further comprise one or more apparatuses for delivery of a composition to a subject or for otherwise handling a composition of the invention. By way of nonlimiting example, a kit may include an apparatus that is a syringe, an eye dropper, a ballistic particle applicator (e.g., applicators disclosed in U.S. Pat. Nos. 5,797,898, 5,770,219 and 5,783,208, and U.S. Patent Application 2005/0065463), a scoopula, a microslide cover, a test strip holder or cover, and such like.

A detection reagent for labeling a component of the kit may optionally be comprised in a kit for performing a method of the present invention. In particular embodiments, the labeling or detection reagent is selected from a group comprising reagents used commonly in the art and including, without limitation, radioactive elements, enzymes, molecules which absorb light in the UV range, and fluorophores such as fluorescein, rhodamine, auramine, Texas Red, AMCA blue and Lucifer Yellow. In other embodiments, a kit is provided comprising one or more container means and a BST protein agent already labeled with a detection reagent selected from a group comprising a radioactive element, an enzyme, a molecule which absorbs light in the UV range, and a fluorophore.

When reagents and/or components comprising a kit are provided in a lyophilized form (lyophilisate) or as a dry powder, the lyophilisate or powder can be reconstituted by the addition of a suitable solvent. In particular embodiments, the solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

### XIV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Pathway-Guided Analysis Identifies Myc-Dependent Alternative Pre-mRNA Splicing in Aggressive Prostate Cancers

Alternative pre-mRNA splicing is a regulated process that greatly diversifies gene products by changing the exons incorporated into mRNA. This process is dysregulated in cancers. Here, the inventors studied exon usage in aggressive prostate cancers and linked exon incorporation decisions to cancer driver genes. Through computational and experimental studies, the inventors found that a strong cancer driver gene, Myc, was linked to exon changes in genes that themselves regulate alternative splicing. These exons often encoded premature stop codons that would decrease gene expression, suggestive of a Myc-driven auto-regulatory loop to help control levels of splicing regulatory proteins.

Alternative pre-mRNA splicing is a regulated process that governs exon choice and greatly diversifies the proteome. It is an essential process that contributes to development, tissue specification, and homeostasis and is often dysregulated in disease states (1). In cancer, this includes growth signaling, epithelial-to-mesenchymal transition, resistance to apoptosis, and treatment resistance (2). In prostate cancer, our area of interest, the most notable splicing change is the emergence of the ligand-independent androgen receptor ARV7 isoform in response to hormone deprivation (3). Other examples include pro-angiogenic splice variants of VEGFA (4), tumorigenic variants of the transcription factors ERG and KLF6 (5, 6), and anti-apoptotic splicing of BCL2L2 (7, 8). However, the intersection of upstream oncogenic signaling, pre-mRNA splicing, and the biological processes affected by those splicing events has not been defined at a global level.

Prostate cancers progress from hormone-responsive, localized disease to hormone-independent, metastatic disease accompanied by changes in gene expression and mutations that confer cell autonomous growth and therapeutic resistance (9). The study of disease progression from primary prostate adenocarcinoma (PrAd) to metastatic, castration-resistant prostate cancer (mCRPC) and treatment-related neuroendocrine prostate cancer (NEPC) has been aided by large-scale genomic and transcriptomic studies of patient samples representing each form of the disease (10-13). Examples of driver alterations found in precursor lesions and primary tumors include TMPRSS2-ERG translocations and PTEN loss (14). Metastatic tumors are characterized by Myc and AR amplification (15, 16). NEPC includes near-universal loss of TP53 signaling by inactivation mutation as well as chromosomal loss of RB1 (17). Sequencing efforts and subsequent functional experiments have identified prostate cancer driver alterations and defined the impact of gene expression networks on prostate cancer phenotypes. These studies have led to the successful development of new therapeutics targeting AR signaling and DNA repair in advanced disease (18, 19).

Prostate cancer progression is also associated with shifts in alternative pre-mRNA splicing patterns, but this process is not well-understood (20). Investigations of global changes in exon usage in prostate cancer have focused on stage- or race-specific comparisons (21-25). Comparisons of tumor-adjacent benign material and PrAd identified intron retention and exon skipping events in the biomarkers KLK3 and AMACR, respectively (22). Others studying NEPC and PrAd have shown that a network of splicing events controlled by the Serine-Arginine RNA-binding protein SRRM4 contributes to the neuroendocrine phenotype (26-28). Comparisons of European American and African American (AA) PrAd samples identified an AA-specific splice variant of PIK3CD that enhanced AKT/mTOR signaling (23). How these splicing alterations connect to the driver alterations described above remains to be explored.

The accumulation of RNA-Seq data in large databases presents a unique opportunity to conduct an analysis of alternative splicing across the full range of prostate cancer disease states. For our study, the inventors prepared a unified dataset of large, publically-available RNA-Seq datasets representing normal tissue, tumor-adjacent benign tissue, primary adenocarcinoma, metastatic castration-resistant adenocarcinoma, and treatment-related metastatic NEPC. However, handling datasets of this size requires splicing analysis software with greater efficiency than what is currently available. To analyze these hundreds of datasets, the inventors created a new version of the rMATS software (dubbed rMATS-turbo) that can handle this volume of RNA-Seq data (29, 30).

The inventors identify a high-confidence set of exons whose incorporation varies across prostate cancer disease states. By combining expression-level and exon-level analyses, the inventors developed a pathway-guided strategy to examine the impact of oncogenic pathways on incorporation of these exons. This correlational analysis implicates Myc, mTOR, and E2F signaling in the control of exon choice in spliceosomal proteins. To further investigate the contributions of Myc signaling to exon choice, the inventors developed unique engineered human prostate cell lines with regulated Myc expression. Functional experiments in these cell lines identify Myc-dependent exons and experimentally confirm that cassette exon choice in many splicing regulatory proteins is responsive to Myc expression level. These exons often encode frameshifts or premature termination codons that would result in nonsense mediated decay. The inventors show that an ultra-conserved, nonsense-mediated decay determinant exon in the RNA-binding protein SRSF3 is particularly responsive to Myc signaling. These results implicate Myc signaling as a regulator of alternative splicing coupled nonsense-mediated decay (AS-NMD) as part of a program of growth control.

### A. Results

### 1. Exon-Level Analysis Defines the Landscape of Alternative Pre-mRNA Splicing Across the Prostate Cancer Disease Spectrum

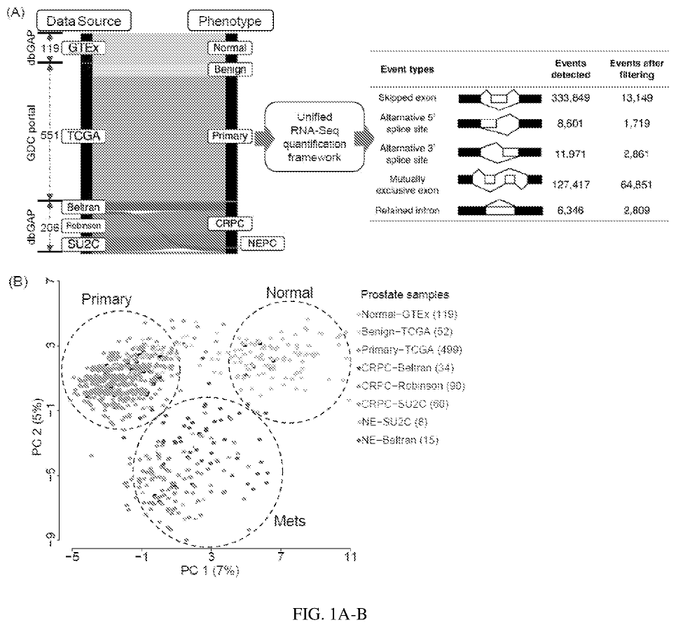

The inventors combined RNA-Seq data from disparate published datasets representing 876 samples of normal tissue, benign tumor-adjacent material, primary adenocarcinoma, metastatic castration-resistant adenocarcinoma (mCRPC), and treatment-related neuroendocrine prostate cancer (NEPC) (FIG. 1A) (10-13, 31, 32). Meta-analyses of RNA-Seq data with gene- or isoform-level counts are subject to confounding batch effects and rely on existing isoform annotation (33). Exon-level analysis, however, uses a ratio-based methodology to estimate exon incorporation, which may be more robust against batch effects and confounding factors in large-scale RNA-Seq datasets (34-37). In addition, exon-level analysis can detect novel exon-exon junctions and is thus independent of previous annotation.

To facilitate alternative splicing analysis in this and other large RNA-Seq datasets, the inventors developed rMATS-turbo (a.k.a. rMATS 4.0.2), a new computational pipeline that permits the efficient capture, storage, and analysis of splicing information from very large-scale raw RNA-Seq data. This improved pipeline refactors the original ratio-based rMATS software that the inventors developed for splicing analysis in RNA-Seq data to optimize it for very large-scale RNA-Seq datasets and is now available for public use (29, 30). It offers significant improvements in speed and data storage efficiency.

The inventors applied rMATS-turbo to the combined RNA-Seq dataset and identified over 330,000 different cassette exons across all prostate samples. Previous estimates of the diversity of splicing events in human cells vary, but are generally of the same order of magnitude (38). The inventors also identified tens of thousands of additional alternative splicing events (FIG. 1A), including alternative 5′ and 3′ splice sites, mutually exclusive exons, and retained introns. For this study, the inventors focused on cassette exons, as these are the most well-defined type of alternative splicing event. The inventors should note that although the rMATS-turbo software detected numerous mutually exclusive exons, most of these events were in fact part of more complex alternative splicing events, thus the inventors did not include these mutually exclusive exons in downstream analyses.

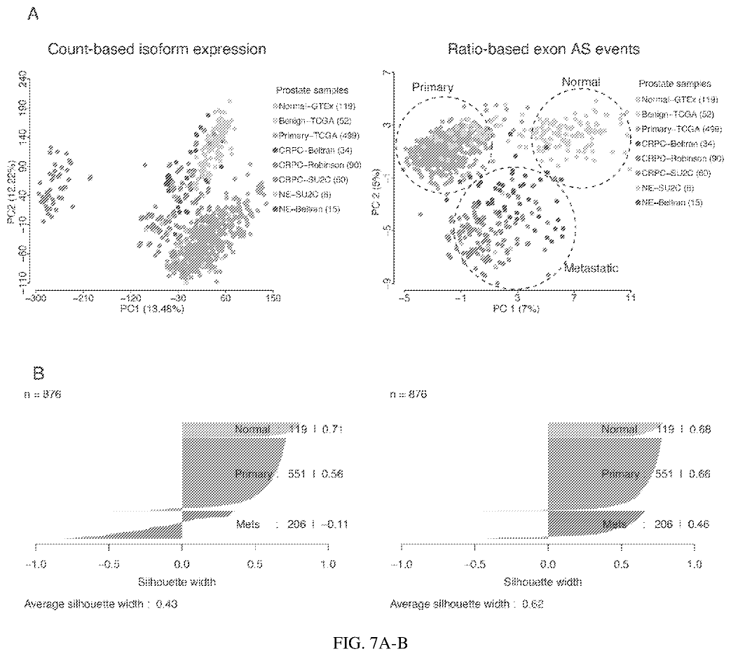

Filtering of these exons for coverage (≥10 splice junction reads per event), cross-sample variance (range of PSI>5%, mean skipping or inclusion>5%) and commonality (events detected in ≥1% of all samples) produced a set of 13,149 high-confidence exons with variable incorporation across samples (see Methods section). Principal component analysis (PCA) of this exon usage matrix grouped samples of the same disease phenotype regardless of dataset (FIG. 1B). By comparison, a similar unsupervised analysis of isoform-level count-based metric from the same meta-dataset grouped samples more by dataset of origin than disease phenotype (FIGS. 7A-B). This result is consistent with prior observations that the exon-level splicing analysis is more robust against batch effects and other confounding factors in large-scale RNA-Seq datasets (35-37).

### 2. Combining Gene Pathway Analysis and Exon Usage Identifies Exon Correlates of Oncogenic Signaling

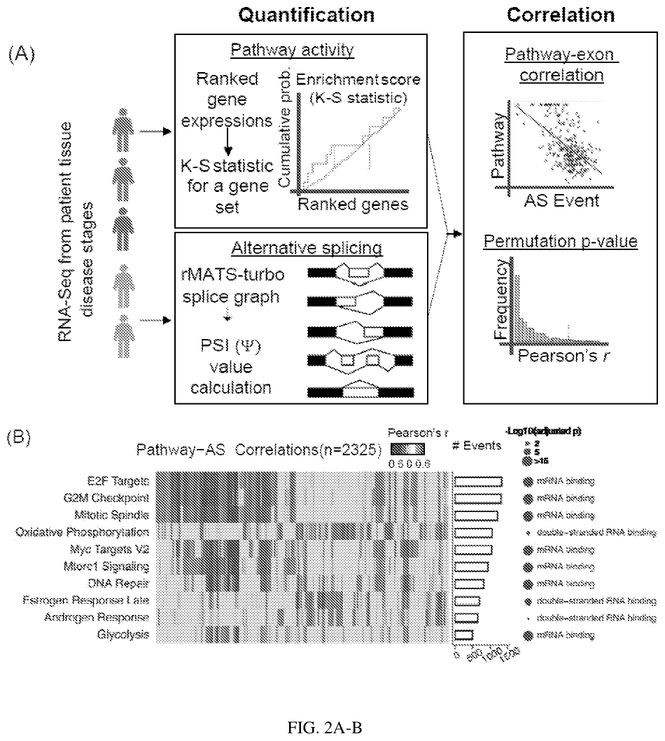

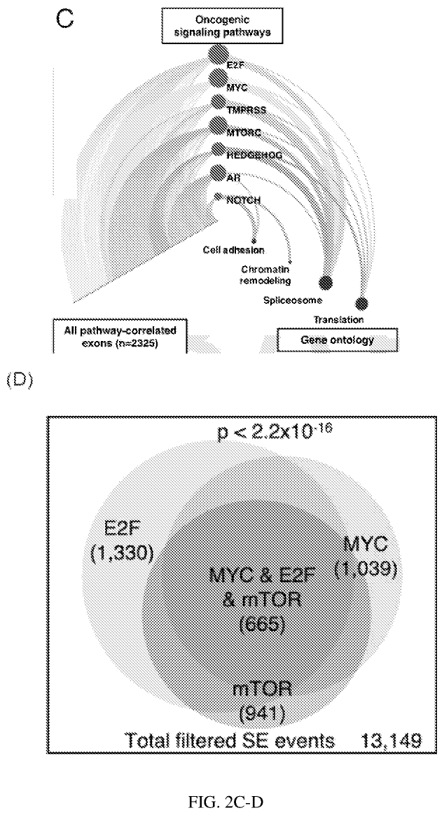

Genomic studies of prostate cancer have identified driver alterations associated with disease progression (39). The inventors sought to define how the variable cassette exons the inventors identified and the biological processes they participate in might relate to these oncogenic signals. Instead of selecting single oncogenes for study, the inventors developed PEGASAS (Pathway Enrichment-Guided Activity Study of Alternative Splicing), a pathway-guided analytic strategy that uses gene signatures to estimate the activities of signaling pathways and to discover potential downstream exon changes (FIG. 2A). Gene signature-based analyses use an ensemble of features (a set of genes collectively) to estimate pathway activity and outperform single gene measurements (40). To mitigate potential batch effects in the expression data, the inventors utilized a rank-based metric to calculate the signature score, providing a more robust measure of pathway activity as it is in essence normalized on a per sample basis (41).

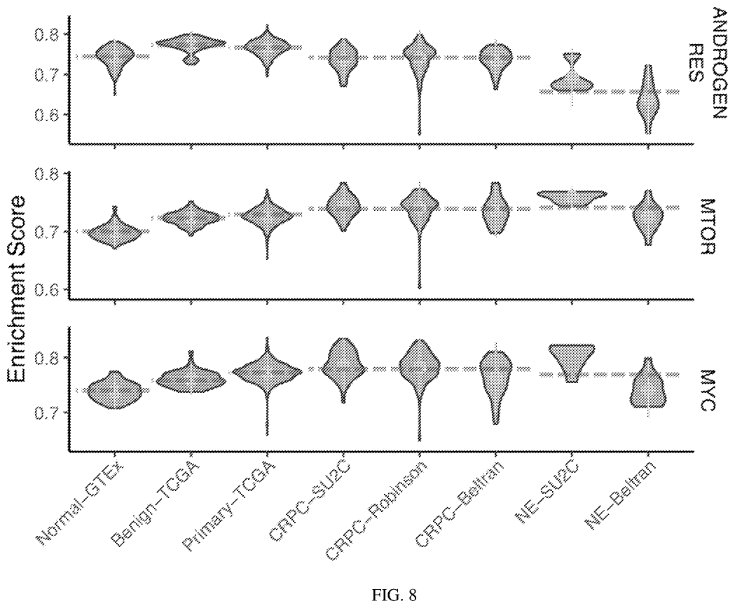

The inventors employed the hallmark gene signature sets maintained by the Molecular Signatures Database (MSigDB) (42). These fifty sets represent a diverse and well-validated array of cellular functions and signaling pathways. To assess the performance of these signatures in the combined dataset, the inventors examined signature scores for the AR, Myc Targets V2, and MTOR gene sets across five different prostate phenotypes. Consistent with previously reported observations of pathway activation in prostate cancer progression, the androgen response gene signature scores the inventors measured were lowest in NEPC samples (FIG. 8A). Similarly, MTOR and Myc signature scores were higher in mCRPC samples than in normal tissues.

The inventors then scored each sample in the meta-dataset for all fifty pathways and correlated this score with the data matrix of over 13,000 variable cassette exons (Dataset S1). After filtering for correlation strength and false discovery rate, each pathway returned between 11 and 1,330 exon correlates (Dataset S1). The ten gene sets that returned the greatest number of exon correlates with a Pearson's correlation coefficient greater than 0.3 or less than −0.3 are shown (FIG. 2B). Nine out of ten of these gene sets had exon correlates found in genes with strong functional enrichment by gene ontology (adjusted p-value<0.05).

3. Cassette Exons Correlating with Myc, E2F, and MTOR Signaling are Enriched in Splicing-Related Genes

The inventors next examined the biological processes specified by the genes containing the variant exons correlated with prostate cancer-relevant Hallmark signaling pathways (FIG. 2C). The inventors also added a signature that describes transcriptional activity due to TMPRSS signaling as this common prostate cancer alteration is not represented in the Hallmark set (43). Here the inventors represent the network of data as a hive plot to show how exons (left axis) correlate with signaling pathways (middle axis) and the functional enrichment of genes containing those correlated exons (right axis) (44). Gene ontology analysis indicated that the relatively small number of exons correlated with AR or Notch were modestly enriched in cell adhesion and chromatin remodeling processes. Surprisingly, the numerous exon correlates of Myc, E2F, and MTOR were strongly enriched in genes related to the spliceosome and alternative pre-mRNA splicing. In addition, the overlap in the exon sets correlated with Myc, E2F, and MTOR was striking, with 50-60% of exons held in common (FIG. 2D). These pathways play central roles in growth control and are frequently co-dysregulated in human cancers, so a shared set of exons might be expected from a correlation analysis.

### 4. Myc-Correlated Exons are Found in the Oncogenes SRSF3 and HRAS

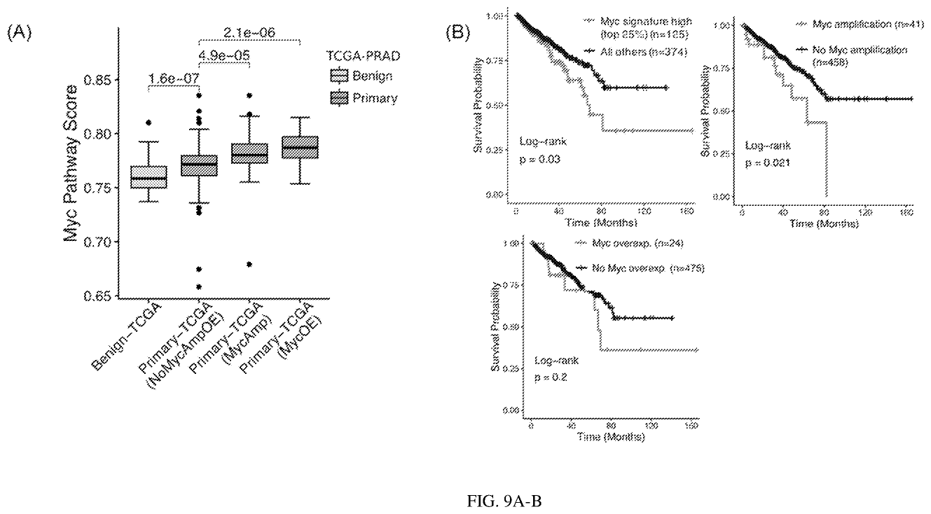

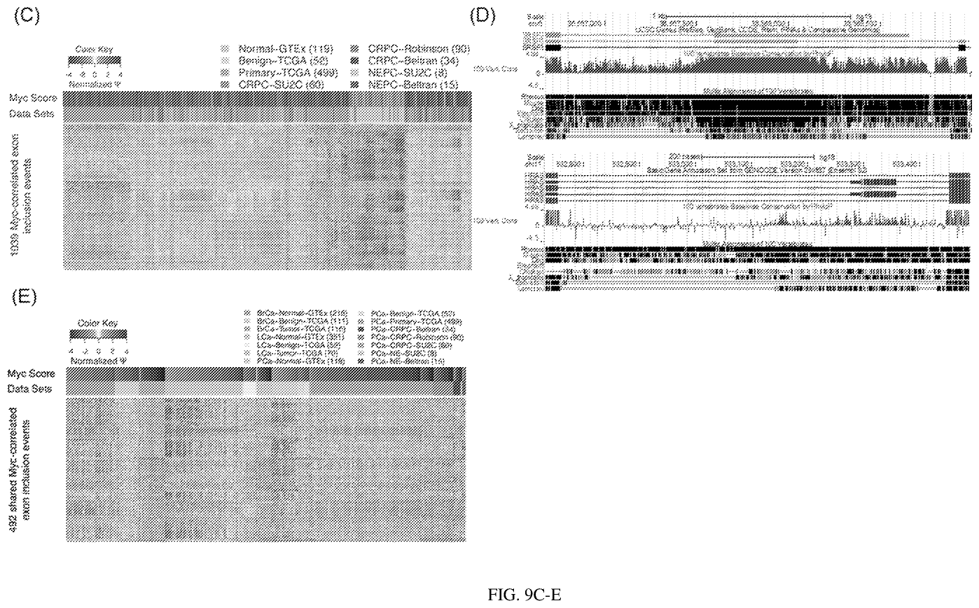

Given the centrality of Myc signaling in tumorigenesis, tumor maintenance, and tumor progression in a multitude of tissue lineages (45, 46) including the prostate, this pathway was selected for further investigation (15, 47, 48). The validity of these correlational results critically depends on the integrity of the underlying gene signature used to produce them. The inventors therefore performed additional validation steps on the “MYC Targets V2” hallmark signature by examining its performance in The Cancer Genome Atlas prostate adenocarcinoma RNA-Seq dataset (TCGA-PRAD) that has accompanying patient outcomes data (32). The inventors noted that samples with genomic amplifications of Myc had higher signature scores on average, as did samples that overexpressed Myc at the mRNA level (FIG. 9A). To examine if these relatively small changes in signature score had clinical relevance, the inventors performed Kaplan-Meier survival analyses using the “MYC Targets V2” signature, Myc genomic amplification status, or Myc single gene overexpression status as strata. The Mye gene signature was equally predictive of overall survival as genomic amplification status and outperformed single gene expression stratification (FIG. 9B).

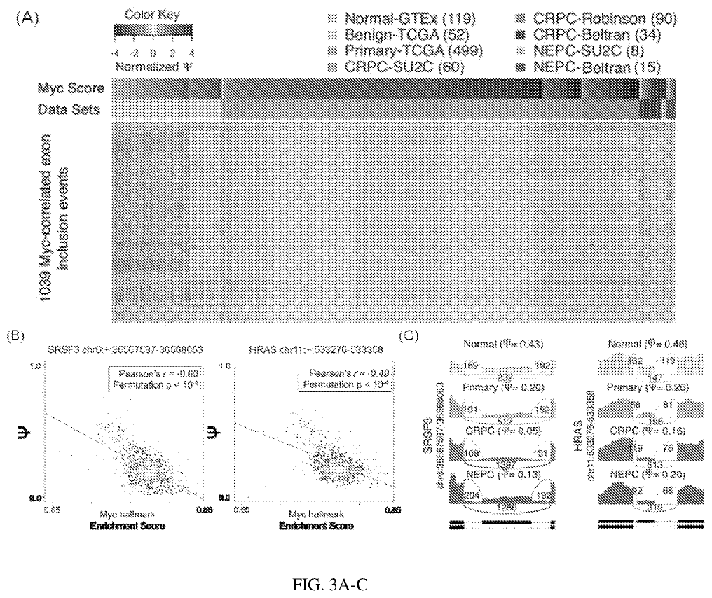

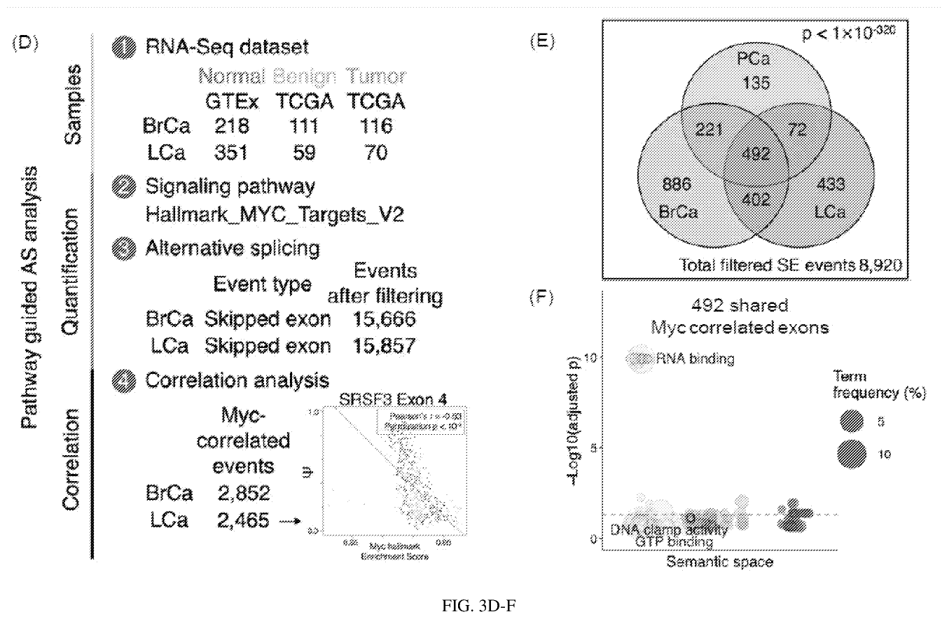

Convinced of the performance of the Myc signature by these additional tests, the inventors performed further analysis of the 1,039 Myc-correlated exons the inventors identified in the prostate meta-dataset (FIG. 3A and Tables 1a-1d). Unsupervised clustering of these 1,039 exons also grouped the samples by phenotype (FIG. 9C), identifying patterns in Myc-dependent exon incorporation that varied accordingly.

Two examples among the most strongly Myc-correlated cassette exons from this analysis are found in SRSF3 and HRAS (FIG. 3B). Incorporation of the identified alternate exon in SRSF3 is anti-correlated with the Myc signature score (FIG. 3B, left panel). When examined by cancer phenotype, incorporation of this exon decreases as prostate cancer progresses from normal tissue to primary tumor and is even lower in mCRPC samples (FIG. 3C, left panel). The Myc signature score increased between normal healthy donors (GTEx) and tumor-adjacent normal (TCGA-PRAD), consistent with field cancerization and tumor-stromal interaction effects on gene expression reported previously by others (49). Incorporation of this exon in NEPC is slightly higher, consistent with the Myc signature scores in these samples (FIG. 8A).

SRSF3 is a serine-arginine splicing factor that can act as a proto-oncogene and also participates in transcription termination and DNA repair (50-53). The exon in question is ultra-conserved throughout evolution and contains an in-frame stop codon. Also known as a poison exon, this sequence functions as a premature termination codon (PTC) (FIG. 9D, top panel). Incorporation of this PTC has been shown previously to reduce SRSF3 expression levels by inducing nonsense-mediated decay (NMD) of the transcript (54, 55). This data suggests increased Myc signaling leads to increased exon skipping, reduced NMD, and increased expression of SRSF3.

A cassette exon in HRAS was also anti-correlated with Myc activity (FIG. 3B, right panel). When examined by cancer phenotype, exon skipping increased with tumor progression (FIG. 3C, right panel). HRAS is a well-known oncogene that cooperates with Myc to induce carcinogenesis in multiple tissues (56, 57). Inclusion of the cassette exon and the stop codon it contains results in the truncated HRAS p19 product instead of the p21 form (58). HRASp19 lacks the cysteine residues in the carboxy-terminal domain of HRASp21 required for nuclear translocation and RAS-driven transformation and may function instead as a tumor suppressor (58, 59). This exon is conserved in mammals (FIG. 9D, bottom panel). Incorporation of this exon is anti-correlated with Myc activity, suggesting that Myc can drive increased expression of oncogenic HRAS by affecting its splicing.

### 5. Myc-Correlated Exons in Prostate Cancers are Highly Conserved in Breast and Lung Adenocarcinomas

To determine if the observed effects of Myc activity on splicing were prostate-cancer specific, the inventors performed a similar correlation analysis on a second hormone-dependent malignancy, breast adenocarcinoma, and a third hormone-independent epithelial malignancy, lung adenocarcinoma. The normal tissue and cancer RNA-Seq datasets for this analysis were drawn from The Cancer Genome Atlas (TCGA-BRCA and TCGA-LUAD) datasets and the Genotype-Tissue Expression (GTEx) collection of normal tissue (31, 60, 61). The inventors performed a similar correlation between Myc signature score and exon usage as described above (FIG. 3D). The Myc signature scores in breast and lung tissues behaved similarly to those in the prostate tissues, with increases in score at each step when moving from normal to tumor-adjacent normal to carcinoma (FIG. 9E). The inventors identified 2,852 Myc-correlated cassette exons in breast samples and 2,465 in lung samples using the same filtering criteria for the prostate study (FIG. 9F). The exon list includes the same anti-correlated exon in SRSF3, as shown for lung samples (FIG. 3D, fourth panel). Intersecting this set with the inventors' previously-defined set of Myc-responsive prostate cancer exons (FIG. 3A), the inventors found extensive overlap and similar exon incorporation behavior in the three sets (FIG. 3E). The triple intersection was even more strongly enriched for RNA-binding proteins (FIG. 3F). This analysis suggests the exon incorporation response to Myc overexpression is conserved across these cancers.

6. Creation of an Engineered Model of Advanced Prostate Cancer with Regulated Myc Expression from Benign Human Prostate Cells to Define Myc-Dependent Exon Events

Correlation analysis strongly implicates Myc, E2F, and MTOR signaling in the control of exons related to alternative pre-mRNA splicing but cannot define the individual contribution of each pathway to the observed phenotype. The inventors therefore sought to determine if the Myc-correlated splicing effects the inventors observed were indeed Myc-dependent.

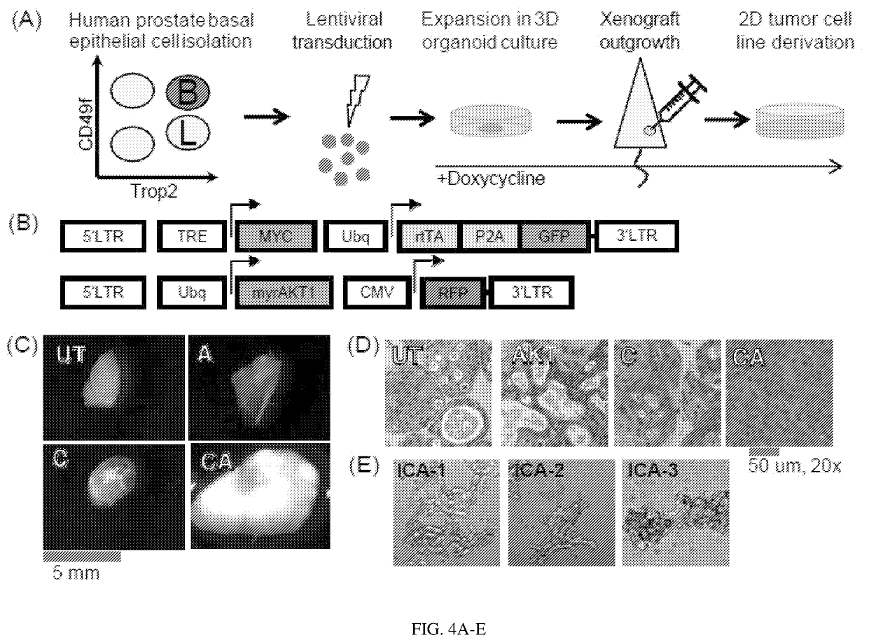

Numerous studies of the effect of Myc overexpression have described large numbers of Myc target genes with significant tissue heterogeneity (62, 63). The presence of complex background genetics, undefined driver alterations, and tissue culture-specific phenomena further complicate the study of Myc biology (64). The inventors therefore constructed a model of advanced prostate cancer by the transformation of benign human prostate epithelial cells with defined oncogenes (FIG. 4A) (65). The inventors have previously shown that the enforced expression of Myc and myristoylated (activated) AKT1 (myrAKT1) generates androgen receptor-independent adenocarcinoma (66, 67). MyrAKT1 is included to phenocopy the activation of AKT1 that follows deletion of the tumor suppressor PTEN, a common event in prostate cancer tumorigenesis. Here, the inventors cloned the Myc cDNA into a doxycycline-inducible promoter lentiviral construct whereas MyrAKT1 was constitutively expressed (FIG. 4B and Methods).

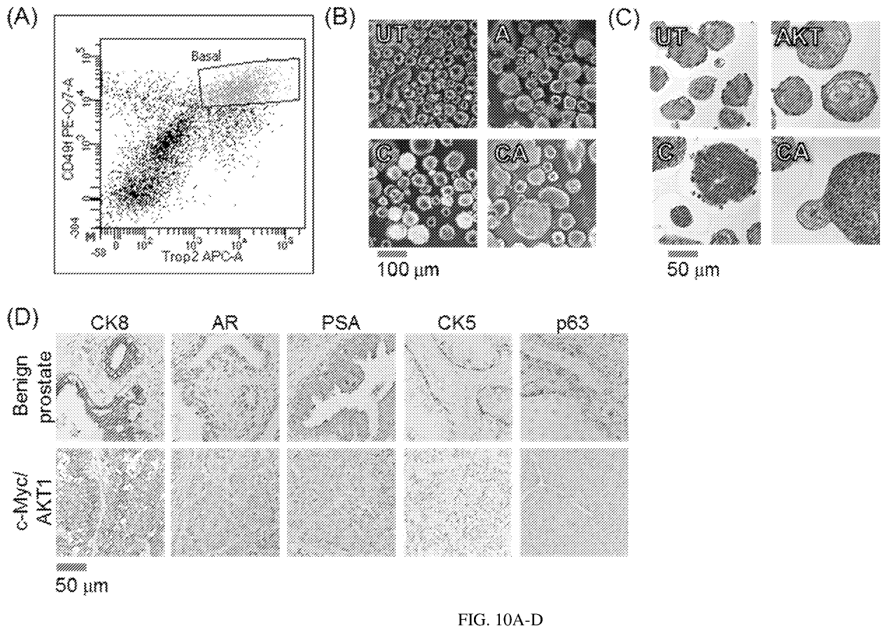

After lentiviral transduction of isolated human prostate basal cells (FIG. 10A), the inventors initiated the organoid culture and subsequent subcutaneous xenograft tumor outgrowth in immunocompromised mice in the constant presence of the drug (FIG. 10B-C). As previously reported, only doubly-transduced cells resulted in tumor outgrowth (FIG. 4C). The histologic appearance and marker expression patterns of the xenograft outgrowths were similar to those previously published with constitutive constructs (FIG. 4D and FIG. 10D). The xenograft outgrowths were dissociated, plated in tissue culture conditions with doxycycline to initiate autonomously growing cell lines (FIG. 4E). The inventors repeated the entire procedure to generate three independent cell lines from the prostate epithelium of three different human specimens.

### 7. Myc Withdrawal Leads to Decreased Expression of Splicing-Related Genes

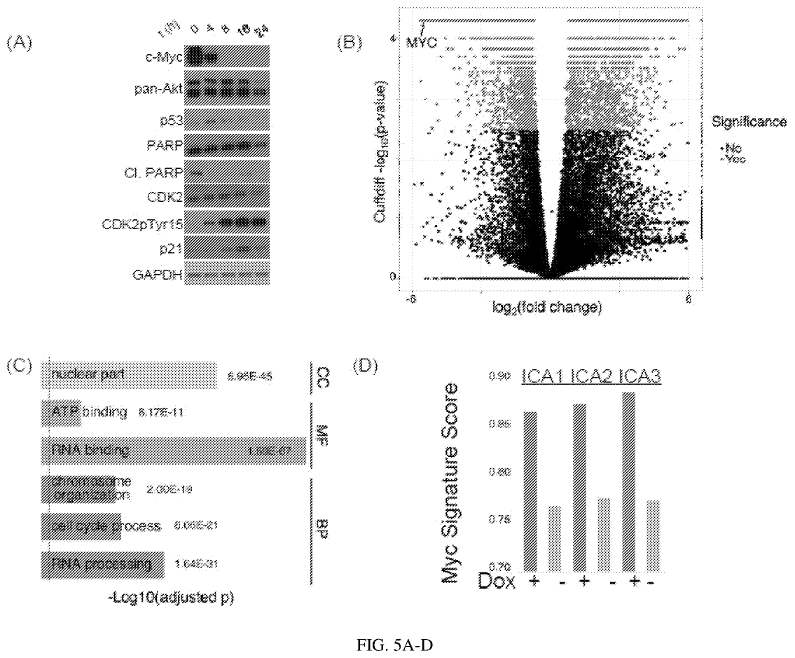

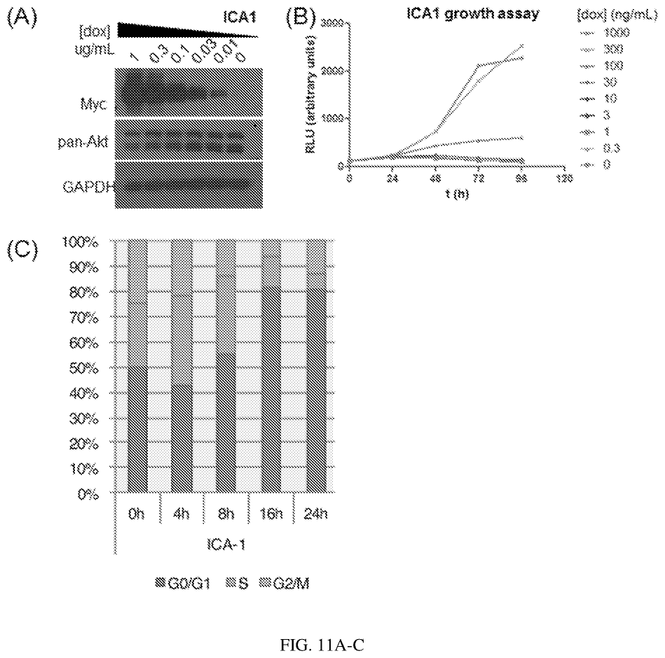

Withdrawal of doxycycline from the Myc/myrAKT1 cell lines resulted in the rapid, dose-dependent loss of Myc protein expression, consistent with its previously-reported short half-life (FIG. 5A and FIG. 11A) (68). The cells also rapidly slowed their growth with increased G0/G1 fraction at 24 hours (FIG. 11B-C). They adopted a senescent-like phenotype after prolonged Myc withdrawal with upregulation of P21 (FIG. 5A). A similar consequence of Myc withdrawal in oncogene-addicted transformed cells has been previously reported (69).

The inventors performed RNA-Seq on samples from Myc-high and Myc-low conditions to define Myc-dependent genes and exons in this model system. These samples were sequenced with high read depth (>100M reads) to enable accurate quantification of alternative splicing in downstream analysis. Primary analysis of the RNA expression data showed that thousands of genes were highly responsive to Myc withdrawal (CuffDiff q-value<0.05) (FIG. 5B). Gene ontology analysis identified enrichment of several growth-related biological processes among the Myc-dependent genes (FIG. 5C). Of note, genes involved in RNA processing were among the most highly enriched in this subset. This is consistent with previous reports of Myc's broad control of the growth phenotype. The regulated Myc expression system also allowed us to independently validate the Myc signature score the inventors used in the correlation analysis (FIG. 5D).

### 8. Experimentation Confirms Myc-Regulated Exons are Enriched in Splicing-Related Proteins and Often Encode Premature Termination Codons

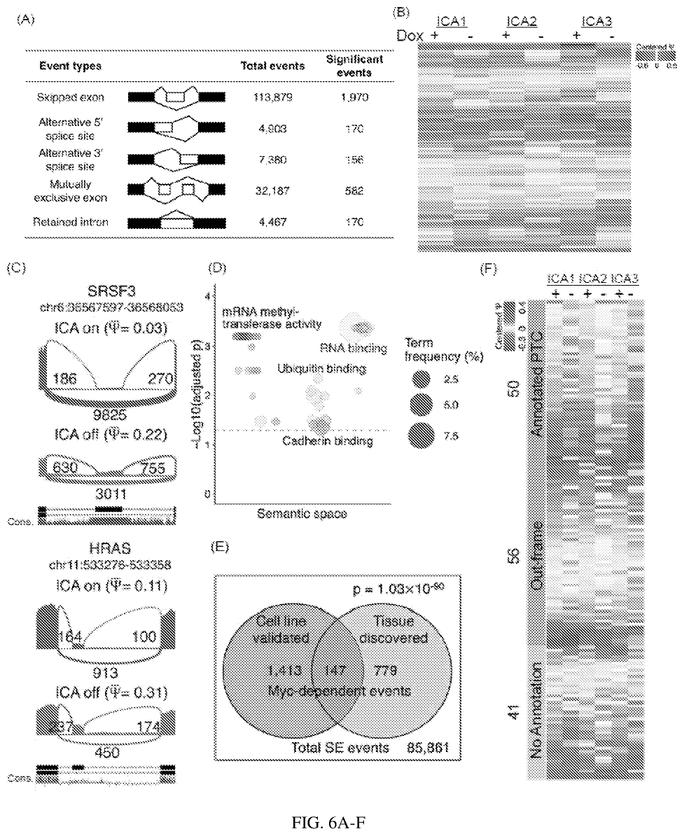

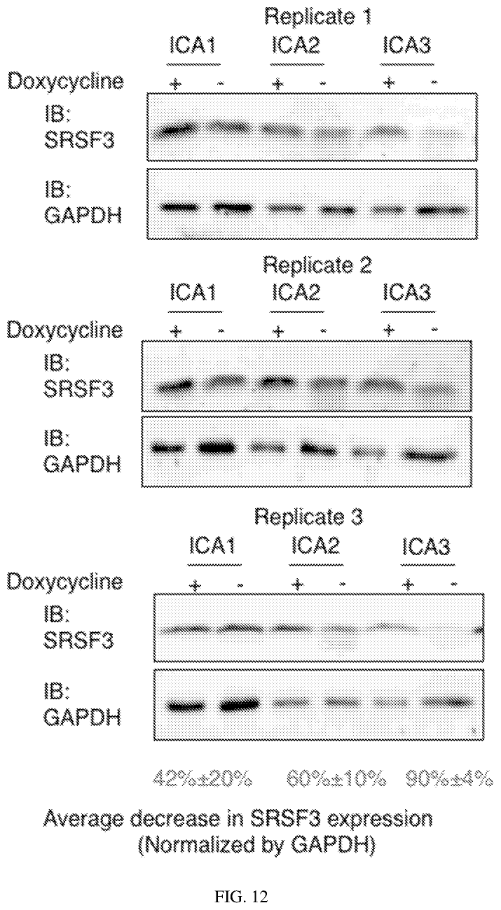

The inventors applied rMATS-turbo to analyze Myc-regulated exon usage in the engineered cell lines. To accommodate the paired nature of the dataset (comparing Myc-high and Myc-low conditions for each), the inventors employed the PAIRADISE statistical test to the rMATS-turbo output (70). After filtering for coverage (≥10 splice junction reads per event), effect size ((|deltaPSI|>5%), and false discovery rate (FDR<5%), this analysis yielded 1,970 cassette exons that significantly changed incorporation in response to Myc withdrawal (FIGS. 6A-B. Tables 1a-1d). The inventors note that among the Myc-dependent exons the inventors again identified the alternative exons in SRSF3 and HRAS described above, experimentally demonstrating that their incorporation is dependent on Myc signaling (FIG. 6C). The relative incorporation of the poison exon in SRSF3 increased when Myc was withdrawn, which would act to decrease the amount of SRSF3 protein in response to oncogene loss. The inventors confirmed by immunoblotting that SRSF3 protein levels decreased relative to the housekeeping protein GAPDH in this experimental setting (FIG. 12A).

Similar to the correlational data from the patient specimens, the Myc-dependent exons were strikingly enriched in genes affecting RNA splicing-related processes (FIG. 6D). Intersecting this set of exons with the Myc-correlated exons in patient tissue identified 147 common exons (FIG. 6E), a highly-significant overlap (p=1.03×10⁻⁹⁰). The remaining exons may not be responsive to short-term withdrawal of Myc in the cell line model or may be correlated with other signaling derangements that often accompany Myc deregulation in patient cancers (e.g. E2F or MTOR).

To look for additional Myc-regulated exons in clinically-important genes beyond SRSF3 and HRAS, the inventors cross-referenced the 147-exon list with the MSKCC-IMPACT cancer driver panel (71). This search identified four additional exons in three genes: SMARCA4, PBRM1, and TBX3 (FIG. 12B). SMARCA4 and PBRM1 are both involved in chromatin remodeling, and TBX3 is a transcription factor that promotes tumor invasion (72, 73). However, the change in exon incorporation was relatively modest and correlation with protein level would be necessary before further study.

Alternative pre-mRNA splicing can regulate transcript levels through the incorporation or skipping of nonsense-mediated decay determinant exons (74). The inventors reasoned that Myc-driven exon choice in splicing proteins could contribute to the regulation of their expression levels. To examine the functional outcome of Myc-driven splicing changes on nonsense-mediated decay, the inventors annotated the 147 exons in the patient data-cell line intersection for premature termination codons (PTCs) and frameshifts (FIG. 6F, Tables 1a-1d). These 147 exons correspond to 124 genes, 30 of which were RNA-binding proteins by GO designation. The inventors annotated all these exons using the Ensembl database to identify those that contained verified PTCs. The inventors supplemented this annotation by parsing the 10 remaining exons to identify those predicted to produce a frameshift within the coding sequence of the parent mRNA transcript. The inventors found that 36 of the 43 exons in RNA-binding genes encode a PTC, a frameshift, or both. These exons represent a set of Myc-responsive sequences that act to regulate transcript abundance of proteins involved in alternative pre-mRNA splicing.

### B. Tables

TABLE 1a
Pathway AS Tissue

| AS Events | AS Events | AS Events |
|---|---|---|
| SCAMP5_chr15_+_75308933_{—} | TMEM8B_chr9_+_35834457_{—} | SLC6A6_chr3_+_14509595_{—} |
| 75309090_75305146_75310137 | 35834647_35829952_35845971 | 14509720_14509464_14513712 |
| RAB27A_chr15_−_55529513_{—} | NASP_chr1_+_46066056_{—} | C14orf159_chr14_+_91690022_{—} |
| 55529640_55527154_55562362 | 46066129_46056942_46067926 | 91690139_91681900_91691027 |
| GAS6-AS1_chr13_+_114537550_{—} | ZFR_chr5_−_32380180_{—} | INPP5K_chr17_−_1416746_{—} |
| 114537737_114536552_114541049 | 32380278_32379316_32385613 | 1416848_1413093_1417165 |
| GALNS_chr16_−_88909113_{—} | TMEM184B_chr22_−_38642811_{—} | MICAL3_chr22_−_18355512_{—} |
| 88909237_88908379_88923165 | 38642891_38642106_38643775 | 18355620_18354789_18368643 |
| SPIN1_chr9_+_91033692_{—} | TM7SF2_chr11_+_64883246_{—} | RP11-231C18.3_chr4_+_54280781_{—} |
| 91033866_91003453_91041296 | 64883294_64882866_64883364 | 54280889_54266006_54292038 |
| NAA16_chr13_+_41936866_{—} | PPIP5K2_chr5_+_102518934_{—} | ALKBH3_chr11_+_43913590_{—} |
| 41937009_41936295_41941574 | 102519108_102515889_102520372 | 43913679_43911378_43923065 |
| RWDD2B_chr21_−_30380560_{—} | MARK3_chr14_+_103966492_{—} | MCCC1_chr3_−_182810196_{—} |
| 30380628_30380444_30380715 | 103966537_103958371_103969218 | 182810333_182804576_182812346 |
| ENOSF1_chr18_−_691067_{—} | AKR7A3_chr1_−_19611511_{—} | FLNA_chrX_−_153585618_{—} |
| 691106_690631_693881 | 19611608_19611279_19612381 | 153585642_153583440_153585801 |
| IFT122_chr3_+_129180070_{—} | SLC25A29_chr14_−_100761962_{—} | FAT1_chr4_−_187511521_{—} |
| 129180147_129170841_129182402 | 100762034_100759714_100765178 | 187511557_187510374_187516842 |
| BIN1_chr2_−_127825738_{—} | ECHDC2_chr1_−_53370705_{—} | SLC38A5_chrX_−_48324616_{—} |
| 127825831_127821594_127826499 | 53372283_53364896_53373539 | 48324709_48321365_48325185 |
| STIM1_chr11_+_4105902_{—} | IQCB1_chr3_−_121518042_{—} | ATG2A_chr11_−_64671061_{—} |
| 4105995_4104728_4107706 | 121518221_121509062_121526190 | 64671132_64670836_64673034 |
| ECHDC2_chr1_−_53379566_{—} | DNASE1_chr16_+_3705849_{—} | TSTD1_chr1_−_161008340_{—} |
| 53379767_53377462_53387224 | 3705938_3705521_3706102 | 161008463_161007865_161008669 |
| TINF2_chr14_−_24711095_{—} | TMEM175_chr4_+_942298_{—} | USP9Y_chrY_+_14930354_{—} |
| 24711127_24710982_24711346 | 942403_941942_944208 | 14930545_14928279_14945613 |
| MAGIX_chrX_+_49021245_{—} | ITGA7_chr12_−_56093653_{—} | CRAT_chr9_−_131871457_{—} |
| 49021428_49021127_49021527 | 56093773_56092701_56094045 | 131871556_131870356_131872761 |
| PRUNE2_chr9_−_79256887_{—} | GGCT_chr7_−_30540151_{—} | CDK10_chr16_+_89753996_{—} |
| 79256896_79253204_79259654 | 30540297_30538554_30544184 | 89754223_89753205_89755659 |
| SRSF11_chr1_+_70697541_{—} | DDX46_chr5_+_134125795_{—} | CALD1_chr7_+_134620438_{—} |
| 70697658_70694238_70697950 | 134125912_134124281_134126159 | 134620516_134618141_134625842 |
| PRPF39_chr14_+_45579096_{—} | PRKRA_chr2_−_179310152_{—} | BZW2_chr7_+_16725548_{—} |
| 45579196_45578983_45579296 | 179310262_179309227_179312231 | 16725665_16722470_16729421 |
| NUDT2_chr9_+_34336226_{—} | PHLDB2_chr3_+_111667778_{—} | IFT22_chr7_−_100961404_{—} |
| 34336339_34329597_34338710 | 111667922_111664204_111672776 | 100961494_100959823_100964951 |
| INO80E_chr16_+_30013756_{—} | CARD19_chr9_+_95874160_{—} | ARFIP2_chr11_−_6499808_{—} |
| 30014334_30012851_30016541 | 95874220_95873003_95874499 | 6499882_6499428_6499967 |
| FLAD1_chr1_+_154960580_{—} | USP3_chr15_+_63821212_{—} | TP53BP1_chr15_−_43738963_{—} |
| 154961325_154956542_154965188 | 63821365_63797029_63824845 | 43739108_43738788_43739563 |
| BCAR1_chr16_−_75270779_{—} | KANK2_chr19_−_11306231_{—} | METTL26_chr16_−_685517_{—} |
| 75270896_75269884_75276367 | 11306496_11305266_11308160 | 685774_685340_686093 |
| RPS6KB1_chr17_+_58008160_{—} | BNIP2_chr15_−_59970974_{—} | BPTF_chr17_+_65871671_{—} |
| 58008413_58007535_58008982 | 59971033_59970286_59971790 | 65871860_65871136_65882243 |
| MPST_chr22_+_37419792_{—} | WDR77_chr1_−_111984120_{—} | UBE3A_chr15_−_25652213_{—} |
| 37419968_37415815_37420232 | 111984226_111984011_111984646 | 25652409_25650649_25654234 |
| MORF4L2_chrX_−_102939608_{—} | CCSER2_chr10_+_86259630_{—} | EVA1C_chr21_+_33873724_{—} |
| 102939657_102933548_102940098 | 86259715_86185649_86273204 | 33873848_33867480_33887123 |
| DDX39A_chr19_−_14521232_{—} | TMEM50B_chr21_−_34809189_{—} | RUFY1_chr5_+_179013331_{—} |
| 14521417_14521146_14521800 | 34809299_34805109_34811520 | 179013476_179012866_179016546 |
| ACSF3_chr16_+_89180746_{—} | PIGP_chr21_−_38444444_{—} | RBM10_chrX_+_47034417_{—} |
| 89180895_89178654_89187208 | 38444610_38439680_38444733 | 47034491_47032596_47035898 |
| TMPRSS2_chr21_−_42870045_{—} | SON_chr21_+_34944856_{—} | PRMT2_chr21_+_48056350_{—} |
| 42870116_42861520_42880007 | 34944936_34941393_34945613 | 48056459_48055675_48056807 |
| CCHCR1_chr6_−_31122272_{—} | ADA_chr20_−_43251228_{—} | EZH2_chr7_−_148516069_{—} |
| 31122576_31118899_31124507 | 43251293_43249788_43251469 | 148516151_148515209_148516687 |
| RBM5_chr3_+_50137964_{—} | ANXA2_chr15_−_60689456_{—} | LMF1_chr16_−_905106_{—} |
| 50138038_50137484_50140515 | 60689537_60688626_60690141 | 906055_904706_918943 |
| NMRAL1_chr16_−_4521314_{—} | SMTN_chr22_+_31496870_{—} | RCC1_chr1_+_28857034_{—} |
| 4521450_4519466_4524093 | 31497035_31495882_31500301 | 28857127_28856451_28858314 |
| ADD3_chr10_+_111892062_{—} | RSRC2_chr12_−_122998313_{—} | C17orf62_chr17_−_80407306_{—} |
| 111892158_111890244_111893083 | 122998421_122995735_122999651 | 80407832_80407168_80408575 |
| IFT22_chr7_−_100962236_{—} | RNASE1_chr14_−_21270407_{—} | BET1_chr7_−_93623663_{—} |
| 100962344_100961494_100964951 | 21270490_21270252_21270955 | 93623697_93605412_93625576 |
| CD151_chr11_+_834529_{—} | TNS1_chr2_−_218695089_{—} | IGFLR1_chr19_−_36230610_{—} |
| 834745_833026_836062 | 218695113_218694605_218696177 | 36230987_36230527_36231924 |
| ACY1_chr3_+_52019222_{—} | LMBRIL_chr12_−_49500436_{—} | CAMK2G_chr10_−_75577966_{—} |
| 52019287_52018174_52019376 | 49500529_49499740_49500743 | 75578653_75577312_75581439 |
| NOL3_chr16_+_67207764_{—} | DPH7_chr9_−_140472028_{—} | RHOC_chr1_−_113247721_{—} |
| 67207949_67204477_67208064 | 140472055_140470619_140473076 | 113247823_113246428_113249699 |
| IMMP1L_chr11_−_31477806_{—} | NEDD4L_chr18_+_56002709_{—} | LAS1L_chrX_−_64744443_{—} |
| 31477933_31455117_31531065 | 56002769_56001124_56008269 | 64744494_64744142_64744844 |
| FMNL3_chr12_−_50040421_{—} | ABCC9_chr12_−_22017372_{—} | COL6A3_chr2_−_238303229_{—} |
| 50040536_50039686_50040668 | 22017411_22015988_22025558 | 238303847_238296827_238305369 |
| CRELD1_chr3_+_9979698_{—} | CDC14B_chr9_−_99277930_{—} | CHD3_chr17_+_7810918_{—} |
| 9979790_9976601_9982533 | 99278074_99266071_99284787 | 7811020_7810806_7811211 |
| TREX1_chr3_+_48508028_{—} | EEF1D_chr8_−_144674816_{—} | AUH_chr9_−_94122560_{—} |
| 48508040_48507708_48508138 | 144675063_144672908_144679517 | 94122722_94118437_94123909 |
| GGT1_chr22_+_25004956_{—} | PDGFA_chr7_−_540067_{—} | TRIP6_chr7_+_100465532_{—} |
| 25005198_25003990_25005931 | 540136_538211_540752 | 100465610_100465228_100465729 |
| GINS1_chr20_+_25394425_{—} | LGMN_chr14_−_93172827_{—} | NISCH_chr3_+_52514894_{—} |
| 25394490_25388531_25405846 | 93172998_93170706_93176016 | 52515053_52514311_52518528 |
| HPS1_chr10_−_100190887_{—} | SORBS1_chr10_−_97174250_{—} | PRR3_chr6_+_30529104_{—} |
| 100191048_100190427_100193696 | 97174412_97170534_97181717 | 30529285_30525989_30529610 |
| NIN_chr14_−_51223209_{—} | PRKAG1_chr12_−_49399525_{—} | SLC27A1_chr19_+_17599656_{—} |
| 51225348_51221585_51226574 | 49399664_49399326_49406844 | 17599748_17598338_17599816 |
| TMEM184A_chr7_−_1589182_{—} | METTL21B_chr12_+_58168411_{—} | NUDT22_chr11_+_63997321_{—} |
| 1589240_1588324_1589489 | 58168550_58166911_58174037 | 63997542_63997043_63997654 |
| SVIL_chr10_−_29797270_{—} | ST7_chr7_+_116830186_{—} | EIF4A2_chr3_+_186502750_{—} |
| 29797318_29788192_29801663 | 116830322_116829447_116830887 | 186502890_186502485_186503671 |
| NCOR2_chr12_−_124812092_{—} | LSM1_chr8_−_38027319_{—} | ATG16L1_chr2_+_234182366_{—} |
| 124812179_124810916_124815390 | 38027435_38021358_38029482 | 234182423_234181698_234183321 |
| CAPN3_chr15_+_42698123_{—} | TMEM175_chr4_+_941496_{—} | ZNF605_chr12_−_133524733_{—} |
| 42698141_42695975_42700408 | 941680_926328_944208 | 133524856_133522383_133532828 |
| CCDC130_chr19_+_13872304_{—} | RPL17_chr18_−_47018105_{—} | ARRB2_chr17_+_4618307_{—} |
| 13872382_13870086_13873112 | 47018203_47017954_47018627 | 4618338_4614039_4619267 |
| SEPT6_chrX_−_118759297_{—} | MCCC1_chr3_−_182746880_{—} | FAM160B2_chr8_+_21947271_{—} |
| 118759359_118754014_118763280 | 182746977_182743592_182751778 | 21947365_21946809_21951950 |
| PRUNE2_chr9_−_79239938_{—} | METTL26_chr16_−_684888_{—} | IDH3A_chr15_+_78449249_{—} |
| 79239974_79229516_79244107 | 684956_684797_686093 | 78449504_78447617_78449889 |
| CCND3_chr6_−_41908107_{—} | FASTKD3_chr5_−_7866758_{—} | TMED5_chr1_−_93624371_{—} |
| 41908323_41905132_42016238 | 7868309_7866096_7869091 | 93624442_93622040_93625685 |
| PIGB_chr15_+_55642896_{—} | DCUN1D5_chr11_−_102953476_{—} | EBF4_chr20_+_2730046_{—} |
| 55643001_55634000_55646995 | 102953568_102937296_102953984 | 2730164_2729276_2730265 |
| METTL26_chr16_−_684888_{—} | ALG13_chrX_+_110964830_{—} | TMEM234_chr1_−_32683037_{—} |
| 685063_684797_686093 | 110964939_110963398_110966020 | 32683178_32682952_32686731 |
| SEPT6_chrX_−_118759297_{—} | LIN7A_chr12_−_81241877_{—} | ADGRG1_chr16_+_57675498_{—} |
| 118759342_118754014_118763280 | 81241954_81239718_81242029 | 57675620_57654048_57684164 |
| SYNE2_chr14_+_64682003_{—} | ACTN2_chr1_+_236898934_{—} | RANBP17_chr5_+_170455875_{—} |
| 64682072_64681188_64682965 | 236899020_236894614_236900421 | 170455993_170395381_170597133 |
| RP11-793H13.8_chr12_+_53858543_{—} | HMGN1_chr21_−_40717755_{—} | LRRFIP2_chr3_−_37132957_{—} |
| 53858636_53856351_53859715 | 40717884_40717200_40720217 | 37133029_37125297_37136282 |
| RHOC_chr1_−_113247721_{—} | UBE3A_chr15_−_25652213_{—} | SEC61A2_chr10_+_12197776_{—} |
| 113248874_113246428_113249699 | 25652375_25650649_25654234 | 12197930_12191960_12198905 |
| ARIH2_chr3_+_48962150_{—} | MEAF6_chr1_−_37962307_{—} | ILF3_chr19_+_10791021_{—} |
| 48962272_48960244_48982568 | 37962337_37962205_37967404 | 10791119_10790603_10791694 |
| BIRC5_chr17_+_76218908_{—} | LSM14B_chr20_+_60702640_{—} | PAM_chr5_+_102309819_{—} |
| 76219073_76212862_76219545 | 60702757_60701495_60704840 | 102310140_102296933_102325975 |
| PPRC1_chr10_+_103904776_{—} | AKAP8_chr19_−_15479877_{—} | MRPL55_chr1_−_228296137_{—} |
| 103904847_103904064_103908128 | 15480035_15479133_15480956 | 228296175_228296019_228296961 |
| TRIQK_chr8_−_93966633_{—} | DNAJB14_chr4_−_100845916_{—} | C17orf62_chr17_−_80407045_{—} |
| 93966792_93929238_93978234 | 100845972_100844342_100851606 | 80407168_80404572_80408575 |
| WDSUB1_chr2_−_160114455_{—} | ALDH2_chr12_+_112213408_{—} | TBL2_chr7_−_72990870_{—} |
| 160114496_160112886_160116320 | 112213524_112204900_112219721 | 72991031_72988843_72992749 |
| CSNK1G3_chr5_+_122941032_{—} | SETD5_chr3_+_9510202_{—} | MRPS18C_chr4_+_84379498_{—} |
| 122941056_122940524_122950035 | 9510259_9506356_9512142 | 84379582_84378111_84380892 |
| KDM6A_chrX_+_44921891_{—} | SRP9_chr1_+_225974563_{—} | MRPS33_chr7_−_140710218_{—} |
| 44921993_44920664_44922666 | 225974687_225971070_225976941 | 140710460_140706335_140714710 |
| MLPH_chr2_+_238443206_{—} | INO80E_chr16_+_30012734_{—} | SPIRE1_chr18_−_12459753_{—} |
| 238443290_238434448_238448990 | 30012851_30012361_30016541 | 12459927_12454482_12463349 |
| ARMC10_chr7_+_102732923_{—} | BIRC5_chr17_+_76212046_{—} | MGEA5_chr10_−_103553669_{—} |
| 102733100_102727211_102737723 | 76212115_76210870_76219545 | 103553755_103552700_103557736 |
| HAUS7_chrX_−_152720334_{—} | MYO18A_chr17_−_27412621_{—} | DCN_chr12_−_91573138_{—} |
| 152720511_152719966_152720999 | 27412666_27409456_27413455 | 91573463_91572362_91576431 |
| SNAPIN_chr1_+_153631613_{—} | DNM1L_chr12_+_32890798_{—} | NAGPA_chr16_−_5078297_{—} |
| 153631660_153631362_153631923 | 32890876_32890095_32891197 | 5078366_5078186_5078880 |
| KIF13A_chr6_−_17790102_{—} | GK5_chr3_−_141903552_{—} | HNRNPC_chr14_−_21731825_{—} |
| 17790141_17788106_17794479 | 141904635_141901891_141904770 | 21731988_21702388_21737456 |
| THTPA_chr14_+_24025951_{—} | FBXO31_chr16_−_87392016_{—} | HNRNPA1_chr12_+_54676862_{—} |
| 24026243_24025552_24027903 | 87392103_87380856_87393900 | 54677018_54676658_54677595 |
| SLC37A4_chr11_−_118897280_{—} | ZCCHC6_chr9_−_88933872_{—} | DNASE1_chr16_+_3705849_{—} |
| 118897398_118896790_118897646 | 88933973_88932191_88934498 | 3705967_3705521_3706102 |
| GPS1_chr17_+_80010253_{—} | FKRP_chr19_+_47251771_{—} | C2CD5_chr12_−_22611417_{—} |
| 80010335_80009840_80011149 | 47251878_47251345_47258668 | 22611519_22610095_22622642 |
| ADAM15_chr1_+_155034379_{—} | G3BP2_chr4_−_76649239_{—} | SLC2A8_chr9_+_130160183_{—} |
| 155034593_155033308_155034720 | 76649360_76587233_76649459 | 130160390_130159565_130162185 |
| MRPL55_chr1_−_228296137_{—} | PARP12_chr7_−_139737514_{—} | ZMIZ1_chr10_+_81070680_{—} |
| 228296722_228296022_228296961 | 139737656_139734131_139741443 | 81070941_81067328_81072398 |
| GUSB_chr7_−_65439905_{—} | POMT2_chr14_−_77767432_{—} | ATP6V0B_chr1_+_44441336_{—} |
| 65440058_65439691_65441001 | 77767592_77765904_77769177 | 44441520_44440779_44441761 |
| KANK2_chr19_−_11306297_{—} | NDUFAF5_chr20_+_13795063_{—} | MRPL1_chr4_+_78828099_{—} |
| 11306496_11305266_11308160 | 13795161_13789548_13797108 | 78828364_78815404_78830419 |
| RPL17_chr18_−_47018105_{—} | THEM4_chr1_−_151862458_{—} | ADD1_chr4_+_2928368_{—} |
| 47018203_47017954_47018644 | 151862690_151861849_151867483 | 2928402_2927839_2929897 |
| COBL_chr7_−_51240152_{—} | FAM136A_chr2_−_70528539_{—} | IDE_chr10_−_94240549_{—} |
| 51240227_51204028_51251798 | 70528735_70528112_70529056 | 94240673_94239178_94243011 |
| AURKA_chr20_−_54963742_{—} | MRPL47_chr3_−_179320439_{—} | SPTAN1_chr9_+_131355261_{—} |
| 54963840_54963258_54966998 | 179320585_179319578_179322314 | 131355321_131353904_131356453 |
| ARHGEF10L_chr1_+_17975048_{—} | PILRB_chr7_+_99950995_{—} | DHX30_chr3_+_47857452_{—} |
| 17975170_17966797_17981130 | 99951106_99950746_99951517 | 47857618_47852201_47859511 |
| NT5C2_chr10_−_104871501_{—} | IGFLR1_chr19_−_36231280_{—} | SCRIB_chr8_−_144889721_{—} |
| 104871562_104866463_104899162 | 36231465_36230527_36231924 | 144889784_144889183_144890778 |
| POFUT2_chr21_−_46697147_{—} | NOL3_chr16_+_67205054_{—} | TMPO_chr12_+_98938007_{—} |
| 46697688_46697057_46698017 | 67205360_67204477_67208064 | 98938127_98931350_98940136 |
| LINC01881_chr2_+_243058333_{—} | LRRFIP1_chr2_+_238622901_{—} | DTX3_chr12_+_57999353_{—} |
| 243058383_243037178_243061115 | 238622919_238617273_238628165 | 57999514_57998641_57999972 |
| TATDN2_chr3_+_10320347_{—} | ZBTB7B_chr1_+_154986490_{—} | BIRC5_chr17_+_76218908_{—} |
| 10320444_10320146_10320568 | 154986569_154975419_154987130 | 76219073_76210870_76219545 |
| ABHD4_chr14_+_23074692_{—} | SLC20A2_chr8_−_42328584_{—} | MKNK2_chr19_−_2039629_{—} |
| 23074772_23072984_23075327 | 42328719_42323435_42329619 | 2039855_2037828_2040132 |
| C16orf58_chr16_−_31510813_{—} | PHPT1_chr9_+_139744954_{—} | MAP3K7_chr6_−_91254270_{—} |
| 31510846_31508271_31512006 | 139745012_139744589_139745206 | 91254351_91246120_91256976 |
| CWC25_chr17_−_36966528_{—} | CASK_chrX_−_41414852_{—} | PTK2B_chr8_+_27303310_{—} |
| 36966653_36966018_36966720 | 41414888_41413168_41416284 | 27303436_27301788_27308265 |
| ROGDI_chr16_−_4851267_{—} | ITGA7_chr12_−_56094045_{—} | RPL17_chr18_−_47018115_{—} |
| 4851322_4850579_4851503 | 56094177_56093773_56094682 | 47018203_47017954_47018644 |
| DIS3L2_chr2_+_233063236_{—} | INO80C_chr18_−_33067349_{—} | UPRT_chrX_+_74514397_{—} |
| 233063486_233028342_233075035 | 33067403_33060527_33077682 | 74514455_74513349_74516176 |
| ADAM15_chr1_+_155034051_{—} | KLHL29_chr2_+_23907268_{—} | RNF146_chr6_+_127606352_{—} |
| 155034122_155033965_155034720 | 23907407_23865720_23914543 | 127606493_127601485_127607194 |
| VRK3_chr19_−_50500760_{—} | NFIX_chr19_+_13198802_{—} | ZNF761_chr19_+_53948253_{—} |
| 50500827_50498532_50504046 | 13198950_13189549_13201112 | 53948376_53935281_53949479 |
| S100A13_chr1_−_153600596_{—} | S100A13_chr1_−_153602925_{—} | KLK2_chr19_+_51380959_{—} |
| 153600713_153600074_153603486 | 153603132_153600713_153603486 | 51381012_51380264_51381659 |
| LRRC75A-AS1_chr17_+_16342894_{—} | STARD4_chr5_−_110837659_{—} | MTSS1L_chr16_−_70699368_{—} |
| 16343017_16342728_16343498 | 110837786_110836814_110842027 | 70699443_70698999_70708208 |
| TANGO2_chr22_+_20040882_{—} | INO80E_chr16_+_30015875_{—} | NEB_chr2_−_152357904_{—} |
| 20041074_20040107_20043465 | 30015978_30012361_30016541 | 152357997_152354232_152359306 |
| CSGALNACT1_chr8_−_19315040_{—} | DENNDIA_chr9_−_126150008_{—} | ACOT9_chrX_−_23752457_{—} |
| 19315327_19297442_19315936 | 126150137_126146192_126165680 | 23752484_23751334_23761241 |
| FBXW8_chr12_+_117387422_{—} | CXorf40B_chrX_−_149102594_{—} | PCBP4_chr3_−_51995956_{—} |
| 117387511_117383333_117402501 | 149102786_149102355_149105650 | 51996104_51995320_51996825 |
| SLMAP_chr3_+_57911571_{—} | DUSP22_chr6_+_335113_{—} | CDC42SE1_chr1_−_151029126_{—} |
| 57911661_57908750_57913022 | 335163_311962_345853 | 151029262_151028469_151031954 |
| COL4A5_chrX_+_107913457_{—} | RBM3_chrX_+_48434202_{—} | AP3S2_chr15_−_90420537_{—} |
| 107913466_107911734_107920729 | 48434471_48434055_48434701 | 90420656_90414778_90431752 |
| PDCD10_chr3_−_167443188_{—} | THAP9-AS1_chr4_−_83816844_{—} | FDPS_chr1_+_155279543_{—} |
| 167443261_167438061_167452593 | 83816927_83816000_83821229 | 155279756_155278756_155279833 |
| PLA2G15_chr16_+_68282471_{—} | METTL22_chr16_+_8738385_{—} | CNDP2_chr18_+_72164743_{—} |
| 68282595_68279456_68283192 | 8738582_8736422_8739964 | 72164830_72163666_72167171 |
| KCTD17_chr22_+_37456862_{—} | RNF19A_chr8_−_101312755_{—} | D2HGDH_chr2_+_242690660_{—} |
| 37456962_37455478_37457578 | 101312879_101300495_101322094 | 242690803_242689709_242694459 |
| DDR1_chr6_+_30853401_{—} | KLK2_chr19_+_51377976_{—} | NDUFV3_chr21_+_44323291_{—} |
| 30853457_30850942_30856464 | 51378136_51376775_51379727 | 44324386_44317157_44328973 |
| EIF4A2_chr3_+_186506098_{—} | EPS8L1_chr19_+_55595134_{—} | QDPR_chr4_−_17493854_{—} |
| 186506209_186505671_186506913 | 55596010_55595012_55597208 | 17493963_17492368_17503341 |
| TNK2_chr3_−_195609156_{—} | PCSK7_chr11_−_117077782_{—} | TRMT2B_chrX_−_100306239_{—} |
| 195609199_195606046_195610027 | 117077876_117077514_117078685 | 100306722_100297301_100306805 |
| ENOSF1_chr18_−_678695_{—} | KIAA1217_chr10_+_24831811_{—} | ARHGEF11_chr1_−_156908209_{—} |
| 678737_677872_683245 | 24833410_24825822_24833909 | 156908305_156907288_156909339 |
| CDIPT_chr16_−_29873906_{—} | SYTL2_chr11_−_85422155_{—} | CORO7_chr16_−_4411749_{—} |
| 29873956_29872580_29874135 | 85422275_85420543_85425455 | 4411804_4411470_4411985 |
| MPDZ_chr9_−_13186268_{—} | CAST_chr5_+_96062457_{—} | LTBP3_chr11_−_65307483_{—} |
| 13186385_13183584_13188782 | 96062563_96058402_96063192 | 65307624_65307352_65307715 |
| FEZ2_chr2_−_36785580_{—} | ANKRD10_chr13_−_111546631_{—} | RANBP3_chr19_−_5957928_{—} |
| 36785656_36782891_36805739 | 111546740_111545610_111558379 | 5957984_5951607_5978071 |
| BRD8_chr5_−_137502206_{—} | EZH1_chr17_−_40871122_{—} | COASY_chr17_+_40714373_{—} |
| 137502416_137501797_137503622 | 40871225_40865407_40872290 | 40714505_40714237_40714629 |
| ERMARD_chr6_+_170153133_{—} | FAM192A_chr16_−_57212621_{—} | ATRIP_chr3_+_48501185_{—} |
| 170153277_170151759_170153959 | 57212764_57207781_57219732 | 48501315_48500853_48501508 |
| ME3_chr11_−_86161342_{—} | RALGAPA1_chr14_−_36017713_{—} | TMEM94_chr17_+_73488319_{—} |
| 86161440_86159297_86176132 | 36017744_36008896_36018315 | 73488418_73487981_73488554 |
| MPRIP_chr17_+_17083920_{—} | IFT22_chr7_−_100961404_{—} | TNFSF10_chr3_−_172229406_{—} |
| 17083983_17083402_17088136 | 100962313_100959823_100964951 | 172229449_172227111_172232650 |
| TCF20_chr22_−_42564614_{—} | P2RX4_chr12_+_121660749_{—} | ABCD4_chr14_−_74759856_{—} |
| 42564709_42557364_42565852 | 121660765_121659969_121666335 | 74760013_74759572_74761850 |
| HM13_chr20_+_30155880_{—} | CTNND1_chr11_+_57558856_{—} | GEMIN8_chrX_−_14044170_{—} |
| 30156083_30149539_30156922 | 57559145_57529518_57561481 | 14044340_14039630_14047895 |
| BICD1_chr12_+_32491719_{—} | NR2C1_chr12_−_95434251_{—} | TSTD1_chr1_−_161008340_{—} |
| 32491913_32490640_32520603 | 95434373_95425264_95442843 | 161008484_161007865_161008669 |
| FAM192A_chr16_−_57212413_{—} | BNIP2_chr15_−_59970946_{—} | PTBP1_chr19_+_805512_{—} |
| 57212764_57207781_57219942 | 59971033_59970286_59971790 | 805569_805187_806407 |
| TBX3_chr12_−_115117717_{—} | ASXL1_chr20_+_30959580_{—} | CARMIL1_chr6_+_25507706_{—} |
| 115117777_115117456_115118683 | 30959677_30956926_30959966 | 25507718_25500463_25509883 |
| OS9_chr12_+_58113881_{—} | EPOR_chr19_−_11493522_{—} | NASP_chr1_+_46082372_{—} |
| 58114046_58112965_58114188 | 11493625_11492781_11493772 | 46082375_46082065_46083134 |
| CACNAID_chr3_+_53752708_{—} | FAAP20_chr1_−_2117442_{—} | ING4_chr12_−_6764803_{—} |
| 53752768_53752415_53753781 | 2117688_2116952_2125077 | 6765079_6762216_6765892 |
| METTL25_chr12_+_82832496_{—} | NUTF2_chr16_+_67881180_{—} | UPP1_chr7_+_48139266_{—} |
| 82832570_82828503_82850505 | 67881359_67880888_67899004 | 48139384_48134424_48141420 |
| TANGO2_chr22_+_20040959_{—} | MAZ_chr16_+_29820860_{—} | ZNF7_chr8_+_146054862_{—} |
| 20041074_20040107_20043465 | 29821085_29820062_29821397 | 146054989_146052994_146062775 |
| MAP2K7_chr19_+_7970692_{—} | NAGPA_chr16_−_5078297_{—} | COMTD1_chr10_−_76995373_{—} |
| 7970740_7968953_7974639 | 5078399_5078186_5078880 | 76995501_76994936_76995592 |
| SRRM1_chr1_+_24973569_{—} | DPF2_chr11_+_65112050_{—} | NOP58_chr2_+_203142934_{—} |
| 24973699_24973280_24975349 | 65112092_65111540_65113136 | 203143052_203142725_203147073 |
| HNRNPH1_chr5_−_179046269_{—} | CCNL2_chr1_−_1328169_{—} | MED24_chr17_−_38191168_{—} |
| 179046408_179045324_179047892 | 1328183_1326245_1328775 | 38191225_38189709_38191369 |
| ZNF384_chr12_−_6781515_{—} | TKFC_chr11_+_61103311_{—} | MARK3_chr14_+_103966492_{—} |
| 6781698_6780004_6782381 | 61103409_61102203_61105412 | 103966537_103964865_103969218 |
| RAD51C_chr17_+_56783238_{—} | PILRA_chr7_+_99987510_{—} | MED31_chr17_−_6553301_{—} |
| 56783383_56780690_56787219 | 99987729_99972056_99995501 | 6553398_6547979_6553675 |
| FOLH1_chr11_−_49228329_{—} | MAPK12_chr22_−_50686120_{—} | TAF1C_chr16_−_84219085_{—} |
| 49228426_49227724_49229843 | 50686205_50685395_50686318 | 84219189_84218595_84220506 |
| ANK3_chr10_−_61905725_{—} | RNF217_chr6_+_125292646_{—} | NAGLU_chr17_+_40692967_{—} |
| 61905779_61898845_61926348 | 125292744_125284572_125366356 | 40693224_40690773_40695045 |
| C12orf73_chr12_−_104347191_{—} | TRMT2B_chrX_−_100306632_{—} | BTF3L4_chr1_+_52525506_{—} |
| 104347312_104345408_104350408 | 100306722_100297301_100306805 | 52525573_52522051_52530496 |
| ABCD4_chr14_−_74764632_{—} | MCEE_chr2_−_71356846_{—} | UFD1L_chr22_−_19462590_{—} |
| 74764772_74763152_74766250 | 71356947_71351673_71357309 | 19462623_19459331_19466605 |
| LMAN2L_chr2_−_97403685_{—} | S100A13_chr1_−_153602925_{—} | PAAF1_chr11_+_73597973_{—} |
| 97403804_97400263_97405590 | 153603132_153600074_153603486 | 73598144_73589864_73598398 |
| GUSB_chr7_−_65444385_{—} | C12orf49_chr12_−_117158100_{—} | CAB39L_chr13_−_49975268_{—} |
| 65444528_65441189_65445210 | 117158252_117155698_117175594 | 49975406_49957077_50007453 |
| HNRNPH3_chr10_+_70097614_{—} | SREK1_chr5_+_65454636_{—} | SPOP_chr17_−_47714120_{—} |
| 70097753_70097090_70098259 | 65454760_65449424_65455046 | 47714171_47700238_47723477 |
| LYNX1_chr8_−_143856758_{—} | LIAS_chr4_+_39469738_{—} | ACTN1_chr14_−_69345705_{—} |
| 143856781_143846558_143857010 | 39470047_39469266_39471638 | 69345786_69345240_69346678 |
| BCLAF1_chr6_−_136590278_{—} | IFT81_chr12_+_110581186_{—} | EVI5L_chr19_+_7921977_{—} |
| 136590441_136589477_136590574 | 110581350_110574665_110584757 | 7922010_7920954_7923076 |
| HDAC6_chrX_+_48678512_{—} | BCS1L_chr2_+_219524759_{—} | OTUD6B_chr8_+_92088819_{—} |
| 48678662_48676516_48681029 | 219524941_219524466_219525661 | 92088959_92086139_92090583 |
| INTS4_chr11_−_77622015_{—} | MACF1_chr1_+_39930766_{—} | GFM2_chr5_−_74026084_{—} |
| 77622198_77618856_77629866 | 39930784_39929358_39934286 | 74026223_74021951_74028846 |
| ARHGEF9_chrX_−_62947070_{—} | RNF167_chr17_+_4843781_{—} | TBC1D1_chr4_+_38053519_{—} |
| 62947299_62944591_62974194 | 4843937_4843525_4844167 | 38053681_38051519_38055819 |
| TMEM11_chr17_−_21114379_{—} | MSRB3_chr12_+_65702308_{—} | PDCD4_chr10_+_112636427_{—} |
| 21114540_21102153_21117403 | 65702435_65672645_65720605 | 112636511_112635828_112640990 |
| C2CD5_chr12_−_22611417_{—} | COQ4_chr9_+_131087421_{—} | MGST1_chr12_+_16507164_{—} |
| 22611519_22610095_22612425 | 131087518_131085426_131088057 | 16507204_16500644_16510538 |
| MDM1_chr12_−_68710360_{—} | MTFR1L_chr1_+_26149728_{—} | GARNL3_chr9_+_130111202_{—} |
| 68710390_68710033_68715126 | 26149814_26149619_26150134 | 130111314_130107756_130116124 |
| MSH5-SAPCD1_chr6_+_31713013_{—} | CROCCP2_chr1_−_16969261_{—} | EXD3_chr9_−_140269063_{—} |
| 31713096_31712358_31715163 | 16969345_16961663_16971140 | 140269215_140268051_140277764 |
| ABCD4_chr14_−_74756729_{—} | PRUNE1_chr1_+_150990942_{—} | FXR1_chr3_+_180688862_{—} |
| 74756821_74756222_74756993 | 150991145_150990380_150997086 | 180688943_180688146_180693100 |
| STX2_chr12_−_131280539_{—} | CUL7_chr6_−_43015684_{—} | DGKD_chr2_+_234377068_{—} |
| 131280665_131276522_131283069 | 43015727_43014845_43015885 | 234377199_234375849_234378016 |
| TMEM14B_chr6_+_10751365_{—} | MCF2L_chr13_+_113745434_{—} | PDLIM5_chr4_+_95506136_{—} |
| 10751467_10749931_10770309 | 113745509_113744042_113748827 | 95506154_95497185_95506715 |
| TTC38_chr22_+_46688099_{—} | VMP1_chr17_+_57911372_{—} | EED_chr11_+_85979497_{—} |
| 46688225_46685796_46688687 | 57911411_57895134_57915655 | 85979603_85977258_85988021 |
| PTGES2_chr9_−_130887057_{—} | KLHDC4_chr16_−_87790004_{—} | CSPP1_chr8_+_67999057_{—} |
| 130887150_130886829_130887522 | 87790083_87788898_87795554 | 67999081_67998345_68005777 |
| MFF_chr2_+_228217229_{—} | MBTD1_chr17_−_49286674_{—} | DGUOK_chr2_+_74184251_{—} |
| 228217289_228212100_228220392 | 49286791_49284366_49294694 | 74184367_74154179_74185272 |
| FNBP1_chr9_−_132686122_{—} | NUP50_chr22_+_45566868_{—} | RIPK2_chr8_+_90775056_{—} |
| 132686275_132671278_132687238 | 45567058_45564127_45567480 | 90775210_90770461_90777568 |
| PXN_chr12_−_120654075_{—} | DBN1_chr5_−_176886603_{—} | PAPD4_chr5_+_78952780_{—} |
| 120654919_120653464_120659425 | 176886741_176886269_176887432 | 78952824_78945013_78964714 |
| TRMU_chr22_+_46739158_{—} | DNAJC19_chr3_−_180704730_{—} | ACIN1_chr14_−_23559190_{—} |
| 46739265_46733841_46742318 | 180704810_180703784_180705810 | 23559310_23551045_23559730 |
| STK40_chr1_−_36833448_{—} | NBEA_chr13_+_36220005_{—} | DPH7_chr9_−_140470160_{—} |
| 36833685_36821034_36851323 | 36220068_36202385_36220395 | 140470344_140469295_140470531 |
| CBR4_chr4_−_169930102_{—} | MPND_chr19_+_4355093_{—} | FAM111A_chr11_+_58910969_{—} |
| 169930205_169928907_169931098 | 4355170_4354417_4357249 | 58913329_58910777_58919222 |
| SNRNP70_chr19_+_49605370_{—} | MRPL22_chr5_+_154330380_{—} | LINC00893_chrX_−_148619164_{—} |
| 49605442_49604728_49607890 | 154330498_154320825_154335930 | 148619310_148615906_148619646 |
| VCL_chr10_+_75871666_{—} | TAX1BP1_chr7_+_27855967_{—} | CENPO_chr2_+_25016720_{—} |
| 75871870_75868914_75873941 | 27856013_27839709_27867356 | 25016834_25016389_25022543 |
| GATA2_chr3_−_128210846_{—} | NFIX_chr19_+_13198802_{—} | SEMA4F_chr2_+_74884979_{—} |
| 128211065_128205919_128211741 | 13198950_13186485_13201112 | 74885078_74884751_74889858 |
| ATAD3B_chr1_+_1425071_{—} | ABI1_chr10_−_27044583_{—} | SVIL_chr10_−_29820930_{—} |
| 1425191_1424654_1425636 | 27044670_27040712_27047990 | 29821101_29820217_29821457 |
| SSBP4_chr19_+_18542162_{—} | RBM38_chr20_+_55968334_{—} | PQLC1_chr18_−_77690227_{—} |
| 18542228_18541740_18542456 | 55968389_55967833_55982598 | 77690311_77679400_77693968 |
| GPS1_chr17_+_80010131_{—} | FDPS_chr1_+_155279579_{—} | CSAD_chr12_−_53554910_{—} |
| 80010335_80009840_80011149 | 155279756_155278756_155279833 | 53554975_53554628_53555056 |
| ZNF445_chr3_−_44499735_{—} | TM2D3_chr15_−_102191898_{—} | RFC5_chr12_+_118455494_{—} |
| 44499856_44497188_44519082 | 102191976_102190364_102192473 | 118455858_118454697_118456876 |
| PPP1R35_chr7_−_100033253_{—} | KTN1_chr14_+_56130672_{—} | TRIQK_chr8_−_93933730_{—} |
| 100033390_100033156_100033470 | 56130759_56128330_56133958 | 93933888_93904302_93966633 |
| IMPDH1_chr7_−_128033775_{—} | MAP4K4_chr2_+_102477286_{—} | TTLL3_chr3_+_9874787_{—} |
| 128033792_128033082_128034331 | 102477448_102476326_102481391 | 9874957_9871079_9876364 |
| SEC11A_chr15_−_85223162_{—} | DDX60L_chr4_−_169382849_{—} | CERS5_chr12_−_50535835_{—} |
| 85223200_85214013_85230855 | 169383191_169379159_169392897 | 50535893_50532400_50536856 |
| ZNF326_chr1_+_90472903_{—} | FNBP1_chr9_−_132686122_{—} | ZNF160_chr19_−_53594665_{—} |
| 90473309_90470803_90475646 | 132686305_132671278_132687238 | 53594782_53589574_53594889 |
| APBB3_chr5_−_139941428_{—} | KLK4_chr19_−_51411614_{—} | NPTN_chr15_−_73884306_{—} |
| 139941434_139941286_139941684 | 51411747_51410342_51411834 | 73884410_73866136_73925465 |
| INTS11_chr1_−_1258560_{—} | SF3B3_chr16_+_70584552_{—} | RNPS1_chr16_−_2314573_{—} |
| 1258667_1257364_1259960 | 70584581_70582345_70588348 | 2314761_2314332_2318055 |
| EIF3M_chr11_+_32610557_{—} | NKTR_chr3_+_42661507_{—} | LINC00963_chr9_+_132264675_{—} |
| 32610681_32605475_32611092 | 42661535_42661200_42662920 | 132265418_132251578_132265601 |
| SMARCA4_chr19_+_11144442_{—} | ST7L_chr1_−_113159434_{—} | SLC37A2_chr11_+_124956099_{—} |
| 11144541_11144193_11144798 | 113159517_113153625_113161530 | 124956156_124955914_124958014 |
| PDIA5_chr3_+_122849326_{—} | WSB1_chr17_+_25624212_{—} | ZNF644_chr1_−_91403828_{—} |
| 122849463_122843190_122864368 | 25624334_25621461_25630392 | 91406866_91383711_91447866 |
| ST3GAL3_chr1_+_44386453_{—} | CAD_chr2_+_27463434_{—} | USP54_chr10_−_75279554_{—} |
| 44386600_44365399_44395803 | 27463485_27463229_27463778 | 75279750_75277505_75283340 |
| NFYB_chr12_−_104525531_{—} | PXN_chr12_−_120653362_{—} | MMS19_chr10_−_99236591_{—} |
| 104525616_104522295_104529225 | 120653464_120653076_120659425 | 99236720_99236501_99237103 |
| KIF21A_chr12_−_39724043_{—} | HNRNPLL_chr2_−_38805061_{—} | IP6K2_chr3_−_48752747_{—} |
| 39724064_39720126_39724547 | 38805144_38804674_38809054 | 48752960_48732854_48754589 |
| TOMM34_chr20_−_43577370_{—} | VIL1_chr2_+_219299248_{—} | LRRFIP1_chr2_+_238647874_{—} |
| 43577518_43572220_43580473 | 219299428_219297674_219300011 | 238647952_238629465_238657006 |
| FAM76B_chr11_−_95512241_{—} | AP4M1_chr7_+_99699868_{—} | COL6A3_chr2_−_238287278_{—} |
| 95512299_95512121_95512770 | 99700016_99699591_99700297 | 238287878_238285987_238289557 |
| SLC12A7_chr5_−_1056696_{—} | MRPL55_chr1_−_228296655_{—} | ARIH2_chr3_+_48982568_{—} |
| 1056711_1053597_1057585 | 228296722_228295570_228296961 | 48982614_48965246_48999044 |
| CYTH1_chr17_−_76692088_{—} | TRNAU1AP_chr1_+_28887624_{—} | ARFGAP2_chr11_−_47194260_{—} |
| 76692091_76688575_76694350 | 28887772_28887244_28887857 | 47194302_47193884_47196565 |
| KLHDC4_chr16_−_87790027_{—} | ZNF548_chr19_+_57904168_{—} | AAMDC_chr11_+_77552064_{—} |
| 87790083_87788898_87795554 | 57904316_57901482_57905555 | 77552106_77532287_77553524 |
| PNPLA8_chr7_−_108161919_{—} | HDAC7_chr12_−_48186320_{—} | PDE2A_chr11_−_72308553_{—} |
| 108161965_108155891_108166472 | 48186452_48185787_48187151 | 72308663_72302400_72316181 |
| RANBP3_chr19_−_5950727_{—} | GGT1_chr22_+_25004956_{—} | CHURC1_chr14_+_65392727_{—} |
| 5950886_5941846_5951403 | 25005198_25003990_25006324 | 65392798_65390844_65398855 |
| ZNF160_chr19_−_53594665_{—} | TBCK_chr4_−_107158647_{—} | ECHDC2_chr1_−_53370705_{—} |
| 53594750_53589574_53594889 | 107158695_107157965_107163626 | 53370762_53370505_53373539 |
| GIT2_chr12_−_110383064_{—} | RFC5_chr12_+_118455799_{—} | NDUFAF6_chr8_+_96056704_{—} |
| 110383154_110377052_110385060 | 118455858_118454697_118456876 | 96056823_96053854_96057772 |
| ATG12_chr5_−_115176193_{—} | SORBS2_chr4_−_186611715_{—} | GALNS_chr16_−_88909437_{—} |
| 115176309_115173461_115177086 | 186611765_186599976_186696380 | 88909607_88909237_88923165 |
| CHKA_chr11_−_67828959_{—} | OXNAD1_chr3_+_16313651_{—} | PLA2G5_chr1_+_20415317_{—} |
| 67829297_67821514_67829419 | 16313828_16313229_16327848 | 20415394_20412720_20417060 |
| KIAA0141_chr5_+_141318420_{—} | NAXD_chr13_+_111274562_{—} | TCF12_chr15_+_57544618_{—} |
| 141318469_141318325_141319070 | 111274713_111267994_111286891 | 57544690_57543621_57545459 |
| SETD3_chr14_−_99927528_{—} | DDX39A_chr19_−_14521359_{—} | GUF1_chr4_+_44690023_{—} |
| 99927677_99925522_99929822 | 14521417_14521146_14521800 | 44690163_44688730_44691302 |
| NDEL1_chr17_+_8366637_{—} | CARS_chr11_−_3068982_{—} | SF1_chr11_−_64540901_{—} |
| 8366672_8363478_8370247 | 3069231_3063486_3078572 | 64540977_64537880_64543969 |
| GOLGA4_chr3_+_37402733_{—} | SLK_chr10_+_105770573_{—} | NDUFB5_chr3_+_179333770_{—} |
| 37402796_37396678_37407570 | 105770666_105768114_105777917 | 179333837_179322727_179334770 |
| METTL13_chr1_+_171759591_{—} | NDUFA7_chr19_−_8384184_{—} | CAMK2G_chr10_−_75599296_{—} |
| 171759756_171757070_171761156 | 8384278_8381529_8386191 | 75599359_75597269_75601926 |
| DPY30_chr2_−_32248745_{—} | LCN10_chr9_−_139635703_{—} | UBE3A_chr15_−_25652213_{—} |
| 32248877_32248543_32254662 | 139635813_139635395_139636332 | 25652284_25650649_25654234 |
| PTDSS2_chr11_+_473894_{—} | RPAIN_chr17_+_5331390_{—} | BCAT2_chr19_−_49310256_{—} |
| 473977_460288_479084 | 5331531_5329619_5335861 | 49310331_49303554_49314240 |
| TIAL1_chr10_−_121339982_{—} | DENND5A_chr11_−_9164949_{—} | PHYKPL_chr5_−_177642276_{—} |
| 121340358_121339522_121341433 | 9165032_9164392_9165643 | 177642431_177641886_177649355 |
| NPEPL1_chr20_+_57280553_{—} | MRPL55_chr1_−_228296137_{—} | HERC4_chr10_−_69718869_{—} |
| 57280625_57276214_57282178 | 228296722_228296019_228296961 | 69718893_69716733_69726439 |
| NFASC_chr1_+_204960419_{—} | DNAJC2_chr7_−_102967778_{—} | PHKG2_chr16_+_30767675_{—} |
| 204960434_204957934_204978684 | 102967825_102967131_102968102 | 30767841_30764878_30767910 |
| ATG9A_chr2_−_220093155_{—} | FHOD3_chr18_+_34267090_{—} | CCDC90B_chr11_−_82989768_{—} |
| 220093204_220092775_220094256 | 34267141_34261533_34273171 | 82989872_82985783_82991183 |
| RAB4B-EGLN2_chr19_+_41289682_{—} | FAM193B_chr5_−_176963358_{—} | RABGAP1L_chr1_+_174927027_{—} |
| 41289745_41286404_41289825 | 176963487_176959642_176964873 | 174927310_174926686_174938413 |
| DPY30_chr2_−_32108454_{—} | ADAM15_chr1_+_155033893_{—} | MAPT_chr17_+_44060543_{—} |
| 32108531_32095021_32142994 | 155033965_155033308_155034720 | 44061296_44055806_44064405 |
| DGUOK_chr2_+_74177727_{—} | KLHL24_chr3_+_183354008_{—} | ANKRD10_chr13_−_111552876_{—} |
| 74177859_74154179_74185272 | 183354099_183353581_183361267 | 111553041_111545610_111558379 |
| ARMCX2_chrX_−_100913446_{—} | D2HGDH_chr2_+_242688279_{—} | GEMIN4_chr17_−_653053_{—} |
| 100913555_100913128_100914742 | 242689344_242684292_242689565 | 653149_651272_655372 |
| DPY30_chr2_−_32117060_{—} | MYH11_chr16_−_15802659_{—} | PALM_chr19_+_740351_{—} |
| 32117203_32095021_32142994 | 15802698_15797980_15808765 | 740483_736078_746284 |
| NR1H3_chr11_+_47280730_{—} | GUSB_chr7_−_65444713_{—} | MTMR12_chr5_−_32233066_{—} |
| 47280810_47270550_47281341 | 65444898_65441189_65445210 | 32233126_32230453_32233878 |
| CLASP2_chr3_−_33638202_{—} | TRDN_chr6_−_123696749_{—} | PIGQ_chr16_+_631198_{—} |
| 33638226_33633988_33644443 | 123696776_123687327_123698860 | 631341_630972_632247 |
| RP11-43F13.1_chr5_−_1630523_{—} | AP1G2_chr14_−_24035770_{—} | PBRM1_chr3_−_52588739_{—} |
| 1630681_1629745_1632959 | 24035895_24035628_24036319 | 52588895_52584833_52592264 |
| ARF1_chr1_+_228284151_{—} | HERC2P9_chr15_+_28900640_{—} | PEX7_chr6_+_137167210_{—} |
| 228284205_228270551_228284778 | 28900837_28900566_28900941 | 137167319_137147607_137187764 |
| GABBR1_chr6_−_29577005_{—} | PHYKPL_chr5_−_177639973_{—} | HMOX2_chr16_+_4533637_{—} |
| 29577156_29576510_29578700 | 177640104_177638971_177641796 | 4533739_4526488_4556895 |
| PDDC1_chr11_−_773427_{—} | ZC3H7A_chr16_−_11862886_{—} | ECHDC2_chr1_−_53372190_{—} |
| 773629_772521_774007 | 11862951_11862357_11864638 | 53372283_53370505_53373539 |
| C1orf116_chr1_−_207198231_{—} | PYROXD2_chr10_−_100147621_{—} | PLPP1_chr5_−_54786787_{—} |
| 207198409_207196825_207205923 | 100147841_100147064_100148110 | 54786942_54771278_54830399 |
| MYL6_chr12_+_56553281_{—} | PRRC2C_chr1_+_171560290_{—} | UHRF2_chr9_+_6492302_{—} |
| 56553406_56552495_56553758 | 171560339_171557644_171560725 | 6492401_6486925_6493825 |
| BCAP29_chr7_+_107256705_{—} | SIRT2_chr19_−_39389018_{—} | GSTM4_chr1_+_110200211_{—} |
| 107256834_107253877_107258772 | 39389065_39384611_39390145 | 110200293_110199901_110200393 |
| LRRC27_chr10_+_134162466_{—} | TRAPPC2_chrX_−_13752162_{—} | TCF3_chr19_−_1632330_{—} |
| 134162561_134161860_134165110 | 13752304_13738101_13752643 | 1632404_1627425_1646353 |
| GNPTAB_chr12_−_102150197_{—} | DNM2_chr19_+_10919244_{—} | METTL6_chr3_−_15457004_{—} |
| 102150344_102147317_102150989 | 10919256_10916643_10922939 | 15457090_15455669_15457278 |
| PMF1_chr1_+_156195347_{—} | ALKBH1_chr14_−_78161080_{—} | INO80E_chr16_+_30012532_{—} |
| 156195459_156182967_156203418 | 78161243_78146313_78170711 | 30014334_30012361_30016541 |
| UVSSA_chr4_+_1374428_{—} | TMEM62_chr15_+_43470804_{—} | PLOD2_chr3_−_145795648_{—} |
| 1374535_1374018_1374667 | 43470909_43461875_43473378 | 145795711_145794682_145796902 |
| NEB_chr2_−_152371331_{—} | PLEKHA1_chr10_+_124187791_{—} | CSNK1A1_chr5_−_148897356_{—} |
| 152371442_152370942_152372972 | 124187936_124186547_124189139 | 148897440_148892772_148899852 |
| IRF6_chr1_−_209975316_{—} | PMPCB_chr7_+_102948042_{—} | COL16A1_chr1_−_32136202_{—} |
| 209975388_209969897_209979291 | 102948155_102944937_102949398 | 32136247_32134456_32137215 |
| PRR3_chr6_+_30529104_{—} | DPP9_chr19_−_4683201_{—} | CD27-AS1_chr12_−_6560058_{—} |
| 30529285_30525227_30529610 | 4683336_4682850_4683488 | 6560146_6557903_6560634 |
| TFDP1_chr13_+_114285937_{—} | B3GALNT2_chr1_−_235652472_{—} | CSAD_chr12_−_53565109_{—} |
| 114286220_114277601_114287434 | 235652573_235647831_235657990 | 53565225_53564286_53565665 |
| CCDC50_chr3_+_191092850_{—} | NUMA1_chr11_−_71723446_{—} | POGK_chr1_+_166815848_{—} |
| 191093378_191087825_191097947 | 71723488_71721900_71723940 | 166815975_166810325_166818174 |
| BPTF_chr17_+_65959448_{—} | PPT2-EGFL8_chr6_+_32122806_{—} | SMYD2_chr1_+_214478529_{—} |
| 65959622_65955991_65960327 | 32122960_32122554_32123464 | 214478593_214454770_214488104 |
| ISOC2_chr19_−_55967002_{—} | SEC31A_chr4_−_83763292_{—} | MINDY3_chr10_−_15880226_{—} |
| 55967212_55966697_55967715 | 83763634_83750211_83765538 | 15880278_15879317_15883424 |
| GAS6_chr13_−_114550998_{—} | ABHD14B_chr3_−_52005475_{—} | SYNE4_chr19_−_36494514_{—} |
| 114551023_114542823_114566547 | 52005908_52004200_52007980 | 36494573_36494422_36496234 |
| APBB3_chr5_−_139941428_{—} | ZDHHC20_chr13_−_21999790_{—} | HEXA_chr15_−_72646031_{—} |
| 139941580_139941286_139941684 | 21999817_21987911_22033192 | 72646078_72645519_72647899 |
| GUK1_chr1_+_228328824_{—} | SPIDR_chr8_+_48641973_{—} | FAM192A_chr16_−_57208531_{—} |
| 228329208_228328064_228333211 | 48642027_48626203_48647868 | 57208600_57207781_57219732 |
| NDUFS8_chr11_+_67799618_{—} | CADPS_chr3_−_62516328_{—} | IGFLR1_chr19_−_36230610_{—} |
| 67799676_67798200_67803719 | 62516487_62503925_62518545 | 36230670_36230527_36231924 |
| CD44_chr11_+_35219667_{—} | UBE2B_chr5_+_133712359_{—} | GPS1_chr17_+_80010250_{—} |
| 35219793_35211612_35232792 | 133712385_133710154_133724015 | 80010335_80009840_80011149 |
| FAM126A_chr7_−_22986570_{—} | ASPH_chr8_−_62594997_{—} | GUSB_chr7_−_65444820_{—} |
| 22986866_22985782_22999874 | 62595042_62593595_62596597 | 65444898_65441189_65445210 |
| BIRC6_chr2_+_32815872_{—} | AGAP3_chr7_+_150817606_{—} | UTP6_chr17_−_30205720_{—} |
| 32816045_32800433_32818981 | 150817832_150817232_150820880 | 30205800_30205346_30207591 |
| SNRNP70_chr19_+_49605370_{—} | TP53I11_chr11_−_44958109_{—} | PTPMT1_chr11_+_47591251_{—} |
| 49605430_49604728_49607890 | 44958240_44957213_44958353 | 47591443_47587538_47593022 |
| BLOC1S6_chr15_+_45895301_{—} | SCAP_chr3_−_47467486_{—} | DNM1_chr9_+_131010202_{—} |
| 45895385_45879723_45897625 | 47467659_47464026_47476497 | 131010214_131009765_131010861 |
| LPIN2_chr18_−_2928588_{—} | TPM1_chr15_+_63356262_{—} | ERMARD_chr6_+_170153382_{—} |
| 2928658_2927809_2929062 | 63356389_63354844_63358094 | 170153468_170151759_170153959 |
| PDLIM7_chr5_−_176918404_{—} | RNASET2_chr6_−_167362053_{—} | TBRG4_chr7_−_45143697_{—} |
| 176918421_176918147_176918807 | 167362113_167360227_167365975 | 45143855_45143042_45144136 |
| MTERF4_chr2_−_242035807_{—} | ZC3H11A_chr1_+_203803064_{—} | ELMOD3_chr2_+_85614220_{—} |
| 242035853_242033847_242036657 | 203803230_203802981_203807093 | 85614348_85604597_85616873 |
| POLL_chr10_−_103345618_{—} | BIN1_chr2_−_127811480_{—} | RAN_chr12_+_131357128_{—} |
| 103345913_103344676_103347002 | 127811588_127808819_127815048 | 131357162_131356671_131357380 |
| RBM5_chr3_+_50147811_{—} | REPS1_chr6_−_139247537_{—} | BTBD1_chr15_−_83697367_{—} |
| 50147896_50147121_50148111 | 139247618_139242261_139251113 | 83697480_83689514_83698887 |
| AKR1A1_chr1_+_46034156_{—} | FOPNL_chr16_−_15976805_{—} | TSNAX_chr1_+_231699210_{—} |
| 46034356_46033849_46034598 | 15976877_15973745_15977864 | 231699375_231697001_231700273 |
| KANSL2_chr12_−_49056342_{—} | RHOC_chr1_−_113247721_{—} | GLT8D1_chr3_−_52738739_{—} |
| 49056437_49054402_49061475 | 113247745_113246428_113249699 | 52738964_52731950_52739462 |
| MICAL3_chr22_−_18310409_{—} | ZNF207_chr17_+_30693683_{—} | FKRP_chr19_+_47251771_{—} |
| 18310547_18305826_18314619 | 30693776_30692506_30694790 | 47251974_47251345_47258668 |
| SYTL1_chr1_+_27676462_{—} | SPAG9_chr17_−_49053223_{—} | EBPL_chr13_−_50243912_{—} |
| 27676623_27676256_27676879 | 49053262_49052308_49054468 | 50243982_50237331_50265389 |
| TAF1D_chr11_−_93466515_{—} | DMPK_chr19_−_46274228_{—} | SLC35B3_chr6_−_8422702_{—} |
| 93466563_93463878_93467790 | 46274314_46273898_46274825 | 8422857_8421061_8428169 |
| ACSF2_chr17_+_48541886_{—} | ACTN1_chr14_−_69345174_{—} | ZBTB1_chr14_+_64983317_{—} |
| 48541960_48541655_48548388 | 69345240_69343957_69345705 | 64983469_64971664_64988204 |
| AKAP8L_chr19_−_15509440_{—} | PPHLN1_chr12_+_42745686_{—} | GOLGB1_chr3_−_121387160_{—} |
| 15509514_15508666_15510112 | 42745851_42729776_42748962 | 121387355_121387005_121388058 |
| ZFAS1_chr20_+_47897439_{—} | METTL26_chr16_−_685611_{—} | NADSYN1_chr11_+_71187078_{—} |
| 47897501_47897107_47905581 | 685774_685340_686093 | 71188484_71185572_71189440 |
| NUDT9_chr4_+_88366473_{—} | SPATA5L1_chr15_+_45699226_{—} | RMDN1_chr8_−_87497100_{—} |
| 88366659_88363067_88370293 | 45699421_45697703_45702569 | 87497190_87492561_87498712 |
| PVT1_chr8_+_129010445_{—} | HAUS2_chr15_+_42852979_{—} | RHOT1_chr17_+_30536368_{—} |
| 129010605_129001537_129021835 | 42853068_42851606_42853467 | 30536464_30535328_30538134 |
| BPTF_chr17_+_65871671_{—} | CYP4A11_chr1_−_47398654_{—} | NDRG2_chr14_−_21492134_{—} |
| 65871860_65871136_65887959 | 47398719_47398509_47399617 | 21492255_21491480_21493187 |
| U2SURP_chr3_+_142742816_{—} | IL18BP_chr11_+_71710273_{—} | NEK1_chr4_−_170476870_{—} |
| 142742860_142741906_142745990 | 71710708_71710065_71710972 | 170477002_170459062_170477082 |
| FAR2P2_chr2_−_131183738_{—} | RBM6_chr3_+_50036872_{—} | SPPL2A_chr15_−_51018269_{—} |
| 131183836_131182731_131185276 | 50036946_50012825_50085677 | 51018323_51017520_51018517 |
| PGAP3_chr17_−_37832866_{—} | SNRPA1_chr15_−_101833229_{—} | THRAP3_chr1_+_36724982_{—} |
| 37833008_37830932_37840849 | 101833377_101827907_101835301 | 36725085_36690106_36748133 |
| POLDIP3_chr22_−_42997975_{—} | MUC20_chr3_+_195449371_{—} | C17orf62_chr17_−_80407049_{—} |
| 42998113_42995799_42998775 | 195449689_195447954_195451550 | 80407168_80404572_80408575 |
| WDR73_chr15_−_85189203_{—} | NEB_chr2_−_152350288_{—} | RPS15_chr19_+_1439604_{—} |
| 85189316_85189067_85189414 | 152350381_152349008_152350674 | 1439825_1438891_1440024 |
| CTBP1_chr4_−_1235112_{—} | EIF2AK4_chr15_+_40324957_{—} | USMG5_chr10_−_105153955_{—} |
| 1235307_1232125_1242703 | 40325002_40322657_40326526 | 105154151_105152223_105155502 |
| MRNIP_chr5_−_179280196_{—} | NFE2L1_chr17_+_46134393_{—} | PRR34-AS1_chr22_+_46451478_{—} |
| 179280276_179275066_179280377 | 46134483_46133960_46134705 | 46451613_46450833_46452726 |
| ABI1_chr10_−_27044583_{—} | TRIP4_chr15_+_64706283_{—} | QTRT2_chr3_+_113786833_{—} |
| 27044670_27040712_27054146 | 64706410_64702027_64710739 | 113786910_113785130_113789472 |
| ZC3H14_chr14_+_89069171_{—} | MMS19_chr10_−_99228722_{—} | TIRAP_chr11_+_126159559_{—} |
| 89069389_89044484_89073586 | 99228861_99228163_99229402 | 126159712_126153048_126160697 |
| TMEM62_chr15_+_43430771_{—} | TBXAS1_chr7_+_139482464_{—} | NPRL3_chr16_−_174935_{—} |
| 43430817_43427847_43438690 | 139482528_139478270_139487136 | 175072_169254_188148 |
| FKRP_chr19_+_47251771_{—} | SS18_chr18_−_23615794_{—} | KIF21A_chr12_−_39711874_{—} |
| 47252141_47251345_47258668 | 23615887_23615091_23618518 | 39712003_39705355_39713707 |
| ATP8A2P1_chr10_+_37598024_{—} | MAN2B1_chr19_−_12769241_{—} | EZH1_chr17_−_40869993_{—} |
| 37598108_37553565_37604623 | 12769321_12769158_12772073 | 40870085_40865407_40872290 |
| FAXDC2_chr5_−_154214184_{—} | TEP1_chr14_−_20840891_{—} | SERPINC1_chr1_−_173884155_{—} |
| 154214288_154210482_154214402 | 20841016_20839791_20841169 | 173884324_173884057_173886356 |
| VCAN_chr5_+_82815167_{—} | GALM_chr2_+_38925507_{—} | NUP35_chr2_+_184016235_{—} |
| 82818128_82808215_82841355 | 38925578_38917036_38956697 | 184016377_183998351_184022171 |
| ATXN2L_chr16_+_28847646_{—} | ADAM15_chr1_+_155034379_{—} | CCSER2_chr10_+_86259630_{—} |
| 28847811_28847497_28848048 | 155034451_155033308_155034720 | 86259715_86237420_86273204 |
| TRIM14_chr9_−_100872350_{—} | EIF2D_chr1_−_206772816_{—} | ABHD16A_chr6_−_31669850_{—} |
| 100872516_100872266_100881263 | 206772966_206772446_206773616 | 31669907_31668805_31670926 |
| CD44_chr11_+_35229651_{—} | PTRH2_chr17_−_57776231_{—} | ZNF761_chr19_+_53948253_{—} |
| 35229753_35211612_35232792 | 57777550_57775339_57784731 | 53948376_53935281_53950448 |
| TMEM8B_chr9_+_35835007_{—} | NSMF_chr9_−_140350080_{—} | SGSM3_chr22_+_40797596_{—} |
| 35835215_35829952_35841130 | 140350086_140349759_140350862 | 40797679_40796817_40798142 |
| AP2A2_chr11_+_1000431_{—} | FAM131A_chr3_+_184056174_{—} | FAM219B_chr15_−_75197322_{—} |
| 1000598_994245_1003721 | 184056317_184053911_184059511 | 75197400_75197053_75197494 |
| CD27-AS1_chr12_−_6559639_{—} | ZNF638_chr2_+_71649943_{—} | ZFYVE21_chr14_+_104196129_{—} |
| 6559874_6557903_6560634 | 71651168_71645769_71651779 | 104196183_104195519_104198956 |
| DGUOK_chr2_+_74166036_{—} | ADGRL2_chr1_+_82418670_{—} | FAM193B_chr5_−_176959156_{—} |
| 74166149_74154179_74185272 | 82418709_82417826_82421560 | 176959201_176958522_176959443 |
| TBC1D15_chr12_+_72288104_{—} | FAM193B_chr5_−_176974168_{—} | PRUNE2_chr9_−_79234255_{—} |
| 72288155_72287104_72288465 | 176974229_176966148_176981249 | 79234303_79229516_79239938 |
| SYNE1_chr6_−_152451854_{—} | TBP_chr6_+_170876313_{—} | PTCD3_chr2_+_86346666_{—} |
| 152451913_152443811_152453256 | 170876593_170876097_170878699 | 86346737_86346167_86348601 |
| NSUN5P2_chr7_−_72422690_{—} | AFMID_chr17_+_76201683_{—} | CSDE1_chr1_−_115284147_{—} |
| 72422834_72420735_72425163 | 76201834_76200981_76202026 | 115284294_115282511_115300545 |
| HSD17B6_chr12_+_57157001_{—} | WNK1_chr12_+_988738_{—} | KLC1_chr14_+_104158695_{—} |
| 57157198_57146102_57167617 | 989197_987527_989886 | 104158762_104153548_104166991 |
| HINFP_chr11_+_119002952_{—} | SYNE4_chr19_−_36497324_{—} | FAR2P3_chr2_+_131452809_{—} |
| 119003007_119002692_119003205 | 36497573_36496339_36498026 | 131452907_131451369_131453914 |
| DPP8_chr15_−_65748049_{—} | NUDT2_chr9_+_34336297_{—} | TNK2_chr3_−_195612283_{—} |
| 65748179_65746753_65748562 | 34336339_34329597_34338710 | 195612414_195611904_195613847 |
| AHSA2_chr2_+_61410681_{—} | SPTLC1_chr9_−_94841715_{—} | HEMK1_chr3_+_50615256_{—} |
| 61410797_61408540_61411846 | 94841848_94830380_94842297 | 50615306_50615004_50617274 |
| ARFIP1_chr4_+_153791904_{—} | PHYKPL_chr5_−_177650320_{—} | TEAD2_chr19_−_49859215_{—} |
| 153792000_153784866_153793603 | 177650452_177649935_177651616 | 49859227_49858676_49860508 |
| GSKIP_chr14_+_96846024_{—} | NFIB_chr9_−_14116206_{—} | SMPDL3A_chr6_+_123116821_{—} |
| 96846092_96829905_96848583 | 14116345_14088325_14120438 | 123117035_123110603_123117968 |
| FBLN2_chr3_+_13663274_{—} | TTC7A_chr2_+_47185633_{—} | SGCA_chr17_+_48244942_{—} |
| 13663415_13661331_13667944 | 47185691_47184146_47202111 | 48245097_48244848_48245307 |
| ARID4B_chr1_−_235377083_{—} | CPNE4_chr3_−_131753410_{—} | CAPN10_chr2_+_241535350_{—} |
| 235377341_235359430_235383107 | 131753603_131624288_131756400 | 241535569_241534721_241535735 |
| RILP_chr17_−_1550199_{—} | AC005154.6_chr7_−_30590933_{—} | MRPS28_chr8_−_80915233_{—} |
| 1550283_1549913_1551128 | 30591095_30590397_30603346 | 80915415_80831383_80942270 |
| BRD2_chr6_+_32942797_{—} | RP4-539M6.19_chr22_+_30811936_{—} | CCAR1_chr10_+_70516029_{—} |
| 32942889_32942542_32943160 | 30812076_30806668_30812222 | 70516240_70515293_70517050 |
| CAST_chr5_+_96062497_{—} | PHYKPL_chr5_−_177658669_{—} | HEATR5B_chr2_−_37217790_{—} |
| 96062563_96058402_96063192 | 177658867_177658524_177659492 | 37217942_37216002_37227728 |
| EIF4A2_chr3_+_186506098_{—} | MICAL3_chr22_−_18295272_{—} | SRSF1_chr17_−_56082758_{—} |
| 186506205_186505671_186506913 | 18295323_18293579_18299455 | 56082961_56082402_56083161 |
| SLTM_chr15_−_59204761_{—} | CNDP2_chr18_+_72164743_{—} | PTBP1_chr19_+_806407_{—} |
| 59204809_59193486_59209133 | 72164830_72163666_72167116 | 806556_799443_808359 |
| RNH1_chr11_−_504823_{—} | MBNL1_chr3_+_152174055_{—} | ARVCF_chr22_−_19958738_{—} |
| 504996_502181_507112 | 152174150_152165562_152177059 | 19958858_19958266_19959408 |
| PCSK7_chr11_−_117078369_{—} | STYXL1_chr7_−_75643059_{—} | GOLGA8A_chr15_−_34727583_{—} |
| 117078451_117077876_117078685 | 75643205_75634722_75651168 | 34727672_34699936_34729598 |
| ECHDC2_chr1_−_53370705_{—} | FAR2P3_chr2_+_131453039_{—} | GAA_chr17_+_78075609_{—} |
| 53370762_53364896_53373539 | 131453164_131451369_131453914 | 78075724_78075424_78078353 |
| SFXN2_chr10_+_104487431_{—} | PDDC1_chr11_−_773521_{—} | SBF1_chr22_−_50895462_{—} |
| 104487548_104486914_104488187 | 773629_772521_774007 | 50895540_50895102_50897683 |
| ARIH2_chr3_+_48962150_{—} | FAM221A_chr7_+_23731801_{—} | WRNIP1_chr6_+_2770353_{—} |
| 48962404_48960244_48964894 | 23734532_23731215_23740404 | 2770595_2766678_2779496 |
| FAM206A_chr9_+_111698587_{—} | MAN2B1_chr19_−_12769241_{—} | MFSD11_chr17_+_74739479_{—} |
| 111698717_111697969_111712786 | 12769324_12769158_12772073 | 74739538_74738355_74740403 |
| APLP2_chr11_+_129993506_{—} | GOSR2_chr17_+_45012394_{—} | TXNDC16_chr14_−_52929529_{—} |
| 129993674_129992408_129996594 | 45012591_45009565_45015964 | 52929688_52923892_52936754 |
| CPNE4_chr3_−_131753410_{—} | C11orf49_chr11_+_47013093_{—} | NPHP3_chr3_−_132415574_{—} |
| 131754286_131624288_131756400 | 47013198_46958402_47073938 | 132415657_132413809_132416103 |
| DFFB_chr1_+_3782847_{—} | EPB41L2_chr6_−_131201283_{—} | RANBP3_chr19_−_5950727_{—} |
| 3782962_3782564_3784537 | 131201346_131191266_131206235 | 5950883_5941846_5951403 |
| PCBP4_chr3_−_51996825_{—} | EPB41_chr1_+_29386933_{—} | YPEL5_chr2_+_30371110_{—} |
| 51996908_51996104_52001341 | 29386996_29385157_29391493 | 30371407_30369928_30379493 |
| TAMM41_chr3_−_11868191_{—} | NT5C2_chr10_−_104860508_{—} | ARFGAP2_chr11_−_47194260_{—} |
| 11868279_11858811_11871187 | 104860700_104859776_104860801 | 47194302_47193884_47197401 |
| ATP2A2_chr12_+_110785197_{—} | PLXNB2_chr22_−_50733147_{—} | MORF4L2_chrX_−_102939608_{—} |
| 110785258_110784126_110788096 | 50733207_50729026_50745981 | 102939657_102931979_102940098 |
| IMMP1L_chr11_−_31484718_{—} | RCC1_chr1_+_28834639_{—} | KLK4_chr19_−_51411614_{—} |
| 31484852_31455117_31531065 | 28834672_28832596_28843236 | 51411751_51410342_51411834 |
| AFMID_chr17_+_76200736_{—} | CPSF7_chr11_−_61188663_{—} | RBM26_chr13_−_79927287_{—} |
| 76200822_76198832_76200908 | 61188729_61188045_61188861 | 79927359_79918929_79928573 |
| SLC4A7_chr3_−_27472788_{—} | TMEM62_chr15_+_43430771_{—} | TMEM214_chr2_+_27260657_{—} |
| 27473160_27465643_27475406 | 43430817_43427847_43440952 | 27260760_27260570_27261013 |
| MCRIP2_chr16_+_696471_{—} | OARD1_chr6_−_41036579_{—} | EXOC7_chr17_−_74086409_{—} |
| 696608_692249_697416 | 41036692_41035176_41038870 | 74086562_74085401_74090494 |
| GUSB_chr7_−_65444820_{—} | ZC3H14_chr14_+_89069171_{—} | XPA_chr9_−_100444481_{—} |
| 65444898_65444528_65445210 | 89069404_89044484_89073586 | 100444712_100437869_100447204 |
| SRSF7_chr2_−_38976039_{—} | AMDHD2_chr16_+_2577573_{—} | NRDC_chr1_−_52302040_{—} |
| 38976488_38975795_38976670 | 2577616_2571124_2577773 | 52302110_52301884_52305897 |
| TOM1L2_chr17_−_17761082_{—} | SRSF1_chr17_−_56082774_{—} | TMEM5_chr12_+_64176320_{—} |
| 17761169_17754266_17764789 | 56082961_56082402_56083161 | 64176484_64174954_64178749 |
| HACL1_chr3_−_15628031_{—} | NSUN5P1_chr7_+_75043917_{—} | DCAF6_chr1_+_167988782_{—} |
| 15628109_15624496_15631046 | 75044076_75042210_75044162 | 167988905_167974031_167992225 |
| LRRFIP2_chr3_−_37146945_{—} | HMOX2_chr16_+_4533637_{—} | CEP70_chr3_−_138291700_{—} |
| 37147014_37138151_37162982 | 4533739_4526488_4555484 | 138291774_138256189_138310695 |
| RPS24_chr10_+_79799961_{—} | EZH2_chr7_−_148543561_{—} | SEC16A_chr9_−_139340096_{—} |
| 79799983_79797062_79800372 | 148543690_148529842_148581255 | 139340171_139338352_139341306 |
| AASDH_chr4_−_57211292_{—} | SUMF2_chr7_+_56141806_{—} | GGT5_chr22_−_24628018_{—} |
| 57211456_57204957_57215428 | 56141911_56140804_56142278 | 24628176_24627498_24640520 |
| FAM13B_chr5_−_137281916_{—} | RDH13_chr19_−_55559413_{—} | WBP1_chr2_+_74685958_{—} |
| 137282000_137281686_137284643 | 55559625_55558856_55559696 | 74686244_74685798_74686769 |
| LRRC23_chr12_+_7015008_{—} | UIMC1_chr5_−_176396601_{—} | CBWD1_chr9_−_146101_{—} |
| 7015118_7014923_7015572 | 176396707_176385155_176402396 | 146158_135030_154708 |
| TOR1AIP2_chr1_−_179834570_{—} | MXI1_chr10_+_112004585_{—} | PARP6_chr15_−_72541585_{—} |
| 179834989_179821946_179846373 | 112004631_111988079_112038937 | 72541655_72535040_72542360 |
| TNC_chr9_−_117826925_{—} | MIF4GD_chr17_−_73265427_{—} | MPRIP_chr17_+_17078606_{—} |
| 117827198_117804620_117835881 | 73265550_73264273_73266194 | 17078726_17077389_17079739 |
| RPS6KB2_chr11_+_67199826_{—} | GSTM4_chr1_+_110200154_{—} | AGTRAP_chr1_+_11806183_{—} |
| 67199963_67198986_67200070 | 110200293_110199901_110200393 | 11806280_11805894_11807496 |
| PYROXD1_chr12_+_21605014_{—} | POLR2J3_chr7_−_102185152_{—} | OFD1_chrX_+_13771486_{—} |
| 21605088_21602625_21608065 | 102185223_102182109_102207028 | 13771560_13769487_13774696 |
| FAM192A_chr16_−_57208080_{—} | TCF25_chr16_+_89944868_{—} | NMRK1_chr9_−_77693241_{—} |
| 57208198_57207781_57219732 | 89945035_89940267_89949758 | 77693498_77692496_77698002 |
| THAP9-AS1_chr4_−_83819141_{—} | SAR1B_chr5_−_133967766_{—} | RANBP3_chr19_−_5957917_{—} |
| 83819215_83816000_83821229 | 133967885_133959727_133968417 | 5957984_5933490_5978071 |
| PARD3_chr10_−_34625126_{—} | FAM192A_chr16_−_57212413_{—} | NOC4L_chr12_+_132631825_{—} |
| 34625171_34620272_34626202 | 57212764_57207781_57219732 | 132631933_132630210_132635525 |
| GIT1_chr17_−_27905979_{—} | EPB41L2_chr6_−_131199243_{—} | HYAL2_chr3_−_50358796_{—} |
| 27906006_27905797_27908355 | 131199390_131191266_131206235 | 50359204_50357966_50360083 |
| PDE4DIP_chr1_−_144871695_{—} | ZNF273_chr7_+_64377407_{—} | NOSTRIN_chr2_+_169712395_{—} |
| 144871881_144866723_144873876 | 64377496_64363797_64377958 | 169712720_169711970_169713199 |
| DMD_chrX_−_31144758_{—} | NEB_chr2_−_152359306_{—} | C12orf73_chr12_−_104348652_{—} |
| 31144790_31140047_31152218 | 152359399_152350767_152359862 | 104348746_104345408_104350408 |
| FAM219B_chr15_−_75197322_{—} | ABHD16A_chr6_−_31669050_{—} | HAPLN3_chr15_−_89422648_{—} |
| 75197380_75197053_75197494 | 31669117_31668805_31670926 | 89422683_89422500_89424587 |
| RBM6_chr3_+_50004902_{—} | BRD8_chr5_−_137502206_{—} | PKD1_chr16_−_2141423_{—} |
| 50006181_50000118_50085677 | 137502299_137501797_137503622 | 2141598_2141175_2141781 |
| PRPF39_chr14_+_45565626_{—} | JMJD6_chr17_−_74717344_{—} | MATR3_chr5_+_138642927_{—} |
| 45565961_45565431_45566089 | 74717433_74716580_74717879 | 138644016_138629494_138650363 |
| EPN3_chr17_+_48613389_{—} | DONSON_chr21_−_34954470_{—} | ARL4A_chr7_+_12727259_{—} |
| 48613560_48610205_48613781 | 34954552_34954361_34956895 | 12727353_12726668_12727790 |
| TBC1D23_chr3_+_100030676_{—} | THYN1_chr11_−_134118702_{—} | GSN_chr9_+_124062333_{—} |
| 100030721_100029386_100034942 | 134118853_134118378_134119060 | 124062404_124043840_124064240 |
| GORAB_chr1_+_170505450_{—} | NAPG_chr18_+_10530766_{—} | PLD3_chr19_+_40871459_{—} |
| 170505562_170501425_170508350 | 10530834_10526155_10539758 | 40871492_40854675_40872325 |
| FAM221A_chr7_+_23731801_{—} | KIAA0895L_chr16_−_67215508_{—} | SLTM_chr15_−_59182479_{—} |
| 23734532_23731215_23737810 | 67215600_67214569_67217164 | 59182660_59181753_59185095 |
| NT5C2_chr10_−_104871501_{—} | C12orf73_chr12_−_104348652_{—} | NDRG2_chr14_−_21492188_{—} |
| 104871562_104866463_104934614 | 104348746_104347312_104350408 | 21492255_21491480_21493835 |
| UHRF2_chr9_+_6495584_{—} | SUN1_chr7_+_889559_{—} | RABGAP1L_chr1_+_174846529_{—} |
| 6497103_6493932_6497197 | 889670_889240_891586 | 174846743_174781098_174926593 |
| TMEM50B_chr21_−_34819324_{—} | ING3_chr7_+_120604795_{—} | NPRL3_chr16_−_174935_{—} |
| 34819447_34811604_34821088 | 120604892_120595678_120606679 | 175072_169254_180520 |
| KIAA1191_chr5_−_175786483_{—} | MANBAL_chr20_+_35927165_{—} | PCBP2_chr12_+_53861588_{—} |
| 175786570_175782752_175786813 | 35927282_35918089_35929610 | 53861627_53861077_53862563 |
| MAP3K6_chr1_−_27691151_{—} | AP1B1_chr22_−_29725700_{—} | SORBS2_chr4_−_186605907_{—} |
| 27691175_27690885_27691263 | 29725709_29724884_29726366 | 186605996_186599976_186611715 |
| CD151_chr11_+_834457_{—} | WDR27_chr6_−_170063658_{—} | PCBP2_chr12_+_53861588_{—} |
| 834591_833026_836062 | 170063745_170060862_170064261 | 53861627_53861077_53862560 |
| RP11-532F12.5_chr15_−_41130740_{—} | SREK1_chr5_+_65451892_{—} | MXRA7_chr17_−_74679928_{—} |
| 41130884_41128480_41136376 | 65454760_65449424_65455046 | 74680009_74676961_74681153 |
| ING4_chr12_−_6764803_{—} | ATP5C1_chr10_+_7848936_{—} | EXOC7_chr17_−_74086409_{—} |
| 6765079_6762562_6765892 | 7848973_7844817_7849621 | 74086478_74085401_74090494 |
| C1orf159_chr1_−_1019294_{—} | GUSB_chr7_−_65444713_{—} | DMPK_chr19_−_46283580_{—} |
| 1019391_1018367_1019732 | 65444898_65444528_65445210 | 46283639_46283127_46285450 |
| TP53BP1_chr15_−_43738963_{—} | KIAA1191_chr5_−_175786464_{—} | STON1_chr2_+_48757851_{—} |
| 43739112_43738788_43739563 | 175786570_175782752_175786813 | 48757968_48757355_48807725 |
| RHOT2_chr16_+_719552_{—} | ZNF271P_chr18_+_32870973_{—} | ERBIN_chr5_+_65370851_{—} |
| 719606_718699_720122 | 32871196_32870355_32885939 | 65371058_65350779_65372143 |
| CTNND1_chr11_+_57558963_{—} | MAP3K4_chr6_+_161529982_{—} | SVIL_chr10_−_29815889_{—} |
| 57559145_57529518_57561481 | 161530073_161529891_161530786 | 29815985_29813644_29818633 |
| RUBCN_chr3_−_197417944_{—} | OSBPL9_chr1_+_52135105_{—} | ARFGEF2_chr20_+_47558400_{—} |
| 197418019_197411088_197420585 | 52135184_52117713_52179674 | 47558524_47538547_47567859 |
| NRAP_chr10_−_115402692_{—} | ADGRG2_chrX_−_19055715_{—} | UIMC1_chr5_−_176396053_{—} |
| 115402797_115401231_115405583 | 19055754_19054095_19058306 | 176396292_176385155_176396601 |
| STX8_chr17_−_9460750_{—} | CCNDBP1_chr15_+_43482252_{—} | BCS1L_chr2_+_219524759_{—} |
| 9460845_9408479_9471687 | 43482349_43481478_43482522 | 219524968_219524466_219525661 |
| ERMP1_chr9_−_5791233_{—} | ZDHHC9_chrX_−_128977234_{—} | PEX2_chr8_−_77898422_{—} |
| 5791282_5787593_5797816 | 128977302_128963117_128977671 | 77898532_77896431_77912225 |
| C9orf3_chr9_+_97848211_{—} | CD151_chr11_+_834529_{—} | RHBDD2_chr7_+_75510682_{—} |
| 97848401_97845001_97848963 | 834591_833022_836062 | 75510804_75508578_75511146 |
| SVIL_chr10_−_29824917_{—} | C2CD5_chr12_−_22611417_{—} | SLAIN2_chr4_+_48396592_{—} |
| 29824998_29820217_29839525 | 22611489_22610095_22612425 | 48396670_48385801_48422141 |
| CXXC1_chr18_−_47813792_{—} | ORMDL1_chr2_−_190647739_{—} | KIAA1217_chr10_+_24831621_{—} |
| 47813878_47813228_47813956 | 190647849_190647328_190648994 | 24831699_24825822_24833909 |
| MMS19_chr10_−_99236591_{—} | FAN1_chr15_+_31202816_{—} | SCRIB_chr8_−_144886737_{—} |
| 99236676_99236501_99237103 | 31203018_31200461_31206060 | 144886995_144886326_144887103 |
| MARK3_chr14_+_103964838_{—} | C1orf116_chr1_−_207200838_{—} | NENF_chr1_+_212615887_{—} |
| 103964865_103958371_103966492 | 207201024_207196825_207205923 | 212615967_212606462_212617680 |
| UIMC1_chr5_−_176395555_{—} | PHLDB2_chr3_+_111671418_{—} | ACCS_chr11_+_44094893_{—} |
| 176396292_176385155_176402396 | 111671559_111664204_111672776 | 44095067_44092865_44096161 |
| MBD2_chr18_−_51714083_{—} | METTL26_chr16_−_685280_{—} | TPM1_chr15_+_63356262_{—} |
| 51714207_51692536_51715243 | 685340_684797_686093 | 63356341_63354844_63358094 |
| NAP1L4_chr11_−_2970456_{—} | TMEM120A_chr7_−_75616855_{—} | YBX3_chr12_−_10862506_{—} |
| 2970494_2966876_2972488 | 75616920_75616746_75617035 | 10862713_10856747_10865809 |
| EPOR_chr19_−_11493522_{—} | SLTM_chr15_−_59191667_{—} | C1orf52_chr1_−_85724617_{—} |
| 11493658_11492781_11493772 | 59192082_59191051_59193458 | 85724744_85724405_85725040 |
| FBXL6_chr8_−_145581098_{—} | SLC37A4_chr11_−_118897312_{—} | SEC31A_chr4_−_83752089_{—} |
| 145581162_145580781_145581287 | 118897398_118896790_118897646 | 83752128_83750211_83765538 |
| MBOAT2_chr2_−_9002400_{—} | TRPT1_chr11_−_63992266_{—} | SNHG16_chr17_+_74555026_{—} |
| 9002452_9000894_9002719 | 63992442_63992189_63992970 | 74555125_74553939_74557369 |
| POFUT2_chr21_−_46685936_{—} | CENPX_chr17_−_79977385_{—} | WNK2_chr9_+_96069058_{—} |
| 46686142_46685550_46687504 | 79977570_79977257_79977733 | 96069103_96060349_96070609 |
| LRRC75A-AS1_chr17_+_16342841_{—} | RBM4_chr11_+_66410920_{—} | DCAF8_chr1_−_160231074_{—} |
| 16343017_16342728_16343498 | 66411611_66407594_66413497 | 160231148_160213824_160232238 |
| AASS_chr7_−_121722841_{—} | KLK2_chr19_+_51380962_{—} | SLC2A8_chr9_+_130159654_{—} |
| 121722945_121721649_121726065 | 51381012_51380264_51381659 | 130159817_130159565_130162185 |
| ABI2_chr2_+_204260381_{—} | C17orf62_chr17_−_80407306_{—} | STAG2_chrX_+_123224703_{—} |
| 204260503_204259569_204267298 | 80407356_80407168_80408575 | 123224814_123224614_123227867 |
| SORBS1_chr10_−_97175225_{—} | HMGN1_chr21_−_40717755_{—} | ENOSF1_chr18_−_677742_{—} |
| 97175942_97174619_97181717 | 40719218_40717200_40719304 | 677872_675402_678695 |
| FKBP14_chr7_−_30059828_{—} | HNRNPC_chr14_−_21731469_{—} | COQ6_chr14_+_74422153_{—} |
| 30059920_30058739_30062280 | 21731741_21702388_21737456 | 74422212_74420272_74422507 |
| CCNL2_chr1_−_1326676_{—} | CTNND1_chr11_+_57558965_{—} | SIRT1_chr10_+_69665919_{—} |
| 1326955_1326245_1328058 | 57559145_57529518_57561481 | 69666044_69651312_69666546 |
| CD151_chr11_+_834529_{—} | CBY1_chr22_+_39052960_{—} | SSBP3_chr1_−_54723741_{—} |
| 834803_833026_836062 | 39053148_39052755_39064021 | 54723822_54722859_54747110 |
| LIMS2_chr2_−_128411997_{—} | MXRA7_chr17_−_74679928_{—} | MYO18A_chr17_−_27443461_{—} |
| 128412118_128400647_128432587 | 74680009_74676961_74684194 | 27443473_27442858_27445062 |
| RGS14_chr5_+_176798475_{—} | KIAA1324_chr1_+_109716309_{—} | TNS1_chr2_−_218694566_{—} |
| 176798590_176798397_176798873 | 109716459_109716201_109727666 | 218694605_218686661_218696177 |
| C11orf80_chr11_+_66523823_{—} | NAGLU_chr17_+_40689415_{—} | FLOT2_chr17_−_27212874_{—} |
| 66523976_66515988_66526513 | 40689563_40688673_40690356 | 27212965_27211333_27215962 |
| DDR1_chr6_+_30853401_{—} | MAGOHB_chr12_−_10765238_{—} | ACBD4_chr17_+_43214385_{—} |
| 30853457_30850760_30856464 | 10765577_10763279_10766037 | 43214506_43214143_43214734 |
| SLC38A5_chrX_−_48324401_{—} | NDUFAF1_chr15_−_41686302_{—} | DHRS11_chr17_+_34956099_{—} |
| 48324480_48321365_48325185 | 41686556_41680720_41687056 | 34956192_34955479_34956400 |
| OSGEP_chr14_−_20919415_{—} | THOC5_chr22_−_29927066_{—} | AGTRAP_chr1_+_11806044_{—} |
| 20919611_20917425_20920132 | 29927099_29925228_29927819 | 11806280_11805894_11807496 |
| TIAL1_chr10_−_121336358_{—} | SNAP23_chr15_+_42820459_{—} | TMEM260_chr14_+_57088248_{—} |
| 121336417_121336262_121336591 | 42820618_42807552_42837333 | 57088420_57085481_57092099 |
| SNHG11_chr20_+_37076572_{—} | KMT2D_chr12_−_49417835_{—} | FRG1_chr4_+_190874222_{—} |
| 37076736_37076266_37077304 | 49417883_49416658_49418360 | 190874280_190873442_190878552 |
| PTGR2_chr14_+_74345798_{—} | SEC14L2_chr22_+_30795631_{—} | YTHDC2_chr5_+_112860677_{—} |
| 74346008_74343871_74346757 | 30795707_30793159_30802330 | 112860874_112851059_112868575 |
| SETD4_chr21_−_37429681_{—} | GEMIN7_chr19_+_45583164_{—} | RPL18A_chr19_+_17972101_{—} |
| 37429775_37429502_37431113 | 45583287_45582635_45593364 | 17972116_17970783_17972902 |
| FANCG_chr9_−_35078137_{—} | NT5C3B_chr17_−_39991839_{—} | ZSWIM7_chr17_−_15880892_{—} |
| 35078340_35077396_35078601 | 39991894_39991524_39992110 | 15881014_15880406_15881357 |
| EXOSC1_chr10_−_99198419_{—} | MBNL1_chr3_+_152173330_{—} | SMTN_chr22_+_31489769_{—} |
| 99198460_99197507_99200927 | 152173366_152165562_152174055 | 31489862_31487468_31491288 |
| RCOR3_chr1_+_211486061_{—} | IFT20_chr17_−_26659171_{—} | IGF1_chr12_−_102811732_{—} |
| 211486303_211477482_211486765 | 26659207_26659013_26662365 | 102811781_102796344_102813286 |
| CTTN_chr11_+_70268614_{—} | BSG_chr19_+_579523_{—} | DCAF6_chr1_+_167992225_{—} |
| 70268737_70266616_70269045 | 579656_572701_580645 | 167992285_167974031_168007608 |
| STAU2_chr8_−_74621266_{—} | SLC25A45_chr11_−_65144803_{—} | TMX2_chr11_+_57505257_{—} |
| 74621397_74601048_74659017 | 65144894_65144547_65146846 | 57505498_57505140_57505825 |
| MYL6_chr12_+_56556346_{—} | FANCL_chr2_−_58431264_{—} | EPB41L1_chr20_+_34783250_{—} |
| 56556423_56556115_56556638 | 58431361_58393009_58449076 | 34783286_34782282_34785780 |
| TPM1_chr15_+_63353396_{—} | NADSYN1_chr11_+_71191264_{—} | RNF7_chr3_+_141461485_{—} |
| 63353472_63353138_63353911 | 71191320_71189515_71191800 | 141461749_141457358_141464000 |
| IGFN1_chr1_+_201193806_{—} | DPH7_chr9_−_140470760_{—} | PLA2G6_chr22_−_38521645_{—} |
| 201194002_201187786_201194951 | 140470854_140470619_140471921 | 38521698_38519265_38522377 |
| SRSF4_chr1_−_29492401_{—} | MADD_chr11_+_47310518_{—} | STIM2_chr4_+_27023115_{—} |
| 29492576_29487029_29508157 | 47310578_47308085_47310941 | 27023234_27019606_27024140 |
| PSMA3-AS1_chr14_−_58752308_{—} | NSMF_chr9_−_140350080_{—} | GUK1_chr1_+_228328824_{—} |
| 58752474_58734111_58758352 | 140350086_140348895_140350862 | 228328989_228328064_228333211 |
| CBWD1_chr9_−_151304_{—} | TMX2_chr11_+_57505384_{—} | TRIM26_chr6_−_30172432_{—} |
| 151427_146158_152033 | 57505498_57505140_57505825 | 30172542_30166930_30181081 |
| ACIN1_chr14_−_23536522_{—} | MAPKAP1_chr9_−_128268588_{—} | APP_chr21_−_27369674_{—} |
| 23537880_23535217_23538684 | 128268696_128246862_128305337 | 27369731_27354790_27372329 |
| BAZ2B_chr2_−_160253584_{—} | PLA2G6_chr22_−_38523413_{—} | TIMM17B_chrX_−_48752634_{—} |
| 160253611_160252345_160253855 | 38523465_38522456_38524275 | 48752737_48752384_48754041 |
| CLSTN1_chr1_−_9816538_{—} | TMEM69_chr1_+_46156645_{—} | TRIM16_chr17_−_15554404_{—} |
| 9816568_9815367_9833329 | 46156782_46153947_46158875 | 15555260_15546130_15580489 |
| NAPG_chr18_+_10534462_{—} | MAPK10_chr4_−_87010358_{—} | NUCB2_chr11_+_17308180_{—} |
| 10534493_10526155_10539758 | 87010430_86989108_87022204 | 17308264_17298375_17316870 |
| HOOK2_chr19_−_12876642_{—} | RP11-43F13.1_chr5_−_1626900_{—} | TBRG4_chr7_−_45143697_{—} |
| 12876648_12876538_12876740 | 1627041_1602850_1632959 | 45143855_45143042_45145039 |
| LMAN2L_chr2_−_97399255_{—} | DYSF_chr2_+_71776479_{—} | FAM45A_chr10_+_120864275_{—} |
| 97399338_97377762_97400145 | 71776521_71766369_71778170 | 120864534_120863709_120867479 |
| PRMT1_chr19_+_50183128_{—} | IL4R_chr16_+_27352390_{—} | LRRFIP2_chr3_−_37163125_{—} |
| 50183182_50180573_50183743 | 27352634_27351594_27353441 | 37163182_37138151_37170553 |
| ARIH2_chr3_+_48982414_{—} | XAF1_chr17_+_6662574_{—} | CD47_chr3_−_107768465_{—} |
| 48982614_48956431_48999044 | 6662838_6661543_6662980 | 107768498_107766139_107769424 |
| DCAF10_chr9_+_37857237_{—} | PQLC1_chr18_−_77690227_{—} | KLK15_chr19_−_51329876_{—} |
| 37857348_37854979_37860044 | 77690311_77679400_77703328 | 51330013_51329204_51340370 |
| ZNF263_chr16_+_3335058_{—} | GPBP1_chr5_+_56532939_{—} | LETMD1_chr12_+_51445874_{—} |
| 3335239_3334205_3336022 | 56532999_56531859_56542126 | 51445990_51442261_51450132 |
| FCGRT_chr19_+_50024951_{—} | RIMS2_chr8_+_105025669_{—} | PBRM1_chr3_−_52718871_{—} |
| 50025070_50016730_50027763 | 105025849_105001649_105026733 | 52718930_52713742_52719764 |
| TNS1_chr2_−_218695089_{—} | SMUG1_chr12_−_54582298_{—} | PPP4C_chr16_+_30092520_{—} |
| 218695113_218686661_218696177 | 54582380_54577743_54582734 | 30092631_30087796_30093804 |
| TMEM222_chr1_+_27657475_{—} | TMEM175_chr4_+_942198_{—} | SIRT2_chr19_−_39389018_{—} |
| 27657628_27657295_27658560 | 942403_941942_944208 | 39389065_39380784_39390145 |
| USP3_chr15_+_63826001_{—} | ADAM15_chr1_+_155033890_{—} | RP11-529K1.3_chr16_+_70346511_{—} |
| 63826117_63824906_63829223 | 155033965_155033308_155034720 | 70346560_70333257_70348804 |
| MBNL1_chr3_+_152164492_{—} | WDR19_chr4_+_39196163_{—} | RAB4A_chr1_+_229422232_{—} |
| 152164546_152163328_152165408 | 39196279_39191401_39201097 | 229422313_229407117_229431594 |
| ABCD4_chr14_−_74759856_{—} | CD99L2_chrX_−_149996778_{—} | SLC38A6_chr14_+_61519066_{—} |
| 74759951_74759572_74761850 | 149998077_149984551_149999703 | 61519703_61518853_61545547 |
| PIGB_chr15_+_55642896_{—} | ME3_chr11_−_86168407_{—} | SRSF7_chr2_−_38976039_{—} |
| 55643110_55634000_55646995 | 86168801_86161440_86176132 | 38976315_38975795_38976670 |
| ALAS2_chrX_−_55054183_{—} | HNRNPH1_chr5_−_179042546_{—} | PLD3_chr19_+_40871459_{—} |
| 55054290_55052448_55057374 | 179042596_179041960_179043126 | 40871492_40854631_40872325 |
| DROSHA_chr5_−_31531556_{—} | CADM2_chr3_+_86028313_{—} | PPIL3_chr2_−_201742219_{—} |
| 31531632_31529212_31532096 | 86028433_86010797_86114754 | 201742327_201741760_201746143 |
| NADK_chr1_−_1688177_{—} | SMARCC2_chr12_−_56558086_{—} | LMAN2L_chr2_−_97402873_{—} |
| 1688321_1688047_1688619 | 56558152_56557549_56558431 | 97402954_97400263_97403685 |
| PIGT_chr20_+_44047411_{—} | MYO6_chr6_+_76604530_{—} | TIMM17B_chrX_−_48752634_{—} |
| 44047619_44045334_44047934 | 76604557_76602407_76617321 | 48752784_48752384_48754041 |
| NMRAL1_chr16_−_4521314_{—} | SORBS2_chr4_−_186605907_{—} | ADGRG1_chr16_+_57675502_{—} |
| 4521450_4519699_4524093 | 186606000_186599976_186696380 | 57675620_57654048_57684164 |
| INSR_chr19_−_7150507_{—} | MRPS33_chr7_−_140710218_{—} | YLPM1_chr14_+_75290958_{—} |
| 7150543_7143101_7152736 | 140710417_140706335_140714710 | 75291010_75287840_75295915 |
| METTL22_chr16_+_8738413_{—} | TUFT1_chr1_+_151534566_{—} | MARK3_chr14_+_103964838_{—} |
| 8738582_8736422_8739964 | 151534641_151512902_151536379 | 103964865_103958371_103969218 |
| BTBD3_chr20_+_11898425_{—} | HNRNPC_chr14_−_21731469_{—} | ERP29_chr12_+_112457559_{—} |
| 11898659_11871602_11898981 | 21731495_21702388_21737456 | 112457698_112451413_112459953 |
| LUC7L_chr16_−_258599_{—} | AP2M1_chr3_+_183898432_{—} | SORBS2_chr4_−_186605907_{—} |
| 258663_258187_270647 | 183898529_183898039_183898636 | 186605996_186599976_186696380 |
| FAM189B_chr1_−_155224190_{—} | RBM4B_chr11_−_66433742_{—} | CDC25B_chr20_+_3783376_{—} |
| 155224247_155223523_155224443 | 66433964_66433049_66436085 | 3783455_3783023_3783556 |
| ARMCX2_chrX_−_100914404_{—} | EMC8_chr16_−_85813984_{—} | NCOR2_chr12_−_124811954_{—} |
| 100914487_100913128_100914742 | 85814079_85813473_85814816 | 124812179_124810916_124815390 |
| CASP6_chr4_−_110617565_{—} | TMEM175_chr4_+_941496_{—} | ARHGAP44_chr17_+_12890417_{—} |
| 110617642_110615856_110618777 | 941942_926328_944208 | 12890469_12888225_12893348 |
| IQCB1_chr3_−_121507130_{—} | SVIL_chr10_−_29824917_{—} | RALGAPA2_chr20_−_20656879_{—} |
| 121507279_121500721_121508919 | 29824998_29822387_29839525 | 20656932_20634231_20693017 |
| COQ5_chr12_−_120964259_{—} | RIC8B_chr12_+_107273552_{—} | UBE2Q2_chr15_+_76182775_{—} |
| 120964349_120960166_120966742 | 107273725_107254190_107279681 | 76182824_76175765_76191767 |
| TRAK2_chr2_−_202265740_{—} | C6orf136_chr6_+_30615382_{—} | REXO4_chr9_−_136277897_{—} |
| 202265817_202264216_202272125 | 30615623_30615080_30617334 | 136278041_136276193_136279784 |
| TRA2A_chr7_−_23570799_{—} | DST_chr6_−_56329482_{—} | NGLY1_chr3_−_25761504_{—} |
| 23570889_23562051_23571407 | 56329554_56328562_56330875 | 25761682_25761126_25770623 |
| PI4KB_chr1_−_151298647_{—} | PKP4_chr2_+_159533250_{—} | HEXA_chr15_−_72646031_{—} |
| 151298849_151297393_151299746 | 159533379_159530512_159535092 | 72646078_72643575_72647899 |
| CAMK1_chr3_−_9802339_{—} | THTPA_chr14_+_24025951_{—} | NENF_chr1_+_212615906_{—} |
| 9802452_9801434_9803144 | 24026248_24025552_24027903 | 212615967_212606462_212617680 |
| BRE_chr2_+_28550140_{—} | NMRAL1_chr16_−_4516527_{—} | GARNL3_chr9_+_130126867_{—} |
| 28550314_28521358_28561316 | 4516686_4516403_4519504 | 130127055_130119656_130127578 |
| PRC1_chr15_−_91512308_{—} | TRPM2_chr21_+_45846892_{—} | TNFAIP2_chr14_+_103590711_{—} |
| 91512350_91510432_91512676 | 45846994_45846619_45855013 | 103590843_103589820_103592646 |
| SVIL_chr10_−_29815889_{—} | FAM189B_chr1_−_155221523_{—} | GUK1_chr1_+_228329326_{—} |
| 29815985_29813644_29819535 | 155221699_155220955_155223415 | 228329530_228328989_228333211 |
| RRBP1_chr20_−_17660643_{—} | MYH11_chr16_−_15878554_{—} | CCNL2_chr1_−_1328058_{—} |
| 17660720_17641173_17662672 | 15878575_15876334_15880486 | 1328183_1326245_1328775 |
| SLC38A10_chr17_−_79223869_{—} | ANK2_chr4_+_114293688_{—} | LRRC23_chr12_+_7019053_{—} |
| 79223893_79220861_79225292 | 114293781_114290961_114294245 | 7019190_7016609_7023054 |
| ZNHIT3_chr17_+_34842778_{—} | SYNE4_chr19_−_36497324_{—} | TLE1_chr9_−_84248262_{—} |
| 34842810_34842629_34848656 | 36497573_36496339_36499455 | 84248279_84235472_84267128 |
| TM2D1_chr1_−_62152463_{—} | ZNF7_chr8_+_146054427_{—} | RAB11FIP3_chr16_+_541133_{—} |
| 62152567_62149218_62160368 | 146054475_146052994_146062778 | 541268_539000_546823 |
| GTPBP10_chr7_+_89984399_{—} | AP2B1_chr17_+_33997875_{—} | IDS_chrX_−_148583604_{—} |
| 89984544_89983863_90001468 | 33997917_33984810_33998772 | 148583707_148582568_148585686 |
| C11orf80_chr11_+_66590034_{—} | MGAT1_chr5_−_180222655_{—} | INO80E_chr16_+_30015888_{—} |
| 66590145_66583670_66595736 | 180222875_180220097_180229679 | 30015978_30012361_30016541 |
| NPEPPS_chr17_+_45654410_{—} | MVK_chr12_+_110016969_{—} | PPFIA1_chr11_+_70228193_{—} |
| 45654526_45646860_45656755 | 110017087_110013950_110017606 | 70228264_70224301_70229108 |
| TAMM41_chr3_−_11874476_{—} | ZNF7_chr8_+_146054427_{—} | SMARCA2_chr9_+_2170418_{—} |
| 11874625_11871338_11880695 | 146054475_146052994_146062775 | 2170472_2161903_2181570 |
| DNAJC25_chr9_+_114405136_{—} | RIOK3_chr18_+_21047362_{—} | SUOX_chr12_+_56395669_{—} |
| 114405374_114394023_114409386 | 21047490_21046240_21053392 | 56395732_56391507_56395995 |
| INO80E_chr16_+_30014756_{—} | JOSD2_chr19_−_51010830_{—} | CAMLG_chr5_+_134079676_{—} |
| 30014847_30012851_30015888 | 51010956_51009829_51013542 | 134079742_134074482_134086448 |
| TPT1-AS1_chr13_+_45964892_{—} | PLD3_chr19_+_40871459_{—} | MRPL33_chr2_+_27997290_{—} |
| 45965037_45963955_45965166 | 40871492_40854675_40872290 | 27997397_27995559_28002299 |
| LINC01006_chr7_−_156398351_{—} | GAS8_chr16_+_90102040_{—} | PFDN1_chr5_−_139680000_{—} |
| 156398458_156398074_156433229 | 90102095_90099332_90102788 | 139680167_139661118_139682625 |
| FAM45A_chr10_+_120864275_{—} | PAAF1_chr11_+_73598084_{—} | AARSD1_chr17_−_41105740_{—} |
| 120864534_120863709_120867459 | 73598144_73589864_73598398 | 41105795_41103911_41106892 |
| LUC7L2_chr7_+_139059146_{—} | PBRM1_chr3_−_52592264_{—} | PQLC1_chr18_−_77693968_{—} |
| 139059217_139045068_139060807 | 52592429_52584833_52595782 | 77694022_77664183_77703328 |
| PITPNM2_chr12_−_123474333_{—} | EPN3_chr17_+_48613389_{—} | UBAP2L_chr1_+_154241837_{—} |
| 123474495_123473419_123475091 | 48613560_48610346_48613781 | 154241888_154241430_154243356 |
| MMP19_chr12_−_56234889_{—} | TRA2A_chr7_−_23561750_{—} | FAM111A_chr11_+_58910969_{—} |
| 56235020_56234666_56236136 | 23562051_23561459_23570799 | 58913329_58910777_58916308 |
| STEAP4_chr7_−_87920230_{—} | P4HB_chr17_−_79813328_{—} | PPP4R3A_chr14_−_91932604_{—} |
| 87920329_87913586_87936106 | 79813381_79805223_79817056 | 91932760_91931763_91937180 |
| USP15_chr12_+_62768170_{—} | PHPT1_chr9_+_139744957_{—} | GLB1L3_chr11_+_134183289_{—} |
| 62768316_62749256_62775270 | 139745012_139744589_139745206 | 134183368_134182781_134183834 |
| ATP6V0B_chr1_+_44441471_{—} | LMBRIL_chr12_−_49500293_{—} | SORBS1_chr10_−_97174250_{—} |
| 44441520_44440779_44441761 | 49500529_49499740_49500743 | 97174619_97170534_97181717 |
| EIF2D_chr1_−_206773086_{—} | CRAT_chr9_−_131870686_{—} | NDRG2_chr14_−_21492144_{—} |
| 206773190_206772446_206773616 | 131870757_131870356_131872761 | 21492255_21491480_21493187 |
| CPNE1_chr20_−_34246851_{—} | NAGK_chr2_+_71302691_{—} | SECISBP2_chr9_+_91954778_{—} |
| 34246904_34220845_34252681 | 71302772_71300724_71304687 | 91954868_91953495_91956261 |
| PCBP4_chr3_−_51996825_{—} | CD40_chr20_+_44750362_{—} | DCAF8_chr1_−_160231074_{—} |
| 51996908_51995320_52001341 | 44750537_44747033_44750871 | 160231140_160213824_160232238 |
| SEC31A_chr4_−_83763337_{—} | MGRN1_chr16_+_4730032_{—} | AASS_chr7_−_121741424_{—} |
| 83763634_83750211_83765538 | 4730098_4727573_4731550 | 121741492_121738920_121741674 |
| TARBP2_chr12_+_53898918_{—} | RAD52_chr12_−_1035961_{—} | PHKA2_chrX_−_18920909_{—} |
| 53899046_53898599_53899432 | 1036112_1034691_1036310 | 18921028_18919721_18923875 |
| WBP1_chr2_+_74685958_{—} | NEO1_chr15_+_73567032_{—} | WBP1_chr2_+_74686123_{—} |
| 74686046_74685798_74686564 | 73567065_73566346_73570471 | 74686225_74685798_74686769 |
| AK2_chr1_−_33497144_{—} | SELENOP_chr5_−_42810792_{—} | LIMCH1_chr4_+_41640948_{—} |
| 33497262_33490168_33502336 | 42810880_42801433_42811937 | 41640984_41621457_41646516 |
| ANO8_chr19_−_17444230_{—} | NPEPPS_chr17_+_45654446_{—} | SLC20A1_chr2_+_113410465_{—} |
| 17444363_17444048_17444498 | 45654526_45646860_45656755 | 113410524_113410375_113414698 |
| DNAJC14_chr12_−_56223272_{—} | SLC25A16_chr10_−_70276840_{—} | NOL8_chr9_−_95082224_{—} |
| 56223420_56222498_56224479 | 70277022_70276600_70287002 | 95082661_95081638_95083949 |
| TRMT11_chr6_+_126329802_{—} | PMS2P5_chr7_+_74312525_{—} | APTX_chr9_−_32988080_{—} |
| 126329891_126329618_126332398 | 74312628_74312349_74313767 | 32988127_32987844_32989756 |
| PIGX_chr3_+_196453525_{—} | RPS3A_chr4_+_152021636_{—} | RPAIN_chr17_+_5329290_{—} |
| 196453642_196449427_196454793 | 152021740_152020866_152024022 | 5329619_5326149_5335861 |
| VTI1B_chr14_−_68129745_{—} | XPNPEP3_chr22_+_41266051_{—} | CTNND1_chr11_+_57556508_{—} |
| 68129907_68126639_68141091 | 41266143_41265119_41277773 | 57556627_57529518_57563048 |
| PEX1_chr7_−_92138642_{—} | ACCS_chr11_+_44094996_{—} | MYO18A_chr17_−_27406744_{—} |
| 92138725_92136440_92140257 | 44095085_44092865_44096161 | 27408018_27401932_27409333 |
| DGUOK_chr2_+_74184251_{—} | SLC38A6_chr14_+_61514868_{—} | SYNGR2_chr17_+_76167590_{—} |
| 74184367_74177859_74185272 | 61515015_61512885_61517229 | 76167730_76167135_76167819 |
| MAN2C1_chr15_−_75655550_{—} | ABI1_chr10_−_27047990_{—} | PICALM_chr11_−_85689112_{—} |
| 75655631_75655089_75656339 | 27048167_27040712_27054146 | 85689136_85685855_85692171 |
| SRSF6_chr20_+_42087792_{—} | PRPF40A_chr2_−_153571063_{—} | FLNB_chr3_+_58127584_{—} |
| 42088060_42087149_42088410 | 153571143_153551136_153572508 | 58127656_58124256_58128376 |
| CYP20A1_chr2_+_204137368_{—} | DMTN_chr8_+_21924595_{—} | RPS3A_chr4_+_152022126_{—} |
| 204137471_204131404_204143295 | 21924670_21924404_21925037 | 152022314_152020866_152024022 |
| ZNF706_chr8_−_102214560_{—} | HNRNPAB_chr5_+_177637132_{—} | USP54_chr10_−_75280665_{—} |
| 102214675_102213971_102217662 | 177637273_177636448_177637553 | 75280785_75277505_75283340 |
| ZNF160_chr19_−_53594665_{—} | INO80E_chr16_+_30014970_{—} | WBP1_chr2_+_74686564_{—} |
| 53594806_53589574_53594889 | 30014991_30012851_30015888 | 74686679_74685798_74686769 |
| SYNE1_chr6_−_152466621_{—} | ITPA_chr20_+_3193964_{—} | ZNF263_chr16_+_3335680_{—} |
| 152466690_152464900_152469179 | 3194072_3193872_3194630 | 3335754_3334205_3336022 |
| PLG_chr6_+_161135825_{—} | KIAA1524_chr3_−_108304512_{—} | PPOX_chr1_+_161137160_{—} |
| 161135946_161134157_161137676 | 108304596_108304059_108308120 | 161137276_161137024_161140409 |
| FAM126A_chr7_−_22986651_{—} | AP1G2_chr14_−_24035024_{—} | VEZT_chr12_+_95635939_{—} |
| 22986866_22985782_22999874 | 24035101_24034910_24035272 | 95636040_95611662_95645715 |
| MSI2_chr17_+_55754347_{—} | IFT172_chr2_−_27688612_{—} | COMTD1_chr10_−_76995024_{—} |
| 55754420_55752487_55756909 | 27688749_27686048_27693794 | 76995130_76994936_76995592 |
| PRUNE1_chr1_+_150991032_{—} | ACOT9_chrX_−_23731251_{—} | INTS11_chr1_−_1258271_{—} |
| 150991145_150981147_150997086 | 23731325_23726061_23739997 | 1258667_1257364_1259960 |
| TTC31_chr2_+_74717151_{—} | MBNL1_chr3_+_152173330_{—} | MRPL55_chr1_−_228296137_{—} |
| 74717254_74710537_74717370 | 152173366_152165562_152177059 | 228296175_228296022_228296655 |
| WBP5_chrX_+_102612010_{—} | CD47_chr3_−_107768465_{—} | DMWD_chr19_−_46287898_{—} |
| 102612089_102611534_102612542 | 107768498_107766139_107776323 | 46287973_46287548_46288851 |
| IFT88_chr13_+_21237401_{—} | ASRGL1_chr11_+_62123796_{—} | APLP2_chr11_+_130007150_{—} |
| 21237525_21230569_21237636 | 62123939_62105639_62124458 | 130007186_130003623_130010292 |
| PRPF39_chr14_+_45565798_{—} | DNAJC10_chr2_+_183604271_{—} | MPST_chr22_+_37419792_{—} |
| 45565961_45565431_45566089 | 183604436_183601113_183605025 | 37419968_37415913_37420232 |
| MYBPC1_chr12_+_102076381_{—} | BRD8_chr5_−_137495243_{—} | NGB_chr14_−_77735557_{—} |
| 102076440_102074307_102078159 | 137495288_137492956_137495757 | 77735669_77734928_77737191 |
| PWWP2A_chr5_−_159507657_{—} | CDC42SE1_chr1_−_151029131_{—} | NISCH_chr3_+_52514894_{—} |
| 159507777_159505175_159520107 | 151029262_151028469_151031954 | 52515125_52514311_52518528 |
| SDHC_chr1_+_161293403_{—} | IMMP2L_chr7_−_111172573_{—} | CAMK2G_chr10_−_75585036_{—} |
| 161293460_161284215_161310383 | 111172641_111161505_111201906 | 75585105_75583842_75587846 |
| IFI44_chr1_+_79126238_{—} | HMGN1_chr21_−_40719304_{—} | HEMK1_chr3_+_50616251_{—} |
| 79126376_79125168_79128388 | 40719409_40717884_40720217 | 50616357_50615004_50617274 |
| GOLGA2P7_chr15_−_84873982_{—} | OGDH_chr7_+_44695916_{—} | TMEM185A_chrX_−_148692969_{—} |
| 84874071_84873727_84898603 | 44695961_44687133_44706334 | 148693146_148682143_148713225 |
| RP11-407N17.3_chr14_+_39790131_{—} | IFT172_chr2_−_27688277_{—} | SEC31A_chr4_−_83752089_{—} |
| 39790260_39788495_39796067 | 27688385_27686048_27693794 | 83752128_83750211_83763292 |
| CDC42SE1_chr1_−_151029131_{—} | IFT88_chr13_+_21141808_{—} | CHID1_chr11_−_908541_{—} |
| 151029262_151027602_151031954 | 21141924_21141395_21148518 | 908645_904859_910774 |
| MYO9B_chr19_+_17321141_{—} | DIAPH2_chrX_+_95990756_{—} | TRMT2B_chrX_−_100306632_{—} |
| 17321189_17320518_17321523 | 95990789_95940189_95993584 | 100306722_100290672_100306805 |
| ENTPD6_chr20_+_25187714_{—} | FAM193B_chr5_−_176958953_{—} | HMGN1_chr21_−_40717755_{—} |
| 25188033_25176503_25190484 | 176959132_176958522_176959443 | 40717884_40717200_40719304 |
| CD27-AS1_chr12_−_6560035_{—} | PRDX5_chr11_+_64087205_{—} | FGFR1_chr8_−_38287199_{—} |
| 6560146_6557903_6560634 | 64087340_64085858_64088336 | 38287466_38285953_38314873 |
| FBXO38_chr5_+_147806775_{—} | ABHD14B_chr3_−_52005475_{—} | NSFL1C_chr20_−_1436358_{—} |
| 147807510_147805264_147812986 | 52005714_52004200_52007980 | 1436364_1435777_1438844 |
| PLEKHA1_chr10_+_124187791_{—} | NOL8_chr9_−_95082224_{—} | RCOR3_chr1_+_211486061_{—} |
| 124187832_124186547_124189139 | 95082419_95081638_95083949 | 211486177_211477482_211486765 |
| MFSD3_chr8_+_145735742_{—} | FAM114A1_chr4_+_38870018_{—} | CYP2U1_chr4_+_108859193_{—} |
| 145735890_145735398_145735980 | 38870167_38869455_38879691 | 108859332_108853289_108866125 |
| HNRNPH1_chr5_−_179046269_{—} | SLC15A4_chr12_−_129294487_{—} | RBM3_chrX_+_48434309_{—} |
| 179046361_179045324_179047892 | 129294656_129294018_129299319 | 48434471_48434055_48434701 |
| UCKL1_chr20_−_62575005_{—} | DCUN1D4_chr4_+_52775085_{—} | ENOSF1_chr18_−_677344_{—} |
| 62575022_62572561_62575749 | 52775141_52765544_52777235 | 677444_675402_678695 |
| NTAN1_chr16_−_15141853_{—} | KANK2_chr19_−_11306725_{—} | WASHC3_chr12_−_102443966_{—} |
| 15141956_15141407_15149747 | 11306903_11305266_11308160 | 102444069_102439897_102455025 |
| MCAT_chr22_−_43533086_{—} | ARHGEF1_chr19_+_42402094_{—} | MMS19_chr10_−_99221252_{—} |
| 43533304_43529492_43537167 | 42402262_42400555_42402590 | 99221379_99220764_99221584 |
| S100A4_chr1_−_153517945_{—} | LZTR1_chr22_+_21349656_{—} | GMFB_chr14_−_54948839_{—} |
| 153517994_153517285_153518228 | 21349773_21349315_21350034 | 54948920_54948176_54950388 |
| SHROOM1_chr5_−_132161968_{—} | DECR2_chr16_+_456889_{—} | ACOT9_chrX_−_23752457_{—} |
| 132162279_132161874_132166285 | 456952_456833_457424 | 23752484_23751334_23754035 |
| PDCD2_chr6_−_170889089_{—} | SYNE4_chr19_−_36497503_{—} | QTRT2_chr3_+_113786833_{—} |
| 170889193_170888058_170892144 | 36497573_36496339_36499455 | 113786910_113775711_113789472 |
| CD47_chr3_−_107769424_{—} | RP11-452C8.1_chr4_+_80611141_{—} | DNAJC8_chr1_−_28556641_{—} |
| 107769449_107766139_107776323 | 80611230_80589281_80617566 | 28556763_28555534_28559431 |
| SPOUT1_chr9_−_131586025_{—} | INO80E_chr16_+_30012532_{—} | FOXRED1_chr11_+_126144825_{—} |
| 131586173_131585112_131586350 | 30015978_30012361_30016541 | 126144916_126143349_126145221 |
| ZDHHC17_chr12_+_77214837_{—} | SERPINA1_chr14_−_94854896_{—} | EHBP1_chr2_+_63215065_{—} |
| 77214947_77209799_77216185 | 94855000_94849578_94856793 | 63215173_63206470_63217850 |
| METTL22_chr16_+_8728895_{—} | RP11-597D13.9_chr4_+_159098063_{—} | TPM1_chr15_+_63353911_{—} |
| 8728936_8722967_8729024 | 159098198_159092632_159100812 | 63353987_63353472_63354413 |
| SDK1_chr7_+_4215406_{—} | MRPL55_chr1_−_228296655_{—} | COPS7A_chr12_+_6837091_{—} |
| 4215466_4213989_4218116 | 228296722_228296019_228296961 | 6837167_6833984_6837388 |
| KLK12_chr19_−_51537680_{—} | ARHGAP12_chr10_−_32128232_{—} | TRIP10_chr19_+_6746039_{—} |
| 51537896_51537395_51538061 | 32128247_32120728_32128564 | 6746207_6745005_6746462 |
| WASF3_chr13_+_27254171_{—} | GPS1_chr17_+_80010131_{—} | PSMC5_chr17_+_61905188_{—} |
| 27254338_27246126_27255190 | 80010335_80009400_80011149 | 61905283_61904874_61905497 |
| RNPEP_chr1_+_201958510_{—} | TCF3_chr19_−_1615283_{—} | ANKRD10_chr13_−_111546631_{—} |
| 201958659_201958172_201965274 | 1615484_1612432_1615684 | 111546740_111546549_111558379 |
| USP28_chr11_−_113702641_{—} | CXorf40B_chrX_−_149102594_{—} | ROGDI_chr16_−_4851050_{—} |
| 113702715_113700067_113704141 | 149102791_149102355_149105650 | 4851322_4850579_4851503 |
| TMTC4_chr13_−_101308634_{—} | MICAL3_chr22_−_18286576_{—} | C11orf80_chr11_+_66589113_{—} |
| 101308722_101294565_101315217 | 18286627_18274067_18291609 | 66589235_66583670_66595736 |
| FOLH1_chr11_−_49179503_{—} | IKBKAP_chr9_−_111681090_{—} | TPM1_chr15_+_63335904_{—} |
| 49179595_49178359_49186256 | 111681181_111679950_111685121 | 63336030_63335142_63336225 |
| FAM13A_chr4_−_89726161_{—} | CNDP2_chr18_+_72164743_{—} | KIAA1468_chr18_+_59947592_{—} |
| 89726203_89709083_89744126 | 72164961_72163666_72167171 | 59947675_59947089_59947878 |
| AFMID_chr17_+_76201173_{—} | RPLPO_chr12_−_120636356_{—} | SNRPA1_chr15_−_101826418_{—} |
| 76201271_76200981_76202026 | 120636573_120635265_120636656 | 101826498_101826006_101827112 |
| VPS29_chr12_−_110937339_{—} | HNRNPK_chr9_−_86588816_{—} | HNRNPDL_chr4_−_83346715_{—} |
| 110937351_110931036_110939853 | 86588888_86588314_86589431 | 83346820_83346036_83347189 |
| CLTC_chr17_+_57764361_{—} | BCS1L_chr2_+_219524759_{—} | CPNE1_chr20_−_34218662_{—} |
| 57764382_57763169_57767996 | 219524889_219524466_219526128 | 34218717_34218412_34218822 |
| ANKS3_chr16_−_4776979_{—} | NT5C3A_chr7_−_33075545_{—} | FDPS_chr1_+_155279543_{—} |
| 4777178_4776781_4779980 | 33075600_33066527_33102179 | 155279756_155278867_155279833 |
| AC004381.6_chr16_+_20835760_{—} | LINC00963_chr9_+_132264675_{—} | PTPRF_chr1_+_44067741_{—} |
| 20835849_20833224_20837154 | 132265418_132255874_132265601 | 44067768_44064584_44069086 |
| IDS_chrX_−_148583604_{—} | NFU1_chr2_−_69650713_{—} | GALK2_chr15_+_49611168_{—} |
| 148583707_148582568_148584841 | 69650849_69646736_69659033 | 49611313_49584734_49611800 |
| GREB1_chr2_+_11778796_{—} | USF2_chr19_+_35760705_{—} | PPP4R1_chr18_−_9562919_{—} |
| 11778935_11778042_11780416 | 35760906_35760602_35761349 | 9563044_9562073_9563375 |
| ANKRD10_chr13_−_111546455_{—} | PANX2_chr22_+_50613860_{—} | ADAM15_chr1_+_155033890_{—} |
| 111546549_111545610_111558379 | 50613911_50609385_50615367 | 155033965_155033308_155034379 |
| RP11-793H13.8_chr12_+_53858543_{—} | TSPAN17_chr5_+_176078754_{—} | HDHD2_chr18_−_44663605_{—} |
| 53858636_53856351_53861004 | 176078901_176074703_176079743 | 44663667_44662820_44676748 |
| DOLPP1_chr9_+_131848417_{—} | HAPLN3_chr15_−_89422648_{—} | SRRT_chr7_+_100480385_{—} |
| 131848546_131847895_131848987 | 89423841_89422500_89424587 | 100480711_100479862_100481690 |
| SZRD1_chr1_+_16717869_{—} | FAM192A_chr16_−_57209450_{—} | ZNF326_chr1_+_90473170_{—} |
| 16717919_16693803_16721532 | 57209612_57207781_57219732 | 90473309_90470803_90475646 |
| APBB1_chr11_−_6423206_{—} | MTRF1L_chr6_−_153312319_{—} | NHLRC3_chr13_+_39618226_{—} |
| 6423212_6422918_6423311 | 153312551_153311230_153313991 | 39618318_39613848_39621176 |
| HCFC1R1_chr16_−_3073474_{—} | TMEM198_chr2_+_220412227_{—} | ABCA5_chr17_−_67281528_{—} |
| 3073531_3073362_3073847 | 220412803_220409615_220413873 | 67281561_67280213_67282038 |
| C20orf24_chr20_+_35236292_{—} | MTA1_chr14_+_105912003_{—} | RBM39_chr20_−_34328446_{—} |
| 35236403_35236221_35240412 | 105912189_105911848_105916394 | 34328519_34326939_34328745 |
| TCF3_chr19_−_1615283_{—} | TRIT1_chr1_−_40318402_{—} | SEMA4G_chr10_+_102729448_{—} |
| 1615514_1612432_1615684 | 40318548_40315933_40319641 | 102729681_102729348_102732285 |
| GPS1_chr17_+_80010134_{—} | TXNDC11_chr16_−_11794307_{—} | PMM2_chr16_+_8898623_{—} |
| 80010335_80009840_80011149 | 11794420_11792181_11815432 | 8898700_8895767_8900172 |
| MITD1_chr2_−_99789876_{—} | HNRNPA2B1_chr7_−_26230612_{—} | POLRID_chr13_+_28195473_{—} |
| 99789965_99788109_99790377 | 26230748_26230080_26232114 | 28195574_28195277_28222515 |
| CTPS1_chr1_+_41473120_{—} | LICAM_chrX_−_153128822_{—} | SLC9A8_chr20_+_48467298_{—} |
| 41473217_41471766_41474330 | 153128834_153128349_153128931 | 48467381_48466217_48471974 |
| LUC7L2_chr7_+_139060254_{—} | RHOC_chr1_−_113247674_{—} | MAGOHB_chr12_−_10761696_{—} |
| 139060329_139045068_139060807 | 113247790_113246428_113249699 | 10761982_10760535_10762429 |
| IL17RC_chr3_+_9970263_{—} | WBP1_chr2_+_74686604_{—} | ACTR3B_chr7_+_152508781_{—} |
| 9970314_9970170_9971535 | 74686689_74685798_74686769 | 152508818_152498816_152511634 |
| CPNE1_chr20_−_34243123_{—} | C5orf38_chr5_+_2752735_{—} | IL32_chr16_+_3115621_{—} |
| 34243266_34220845_34252681 | 2752868_2752578_2755142 | 3115688_3115495_3115784 |
| HEXDC_chr17_+_80385905_{—} | SEC31A_chr4_−_83782783_{—} | DOCK7_chr1_−_63010617_{—} |
| 80386003_80382379_80386433 | 83782861_83778917_83783686 | 63010710_63009416_63018402 |
| C17orf49_chr17_+_6920268_{—} | KIF24_chr9_−_34271806_{—} | REPS1_chr6_−_139247537_{—} |
| 6920333_6920004_6920575 | 34271928_34263170_34286614 | 139247615_139242261_139251113 |
| TARBP1_chr1_−_234534127_{—} | ZFAND6_chr15_+_80390757_{—} | SPTAN1_chr9_+_131371929_{—} |
| 234534299_234529583_234536926 | 80390920_80364989_80412669 | 131371944_131371563_131373992 |
| CHTF8_chr16_−_69154955_{—} | CTNND1_chr11_+_57556508_{—} | TMEM14B_chr6_+_10751365_{—} |
| 69155073_69154552_69155338 | 57556627_57529518_57561481 | 10751467_10749931_10755374 |
| FIZ1_chr19_−_56106985_{—} | MKNK1_chr1_−_47051545_{—} | L3HYPDH_chr14_−_59941156_{—} |
| 56107100_56105012_56108937 | 47051646_47049001_47059784 | 59941244_59939808_59942586 |
| METTL26_chr16_−_684888_{—} | LRRC27_chr10_+_134155716_{—} | PDLIM7_chr5_−_176918807_{—} |
| 684956_684797_685611 | 134155775_134151199_134158001 | 176918996_176918421_176919405 |
| TPM1_chr15_+_63336225_{—} | UBE2D4_chr7_+_43982424_{—} | HEXDC_chr17_+_80398348_{—} |
| 63336351_63336030_63349183 | 43982456_43978093_43988230 | 80398489_80397589_80398872 |
| TEX264_chr3_+_51708286_{—} | LASIL_chrX_−_64748139_{—} | ZDHHC8P1_chr22_−_23744223_{—} |
| 51708578_51705304_51718428 | 64748249_64744930_64749091 | 23744342_23744140_23744547 |
| FARS2_chr6_+_5404774_{—} | SH3D21_chr1_+_36773709_{—} | C20orf24_chr20_+_35236292_{—} |
| 5404934_5369415_5431273 | 36773810_36773426_36773989 | 35236403_35236221_35238003 |
| MID1_chrX_−_10588329_{—} | BTBD1_chr15_−_83718824_{—} | ST6GALNAC1_chr17_−_74633681_{—} |
| 10588459_10535643_10851672 | 83718930_83710677_83725140 | 74633818_74625793_74639589 |
| DMPK_chr19_−_46274607_{—} | SEC31A_chr4_−_83819141_{—} | WIPI2_chr7_+_5232748_{—} |
| 46274654_46273898_46274825 | 83819215_83803093_83821229 | 5232802_5230124_5254165 |
| SMPDL3A_chr6_+_123122454_{—} | WRNIP1_chr6_+_2770428_{—} | BRD8_chr5_−_137497484_{—} |
| 123122551_123118113_123124808 | 2770595_2766678_2779496 | 137497553_137496757_137498818 |
| EXOC1_chr4_+_56755053_{—} | RAB4B-EGLN2_chr19_+_41285923_{—} | BCLAF1_chr6_−_136595100_{—} |
| 56755098_56750094_56756388 | 41286004_41284296_41292569 | 136595327_136594325_136596669 |
| NAA25_chr12_−_112487286_{—} | OSR2_chr8_+_99960400_{—} | IP6K2_chr3_−_48732126_{—} |
| 112487415_112486247_112491361 | 99960649_99957051_99961066 | 48732257_48731673_48732522 |
| RCOR3_chr1_+_211485696_{—} | PLCB4_chr20_+_9353693_{—} | CHCHD4_chr3_−_14163416_{—} |
| 211485829_211477482_211486061 | 9353751_9353050_9360700 | 14163586_14158024_14166154 |
| NUP50_chr22_+_45566868_{—} | GMPR2_chr14_+_24703312_{—} | FAAH_chr1_+_46871709_{—} |
| 45567024_45564127_45567480 | 24703447_24702804_24704942 | 46871750_46871466_46874130 |
| GS1-124K5.12_chr7_−_66041741_{—} | FLNB_chr3_+_58127584_{—} | FOXRED1_chr11_+_126144821_{—} |
| 66041916_66038537_66057243 | 58127623_58124256_58128376 | 126144916_126143349_126145221 |
| SNHG6_chr8_−_67834848_{—} | HSH2D_chr19_+_16266451_{—} | PPP1R12A_chr12_−_80173100_{—} |
| 67834960_67834627_67837671 | 16266573_16265301_16268019 | 80173131_80172380_80175594 |
| STYXL1_chr7_−_75630207_{—} | AKAP9_chr7_+_91671359_{—} | KIAA1468_chr18_+_59947006_{—} |
| 75630320_75625917_75633075 | 91671500_91670212_91671981 | 59947089_59942706_59947592 |
| GPATCH2_chr1_−_217668356_{—} | F3_chr1_−_95006127_{—} | TSC2_chr16_+_2127598_{—} |
| 217668417_217665092_217671697 | 95006622_95005924_95007092 | 2127727_2126586_2129032 |
| MINOS1_chr1_+_19927280_{—} | ALKBH6_chr19_−_36504229_{—} | SORBS1_chr10_−_97135729_{—} |
| 19927465_19923603_19949967 | 36504324_36503991_36505076 | 97135813_97127456_97141441 |
| MRPL55_chr1_−_228296137_{—} | SHC1_chr1_−_154945768_{—} | TTC38_chr22_+_46665038_{—} |
| 228296722_228296019_228296849 | 154945984_154941924_154946723 | 46665186_46664488_46668231 |
| PPP2R2D_chr10_+_133748397_{—} | CLASP2_chr3_−_33600616_{—} | THUMPD2_chr2_−_39984387_{—} |
| 133748510_133748059_133753534 | 33600667_33592887_33600798 | 39984508_39983100_39988470 |
| SLC22A17_chr14_−_23817099_{—} | ZNF213-AS1_chr16_−_3182850_{—} | FAM104A_chr17_−_71223303_{—} |
| 23817205_23816940_23817369 | 3182946_3182104_3184630 | 71223321_71205907_71228224 |
| HS1BP3_chr2_−_20840732_{—} | CC2D2A_chr4_+_15480842_{—} | POSTN_chr13_−_38145505_{—} |
| 20840940_20838412_20845099 | 15480952_15480430_15482327 | 38145595_38143958_38151889 |
| OCIAD1_chr4_+_48833243_{—} | DTNB_chr2_−_25606704_{—} | BCS1L_chr2_+_219524759_{—} |
| 48833513_48833069_48834636 | 25606758_25602192_25610157 | 219524889_219524466_219525661 |
| MAP4_chr3_−_47910703_{—} | ACAA1_chr3_−_38173086_{—} | METTL26_chr16_−_685611_{—} |
| 47910817_47899002_47912302 | 38173129_38170879_38173416 | 685774_684797_686093 |
| CERS5_chr12_−_50538551_{—} | STX3_chr11_+_59564755_{—} | LUC7L3_chr17_+_48814647_{—} |
| 50538752_50537840_50560883 | 59564869_59562955_59568327 | 48814840_48814387_48817666 |
| FAR2P2_chr2_−_131183486_{—} | EWSR1_chr22_+_29687550_{—} | MEMO1_chr2_−_32118310_{—} |
| 131183611_131182731_131185276 | 29687588_29684775_29688125 | 32118390_32117203_32142994 |
| METTL26_chr16_−_685517_{—} | NUP88_chr17_−_5307418_{—} | RABGEF1_chr7_+_66233817_{—} |
| 685774_684797_686093 | 5307566_5302970_5308376 | 66234012_66205779_66236869 |
| SRPK2_chr7_−_104909252_{—} | WBP1_chr2_+_74686604_{—} | TMEM144_chr4_+_159133212_{—} |
| 104909316_104844232_105029094 | 74686689_74686225_74686769 | 159133372_159132726_159133759 |
| POLG_chr15_−_89875448_{—} | TSFM_chr12_+_58186768_{—} | PFDN5_chr12_+_53690213_{—} |
| 89875605_89873507_89876326 | 58186856_58180945_58189959 | 53690335_53689423_53691828 |
| SREBF1_chr17_−_17720861_{—} | NT5C2_chr10_−_104860419_{—} | MTMR3_chr22_+_30419445_{—} |
| 17720905_17720771_17721009 | 104860700_104859776_104860801 | 30419472_30418686_30421618 |
| SUGP2_chr19_−_19102148_{—} | IFFO1_chr12_−_6660563_{—} | RNH1_chr11_−_504823_{—} |
| 19102362_19101958_19105174 | 6660669_6660167_6664422 | 504996_502249_507112 |
| MRPL55_chr1_−_228296137_{—} | DDX39B_chr6_−_31506923_{—} | SRRT_chr7_+_100478316_{—} |
| 228296209_228296019_228296655 | 31507051_31504460_31508098 | 100478390_100473333_100478905 |
| CAP2_chr6_+_17514079_{—} | DMPK_chr19_−_46274228_{—} | IFT88_chr13_+_21141808_{—} |
| 17514185_17507543_17539499 | 46274318_46273898_46274825 | 21142136_21141395_21148518 |
| DMKN_chr19_−_36000812_{—} | CRAT_chr9_−_131870686_{—} | SLC27A3_chr1_+_153750236_{—} |
| 36000863_35996667_36001085 | 131870757_131870356_131871457 | 153750361_153749696_153750636 |
| RHOT1_chr17_+_30536368_{—} | PML_chr15_+_74324912_{—} | TMEM183A_chr1_+_202986883_{—} |
| 30536464_30535328_30551634 | 74325056_74317268_74325496 | 202987047_202985268_202987608 |
| CTNND1_chr11_+_57558856_{—} | APP_chr21_−_27369674_{—} | JMJD6_chr17_−_74717344_{—} |
| 57559145_57529591_57561481 | 27369731_27354790_27394155 | 74717438_74716580_74717879 |
| MLKL_chr16_−_74710198_{—} | CAMK2G_chr10_−_75577966_{—} | ILF3_chr19_+_10789288_{—} |
| 74710323_74709662_74712796 | 75578653_75577312_75579289 | 10789380_10787986_10789772 |
| HMOX2_chr16_+_4533637_{—} | SYNE4_chr19_−_36498026_{—} | ENOSF1_chr18_−_678695_{—} |
| 4533755_4526488_4556895 | 36498170_36496339_36499455 | 678737_675402_683245 |
| AFMID_chr17_+_76201683_{—} | ZFX_chrX_+_24169807_{—} | CANT1_chr17_−_76996181_{—} |
| 76201819_76200981_76202026 | 24169917_24167911_24190831 | 76996352_76994045_77001453 |
| RP11-20123.3_chr16_+_2577561_{—} | TOP3B_chr22_−_22325351_{—} | TPCN2_chr11_+_68847301_{—} |
| 2577616_2571124_2577773 | 22325433_22324778_22326248 | 68847351_68846488_68848867 |
| CDK10_chr16_+_89755659_{—} | KLK2_chr19_+_51377976_{—} | NPRL3_chr16_−_167299_{—} |
| 89755732_89753205_89756960 | 51378222_51376775_51379727 | 167374_162774_169124 |
| SYNE4_chr19_−_36497503_{—} | BRD8_chr5_−_137499775_{—} | EPN3_chr17_+_48613409_{—} |
| 36497573_36496339_36497651 | 137499822_137499033_137500008 | 48613560_48610205_48613781 |
| WDR27_chr6_−_170063658_{—} | AGTRAP_chr1_+_11805986_{—} | TRA2B_chr3_−_185649364_{—} |
| 170063745_170062520_170064261 | 11806280_11805894_11807496 | 185649640_185644522_185655612 |
| IGFLR1_chr19_−_36230610_{—} | TSPAN17_chr5_+_176078616_{—} | TAPT1_chr4_−_16172275_{—} |
| 36230989_36230527_36231924 | 176078667_176074703_176079743 | 16172352_16168416_16175826 |
| SLC38A11_chr2_−_165801078_{—} | ZNF223_chr19_+_44567417_{—} | ELMOD3_chr2_+_85582677_{—} |
| 165801144_165796066_165811718 | 44567719_44564994_44570216 | 85582907_85582293_85584089 |
| MPHOSPH8_chr13_+_20244979_{—} | NUP88_chr17_−_5321334_{—} | MICAL3_chr22_−_18367040_{—} |
| 20245104_20244503_20245345 | 5321461_5320002_5322673 | 18367124_18354789_18368643 |
| BCAR1_chr16_−_75271080_{—} | CLSTN1_chr1_−_9797555_{—} | WDFY3_chr4_−_85648013_{—} |
| 75271242_75269884_75276367 | 9797612_9796100_9801151 | 85648064_85645747_85654534 |
| THNSL2_chr2_+_88485069_{—} | PARL_chr3_−_183560085_{—} | MRPL55_chr1_−_228296655_{—} |
| 88485243_88478532_88485416 | 183560235_183558428_183562018 | 228296722_228295570_228296849 |
| FBXO38_chr5_+_147806775_{—} | CTBS_chr1_−_85035613_{—} | DTNB_chr2_−_25678273_{—} |
| 147807285_147805264_147812986 | 85035822_85031695_85036264 | 25678363_25674504_25705664 |
| MAGI3_chr1_+_114224833_{—} | CBFA2T2_chr20_+_32211578_{—} | MTIF3_chr13_−_28015153_{—} |
| 114224924_114223958_114225518 | 32211660_32211102_32212569 | 28015214_28014586_28019224 |
| IFT43_chr14_+_76525669_{—} | POSTN_chr13_−_38148708_{—} | SFXN2_chr10_+_104489075_{—} |
| 76525716_76488737_76548637 | 38148789_38143958_38151889 | 104489151_104488286_104489479 |
| CRELD1_chr3_+_9985585_{—} | PSMC5_chr17_+_61905210_{—} | DCUN1D4_chr4_+_52753288_{—} |
| 9985779_9985199_9986048 | 61905283_61904874_61905497 | 52753410_52752804_52757925 |
| GATSL3_chr22_−_30684694_{—} | PSMC5_chr17_+_61905033_{—} | ARL16_chr17_−_79649472_{—} |
| 30684765_30683549_30685373 | 61905283_61904874_61905497 | 79649705_79649179_79650042 |
| COL16A1_chr1_−_32145404_{—} | CHURC1-FNTB_chr14_+_65392724_{—} | MRPL55_chr1_−_228296137_{—} |
| 32145452_32145286_32145642 | 65392798_65390844_65398855 | 228296175_228296019_228296849 |
| MRPL55_chr1_−_228296137_{—} | TEAD1_chr11_+_12900435_{—} | R3HDM2_chr12_−_57686354_{—} |
| 228296722_228296022_228296849 | 12900447_12886447_12901254 | 57686450_57677839_57689181 |
| PLD3_chr19_+_40871459_{—} | AGA_chr4_−_178357429_{—} | KIF13A_chr6_−_17790102_{—} |
| 40871492_40854675_40871624 | 178357505_178355643_178358558 | 17790141_17788106_17794802 |
| SLC25A16_chr10_−_70276840_{—} | ZMYND8_chr20_−_45841286_{—} | FAM193B_chr5_−_176958953_{—} |
| 70276942_70276600_70287002 | 45841370_45839542_45848908 | 176959201_176958522_176959443 |
| SUN1_chr7_+_889559_{—} | MBD1_chr18_−_47797838_{—} | BCS1L_chr2_+_219525661_{—} |
| 889670_889240_891020 | 47797910_47794033_47799046 | 219526030_219524466_219526128 |
| PPP3CB_chr10_−_75199629_{—} | IFT43_chr14_+_76524984_{—} | SLC25A29_chr14_−_100761962_{—} |
| 75199659_75198178_75204482 | 76525017_76488737_76548637 | 100762330_100759714_100765178 |
| MEIS1_chr2_+_66796181_{—} | TM2D1_chr1_−_62189384_{—} | AHI1_chr6_−_135622545_{—} |
| 66796277_66795888_66798377 | 62189444_62175109_62190573 | 135622677_135621696_135639656 |
| ZNF213-AS1_chr16_−_3182850_{—} | ANKHD1_chr5_+_139851823_{—} | METTL26_chr16_−_684888_{—} |
| 3183195_3182104_3184630 | 139851880_139844361_139862164 | 685063_684797_685611 |
| HMGN1_chr21_−_40719172_{—} | CEP290_chr12_−_88448116_{—} | CRTC1_chr19_+_18854917_{—} |
| 40719218_40717200_40719304 | 88448190_88447523_88449352 | 18854965_18853836_18856632 |
| PBRM1_chr3_−_52588739_{—} | IGHMBP2_chr11_+_68697790_{—} | RTN2_chr19_−_45996417_{—} |
| 52588895_52584833_52595782 | 68697902_68696825_68700766 | 45996636_45992811_45997423 |
| TNPO2_chr19_−_12816823_{—} | UCHL5_chr1_−_192989436_{—} | GUSB_chr7_−_65435268_{—} |
| 12816923_12816582_12817009 | 192989511_192985525_192990226 | 65435353_65432894_65439281 |
| TAX1BP1_chr7_+_27855967_{—} | CD47_chr3_−_107768465_{—} | SUN1_chr7_+_872141_{—} |
| 27856139_27839709_27867356 | 107768498_107766139_107770785 | 872238_856310_878434 |
| CYP2J2_chr1_−_60370037_{—} | TIMM17B_chrX_−_48752634_{—} | NCOA5_chr20_−_44708025_{—} |
| 60370188_60366775_60370542 | 48753255_48752384_48754041 | 44708092_44699175_44718455 |
| ARMCX2_chrX_−_100913446_{—} | GDAP2_chr1_−_118429198_{—} | PCSK5_chr9_+_78749024_{—} |
| 100913511_100913128_100914742 | 118429275_118426249_118439454 | 78749128_78722267_78771960 |
| KPNA1_chr3_−_122176572_{—} | TBC1D1_chr4_+_38054726_{—} | VKORC1_chr16_−_31104632_{—} |
| 122176695_122172848_122180070 | 38054846_38051519_38055819 | 31104803_31102663_31105877 |
| ESYT2_chr7_−_158545471_{—} | TCF20_chr22_−_42564614_{—} | RP11-286N22.8_chr11_+_61205096_{—} |
| 158545534_158542414_158552176 | 42564742_42557364_42565852 | 61205320_61197654_61213412 |
| ARHGEF1_chr19_+_42410608_{—} | NPDC1_chr9_−_139935122_{—} | TERF1_chr8_+_73942570_{—} |
| 42410772_42410177_42410854 | 139935189_139934888_139935513 | 73942630_73939287_73944276 |
| RP4-800G7.2_chr7_+_148985521_{—} | PEX19_chr1_−_160253319_{—} | FLOT2_chr17_−_27211242_{—} |
| 148986548_148984867_148987028 | 160253429_160252899_160254844 | 27211333_27210249_27215962 |
| KCTD20_chr6_+_36411194_{—} | ADIRF_chr10_+_88729956_{—} | CEP95_chr17_+_62512840_{—} |
| 36411307_36410888_36437828 | 88730019_88728362_88730232 | 62512946_62510476_62515438 |
| TSEN15_chr1_+_184041290_{—} | AKAP8L_chr19_−_15509440_{—} | UQCRH_chr1_+_46775568_{—} |
| 184041328_184023997_184041960 | 15509577_15508666_15510112 | 46775716_46769492_46775826 |
| ZNF528-AS1_chr19_−_52900447_{—} | CCNH_chr5_−_86690221_{—} | COASY_chr17_+_40714313_{—} |
| 52900520_52899003_52900714 | 86690301_86688721_86690860 | 40714505_40714237_40714629 |
| ZNF691_chr1_+_43314961_{—} | SF3B1_chr2_−_198283619_{—} | ACAAl_chr3_−_38167652_{—} |
| 43315084_43312328_43316620 | 198283675_198283312_198285151 | 38167832_38167372_38168000 |
| ZNF83_chr19_−_53119970_{—} | COG4_chr16_−_70549770_{—} | ABHD2_chr15_+_89656955_{—} |
| 53120128_53118050_53122188 | 70549943_70548412_70551528 | 89657055_89631794_89694907 |
| MRPL55_chr1_−_228296137_{—} | NACA_chr12_−_57109654_{—} | TRIQK_chr8_−_93966633_{—} |
| 228296175_228296019_228296655 | 57109990_57108471_57113320 | 93966792_93929235_93978234 |
| EDRF1_chr10_+_127417571_{—} | TRA2A_chr7_−_23561739_{—} | HDHD2_chr18_−_44663605_{—} |
| 127417673_127414407_127417926 | 23562051_23561459_23571407 | 44663709_44662820_44676748 |
| RP11-606E8.2_chr11_+_93530700_{—} | IPO9_chr1_+_201804081_{—} | ZNF428_chr19_−_44112656_{—} |
| 93530885_93529706_93535000 | 201804192_201798500_201816406 | 44112755_44112259_44118380 |
| GUSBP11_chr22_−_23982494_{—} | LETMD1_chr12_+_51447594_{—} | AFMID_chr17_+_76201683_{—} |
| 23982610_23981129_24002051 | 51447643_51442968_51450132 | 76201819_76198832_76202026 |
| OARD1_chr6_−_41037814_{—} | KCTD6_chr3_+_58479879_{—} | GPATCH8_chr17_−_42544464_{—} |
| 41037873_41035176_41038870 | 58480135_58477896_58484439 | 42544482_42541912_42552196 |
| MAP3K4_chr6_+_161519309_{—} | ACAD10_chr12_+_112167609_{—} | GON4L_chr1_−_155802622_{—} |
| 161519459_161518208_161522923 | 112167760_112165947_112171726 | 155802741_155796819_155823066 |
| TARS2_chr1_+_150464073_{—} | STAU1_chr20_−_47795658_{—} | RNF115_chr1_+_145683574_{—} |
| 150464138_150463987_150464886 | 47795781_47770608_47804652 | 145683647_145682094_145684584 |
| ZBTB8OS_chr1_−_33100302_{—} | RP11-73M18.2_chr14_+_104040443_{—} | PPM1K_chr4_−_89199687_{—} |
| 33100393_33093145_33116033 | 104040507_104029461_104053610 | 89199794_89198395_89205557 |
| DONSON_chr21_−_34955793_{—} | PKIG_chr20_+_43211225_{—} | TBCE_chr1_+_235596260_{—} |
| 34955972_34954361_34956895 | 43211372_43160619_43218437 | 235596413_235594119_235597518 |
| DHODH_chr16_+_72055022_{—} | SNU13_chr22_−_42078359_{—} | NFASC_chr1_+_204971723_{—} |
| 72055210_72051005_72057063 | 42078591_42076368_42084797 | 204971876_204957934_204978684 |
| GGCX_chr2_−_85779538_{—} | MICAL3_chr22_−_18309219_{—} | HKR1_chr19_+_37835511_{—} |
| 85779690_85779104_85780061 | 18309282_18305826_18314619 | 37835676_37826159_37838091 |
| ABI1_chr10_−_27060003_{—} | AFMID_chr17_+_76200736_{—} | MAPK3_chr16_−_30126915_{—} |
| 27060018_27059274_27065993 | 76200822_76198832_76202026 | 30126966_30126029_30127956 |
| RNFT1_chr17_−_58037428_{—} | C12orf4_chr12_−_4627939_{—} | CSNK1D_chr17_−_80207573_{—} |
| 58037529_58035805_58039900 | 4628046_4614546_4634419 | 80208055_80207478_80209254 |
| TTC7B_chr14_−_91069596_{—} | AFMID_chr17_+_76201520_{—} | ERBIN_chr5_+_65364704_{—} |
| 91069647_91059970_91077085 | 76201599_76200981_76202026 | 65364848_65350779_65372143 |
| RHOQ_chr2_+_46803699_{—} | HNMT_chr2_+_138724666_{—} | CHD1L_chr1_+_146742592_{—} |
| 46803795_46770951_46808066 | 138724956_138722198_138727734 | 146742666_146740537_146747016 |
| NT5C3B_chr17_−_39991839_{—} | INF2_chr14_+_105181620_{—} | NEB_chr2_−_152349866_{—} |
| 39991934_39991524_39992110 | 105181677_105181193_105185131 | 152349959_152349008_152361991 |
| RBPMS_chr8_+_30402010_{—} | AP2M1_chr3_+_183898432_{—} | CTD-2006C1.2_chr19_+_12112588_{—} |
| 30402141_30361953_30404808 | 183898438_183898039_183898636 | 12112637_12098638_12146368 |
| GK_chrX_+_30745582_{—} | TAF1D_chr11_−_93464315_{—} | USO1_chr4_+_76716488_{—} |
| 30745669_30742298_30746848 | 93464382_93463878_93467790 | 76716509_76715054_76720774 |
| DENND6B_chr22_−_50752828_{—} | LRRFIP1_chr2_+_238626404_{—} | FAM84A_chr2_+_14773709_{—} |
| 50752923_50752701_50753033 | 238626452_238617273_238628165 | 14773952_14773061_14774067 |
| SP140L_chr2_+_231265048_{—} | GOLIM4_chr3_−_167758573_{—} | PIGF_chr2_−_46815213_{—} |
| 231265128_231264957_231265669 | 167758657_167754782_167759179 | 46815318_46808730_46819613 |
| DHX58_chr17_−_40255625_{—} | PTBP2_chr1_+_97271974_{—} | IFI44_chr1_+_79126238_{—} |
| 40255816_40254330_40256783 | 97272008_97270495_97272421 | 79126339_79125168_79128388 |
| DLG1_chr3_−_196803456_{—} | SYNE4_chr19_−_36497503_{—} | ZNF83_chr19_−_53119970_{—} |
| 196803556_196802741_196807921 | 36497573_36496339_36498026 | 53120094_53118050_53122188 |
| FRG1_chr4_+_190876191_{—} | LIMS2_chr2_−_128431251_{—} | NUMB_chr14_−_73745988_{—} |
| 190876306_190873442_190878552 | 128431317_128415136_128432587 | 73746132_73744001_73749066 |
| TUSC3_chr8_+_15615299_{—} | SMC5_chr9_+_72961520_{—} | ENOSF1_chr18_−_691203_{—} |
| 15615364_15601121_15621711 | 72961565_72959185_72961981 | 691276_690631_693881 |
| WASH3P_chr15_+_102512798_{—} | PPP1R12A_chr12_−_80173118_{—} | PLA2G5_chr1_+_20416281_{—} |
| 102512897_102501844_102513103 | 80173131_80172380_80175594 | 20416388_20412720_20417060 |
| DGUOK_chr2_+_74173845_{—} | OSTC_chr4_+_109584388_{—} | FDPS_chr1_+_155279579_{—} |
| 74174033_74154179_74185272 | 109584462_109576813_109588402 | 155279756_155278867_155279833 |
| ARL16_chr17_−_79650042_{—} | ZC3H11A_chr1_+_203765420_{—} | ACOX1_chr17_−_73969705_{—} |
| 79650156_79649179_79650565 | 203765729_203764922_203770702 | 73969866_73953647_73974614 |
| NUP85_chr17_+_73225092_{—} | ETHE1_chr19_−_44030352_{—} | SLC35B1_chr17_−_47784704_{—} |
| 73225226_73222252_73227434 | 44030501_44015718_44030666 | 47784806_47784430_47785437 |
| TCOF1_chr5_+_149771106_{—} | BCS1L_chr2_+_219524759_{—} | PLSCR1_chr3_−_146246400_{—} |
| 149771358_149769586_149771519 | 219524889_219524466_219526481 | 146246618_146243434_146251256 |
| CYP4A11_chr1_−_47398654_{—} | MINK1_chr17_+_4762507_{—} | CMTM7_chr3_+_32490945_{—} |
| 47398719_47398509_47400124 | 4762623_4736935_4781611 | 32491044_32483505_32493883 |
| LETMD1_chr12_+_51445874_{—} | RHBDD1_chr2_+_227704124_{—} | RBM41_chrX_−_106356626_{—} |
| 51445990_51442968_51450132 | 227704343_227700803_227729319 | 106356698_106332069_106358581 |
| TDG_chr12_+_104376913_{—} | DAZAP1_chr19_+_1425876_{—} | EEF1D_chr8_−_144663455_{—} |
| 104376996_104376712_104377072 | 1425959_1422395_1428840 | 144663876_144663324_144668388 |
| CA11_chr19_−_49141496_{—} | PIP5K1A_chr1_+_151196846_{—} | CAPRIN2_chr12_−_30873744_{—} |
| 49141550_49141401_49142144 | 151196882_151196755_151199795 | 30873849_30869610_30876192 |
| PLEKHA7_chr11_−_16828690_{—} | PDCD5_chr19_+_33075860_{—} | VPS29_chr12_−_110937339_{—} |
| 16828771_16824842_16834659 | 33075917_33073138_33078158 | 110937351_110934807_110939853 |
| CD47_chr3_−_107770785_{—} | XIRP2_chr2_+_168099078_{—} | ADAM33_chr20_−_3652780_{—} |
| 107770817_107766139_107776323 | 168108457_168098420_168110541 | 3652976_3652632_3653367 |
| RPS24_chr10_+_79797722_{—} | ARL16_chr17_−_79649520_{—} | RP11-903H12.5_chr14_+_21161705_{—} |
| 79797740_79797062_79800372 | 79649705_79649179_79650042 | 21161809_21152917_21167513 |
| RASSF7_chr11_+_563395_{—} | DOCK6_chr19_−_11339973_{—} | ENAH_chr1_−_225692692_{—} |
| 563478_563317_563557 | 11340066_11339711_11342860 | 225692755_225688772_225695652 |
| SEC31A_chr4_−_83783686_{—} | PEX2_chr8_−_77900542_{—} | TXNL4A_chr18_−_77736717_{—} |
| 83783725_83778917_83784470 | 77900574_77896431_77912225 | 77736787_77733856_77748239 |
| RBM6_chr3_+_50012755_{—} | RAB40B_chr17_−_80654264_{—} | DST_chr6_−_56333779_{—} |
| 50012825_50000118_50085677 | 80654729_80622432_80656330 | 56333797_56330993_56334680 |
| AMT_chr3_−_49455599_{—} | UPP1_chr7_+_48142893_{—} | ZNF83_chr19_−_53119970_{—} |
| 49455661_49455406_49456403 | 48143008_48141579_48146469 | 53120068_53118050_53122188 |
| IL32_chr16_+_3115642_{—} | MEF2D_chr1_−_156446285_{—} | C1D_chr2_−_68280192_{—} |
| 3115688_3115495_3115784 | 156446306_156445029_156446803 | 68280418_68274451_68290076 |
| LOXL3_chr2_−_74763835_{—} | TTC7B_chr14_−_91069596_{—} | MRPL22_chr5_+_154330362_{—} |
| 74764055_74763598_74776495 | 91069647_91044652_91077085 | 154330498_154320825_154335930 |
| NUPL2_chr7_+_23235457_{—} | GATAD2A_chr19_+_19612777_{—} | PPFIA1_chr11_+_70197099_{—} |
| 23235534_23226765_23236298 | 19612852_19612225_19613139 | 70197129_70196145_70200406 |
| Clorf43_chr1_−_154186368_{—} | DGUOK_chr2_+_74177727_{—} | MPRIP_chr17_+_17067422_{—} |
| 154186422_154185100_154186932 | 74177859_74174033_74185272 | 17071229_17064670_17075031 |
| RABGGTB_chr1_+_76255836_{—} | UQCRH_chr1_+_46775568_{—} | TUSC3_chr8_+_15615299_{—} |
| 76255959_76255742_76256969 | 46775716_46774799_46775826 | 15615364_15605974_15615538 |
| TTC13_chr1_−_231071903_{—} | TTLL7_chr1_−_84414310_{—} | MRPL48_chr11_+_73554291_{—} |
| 231072010_231069607_231090078 | 84414378_84412965_84415547 | 73554412_73536841_73555851 |
| SEC16A_chr9_−_139339503_{—} | MOGS_chr2_−_74691622_{—} | MTHFD2L_chr4_+_75025771_{—} |
| 139339563_139338352_139340096 | 74691849_74690513_74692022 | 75025805_75023998_75040222 |
| RMND5B_chr5_+_177558237_{—} | SLC2A11_chr22_+_24219907_{—} | TMEM70_chr8_+_74891313_{—} |
| 177558377_177558055_177565108 | 24220056_24219358_24224645 | 74891406_74891096_74893389 |
| SMTN_chr22_+_31496870_{—} | VIPAS39_chr14_−_77907408_{—} | RNF7_chr3_+_141461485_{—} |
| 31496939_31495882_31500301 | 77907516_77904224_77908902 | 141461749_141457351_141462350 |
| PRMT1_chr19_+_50183128_{—} | FAHD2A_chr2_+_96076611_{—} | GOLGA2P7_chr15_−_84872928_{—} |
| 50183182_50180573_50185166 | 96076774_96076334_96078181 | 84873083_84871772_84873177 |
| LAMP2_chrX_−_119572930_{—} | PPP3CC_chr8_+_22396981_{—} | EWSR1_chr22_+_29687553_{—} |
| 119573148_119565317_119575584 | 22397011_22390531_22398127 | 29687588_29684775_29688125 |
| RALY_chr20_+_32619327_{—} | IVNS1ABP_chr1_−_185275616_{—} | TTLL3_chr3_+_9859328_{—} |
| 32619410_32581937_32659871 | 185275757_185274775_185276145 | 9860604_9855029_9867483 |
| EPB41L1_chr20_+_34797409_{—} | FANCL_chr2_−_58390163_{—} | CBR3-AS1_chr21_−_37517276_{—} |
| 34797820_34785963_34800193 | 58390209_58388773_58390568 | 37517374_37505372_37518553 |
| HOOK3_chr8_+_42855413_{—} | SMAD4_chr18_+_48603676_{—} | AC005154.6_chr7_−_30618621_{—} |
| 42855419_42852780_42857249 | 48603789_48603146_48604625 | 30618744_30617707_30618846 |
| FRRS1_chr1_−_100176910_{—} | SHROOM1_chr5_−_132163152_{—} | ABHD11_chr7_−_73152205_{—} |
| 100177088_100176505_100177660 | 132163299_132161874_132166285 | 73152402_73152065_73152657 |
| SEC16A_chr9_−_139339503_{—} | ABLIM3_chr5_+_148622053_{—} | HMGN3_chr6_−_79911779_{—} |
| 139339563_139338352_139341306 | 148622101_148619451_148624443 | 79911872_79911443_79911992 |
| AFMID_chr17_+_76201683_{—} | USP53_chr4_+_120135224_{—} | MADD_chr11_+_47348288_{—} |
| 76201834_76198832_76202026 | 120135377_120134028_120138704 | 47348358_47346128_47350208 |
| PQLC1_chr18_−_77693968_{—} | TMEM267_chr5_−_43476215_{—} | DHX34_chr19_+_47880171_{—} |
| 77694022_77679400_77703328 | 43476360_43454145_43483923 | 47880246_47879817_47880356 |
| MINOS1_chr1_+_19943751_{—} | LRRFIP1_chr2_+_238647874_{—} | STRN3_chr14_−_31398406_{—} |
| 19943830_19923603_19949967 | 238647952_238617273_238657006 | 31398517_31381388_31404368 |
| KIF13A_chr6_−_17771344_{—} | TJP1_chr15_−_30011980_{—} | FAM193B_chr5_−_176958953_{—} |
| 17771449_17765177_17772138 | 30012220_30011342_30012561 | 176959201_176952206_176959443 |
| TTLL3_chr3_+_9874787_{—} | SDCCAG3_chr9_−_139304541_{—} | MYL6_chr12_+_56553370_{—} |
| 9874929_9871079_9876364 | 139304691_139302390_139304779 | 56553406_56552495_56553758 |
| TMEM141_chr9_+_139686162_{—} | ECHDC2_chr1_−_53370705_{—} | ADAT1_chr16_−_75652483_{—} |
| 139686229_139685876_139686398 | 53372283_53370505_53373539 | 75652596_75651170_75654163 |
| DST_chr6_−_56327843_{—} | C11orf80_chr11_+_66523823_{—} | ZNF75D_chrX_−_134429693_{—} |
| 56327954_56325052_56328362 | 66523976_66512390_66526513 | 134429965_134421778_134475692 |
| AGBL5_chr2_+_27291915_{—} | ANXA2_chr15_−_60689456_{—} | MTA1_chr14_+_105934674_{—} |
| 27291962_27291612_27292440 | 60689537_60678285_60690069 | 105934686_105933079_105935803 |
| NTAN1_chr16_−_15141853_{—} | EPN3_chr17_+_48613389_{—} | GTPBP1_chr22_+_39104849_{—} |
| 15141956_15141777_15149747 | 48613639_48610346_48613781 | 39104961_39102152_39111911 |
| DLG1_chr3_−_196802707_{—} | SCAF8_chr6_+_155099134_{—} | D2HGDH_chr2_+_242694045_{—} |
| 196802741_196796131_196803456 | 155099179_155055064_155108994 | 242694161_242690803_242694459 |
| NME4_chr16_+_448207_{—} | FLAD1_chr1_+_154962035_{—} | PTPN6_chr12_+_7060626_{—} |
| 448394_447313_448989 | 154962183_154961325_154962634 | 7060683_7055902_7060771 |
| LUC7L_chr16_−_278331_{—} | PXN_chr12_−_120653362_{—} | RP11-43F13.1_chr5_−_1632758_{—} |
| 278401_277335_279277 | 120653464_120653220_120659425 | 1632866_1629745_1632959 |
| CELF1_chr11_−_47515882_{—} | SRRM2_chr16_+_2818505_{—} | RREB1_chr6_+_7240670_{—} |
| 47515995_47510576_47521004 | 2818600_2818262_2818997 | 7240835_7232140_7246656 |
| POLR3C_chr1_−_145597513_{—} | TPCN1_chr12_+_113726299_{—} | NR4A1_chr12_+_52446279_{—} |
| 145597574_145597054_145598535 | 113726331_113726039_113726562 | 52446429_52437695_52448110 |
| FUK_chr16_+_70503054_{—} | PAAF1_chr11_+_73598075_{—} | TSC2_chr16_+_2132436_{—} |
| 70503226_70502871_70504216 | 73598144_73589864_73598398 | 2132505_2131799_2133695 |
| DTX3_chr12_+_57999126_{—} | PSAP_chr10_−_73583644_{—} | HKR1_chr19_+_37815697_{—} |
| 57999514_57998641_57999972 | 73583653_73581764_73585593 | 37815838_37815167_37838091 |
| CRCP_chr7_+_65595113_{—} | LRRFIP1_chr2_+_238659842_{—} | SHMT2_chr12_+_57625263_{—} |
| 65595235_65592727_65595730 | 238659914_238657967_238661951 | 57625343_57624783_57625495 |
| MACF1_chr1_+_39946591_{—} | FLOT2_chr17_−_27211242_{—} | TMEM25_chr11_+_118404134_{—} |
| 39946702_39945681_39950272 | 27211333_27210249_27212874 | 118404266_118403922_118404571 |
| PREPL_chr2_−_44587757_{—} | TRAPPC2_chrX_−_13752168_{—} | CAPN7_chr3_+_15282959_{—} |
| 44587815_44573529_44588518 | 13752304_13738101_13752643 | 15283095_15282360_15283684 |
| SMPD4_chr2_−_130917085_{—} | SLTM_chr15_−_59191667_{—} | PTCD3_chr2_+_86352917_{—} |
| 130917207_130914969_130918758 | 59192136_59191051_59193458 | 86353003_86352638_86354283 |
| GIPC1_chr19_−_14603668_{—} | PDLIM7_chr5_−_176918807_{—} | SEPT6_chrX_−_118759297_{—} |
| 14603724_14591590_14606848 | 176918977_176918421_176919405 | 118759342_118752749_118763280 |
| CASK_chrX_−_41416284_{—} | CDK7_chr5_+_68551286_{—} | SBDSP1_chr7_+_72301271_{—} |
| 41416353_41414888_41419031 | 68551355_68548278_68553869 | 72301393_72300362_72302181 |
| ITGA6_chr2_+_173366499_{—} | INO80E_chr16_+_30012785_{—} | TMEM185A_chrX_−_148685652_{—} |
| 173366629_173362828_173368818 | 30012851_30012361_30016541 | 148685736_148682143_148713225 |
| DTNA_chr18_+_32464691_{—} | NOD1_chr7_−_30477188_{—} | WIZ_chr19_−_15541822_{—} |
| 32464740_32462165_32470263 | 30477272_30469073_30485756 | 15541993_15538344_15543244 |
| CD74_chr5_−_149782683_{—} | LPAR2_chr19_−_19738562_{—} | MYEF2_chr15_−_48459545_{—} |
| 149782875_149782188_149784242 | 19738778_19738093_19738900 | 48459598_48458325_48460827 |
| CHORDC1_chr11_−_89939915_{—} | ZZEF1_chr17_−_3959509_{—} | CLK3_chr15_+_74914460_{—} |
| 89939978_89939440_89943703 | 3959639_3957489_3961287 | 74914557_74912566_74914834 |
| ATP2B4_chr1_+_203702350_{—} | BCAR1_chr16_−_75298248_{—} | SYNE1_chr6_−_152621775_{—} |
| 203702528_203696699_203708673 | 75298499_75276988_75299722 | 152621916_152615262_152623003 |
| CARS2_chr13_−_111296411_{—} | KXD1_chr19_+_18671229_{—} | ATP11C_chrX_−_138813809_{—} |
| 111296529_111294868_111296731 | 18671360_18668724_18672845 | 138813914_138811121_138820074 |
| GGA1_chr22_+_38021803_{—} | TM2D1_chr1_−_62152463_{—} | NADK_chr1_−_1688217_{—} |
| 38021956_38019640_38025468 | 62152593_62149218_62160368 | 1688321_1688047_1688619 |
| LRRC37B_chr17_+_30372718_{—} | TIAL1_chr10_−_121339982_{—} | RNF10_chr12_+_121008993_{—} |
| 30372837_30362658_30374779 | 121340050_121339522_121341433 | 121009094_121004783_121013433 |
| CENPX_chr17_−_79977516_{—} | ACLY_chr17_−_40052872_{—} | SRSF3_chr6_+_36567597_{—} |
| 79977570_79977257_79977733 | 40052902_40049427_40054001 | 36568053_36566760_36568928 |
| ZSCAN25_chr7_+_99216147_{—} | GLT8D1_chr3_−_52738739_{—} | MAPK10_chr4_−_87019676_{—} |
| 99216274_99214639_99217183 | 52738968_52734512_52739462 | 87020438_86989108_87022204 |
| CD151_chr11_+_834529_{—} | SIRT2_chr19_−_39384458_{—} | NME6_chr3_−_48337608_{—} |
| 834745_833022_836062 | 39384507_39380784_39390145 | 48337726_48336725_48338216 |
| NOL3_chr16_+_67207910_{—} | HGD_chr3_−_120365113_{—} | ST7_chr7_+_116774177_{—} |
| 67207949_67204477_67208064 | 120365213_120360540_120365819 | 116774246_116772014_116776134 |
| CD47_chr3_−_107769424_{—} | RP11-529K1.3_chr16_+_70346511_{—} | DCAF6_chr1_+_168007608_{—} |
| 107769449_107766139_107770785 | 70346560_70333257_70349871 | 168007726_167974031_168012262 |
| NEB_chr2_−_152359862_{—} | KHK_chr2_+_27317344_{—} | HRAS_chr11_−_533276_{—} |
| 152359955_152349008_152361991 | 27317479_27315316_27317667 | 533358_532755_533452 |
| RABGEF1_chr7_+_66204982_{—} | PLXDC1_chr17_−_37235754_{—} | RHBDF1_chr16_−_109296_{—} |
| 66205779_66177211_66236869 | 37235781_37235417_37239711 | 109347_109091_109416 |
| MTO1_chr6_+_74181182_{—} | R3HDM2_chr12_−_57682791_{—} | PDLIM7_chr5_−_176918807_{—} |
| 74181280_74176329_74183087 | 57682823_57677839_57689181 | 176918926_176918421_176919405 |
| ASL_chr7_+_65546789_{—} | BCLAF1_chr6_−_136588166_{—} | CNNM2_chr10_+_104831530_{—} |
| 65546984_65541080_65547354 | 136588313_136582615_136589299 | 104831596_104828479_104835842 |
| EAF2_chr3_+_121573533_{—} | NFYA_chr6_+_41048549_{—} | DPY19L3_chr19_+_32958379_{—} |
| 121573670_121563394_121575857 | 41048636_41046903_41051784 | 32958532_32955690_32959636 |
| PSMA4_chr15_+_78834917_{—} | SVIL_chr10_−_29820930_{—} | ABCA5_chr17_−_67247887_{—} |
| 78834987_78834561_78836531 | 29821101_29820217_29839525 | 67248007_67246762_67249713 |
| TBCE_chr1_+_235564817_{—} | TARSL2_chr15_−_102239533_{—} | DDR1_chr6_+_30852314_{—} |
| 235564902_235543464_235577747 | 102239638_102226265_102241288 | 30852487_30850760_30856464 |
| COCH_chr14_+_31354115_{—} | TRMO_chr9_−_100673199_{—} | PDK1_chr2_+_173433468_{—} |
| 31354247_31353862_31354599 | 100675257_100672898_100675682 | 173433545_173431661_173435453 |
| SRSF2_chr17_−_74731853_{—} | SUPT20H_chr13_−_37599456_{—} | MGST2_chr4_+_140608813_{—} |
| 74731957_74731240_74732235 | 37599574_37598579_37600340 | 140608894_140599796_140624608 |
| C2CD5_chr12_−_22612425_{—} | GOLGA2_chr9_−_131035063_{—} | DHX9_chr1_+_182824711_{—} |
| 22612476_22610095_22622642 | 131035144_131030803_131036128 | 182824848_182823313_182825666 |
| CAMKK2_chr12_−_121682375_{—} | VRK3_chr19_−_50512492_{—} | GMIP_chr19_−_19745315_{—} |
| 121682418_121678672_121682942 | 50512546_50511083_50519280 | 19745512_19744999_19745600 |
| DGUOK_chr2_+_74177753_{—} | CLCC1_chr1_−_109504930_{—} | PILRB_chr7_+_99951517_{—} |
| 74177859_74174033_74185272 | 109505091_109493070_109505982 | 99951635_99951106_99952765 |
| TRA2B_chr3_−_185649364_{—} | SPIN1_chr9_+_91033764_{—} | TSPAN4_chr11_+_862549_{—} |
| 185649640_185643414_185655612 | 91033866_91003453_91041296 | 862741_850367_864436 |
| ZCCHC6_chr9_−_88941285_{—} | OAZ3_chr1_+_151740620_{—} | SLC12A4_chr16_−_67984556_{—} |
| 88941406_88940429_88943254 | 151740709_151739775_151742647 | 67984614_67984396_67985042 |
| PKD1_chr16_−_2163041_{—} | BYSL_chr6_+_41897869_{—} | EPB41_chr1_+_29386933_{—} |
| 2163060_2162964_2163161 | 41898008_41895274_41898372 | 29386996_29379824_29391493 |
| HNRNPM_chr19_+_8527412_{—} | RELL2_chr5_+_141018521_{—} | STAM_chr10_+_17726830_{—} |
| 8527465_8520458_8528380 | 141018588_141018427_141019030 | 17726926_17702547_17730025 |
| ATR_chr3_−_142169307_{—} | PKP4_chr2_+_159535092_{—} | ENTPD6_chr20_+_25187711_{—} |
| 142169444_142168444_142171969 | 159535166_159530512_159536940 | 25188033_25176503_25190484 |
| CYP20A1_chr2_+_204137392_{—} | NGLY1_chr3_−_25761620_{—} | EDEM1_chr3_+_5251859_{—} |
| 204137471_204131404_204143295 | 25761682_25761126_25770623 | 5251933_5249948_5252804 |
| FAM193B_chr5_−_176958953_{—} | TMX2_chr11_+_57505307_{—} | ATL1_chr14_+_51096712_{—} |
| 176959132_176952206_176959443 | 57505498_57505140_57505825 | 51096727_51095180_51098946 |
| GATSL3_chr22_−_30682251_{—} | TIA1_chr2_−_70455475_{—} | CD46_chr1_+_207963597_{—} |
| 30682365_30682087_30682813 | 70455594_70454954_70456190 | 207963690_207959027_207966863 |
| ENOSF1_chr18_−_697239_{—} | NSUN5P1_chr7_+_75042066_{—} | PIGP_chr21_−_38444444_{—} |
| 697355_694334_706469 | 75042210_75039743_75044162 | 38444610_38441924_38444733 |
| CENPS_chr1_+_10493898_{—} | FUS_chr16_+_31198122_{—} | FKRP_chr19_+_47251771_{—} |
| 10494022_10491458_10500403 | 31198157_31196500_31199645 | 47251922_47251345_47258668 |
| PMF1_chr1_+_156195347_{—} | CC2D2A_chr4_+_15480846_{—} | PTS_chr11_+_112100930_{—} |
| 156195459_156182967_156202110 | 15480952_15480430_15482327 | 112100953_112099396_112101348 |
| HACL1_chr3_−_15626754_{—} | SELENOP_chr5_−_42809861_{—} | C17orf62_chr17_−_80407049_{—} |
| 15626849_15624496_15631046 | 42809890_42804875_42811937 | 80407168_80403836_80408575 |
| CD27-AS1_chr12_−_6559506_{—} | ADAM33_chr20_−_3652780_{—} | FAM76B_chr11_−_95512241_{—} |
| 6560146_6557903_6560634 | 3652976_3652632_3653183 | 95512299_95512118_95512770 |
| TMEM136_chr11_+_120198149_{—} | GIGYF2_chr2_+_233565294_{—} | TMEM159_chr16_+_21172298_{—} |
| 120198349_120196077_120200685 | 233565364_233562102_233599864 | 21172329_21170067_21172405 |
| CHTOP_chr1_+_153614721_{—} | TBC1D17_chr19_+_50382579_{—} | INO80E_chr16_+_30015888_{—} |
| 153614905_153610924_153617539 | 50382761_50381829_50383535 | 30015978_30012851_30016541 |
| SARAF_chr8_−_29931392_{—} | FIP1L1_chr4_+_54306748_{—} | FNBP1_chr9_−_132678244_{—} |
| 29931544_29927575_29940362 | 54306775_54294350_54308819 | 132678259_132671278_132687238 |
| SMTN_chr22_+_31492983_{—} | TMEM141_chr9_+_139686077_{—} | IFT20_chr17_−_26659171_{—} |
| 31493046_31491595_31493254 | 139686229_139685876_139686398 | 26659408_26659013_26662365 |
| CRAT_chr9_−_131871041_{—} | IQSEC1_chr3_−_12944272_{—} | MARK3_chr14_+_103966492_{—} |
| 131871182_131870356_131871457 | 12944322_12943022_12949848 | 103966537_103946827_103969218 |
| PLXNB2_chr22_−_50733154_{—} | WDR90_chr16_+_705028_{—} | WNK2_chr9_+_96069058_{—} |
| 50733207_50729026_50745981 | 705147_703803_705306 | 96069103_96061543_96070609 |
| ATG4B_chr2_+_242592721_{—} | SLC20A2_chr8_−_42390314_{—} | CCDC18-AS1_chr1_−_93804712_{—} |
| 242592784_242590750_242592926 | 42390478_42330172_42396680 | 93804831_93791452_93804913 |
| TCERG1_chr5_+_145889629_{—} | TINAGL1_chr1_+_32042734_{—} | RSRC2_chr12_−_123005050_{—} |
| 145889723_145888808_145890003 | 32043059_32042196_32044804 | 123005128_123003598_123005931 |
| MPP3_chr17_−_41894044_{—} | NSRP1_chr17_+_28483557_{—} | FXYD3_chr19_+_35610071_{—} |
| 41894065_41893447_41895413 | 28483680_28445191_28499559 | 35610155_35607263_35611981 |
| YPEL5_chr2_+_30371110_{—} | SRSF4_chr1_−_29486375_{—} | PRPSAP2_chr17_+_18770569_{—} |
| 30371407_30369928_30381484 | 29486570_29485998_29486886 | 18770647_18769265_18775895 |
| TMEM184B_chr22_−_38642790_{—} | PPP2R3C_chr14_−_35585815_{—} | ABCD4_chr14_−_74759856_{—} |
| 38642891_38642106_38643775 | 35585943_35579835_35591107 | 74759982_74759572_74761850 |
| CHD1L_chr1_+_146742592_{—} | LMBRIL_chr12_−_49500327_{—} | UBE2F_chr2_+_238925174_{—} |
| 146742666_146740537_146743831 | 49500529_49499740_49500743 | 238925275_238903451_238933982 |
| ARNT_chr1_−_150814899_{—} | POT1_chr7_−_124469307_{—} | TARBP1_chr1_−_234536598_{—} |
| 150814944_150812130_150818738 | 124469396_124467359_124475332 | 234536694_234534299_234536926 |
| INTS3_chr1_+_153733495_{—} | RAB40C_chr16_+_668199_{—} | TRA2A_chr7_−_23561972_{—} |
| 153733585_153733394_153734045 | 668260_640433_675431 | 23562051_23561459_23571407 |
| LIPF_chr10_+_90425235_{—} | FBXL6_chr8_−_145581116_{—} | ZNF790-AS1_chr19_+_37314632_{—} |
| 90425316_90424248_90427043 | 145581162_145580781_145581287 | 37314723_37292835_37318551 |
| RP11-216L13.19_chr9_+_139703706_{—} | TMEM8B_chr9_+_35834457_{—} | GCFC2_chr2_−_75907318_{—} |
| 139703881_139702778_139704139 | 35834647_35829952_35841130 | 75907440_75900646_75914952 |
| CCDC25_chr8_−_27606011_{—} | UBE2V1_chr20_−_48713019_{—} | MINK1_chr17_+_4795696_{—} |
| 27606115_27605796_27610028 | 48713357_48700791_48729643 | 4795807_4795529_4795950 |
| PI4KB_chr1_−_151282686_{—} | G6PC3_chr17_+_42152336_{—} | PXN_chr12_−_120654075_{—} |
| 151282731_151280277_151288048 | 42152455_42152138_42152677 | 120654384_120653464_120659425 |
| PDCD10_chr3_−_167443188_{—} | SCARB1_chr12_−_125267228_{—} | FXYD3_chr19_+_35610071_{—} |
| 167443261_167438061_167452001 | 125267357_125263132_125270902 | 35610155_35607263_35610266 |
| SLC37A4_chr11_−_118897215_{—} | RPARP-AS1_chr10_+_104211846_{—} | PLCB4_chr20_+_9353693_{—} |
| 118897398_118896790_118897646 | 104211944_104210117_104215093 | 9353751_9351942_9360700 |
| C2CD5_chr12_−_22611417_{—} | ANKRD49_chr11_+_94231067_{—} | ENOSF1_chr18_−_677742_{—} |
| 22611489_22610095_22622642 | 94231130_94230117_94231236 | 677872_675402_683245 |
| IP6K2_chr3_−_48732134_{—} | PAK4_chr19_+_39658869_{—} | FAM114A2_chr5_−_153390800_{—} |
| 48732257_48731673_48732522 | 39658952_39616559_39660171 | 153390880_153382529_153405958 |
| FAM193B_chr5_−_176980168_{—} | CDK10_chr16_+_89755659_{—} | ZC3H11A_chr1_+_203765420_{—} |
| 176980351_176966148_176981249 | 89755714_89753167_89756960 | 203765624_203764922_203770702 |
| PTPRS_chr19_−_5216730_{—} | SVIL_chr10_−_29821457_{—} | ACOT8_chr20_−_44482561_{—} |
| 5216778_5215606_5218430 | 29822387_29820217_29839525 | 44482618_44477314_44485826 |
| ST7_chr7_+_116738666_{—} | RBPMS_chr8_+_30407016_{—} | SNRNP70_chr19_+_49605370_{—} |
| 116738869_116593745_116739815 | 30407102_30361953_30416403 | 49606844_49604728_49607890 |
| THTPA_chr14_+_24025951_{—} | PROC_chr2_+_128180609_{—} | MED17_chr11_+_93527415_{—} |
| 24026513_24025552_24027903 | 128180747_128180517_128180849 | 93527605_93527201_93528073 |
| PELO_chr5_+_52095718_{—} | ZNF506_chr19_−_19904642_{—} | CBY1_chr22_+_39052960_{—} |
| 52096954_52084248_52097242 | 19906469_19903293_19916839 | 39053148_39052755_39061550 |
| PRPF3_chr1_+_150300015_{—} | ADSL_chr22_+_40749076_{—} | CARS2_chr13_−_111302983_{—} |
| 150300111_150298339_150300778 | 40749121_40746039_40754867 | 111303447_111299586_111315797 |
| RPS24_chr10_+_79799958_{—} | ZNF585A_chr19_−_37656496_{—} | DDB2_chr11_+_47256307_{—} |
| 79799983_79797062_79800372 | 37656598_37647257_37660740 | 47256485_47256223_47256820 |
| ENOSF1_chr18_−_677344_{—} | PASK_chr2_−_242047881_{—} | RFC2_chr7_−_73660991_{—} |
| 677444_675402_683245 | 242048222_242047715_242051654 | 73661093_73657576_73663341 |
| MANBAL_chr20_+_35925698_{—} | TARBP1_chr1_−_234536598_{—} | LYPLAL1_chr1_+_219366471_{—} |
| 35925937_35918089_35927165 | 234536694_234529583_234536926 | 219366593_219352588_219383873 |
| CAMK2D_chr4_−_114424091_{—} | MAATS1_chr3_+_119452210_{—} | MRPL10_chr17_−_45906504_{—} |
| 114424133_114421667_114430793 | 119452353_119451323_119456212 | 45906602_45906036_45908825 |
| VPS54_chr2_−_64140958_{—} | DYRK4_chr12_+_4716492_{—} | ECHDC2_chr1_−_53363108_{—} |
| 64141059_64140444_64141315 | 4716553_4714252_4719320 | 53363156_53362269_53364845 |
| VEGFA_chr6_+_43748468_{—} | TRA2A_chr7_−_23561750_{—} | WNK2_chr9_+_96062332_{—} |
| 43748540_43746655_43749692 | 23562051_23561459_23571407 | 96062431_96061543_96070609 |
| UAP1_chr1_+_162562524_{—} | TRMT11_chr6_+_126327987_{—} | TCTN1_chr12_+_111072474_{—} |
| 162562572_162560301_162567581 | 126328100_126320759_126329537 | 111072584_111070364_111074285 |
| KCNMA1_chr10_−_78772276_{—} | PLEKHM2_chr1_+_16047823_{—} | SEPT6_chrX_−_118759297_{—} |
| 78772315_78771801_78778770 | 16047883_16046415_16051811 | 118759342_118750705_118763280 |
| ITGB3BP_chr1_−_63913235_{—} | MTRF1L_chr6_−_153312319_{—} | PNISR_chr6_−_99864224_{—} |
| 63913285_63906774_63919588 | 153312456_153311230_153313991 | 99864304_99862566_99873090 |
| IQCB1_chr3_−_121514303_{—} | GCFC2_chr2_−_75919102_{—} | AHI1_chr6_−_135623849_{—} |
| 121514413_121509062_121526190 | 75919226_75917845_75921366 | 135623955_135621696_135639656 |
| RAB40C_chr16_+_646316_{—} | RHOU_chr1_+_228873419_{—} | FAM13A_chr4_−_89658622_{—} |
| 646448_640433_667207 | 228873478_228871751_228879031 | 89658706_89653349_89660180 |
| MYO6_chr6_+_76608089_{—} | SYNE4_chr19_−_36497651_{—} | RAB25_chr1_+_156035701_{—} |
| 76608128_76602407_76617321 | 36497846_36496339_36499455 | 156035897_156031234_156038060 |
| SMARCD1_chr12_+_50481145_{—} | IP6K2_chr3_−_48752821_{—} | ZMYM4_chr1_+_35827219_{—} |
| 50481268_50480661_50482303 | 48752960_48732854_48754589 | 35827390_35791006_35835629 |
| LSM14A_chr19_+_34717312_{—} | C9orf3_chr9_+_97848211_{—} | NDUFAF6_chr8_+_96048529_{—} |
| 34717369_34712643_34718269 | 97848401_97843062_97848963 | 96048722_96047804_96053797 |
| CCNB1IP1_chr14_−_20783643_{—} | ATP9B_chr18_+_76973961_{—} | ALOX15B_chr17_+_7950611_{—} |
| 20783777_20781960_20784385 | 76974038_76967012_77013380 | 7950697_7950394_7951062 |
| ARMC6_chr19_+_19144939_{—} | SENP5_chr3_+_196654666_{—} | TMCC1_chr3_−_129599151_{—} |
| 19145047_19144739_19153519 | 196654750_196650422_196656503 | 129599402_129551669_129612336 |
| APLP2_chr11_+_130007150_{—} | SARAF_chr8_−_29931392_{—} | ABHD11_chr7_−_73152205_{—} |
| 130007186_130005610_130010292 | 29931567_29927575_29940362 | 73152472_73152065_73152657 |
| NDUFAF6_chr8_+_96048598_{—} | PPT2-EGFL8_chr6_+_32132355_{—} | MBNL2_chr13_+_98018712_{—} |
| 96048722_96047804_96053797 | 32132434_32130399_32134274 | 98018807_98009889_98043575 |
| LINC01578_chr15_+_93426814_{—} | UIMC1_chr5_−_176395555_{—} | CTBP2_chr10_−_126799558_{—} |
| 93426849_93426416_93428744 | 176396292_176385155_176396601 | 126799662_126692061_126848887 |
| RPLPO_chr12_−_120636356_{—} | CANT1_chr17_−_77001453_{—} | ZRANB2_chr1_−_71531360_{—} |
| 120636434_120635265_120636656 | 77001563_76994045_77005745 | 71531435_71530820_71532458 |
| TCTN1_chr12_+_111082771_{—} | SYNE4_chr19_−_36497651_{—} | NFIX_chr19_+_13189426_{—} |
| 111082934_111078322_111085015 | 36497846_36496339_36498026 | 13189549_13186485_13192493 |
| DGUOK_chr2_+_74177711_{—} | ZNF707_chr8_+_144770831_{—} | R3HDM2_chr12_−_57699422_{—} |
| 74177859_74154179_74184251 | 144771471_144766712_144772226 | 57699446_57697000_57704046 |
| DYSF_chr2_+_71740369_{—} | CLK1_chr2_−_201724847_{—} | OSER1-AS1_chr20_+_42843527_{—} |
| 71740462_71739051_71740845 | 201724938_201724469_201725960 | 42843643_42839996_42853460 |
| UBE3A_chr15_−_25652213_{—} | NUP50_chr22_+_45566868_{—} | SMPDL3A_chr6_+_123122454_{—} |
| 25652359_25650649_25654234 | 45567033_45564127_45567480 | 123122551_123118113_123126053 |
| MIS12_chr17_+_5391515_{—} | TBC1D1_chr4_+_38054726_{—} | GOLGA2P7_chr15_−_84873982_{—} |
| 5391909_5390004_5392142 | 38054846_38053681_38055819 | 84874071_84873727_84898653 |
| PAOX_chr10_+_135197463_{—} | ANAPC15_chr11_−_71822487_{—} | COL16A1_chr1_−_32136156_{—} |
| 135197716_135195163_135202459 | 71822542_71822332_71823695 | 32136247_32134456_32137215 |
| ADCK1_chr14_+_78294196_{—} | ITGAE_chr17_−_3626581_{—} | SSFA2_chr2_+_182785323_{—} |
| 78294314_78288861_78325418 | 3626677_3623696_3626981 | 182785389_182784173_182786674 |
| XAF1_chr17_+_6665204_{—} | SEPT2_chr2_+_242256913_{—} | TRPM4_chr19_+_49684605_{—} |
| 6665356_6663920_6665473 | 242257014_242255397_242265407 | 49684718_49675365_49685834 |
| MAPT_chr17_+_44067243_{—} | ZNF239_chr10_−_44063346_{—} | TTC27_chr2_+_33036090_{—} |
| 44067441_44064461_44068825 | 44063469_44053619_44069987 | 33036288_33012216_33042523 |
| SPTAN1_chr9_+_131391403_{—} | HADHB_chr2_+_26477114_{—} | GLI4_chr8_+_144351529_{—} |
| 131391466_131390221_131392599 | 26477186_26467858_26486247 | 144351690_144349654_144356873 |
| HDAC6_chrX_+_48676626_{—} | ANKRD10_chr13_−_111552876_{—} | PRUNE2_chr9_−_79239938_{—} |
| 48676819_48676516_48681029 | 111553008_111545610_111558379 | 79239974_79234303_79244107 |
| SERHL2_chr22_+_42959394_{—} | SNHG5_chr6_−_86387512_{—} | FAM131A_chr3_+_184056174_{—} |
| 42959487_42956271_42967126 | 86387593_86387210_86387671 | 184056317_184055159_184059511 |
| SERPINA1_chr14_−_94854896_{—} | DMTN_chr8_+_21938315_{—} | MRRF_chr9_+_125048317_{—} |
| 94854997_94849578_94856793 | 21938381_21938136_21938623 | 125048445_125047566_125054027 |
| BIRC5_chr17_+_76212744_{—} | ADCY6_chr12_−_49168739_{—} | RPS6KB2_chr11_+_67199823_{—} |
| 76212862_76210870_76219545 | 49168837_49167881_49169085 | 67199963_67198986_67200070 |
| VPS33B_chr15_−_91560192_{—} | TTN_chr2_−_179514280_{—} | ZNF431_chr19_+_21333881_{—} |
| 91560254_91557663_91565383 | 179514358_179510757_179514543 | 21334001_21326446_21349137 |

TABLE 1b
Myc AS CL

| AS Events | AS Events | AS Events |
|---|---|---|
| RP11-111H13.1_chr2_+_99938413_{—} | LRR1_chr14_+_50070269_{—} | KLHL8_chr4_−_88106402_{—} |
| 99938491_99937728_999389 | 50070336_50069186_50080973 | 88106951_88104541_88141569 |
| CYP3A5_chr7_−_99269393_{—} | ANKRD18DP_chr3_−_197792623_{—} | ERMP1_chr9_−_5805610_{—} |
| 99269501_99264688_99270202 | 197792677_197790659_197803666 | 5805785_5801328_5810010 |
| SLC25A3_chr12_+_98989210_{—} | FBXL19_chr16_+_30939787_{—} | RNASEH1-AS1_chr2_+_3607037_{—} |
| 98989439_98987913_98991633 | 30939951_30939284_30941393 | 3607319_3606588_3608906 |
| WNK2_chr9_+_96061351_{—} | PRSS16_chr6_+_27219528_{—} | AKTIP_chr16_−_53536611_{—} |
| 96061543_96060349_96070609 | 27219819_27219043_27220586 | 53536956_53534241_53537058 |
| TP53INP1_chr8_−_95944297_{—} | VEZT_chr12_+_95692637_{—} | DAZAP1_chr19_+_1425876_{—} |
| 95944335_95942956_95952087 | 95692719_95690034_95693940 | 1425959_1422395_1428840 |
| FAM76B_chr11_−_95512241_{—} | SLC25A12_chr2_−_172644080_{—} | PPP1R7_chr2_+_242092890_{—} |
| 95512299_95512121_95512770 | 172644171_172641985_172644298 | 242093019_242089123_242097221 |
| SETD5_chr3_+_9471510_{—} | R3HDM2_chr12_−_57686354_{—} | COX20_chr1_+_245005245_{—} |
| 9471735_9470693_9475528 | 57686432_57682823_57689181 | 245005360_244999058_245005496 |
| CHD2_chr15_+_93541378_{—} | PLA2G12A_chr4_−_110639838_{—} | PEX2_chr8_−_77900542_{—} |
| 93541491_93540633_93541728 | 110639915_110638869_110650757 | 77900574_77896431_77912225 |
| TAF1D_chr11_−_93469306_{—} | EEF1D_chr8_−_144672777_{—} | RNF219_chr13_−_79218996_{—} |
| 93469470_93466563_93470229 | 144672908_144672251_144678257 | 79219132_79216348_79233183 |
| TAF1D_chr11_−_93467790_{—} | SLC27A5_chr19_−_59010151_{—} | NOP14-AS1_chr4_+_2940419_{—} |
| 93467826_93466563_93468128 | 59010282_59010058_59010489 | 2940519_2939869_2948599 |
| TAFID_chr11_−_93466515_{—} | PNPLA8_chr7_−_108128388_{—} | TMEM39A_chr3_−_119176864_{—} |
| 93466585_93463878_93467790 | 108128397_108119823_108131853 | 119176930_119171377_119180808 |
| PPP4R3A_chr14_−_91932604_{—} | MPDU1_chr17_+_7489012_{—} | UPF3B_chrX_−_118974556_{—} |
| 91932760_91931763_91937180 | 7489115_7487283_7489263 | 118974647_118972490_118975038 |
| ZNF644_chr1_−_91403041_{—} | CEP135_chr4_+_56840911_{—} | AKT2_chr19_−_40788123_40788351_{—} |
| 91403647_91383711_91447866 | 56841135_56837574_56846308 | 40771258_40788614 |
| EFCAB11_chr14_−_90390856_{—} | MIR22HG_chr17_−_1617664_{—} | GRAMD1B_chr11_+_123465442_{—} |
| 90391021_90263668_90397884 | 1617747_1617308_1619423 | 123465546_123464874123466649 |
| KLHL17_chr1_+_898716_{—} | PBRM1_chr3_−_52588739_{—} | APOPT1_chr14_+_104040443_{—} |
| 898884_898633_899299 | 52588895_52584833_52595782 | 104040507_104029716_104053610 |
| ANKRD11_chr16_−_89358088_{—} | C21orf33_chr21_+_45560131_{—} | PTK2_chr8_−_141935759_{—} |
| 89358185_89357591_89371613 | 45560224_45557178_45563086 | 141935848_141900868_142010978 |
| HERC6_chr4_+_89304336_{—} | VTI1A_chr10_+_114208139_{—} | PLEKHG4B_chr5_+_179665_{—} |
| 89304532_89300272_89306650 | 114208247_114207225_114208639 | 179869_174213_181628 |
| HERC6_chr4_+_89304383_{—} | MTA1_chr14_+_105915695_{—} | TRPM2-AS_chr21_−_45843258_{—} |
| 89304532_89300272_89306650 | 105915746_105911848_105916394 | 45843364_45834827_45845032 |
| HERC6_chr4_+_89304372_{—} | ASXL1_chr20_+_31017703_{—} | SDCCAG8_chr1_+_243456392_{—} |
| 89304532_89300272_89306650 | 31017856_31017234_31019123 | 243456521_243449699_243468014 |
| ZNF778_chr16_+_89291126_{—} | CABIN1_chr22_+_24447286_{—} | CCDC90B_chr11_−_82989768_{—} |
| 89291210_89288591_89291962 | 24447436_24445682_24451335 | 82989872_82985783_82991183 |
| PMM2_chr16_+_8904935_{—} | HSCB_chr22_+_29139869_{—} | RUFY2_chr10_−_70164427_{—} |
| 8905035_8900264_8905494 | 29139966_29138319_29147228 | 70164601_70161494_70166942 |
| METTL22_chr16_+_8738413_{—} | ESYT2_chr7_−_158545471_{—} | DCUN1D2_chr13_−_114128438_{—} |
| 8738582_8736422_8739964 | 158545534_158542414_158552176 | 114128569_114115451_114144981 |
| MAPK10_chr4_−_87019676_{—} | IL6R_chr1_+_154408444_{—} | USP53_chr4_+_120135224_{—} |
| 87020438_86989108_87022204 | 154408586_154407632_154417522 | 120135377_120134028_120138704 |
| MTHFSD_chr16_−_86585578_{—} | TPP2_chr13_+_103320107_{—} | EVA1C_chr21_+_33825619_{—} |
| 86585752_86582183_86588250 | 103320233_103317286_103326632 | 33825816_33785321_33840003 |
| ME3_chr11_−_86168407_{—} | PLEKHG1_chr6_+_150959215_{—} | SNHG14_chr15_+_25301629_{—} |
| 86168801_86161440_86176132 | 150959261_150921153_150971863 | 25301769_25299114_25304301 |
| SH2D6_chr2_+_85663588_{—} | ZNF397_chr18_+_32825225_{—} | CEP162_chr6_−_84936054_{—} |
| 85663717_85662246_85663959 | 32825315_32823257_32834195 | 84936170_84930889_84937297 |
| SH2D6_chr2_+_85663588_{—} | NSUN5P1_chr7_+_75039888_{—} | TTPAL_chr20_+_43107263_{—} |
| 85663717_85662947_85663959 | 75040009_75039743_75042066 | 43107287_43104631_43108624 |
| ELMOD3_chr2_+_85614220_{—} | COL9A3_chr20_+_61467538_{—} | RP11-284E5.1_chr2_−_191491214_{—} |
| 85614348_85604597_85616873 | 61467685_61467305_61467829 | 191491549_191481844_191512945 |
| CEP162_chr6_−_84928820_{—} | MYCBP2_chr13_−_77700452_{—} | ZC3H11A_chr1_+_203765578_{—} |
| 84928932_84925640_84930774 | 77700677_77699603_77713330 | 203765729_203764903_203786053 |
| COQ2_chr4_−_84188242_{—} | TNKS_chr8_+_9588399_{—} | NOXRED1_chr14_−_77880276_{—} |
| 84188361_84185516_84188738 | 9588545_9584230_9592374 | 77880470_77873988_77889077 |
| NARF_chr17_+_80436446_{—} | MACC1_chr7_−_20238147_{—} | TLE4_chr9_+_82242288_{—} |
| 80436543_80426772_80436675 | 20238293_20204022_20256921 | 82242363_82227633_82267507 |
| HEXDC_chr17_+_80398348_{—} | PIAS1_chr15_+_68481540_{—} | TLE4_chr9_+_82242288_{—} |
| 80398489_80398210_80398872 | 68481608_68480569_68482830 | 82242363_82227633_82267504 |
| RPS24_chr10_+_79799961_{—} | EDRF1_chr10_+_127417571_{—} | ENKD1_chr16_−_67697559_{—} |
| 79799983_79797062_79800372 | 127417673_127414407_127417926 | 67697723_67697459_67697839 |
| SLC25A10_chr17_+_79680291_{—} | STAP2_chr19_−_4324451_{—} | FLOT2_chr17_−_27211242_{—} |
| 79680536_79679549_79681982 | 4324664_4324194_4325212 | 27211333_27210249_27212874 |
| SGSH_chr17_−_78188413_{—} | STK39_chr2_−_168921828_{—} | DNAJC14_chr12_−_56223272_{—} |
| 78188564_78188127_78190830 | 168921891_168920080_168931491 | 56223420_56222498_56224479 |
| RABGGTB_chr1_+_76255636_{—} | ARPC4-TTLL3_chr3_+_9859328_{—} | SRRM2_chr16_+_2806334_{—} |
| 76255959_76253289_76256969 | 9859443_9855029_9867483 | 2806607_2802847_2807472 |
| SYNGR2_chr17_+_76167590_{—} | HCG18_chr6_−_30282045_{—} | VARS_chr6_−_31751990_{—} |
| 76167730_76167135_76167819 | 30282259_30264014_30293785 | 31752085_31750963_31752170 |
| JMJD6_chr17_−_74717344_{—} | MINDY1_chr1_−_150974640_{—} | TBL1X_chrX_+_9621584_{—} |
| 74717433_74716580_74717879 | 150974799_150974258_150978787 | 9621729_9608400_9622254 |
| JMJD6_chr17_−_74717344_{—} | ARTN_chr1_+_44401764_{—} | WASHC5_chr8_−_126095954_{—} |
| 74717438_74716580_74717879 | 44401903_44401388_44401973 | 126096018_126095500_126103856 |
| RECQL5_chr17_−_73646596_{—} | PLCB2_chr15_−_40583218_{—} | SH2D6_chr2_+_85659021_{—} |
| 73646798_73627748_73647265 | 40583384_40583038_40583465 | 85659082_85658391_85660693 |
| PMFBP1_chr16_−_72188109_{—} | TNFSF12-TNFSF13_chr17_+_7463162_{—} | UGT8_chr4_+_115540578_{—} |
| 72188358_72174481_72198662 | 7463210_7463019_7463365 | 115540681_115520130_115544034 |
| RNF121_chr11_+_71698049_{—} | ATP2A3_chr17_−_3831274_{—} | TANK_chr2_+_162080430_{—} |
| 71698157_71693961_71701642 | 3831332_3828735_3831520 | 162080502_162061304_162081141 |
| RNF121_chr11_+_71673197_{—} | RP5-1120P11.1_chr6_−_43968327_{—} | FAM92A_chr8_+_94736737_{—} |
| 71673335_71671937_71693806 | 43968452_43967245_43968779 | 94736823_94722103_94738621 |
| ZRANB2_chr1_−_71531360_{—} | HPS4_chr22_−_26862016_{—} | PAQR5_chr15_+_69677015_{—} |
| 71531435_71530820_71532458 | 26862070_26861517_26862191 | 69677221_69672349_69689806 |
| SRSF11_chr1_+_70697541_{—} | MTERF4_chr2_−_242033691_{—} | TM2D1_chr1_−_62152463_{—} |
| 70697658_70694238_70697950 | 242033847_242029459_242036657 | 62152593_62149218_62160368 |
| SLC25A16_chr10_−_70253205_{—} | GK_chrX_+_30745582_{—} | TIAL1_chr10_−_121339982_{—} |
| 70253327_70252955_70263388 | 30745669_30742298_30746848 | 121340050_121339522_121341433 |
| DNA2_chr10_−_70230245_{—} | TNS1_chr2_−_218677937_{—} | BIN1_chr2_−_127815048_{—} |
| 70230369_70229920_70231547 | 218678027_218677168_218678407 | 127815177_127808488_127816586 |
| MCRIP2_chr16_+_696471_{—} | PAQR6_chr1_−_156215325_{—} | SCAMP4_chr19_+_1918887_{—} |
| 696608_692249_697416 | 156215452_156215029_156215572 | 1918989_1918282_1923068 |
| NFATC3_chr16_+_68248231_{—} | TBC1D7_chr6_−_13325325_{—} | ABHD5_chr3_+_43759162_{—} |
| 68248335_68225678_68260252 | 13325406_13321327_13327018 | 43759349_43756550_43759934 |
| TSNAXIP1_chr16_+_67858485_{—} | LINC00630_chrX_+_102094728_{—} | DUOX1_chr15_+_45440101_{—} |
| 67858682_67855128_67859039 | 102094791_102082072_102100784 | 45440195_45439856_45440469 |
| ING4_chr12_−_6764803_{—} | IMPDH1_chr7_−_128045820_{—} | ADGRF1_chr6_−_46984352_{—} |
| 6765079_6762216_6765892 | 128045919_128043808_128049809 | 46984504_46982580_46988466 |
| FHOD1_chr16_−_67270313_{—} | LRIF1_chr1_−_111493909_{—} | SCART1_chr10_+_135304725_{—} |
| 67270337_67268375_67270459 | 111495437_111492745_111506242 | 135304823_135280857_135311332 |
| TMEM134_chr11_−_67232526_{—} | ARL16_chr17_−_79650042_{—} | CMTM7_chr3_+_32490945_{—} |
| 67232738_67232327_67234782 | 79650156_79649179_79650565 | 32491044_32483505_32493883 |
| HSF4_chr16_+_67201363_{—} | TCOF1_chr5_+_149771106_{—} | TMBIM6_chr12_+_50138195_{—} |
| 67201502_67201125_67201726 | 149771358_149769586_149771519 | 50138325_50135898_50146246 |
| FAM20A_chr17_−_66586264_{—} | WASF1_chr6_−_110499800_{—} | SLC28A3_chr9_−_86928273_{—} |
| 66586490_66551884_66596403 | 110499945_110448832_110500641 | 86928369_86924629_86955488 |
| C17orf58_chr17_−_65989016_{—} | RHBDL3_chr17_+_30611677_{—} | KAT5_chr11_+_65480818_{—} |
| 65989351_65988219_65989539 | 30611836_30594944_30615810 | 65480974_65480529_65481063 |
| AGPAT5_chr8_+_6588231_{—} | KIF13A_chr6_−_17794479_{—} | DOCK9_chr13_−_99478125_{—} |
| 6588347_6566408_6612571 | 17794626_17788106_17794802 | 99478194_99476758_99479083 |
| SREK1_chr5_+_65454636_{—} | HTR3C_chr3_+_183773099_{—} | AC138969.4_chr16_+_16425710_{—} |
| 65454760_65449424_65455046 | 183773144_183770935_183774662 | 16425837_16425620_16427503 |
| SREK1_chr5_+_65451892_{—} | CD47_chr3_−_107770785_{—} | KIAA0895_chr7_−_36396510_{—} |
| 65454760_65449424_65455046 | 107770817_107766139_107776323 | 36397199_36375906_36406524 |
| CDH19_chr18_−_64176231_{—} | WDR11_chr10_+_122612035_{—} | TBCE_chr1_+_235582787_{—} |
| 64176455_64172539_64178804 | 122612147_122611039_122618154 | 235582876_235577933_235590454 |
| CDH19_chr18_−_64176231_{—} | UROS_chr10_−_127477851_{—} | TIA1_chr2_−_70455475_{—} |
| 64176483_64172539_64178804 | 127478097_127477574_127483448 | 70455594_70454954_70456190 |
| USP3_chr15_+_63826001_{—} | ATP13A3_chr3_−_194132927_{—} | KIAA1468_chr18_+_59931961_{—} |
| 63826117_63824906_63845913 | 194133017_194126845_194134487 | 59932085_59931378_59933933 |
| USP3_chr15_+_63826001_{—} | INTS11_chr1_−_1258560_{—} | ZNF83_chr19_−_53119970_{—} |
| 63826117_63824906_63829223 | 1258667_1250998_1259960 | 53120068_53118050_53122188 |
| USP3_chr15_+_63821212_{—} | MED15_chr22_+_20929399_{—} | PRPF38B_chr1_+_109236211_{—} |
| 63821365_63797029_63829223 | 20929519_20922918_20936897 | 109236264_109235489_109238323 |
| USP3_chr15_+_63821212_{—} | CELF6_chr15_−_72608185_{—} | UTAT33_chr2_−_105714656_{—} |
| 63821365_63797029_63824845 | 72608268_72597135_72611953 | 105714720_105713856_105719293 |
| SMURF2_chr17_−_62590108_{—} | PPM1K_chr4_−_89199295_{—} | MFSD6_chr2_+_191298052_{—} |
| 62590222_62589691_62594499 | 89199794_89198395_89205557 | 191298118_191280139_191300702 |
| IRF7_chr11_−_613950_{—} | EPB41L1_chr20_+_34797409_{—} | FIP1L1_chr4_+_54306748_{—} |
| 614037_613865_614475 | 34797820_34785963_34800193 | 54306775_54294350_54308819 |
| SLCO4A1_chr20_+_61299363_{—} | CCDC169_chr13_−_36869914_{—} | CPNE5_chr6_−_36720810_{—} |
| 61299536_61299262_61299828 | 36869994_36857839_36871773 | 36720872_36716044_36724021 |
| UBXN2B_chr8_+_59347416_{—} | FER1L5_chr2_+_97312800_{—} | LCMT1_chr16_+_25162874_{—} |
| 59347507_59347063_59352191 | 97312892_97312190_97315359 | 25162936_25151568_25175918 |
| VRK2_chr2_+_58381888_{—} | BLOC1S6_chr15_+_45890077_{—} | RAPGEF1_chr9_−_134525515_{—} |
| 58381937_58373609_58386483 | 45890089_45879723_45895297 | 134525629_134518804_134526196 |
| CTDSP2_chr12_−_58220778_{—} | ANKRD6_chr6_+_90331640_{—} | ETV4_chr17_−_41610041_{—} |
| 58220906_58220185_58221334 | 90331745_90315824_90333128 | 41610307_41607549_41610554 |
| TMX2_chr11_+_57505384_{—} | RERE_chr1_−_8674619_{—} | CANT1_chr17_−_77001453_{—} |
| 57505498_57505140_57505825 | 8674745_8617582_8684368 | 77001563_76994368_77005745 |
| MYL6_chr12_+_56554409_{—} | KIF13A_chr6_−_17771344_{—} | ZNF345_chr19_+_37367686_{—} |
| 56554454_56554104_56555170 | 17771449_17765177_17772138 | 37367778_37342806_37383719 |
| PSPH_chr7_−_56087292_{—} | ERMAP_chr1_+_43296114_{—} | KCNMB2-AS1_chr3_−_178577276_{—} |
| 56087501_56085072_56088765 | 43296204_43291409_43300708 | 178577411_178559404_178578081 |
| PSPH_chr7_−_56087292_{—} | LRRC8B_chr1_+_90015473_{—} | LINC02014_chr3_−_129810619_{—} |
| 56087427_56085072_56088765 | 90015530_89990581_90024269 | 129810710_129809963_129812990 |
| RBM38_chr20_+_55967452_{—} | NTAN1_chr16_−_15141853_{—} | SLC50A1_chr1_+_155110036_{—} |
| 55967548_55966851_55967709 | 15141956_15141777_15149747 | 155110198_155108852_155110454 |
| USP24_chr1_−_55617584_{—} | TPRA1_chr3_−_127294591_{—} | RP11-977G19.10_chr12_−_56703749_{—} |
| 55617648_55614243_55619542 | 127294652_127294348_127294782 | 56703813_56694004_56704741 |
| EPB41L3_chr18_−_5394675_{—} | SYNE4_chr19_−_36499118_{—} | POU2AF1_chr11_−_111267575_{—} |
| 5394792_5393477_5395065 | 36499269_36498170_36499455 | 111267768_111257017_111268304 |
| HRAS_chr11_−_533276_{—} | LUC7L_chr16_−_278331_{—} | RABGAP1L_chr1_+_174957777_{—} |
| 533358_532755_533452 | 278401_277335_279277 | 174957975_174952042_174958985 |
| ST18_chr8_−_53122589_{—} | TEX9_chr15_+_56676149_{—} | RP11-164J13.1_chr15_+_42682150_{—} |
| 53122677_53119310_53124638 | 56676229_56665702_56704499 | 42682294_42681294_42684836 |
| TOM1L1_chr17_+_52981068_{—} | WARS_chr14_−_100841619_{—} | THAP5_chr7_−_108206273_{—} |
| 52981153_52978284_52990026 | 100841687_100835595_100842596 | 108206466_108205549_108209933 |
| DCUN1D4_chr4_+_52753288_{—} | MFF_chr2_+_228207460_{—} | CD44_chr11_+_35219667_{—} |
| 52753410_52752804_52757925 | 228207535_228205096_228220392 | 35219793_35211612_35226058 |
| VRK3_chr19_−_50512492_{—} | CCDC125_chr5_−_68590619_{—} | PMS2P5_chr7_+_74312525_{—} |
| 50512642_50511083_50519280 | 68590727_68588189_68595838 | 74312628_74312349_74313767 |
| ATP8B4_chr15_−_50214420_{—} | MAPK10_chr4_−_87019676_{—} | ANLN_chr7_+_36456688_{—} |
| 50214602_50212607_50215575 | 87019748_86989108_87022204 | 36456799_36455493_36458852 |
| LRR1_chr14_+_50074117_{—} | ZFP90_chr16_+_68573660_{—} | C19orf47_chr19_−_40840741_{—} |
| 50074839_50069186_50080973 | 68573728_68567758_68591900 | 40840811_40839836_40842016 |
| SNRNP70_chr19_+_49605370_{—} | ZUFSP_chr6_−_116987796_{—} | C15orf52_chr15_−_40631712_{—} |
| 49605442_49604728_49607890 | 116988075_116982009_116989728 | 40631820_40631104_40632105 |
| SNRNP70_chr19_+_49605370_{—} | PAOX_chr10_+_135203093_{—} | LCORL_chr4_−_17887690_{—} |
| 49605430_49604728_49607890 | 135203251_135202572_135204815 | 17887784_17879836_17910716 |
| SNRNP70_chr19_+_49605370_{—} | MINDY1_chr1_−_150974640_{—} | MMS19_chr10_−_99228722_{—} |
| 49606844_49604728_49607890 | 150974646_150974258_150978787 | 99228861_99228163_99229402 |
| BAX_chr19_+_49458943_{—} | HSD3B7_chr16_+_30996973_{—} | RPL18_chr19_−_49120572_{—} |
| 49459090_49458856_49459454 | 30997268_30996618_30997369 | 49120672_49119459_49121047 |
| LUC7L3_chr17_+_48826579_{—} | OPA1_chr3_+_193321288_{—} | MRPL22_chr5_+_154330362_{—} |
| 48826705_48825777_48827861 | 193321346_193311198_193333462 | 154330498_154320825_154335930 |
| LUC7L3_chr17_+_48815497_{—} | GIN1_chr5_−_102432244_{—} | WARS_chr14_−_100841619_{—} |
| 48815560_48814840_48817666 | 102432530_102423876_102440244 | 100841743_100835595_100842596 |
| IP6K2_chr3_−_48732134_{—} | DNM1L_chr12_+_32891197_{—} | TRA2A_chr7_−_23561750_{—} |
| 48732257_48731673_48732522 | 32891230_32890876_32892997 | 23562051_23561459_23571407 |
| IP6K2_chr3_−_48732126_{—} | DLG2_chr11_−_83641370_{—} | ARHGEF37_chr5_+_148996129_{—} |
| 48732257_48731673_48732522 | 83641526_83585531_83673927 | 148996329_148989258_148997738 |
| MYEF2_chr15_−_48435092_{—} | MIB2_chr1_+_1560370_{—} | PLEKHM2_chr1_+_16047823_{—} |
| 48435268_48434333_48441359 | 1560565_1560281_1560665 | 16047883_16046415_16051811 |
| MGRN1_chr16_+_4736259_{—} | LINC00630_chrX_+_102100784_{—} | ZNF33B_chr10_−_43127742_{—} |
| 4736304_4733933_4738796 | 102100948_102094900_102119969 | 43127887_43127472_43132367 |
| ADGRF1_chr6_−_46984352_{—} | CBR3-AS1_chr21_−_37509391_{—} | RTFDC1_chr20_+_55045655_{—} |
| 46988233_46982580_46988466 | 37509474_37506059_37518553 | 55045807_55043822_55046669 |
| PHF21A_chr11_−_45956671_{—} | TBCE_chr1_+_235582731_{—} | GTF2H2C_chr5_+_68858499_{—} |
| 45956797_45955776_45957186 | 235582876_235577933_235590454 | 68858597_68856230_68860926 |
| DNAJA3_chr16_+_4504811_{—} | PRRG2_chr19_+_50087169_{—} | MGAM2_chr7_+_141887549_{—} |
| 4504928_4498849_4505546 | 50087209_50084738_50091753 | 141887634_141885923_141889166 |
| NME4_chr16_+_448207_{—} | ADHFE1_chr8_+_67357452_{—} | RP1-27K12.2_chr6_−_53434220_{—} |
| 448385_447079_448989 | 67357649_67356983_67359498 | 53434403_53429687_53481683 |
| SERINC4_chr15_−_44091507_{—} | ZNF566_chr19_−_36967450_{—} | EDC3_chr15_−_74979431_{—} |
| 44091684_44088425_44092082 | 36967518_36964360_36980387 | 74979520_74948409_74988220 |
| SERINC4_chr15_−_44088813_{—} | PUM1_chr1_−_31452908_{—} | STEAP3_chr2_+_119996938_{—} |
| 44088940_44088425_44092082 | 31453025_31447649_31454158 | 119997006_119981464_120003064 |
| SETMAR_chr3_+_4354581_{—} | ARMC10_chr7_+_102716223_{—} | TIA1_chr2_−_70455475_{—} |
| 4355445_4345210_4357895 | 102716328_102715858_102724128 | 70455594_70454954_70456395 |
| FMNL1_chr17_+_43322985_{—} | NOL3_chr16_+_67207910_{—} | RUFY3_chr4_+_71670064_{—} |
| 43323100_43322783_43323244 | 67207949_67204477_67208064 | 71670133_71668700_71672232 |
| FMNL1_chr17_+_43322998_{—} | PATZ1_chr22_−_31724772_{—} | PRMT7_chr16_+_68381113_{—} |
| 43323100_43322783_43323244 | 31724910_31723295_31731677 | 68381197_68380183_68381533 |
| CENPM_chr22_−_42339613_{—} | APBB2_chr4_−_40937093_{—} | FAM227B_chr15_−_49860441_{—} |
| 42339705_42335200_42341228 | 40937156_40936716_40946881 | 49860543_49834003_49867207 |
| IKBKB_chr8_+_42147673_{—} | ASL_chr7_+_65546789_{—} | WASHC5_chr8_−_126096076_{—} |
| 42147791_42129723_42162704 | 65546984_65541080_65547354 | 126096264_126095500_126103856 |
| ECI2_chr6_−_4119419_{—} | CBY1_chr22_+_39061550_{—} | USH1C_chr11_−_17554801_{—} |
| 4119509_4117685_4125483 | 39061690_39052755_39064021 | 17554869_17553089_17565818 |
| HMGN1_chr21_−_40717755_{—} | ASAH2B_chr10_+_52504887_{—} | PCOLCE_chr7_+_100203298_{—} |
| 40717884_40717200_40719304 | 52505034_52502770_52509102 | 100203435_100202838_100204038 |
| HMGN1_chr21_−_40717755_{—} | RHOC_chr1_−_113247674_{—} | GGCT_chr7_−_30540151_{—} |
| 40719218_40717200_40719304 | 113247790_113246428_113249699 | 30540297_30538554_30544184 |
| MAP3K10_chr19_+_40711027_{—} | C19orf60_chr19_+_18700288_{—} | ESPN_chr1_+_6508952_{—} |
| 40711203_40704462_40715009 | 18700493_18699887_18702917 | 6509151_6508862_6511662 |
| NKIRAS2_chr17_+_40174587_{—} | NUP62_chr19_−_50430950_{—} | RP11-231C18.3_chr4_+_54280781_{—} |
| 40174658_40174490_40175671 | 50431072_50413141_50432606 | 54280889_54266006_54292038 |
| DNAJC7_chr17_−_40155376_{—} | ZNF197_chr3_+_44673596_{—} | SLC25A25-AS1_chr9_−_130879988_{—} |
| 40155575_40152588_40170455 | 44673688_44672713_44673964 | 130880114_130877568_130880201 |
| N4BP2_chr4_+_40101655_{—} | NPM2_chr8_+_21882726_{—} | HHAT_chr1_+_210591497_{—} |
| 40101746_40099189_40103694 | 21882817_21882341_21882946 | 210591669_210578023_210637848 |
| MOCS1_chr6_−_39894922_{—} | CCDC78_chr16_−_774910_{—} | PILRB_chr7_+_99951517_{—} |
| 39895194_39893589_39902033 | 774989_774509_775077 | 99951635_99950893_99952765 |
| ZZEF1_chr17_−_3974632_{—} | JAK2_chr9_+_4985939_{—} | CSGALNACT2_chr10_+_43662451_{—} |
| 3974740_3974218_3975901 | 4986022_4984765_5021962 | 43662546_4365604343671398 |
| PGAP2_chr11_+_3844138_{—} | PLA2G4B_chr15_+_42130944_{—} | SLC38A5_chrX_−_48324616_{—} |
| 3844223_3832654_3845112 | 42131120_42130157_42132355 | 48324709_48321365_48325185 |
| FGFR1_chr8_−_38279314_{—} | INTS8_chr8_+_95862143_{—} | RPRD2_chr1_+_150414356_{—} |
| 38279459_38277253_38280542 | 95862319_95861760_95863780 | 150414434_150413499_150415706 |
| MEAF6_chr1_−_37962147_{—} | POLG_chr15_−_89865192_{—} | TPRKB_chr2_−_73959710_{—} |
| 37962205_37961519_37967404 | 89865246_89862581_89865972 | 73959827_73959412_73964428 |
| KCTD17_chr22_+_37457578_{—} | PHF21A_chr11_−_45956671_{—} | CLTA_chr9_+_36263194_{—} |
| 37457669_37455478_37458564 | 45956708_45955776_45957186 | 36263439_36204176_36265417 |
| MEIS2_chr15_−_37186917_{—} | DDHD1_chr14_−_53518561_{—} | CALD1_chr7_+_134620438_{—} |
| 37187013_37184660_37187351 | 53518645_53513667_53521155 | 134620516_134618141_134625842 |
| SRSF3_chr6_+_36567597_{—} | CDH24_chr14_−_23521178_{—} | TROVE2_chr1_+_193038163_{—} |
| 36568053_36566760_36568928 | 23521292_23519152_23521681 | 193038311_193028906_193044949 |
| SEPT7_chr7_+_35872407_{—} | YTHDC2_chr5_+_112862282_{—} | NUDT6_chr4_−_123833722_{—} |
| 35872503_35840880_35903161 | 112862482_112851059_112868575 | 123833778_123818833_123838655 |
| PSMA6_chr14_+_35778119_{—} | NFX1_chr9_+_33354083_{—} | PLEKHA6_chr1_−_204225929_{—} |
| 35778201_35761758_35782086 | 33354185_33352717_33354848 | 204226016_204219742_204226480 |
| SMIM11A_chr21_+_35757775_{—} | JMJD8_chr16_−_732952_{—} | ZDHHC13_chr11_+_19164524_{—} |
| 35757942_35751815_35772008 | 733087_732874_733166 | 19164670_19138823_19167727 |
| ACACA_chr17_−_35646319_{—} | C19orf48_chr19_−_51302528_{—} | METTL26_chr16_−_685517_{—} |
| 35646430_35641871_35656204 | 51302614_51302215_51305473 | 685774_685340_686093 |
| DONSON_chr21_−_34955793_{—} | CHTF18_chr16_+_845131_{—} | POLR2J3_chr7_−_102183971_{—} |
| 34955972_34954552_34956895 | 845356_844201_845684 | 102184144_102182109_102185152 |
| RBM39_chr20_−_34328446_{—} | IDE_chr10_−_94240549_{—} | DLG3_chrX_+_69717029_{—} |
| 34328519_34326939_34328745 | 94240673_94238546_94243011 | 69717071_69713325_69718369 |
| RBM39_chr20_−_34301623_{—} | HDAC2_chr6_−_114289797_{—} | HTR3E_chr3_+_183819272_{—} |
| 34301779_34301018_34302106 | 114289860_114281182_114291643 | 183819317_183818439_183822574 |
| ZNF195_chr11_−_3383762_{—} | ZSCAN20_chr1_+_33954714_{—} | GORASP1_chr3_−_39144168_{—} |
| 3383832_3383119_3392204 | 33954791_33954251_33955117 | 39144372_39142593_39148969 |
| ZNF195_chr11_−_3383775_{—} | TMTC4_chr13_−_101322613_{—} | STX16_chr20_+_57234678_{—} |
| 3383832_3381795_3392204 | 101322823_101321048_101326997 | 57234690_57227143_57242545 |
| ZNF195_chr11_−_3383775_{—} | FAM210A_chr18_−_13670869_{—} | PGAP3_chr17_−_37829303_{—} |
| 3383832_3383119_3392204 | 13670967_13666712_13671860 | 37829508_37829119_37830245 |
| EVAIC_chr21_+_33829904_{—} | RP11-252A24.2_chr16_−_74383639_{—} | INPP5J_chr22_+_31520127_{—} |
| 33830028_33785321_33840003 | 74383757_74382913_74385937 | 31520249_31519105_31520830 |
| NT5C3A_chr7_−_33075545_{—} | KIAA1456_chr8_+_12848342_{—} | RHOC_chr1_−_113247721_{—} |
| 33075600_33066527_33102179 | 12848540_12809867_12863710 | 113247823_113246428_113249699 |
| APTX_chr9_−_32988080_{—} | ZFX_chrX_+_24195800_{—} | NIPA2_chr15_−_23019799_{—} |
| 32988127_32987729_32989756 | 24195983_24193560_24197299 | 23019856_23014528_23021197 |
| APTX_chr9_−_32988080_{—} | TM6SF1_chr15_+_83791508_{—} | TMEM108_chr3_+_132839723_{—} |
| 32988122_32987729_32989756 | 83791630_83790755_83793423 | 132839799_132838352_132948109 |
| BICD1_chr12_+_32520603_{—} | AC007040.11_chr2_−_71218959_{—} | MTIF2_chr2_−_55481176_{—} |
| 32520679_32490640_32530473 | 71219113_71216946_71220825 | 55481337_55476670_55489451 |
| BPHL_chr6_+_3119518_{—} | COMMD9_chr11_−_36297686_{—} | AKIP1_chr11_+_8933999_{—} |
| 3119746_3119081_3123890 | 36297790_36296322_36298638 | 8934080_8933218_8936372 |
| HSD3B7_chr16_+_30996780_{—} | GIGYF2_chr2_+_233587263_{—} | LCP2_chr5_−_169693811_{—} |
| 30997145_30996618_30997369 | 233587368_233568199_233589283 | 169693895_169689996_169694012 |
| ASXL1_chr20_+_30959580_{—} | EP400_chr12_+_132466033_{—} | SMARCC2_chr12_−_56566720_{—} |
| 30959677_30956926_30959966 | 132466141_132464338_132466637 | 56566813_56566488_56567479 |
| RP1-130H16.18_chr22_−_30684694_{—} | MAP2K7_chr19_+_7970692_{—} | SNHG14_chr15_+_25330336_{—} |
| 30684765_30683549_30688761 | 7970740_7968953_7974639 | 25330469_25328674_25332613 |
| C19orf12_chr19_−_30196180_{—} | CCDC18-AS1_chr1_−_93770944_{—} | CCDC78_chr16_−_774680_{—} |
| 30196301_30193884_30199160 | 93771027_93730329_93802933 | 774806_774509_775077 |
| HM13_chr20_+_30155880_{—} | MPRIP_chr17_+_17067422_{—} | LRP8_chr1_−_53742363_{—} |
| 30156083_30149539_30156922 | 17071229_17064670_17075031 | 53742750_53741425_53746258 |
| CLN3_chr16_−_28497667_{—} | SLC43A1_chr11_−_57265225_{—} | POLR2J3_chr7_−_102183971_{—} |
| 28497845_28495439_28497898 | 57265318_57261644_57268251 | 102184108_102182109_102185152 |
| METTL15_chr11_+_28349641_{—} | GPAA1_chr8_+_145138026_{—} | KIAA1755_chr20_−_36850850_{—} |
| 28349712_28318478_28351942 | 145138206_145137707_145138291 | 36850999_36848170_36851939 |
| SNX17_chr2_+_27595906_{—} | ADGRD1_chr12_+_131487729_{—} | HSD3B7_chr16_+_30998160_{—} |
| 27595980_27594210_27596113 | 131487847_131476937_131488730 | 30998323_30998025_30999088 |
| C5orf38_chr5_+_2752735_{—} | PARP11_chr12_−_3938075_{—} | PUF60_chr8_−_144906482_{—} |
| 2752868_2752578_2755142 | 3938196_3935399_3939055 | 144906569_144904083_144911449 |
| THOC1_chr18_−_260717_{—} | MTMR12_chr5_−_32235067_{—} | ADCY5_chr3_−_123022913_{—} |
| 260759_260304_264025 | 32235235_32230453_32239106 | 123023030_123022066_123033081 |
| PDPK1_chr16_+_2588619_{—} | RP9P_chr7_−_32973425_{—} | NCK2_chr2_+_106497783_{—} |
| 2588772_2588137_2607703 | 32973508_32969720_32982481 | 106498505_106471745_106509437 |
| DTNB_chr2_−_25830104_{—} | N4BP2L2_chr13_−_33020495_{—} | PICALM_chr11_−_85685750_{—} |
| 25830190_25803695_25851039 | 33020617_33018263_33054726 | 85685855_85670103_85692171 |
| CENPJ_chr13_−_25460371_{—} | TTL_chr2_+_113277858_{—} | IVNS1ABP_chr1_−_185275616_{—} |
| 25460523_25459808_25463453 | 113278002_113260758_113286257 | 185275757_185274775_185276145 |
| CENPJ_chr13_−_25460372_{—} | CBR3-AS1_chr21_−_37517276_{—} | MGAM2_chr7_+_141831317_{—} |
| 25460523_25459808_25463453 | 37517374_37509606_37518553 | 141831427_141830871_141831730 |
| KCTD9_chr8_−_25303595_{—} | SEC24C_chr10_+_75526107_{—} | MBOAT7_chr19_−_54691042_{—} |
| 25303766_25298189_25315714 | 75526299_75525968_75526517 | 54691169_54687563_54692070 |
| SRRM1_chr1_+_24973569_{—} | PRKCD_chr3_+_53199119_{—} | ITGB6_chr2_−_161055689_{—} |
| 24973699_24973280_24975349 | 53199231_53195444_53212419 | 161055769_161052931_161056513 |
| ARHGAP17_chr16_−_24939004_{—} | AGTPBP1_chr9_−_88296182_{—} | IQCA1_chr2_−_237327791_{—} |
| 24939053_24931581_24942104 | 88296250_88292497_88307603 | 237327914_237308131_237349682 |
| GSTT1_chr22_−_24379360_{—} | PAOX_chr10_+_135197463_{—} | USP9Y_chrY_+_14834040_{—} |
| 24379450_24376998_24381699 | 135197716_135195163_135202459 | 14834120_14821476_14837045 |
| ATG4B_chr2_+_242591298_{—} | RAPGEF1_chr9_−_134479347_{—} | RGS21_chr1_+_192316442_{—} |
| 242591389_242590750_242593960 | 134479440_134477536_134497182 | 192316519_192286235_192321176 |
| SEPT2_chr2_+_242263823_{—} | RGL2_chr6_−_33262464_{—} | OGDHL_chr10_−_50947706_{—} |
| 242264077_242257014_242265407 | 33262539_33261843_33262753 | 50947885_50946308_50950873 |
| ASB1_chr2_+_239344351_{—} | ATF2_chr2_−_175986171_{—} | DNAJA4_chr15_+_78562838_{—} |
| 239344654_239342336_239352982 | 175986268_175983097_175994865 | 78563019_78557237_78565436 |
| SS18_chr18_−_23664015_{—} | PROM1_chr4_−_15981017_{—} | ISOC2_chr19_−_55967002_{—} |
| 23664139_23662492_23667464 | 15981086_15972705_15981503 | 55967212_55966745_55967715 |
| EIF4E2_chr2_+_233438972_{—} | DMTF1_chr7_+_86783543_{—} | FASTKD1_chr2_−_170425693_{—} |
| 233439051_233431924_233445613 | 86783594_86781871_86783705 | 170425762_170419860_170427513 |
| ZNF142_chr2_−_219516423_{—} | KIN_chr10_−_7825043_{—} | SH2D6_chr2_+_85657103_{—} |
| 219517023_219515249_219520872 | 7825138_7822141_7829782 | 85657138_85656735_85658332 |
| ZNF142_chr2_−_219516423_{—} | CANT1_chr17_−_76996181_{—} | UBXN11_chr1_−_26627416_{—} |
| 219516545_219515249_219520872 | 76996352_76994368_77005745 | 26627544_26624553_26628184 |
| ZMYM5_chr13_−_20411795_{—} | PPIP5K2_chr5_+_102518934_{—} | CRYBB2P1_chr22_+_25851678_{—} |
| 20411961_20409829_20425494 | 102519108_102515889_102520372 | 25851807_25844251_25853225 |
| CARF_chr2_+_203807462_{—} | UBL7_chr15_−_74753008_{—} | UBE2E3_chr2_+_181845668_{—} |
| 203807690_203806703_203817281 | 74753119_74751237_74753377 | 181845700_181845273_181846744 |
| CARF_chr2_+_203807485_{—} | MOCS1_chr6_−_39895067_{—} | HTRA2_chr2_+_74759745_{—} |
| 203807690_203806703_203817281 | 39895194_39893589_39902033 | 74759841_74759052_74759946 |
| PPP1R12B_chr1_+_202412214_{—} | GUSBP11_chr22_−_23982494_{—} | SFT2D2_chr1_+_168205826_{—} |
| 202412255_202411700_202418116 | 23982610_23981129_23994665 | 168205862_168204420_168205949 |
| TANGO2_chr22_+_20043465_{—} | SRRM1_chr1_+_24989673_{—} | ARHGAP22_chr10_−_49687678_{—} |
| 20043536_20040107_20049052 | 24989715_24989295_24993305 | 49687807_49667934_49763507 |
| MINOS1_chr1_+_19927280_{—} | DCTD_chr4_−_183837571_{—} | TEX30_chr13_−_103419622_{—} |
| 19927465_19923603_19949967 | 183837692_183836728_183838440 | 103419828_103418930_103420609 |
| PGAP1_chr2_−_197786826_{—} | RAD52_chr12_−_1023576_{—} | MYO3B_chr2_+_171071238_{—} |
| 197786910_197784874_197791193 | 1023698_1023287_1025509 | 171071338_171070993_171073828 |
| TNK2_chr3_−_195612283_{—} | B4GALT6_chr18_−_29237938_{—} | SETD9_chr5_+_56206868_{—} |
| 195612414_195611904_195613847 | 29238052_29225442_29246218 | 56206938_56206221_56208837 |
| NABP1_chr2_+_192547217_{—} | CSF2RB_chr22_+_37332590_{—} | GSTM1_chr1_+_110230738_{—} |
| 192547321_192546743_192548454 | 37332694_37331729_37333418 | 110230867_110230531_110231294 |
| EIF4A2_chr3_+_186506098_{—} | RP11-793H13.8_chr12_+_53858543_{—} | PCGF3_chr4_+_726232_{—} |
| 186506205_186505671_186506913 | 53858636_53856351_53861004 | 726287_724899_727460 |
| SNX25_chr4_+_186260532_{—} | TBPL1_chr6_+_134274322_{—} | TPCN1_chr12_+_113722522_{—} |
| 186260664_186253913_186263129 | 134274563_134273868_134301219 | 113722576_113722442_113723693 |
| TRA2B_chr3_−_185649364_{—} | JARID2_chr6_+_15410454_{—} | MTIF2_chr2_−_55493516_{—} |
| 185649640_185643414_185655612 | 15410596_15374483_15452236 | 55493654_55491001_55494704 |
| TRA2B_chr3_−_185649364_{—} | RP1-27K12.2_chr6_−_53437997_{—} | GPALPP1_chr13_+_45603373_{—} |
| 185649640_185644522_185655612 | 53438073_53434403_53481683 | 45606242_45602138_45607370 |
| NOP16_chr5_−_175813840_{—} | CBR3-AS1_chr21_−_37518553_{—} | AC092675.3_chr2_−_98949903_{—} |
| 175813927_175812328_175815235 | 37518653_37509474_37528514 | 98949966_98949767_98950385 |
| NOP16_chr5_−_175813840_{—} | PLEKHG7_chr12_+_93162758_{—} | NAXD_chr13_+_111277536_{—} |
| 175813944_175812328_175815235 | 93162856_93157970_93163863 | 111277625_111276626_111279785 |
| CEP44_chr4_+_175238485_{—} | FAM114A2_chr5_−_153417852_{—} | CABIN1_chr22_+_24558033_{—} |
| 175239816_175237718_175252632 | 153417994_153414527_153418416 | 24558183_24552544_24560367 |
| NPRL3_chr16_−_174935_{—} | ADGRE5_chr19_+_14507153_{—} | RIMKLB_chr12_+_8852380_{—} |
| 175072_169254_180520 | 14507285_14501891_14508469 | 8853210_8850893_8866406 |
| BABAM1_chr19_+_17384712_{—} | SLAMF7_chr1_+_160720093_{—} | RGL3_chr19_−_11515998_{—} |
| 17384833_17382464_17387303 | 160720213_160719883_160721975 | 11516102_11515907_11517094 |
| MPRIP_chr17_+_17083920_{—} | COQ6_chr14_+_74428439_{—} | LTBP3_chr11_−_65307483_{—} |
| 17083983_17083402_17088136 | 74428606_74428273_74429672 | 65307624_65307352_65307715 |
| EPS15L1_chr19_−_16472589_{—} | SYTL1_chr1_+_27676462_{—} | SFMBT1_chr3_−_53077151_{—} |
| 16472795_16466662_16495939 | 27676623_27676256_27676879 | 53077270_53003274_53078961 |
| OXNAD1_chr3_+_16313651_{—} | TEX9_chr15_+_56681515_{—} | ARNTL2_chr12_+_27529278_{—} |
| 16313828_16313229_16327848 | 56681598_56676229_56683440 | 27529320_27521345_27533179 |
| MAP3K4_chr6_+_161529982_{—} | LINC00174_chr7_−_65941245_{—} | CDS1_chr4_+_85564176_{—} |
| 161530073_161529891_161530786 | 65941314_65915223_65943995 | 85564296_85562143_85569709 |
| ANKRD28_chr3_−_15855746_{—} | GOLGA4_chr3_+_37327504_{—} | ZFAND6_chr15_+_80367505_{—} |
| 15855816_15836813_15900883 | 37327552_37323763_37330725 | 80367657_80364989_80390757 |
| NDE1_chr16_+_15793585_{—} | LINC00893_chrX_−_148617478_{—} | BCL11A_chr2_−_60695866_{—} |
| 15793711_15790717_15818047 | 148617514_148615906_148617705 | 60695968_60689559_60773105 |
| DNAJB6_chr7_+_157202488_{—} | RP3-449017.1_chr22_−_38765990_{—} | PTBP1_chr19_+_805512_{—} |
| 157208223_157178305_157208709 | 38766050_38765384_38776374 | 805569_805187_806407 |
| CCNL1_chr3_−_156869517_{—} | SGSM1_chr22_+_25270381_{—} | FRMD8_chr11_+_65161043_{—} |
| 156869719_156868170_156869965 | 25270546_25264822_25272543 | 65161145_65156999_65161511 |
| MIB2_chr1_+_1564764_{—} | ZNF35_chr3_+_44694048_{—} | MED24_chr17_−_38191168_{—} |
| 1564946_1564691_1565018 | 44694193_44692751_44700192 | 38191225_38189709_38191369 |
| MIB2_chr1_+_1561746_{—} | TMEM176B_chr7_−_150491048_{—} | ATP13A2_chr1_−_17312957_{—} |
| 1562134_1561033_1562216 | 150491159_150490688_150493453 | 17313127_17312853_17313299 |
| INTS3_chr1_+_153733495_{—} | ARMC10_chr7_+_102737723_{—} | FOXM1_chr12_−_2974520_{—} |
| 153733585_153733394_153734045 | 102737795_102733100_102738745 | 2974565_2973918_2975558 |
| THEM4_chr1_−_151862458_{—} | WDSUB1_chr2_−_160116320_{—} | NDUFS1_chr2_−_207017142_{—} |
| 151862690_151861849_151867483 | 160116354_160112886_160132056 | 207017234_207014649_207018341 |
| PDXDC1_chr16_+_15095632_{—} | FAM228B_chr2_+_24360778_{—} | YIPF3_chr6_−_43480806_{—} |
| 15095744_15092262_15098043 | 24360970_24358057_24362239 | 43480836_43480612_43481096 |
| TSC22D2_chr3_+_150155984_{—} | VPS13A_chr9_+_79898270_{—} | STEAP3_chr2_+_119988195_{—} |
| 150156141_150129095_150174862 | 79898387_79897190_79898462 | 119988610_119981464_120003064 |
| REPIN1_chr7_+_150067848_{—} | UBE3A_chr15_−_25652213_{—} | AIM1L_chr1_−_26662585_{—} |
| 150067973_150066957_150068319 | 25652375_25650649_25654234 | 26662698_26658109_26662828 |
| REPIN1_chr7_+_150067848_{—} | KIF27_chr9_−_86474070_{—} | IFT172_chr2_−_27668611_{—} |
| 150067973_150066957_150068316 | 86474286_86468750_86482598 | 27668710_27668316_27668795 |
| REPIN1_chr7_+_150067848_{—} | MAX_chr14_−_65568263_{—} | THAP9-AS1_chr4_−_83819141_{—} |
| 150067973_150066030_150068319 | 65568290_65560533_65569021 | 83819215_83816927_83821229 |
| REPIN1_chr7_+_150067848_{—} | WASH3P_chr15_+_102506155_{—} | BRF1_chr14_−_105707601_{—} |
| 150067973_150066030_150068316 | 102506426_102501844_102512798 | 105707751_105695250_105718843 |
| REPIN1_chr7_+_150067802_{—} | MYEF2_chr15_−_48444430_{—} | HNRNPH1_chr5_−_179061065_{—} |
| 150067973_150066957_150068316 | 48444481_48444139_48445988 | 179061158_179050165_179061449 |
| TMEM185A_chrX_−_148685652_{—} | MIB2_chr1_+_1562029_{—} | WEE2-AS1_chr7_−_141437182_{—} |
| 148685736_148682143_148713225 | 1562134_1561033_1562216 | 141437367_141427945_141437997 |
| EZH2_chr7_−_148529725_{—} | MYO18A_chr17_−_27412621_{—} | LINC01607_chr8_+_80712795_{—} |
| 148529842_148526940_148543561 | 27412666_27409456_27413455 | 80712925_80696001_80714721 |
| SHPRH_chr6_−_146272926_{—} | LSP1_chr11_+_1902661_{—} | ST7_chr7_+_116738666_{—} |
| 146273059_146271618_146273484 | 1902826_1901454_1904648 | 116738869_116655068_116739815 |
| TCERG1_chr5_+_145889629_{—} | RP1-27K12.2_chr6_−_53434220_{—} | HSD3B7_chr16_+_30996973_{—} |
| 145889723_145888808_145890003 | 53434382_53429687_53481683 | 30997145_30996618_30997743 |
| DDX39A_chr19_−_14521232_{—} | SPIDR_chr8_+_48639761_{—} | DNAH2_chr17_+_7708275_{—} |
| 14521417_14521146_14521800 | 48639855_48626203_48647868 | 7708392_7707784_7708569 |
| DDX39A_chr19_−_14521359_{—} | RP4-800G7.2_chr7_+_148987028_{—} | USMG5_chr10_−_105155502_{—} |
| 14521417_14521146_14521800 | 148987129_148984867_148989301 | 105155789_105152223_105156165 |
| GK5_chr3_−_141903552_{—} | SYNRG_chr17_−_35939227_{—} | PIK3IP1_chr22_−_31685300_{—} |
| 141904635_141901891_141904770 | 35939530_35937711_35944755 | 31685379_31679274_31685484 |
| GNPTG_chr16_+_1407700_{—} | AFMID_chr17_+_76201683_{—} | DGKD_chr2_+_234377068_{—} |
| 1407833_1402307_1411743 | 76201819_76201599_76202026 | 234377199_234375849_234378016 |
| SPOPL_chr2_+_139312008_{—} | NCK1_chr3_+_136586272_{—} | JMJD8_chr16_−_732957_{—} |
| 139312105_139310251_139316591 | 136586367_136581195_136646825 | 733087_732874_733166 |
| NDUFAF5_chr20_+_13795063_{—} | PEAK1_chr15_−_77657504_{—} | SPTBN5_chr15_−_42180125_{—} |
| 13795161_13789548_13797108 | 77657567_77578846_77712347 | 42180283_42179639_42182286 |
| BRD8_chr5_−_137493330_{—} | CARM1_chr19_+_11032050_{—} | TBC1D3K_chr17_+_36291898_{—} |
| 137493583_137492956_137495243 | 11032119_11031803_11032290 | 36291964_36291514_36292427 |
| BCLAF1_chr6_−_136590278_{—} | GPR107_chr9_+_132872648_{—} | OCRL_chrX_+_128691837_{—} |
| 136590441_136589477_136590574 | 132872705_132869818_132887193 | 128691927_128691412_128692609 |
| MUM1_chr19_+_1358586_{—} | PISD_chr22_−_32021748_{—} | ACIN1_chr14_−_23559190_{—} |
| 1358699_1358463_1360134 | 32022002_32017871_32034351 | 23559310_23551045_23559730 |
| MUM1_chr19_+_1358586_{—} | TMPRSS5_chr11_−_113560486_{—} | CHKA_chr11_−_67848870_{—} |
| 1358692_1358463_1360134 | 113560639_113558996_113565199 | 67848924_67842297_67864485 |
| PPP2R2D_chr10_+_133748397_{—} | FAIM_chr3_+_138329445_{—} | SNHG1_chr11_−_62621023_{—} |
| 133748510_133748059_133753534 | 138329879_138327779_138340248 | 62621062_62620587_62622359 |
| CCNL2_chr1_−_1328058_{—} | PHPT1_chr9_+_139744954_{—} | PICALM_chr11_−_85689112_{—} |
| 1328183_1326245_1328775 | 139745012_139744589_139745206 | 85689136_85687725_85692171 |
| IRF1_chr5_−_131821942_{—} | CD44_chr11_+_35219667_{—} | CENPO_chr2_+_25016720_{—} |
| 131822065_131821408_131822248 | 35219793_35211612_35236398 | 25016834_25016389_25022543 |
| ARHGAP44_chr17_+_12890417_{—} | TCP11L1_chr11_+_33076138_{—} | IFT80_chr3_−_160109333_{—} |
| 12890469_12888225_12893348 | 33076271_33065482_33079464 | 160109431_160095328_160116933 |
| ZNF490_chr19_−_12720683_{—} | N6AMT1_chr21_−_30252191_{—} | PROX1-AS1_chr1_−_214016825_{—} |
| 12720788_12720016_12721377 | 30252275_30250655_30254481 | 214016911_214005965_214068005 |
| CHCHD6_chr3_+_126499182_{—} | ATM_chr11_+_108095510_{—} | SH3D19_chr4_−_152065371_{—} |
| 126499273_126452065_126571489 | 108095598_108093913_108098321 | 152065440_152065202_152069073 |
| METTL10_chr10_−_126450868_{—} | PGAP2_chr11_+_3832479_{—} | RFC5_chr12_+_118455494_{—} |
| 126451127_126449072_126453960 | 3832654_3829881_3844842 | 118455858_118454697_118456876 |
| C5orf63_chr5_−_126392088_{—} | CASD1_chr7_+_94164620_{—} | MUTYH_chr1_−_45804178_{—} |
| 126392198_126388361_126394573 | 94164835_94163114_94166783 | 45804328_45800183_45805570 |
| TRMT11_chr6_+_126327987_{—} | CTBP2_chr10_−_126811225_{—} | ABCB9_chr12_−_123425353_{—} |
| 126328100_126320759_126329537 | 126811437_126799662_126848425 | 123425542_123424831_123428937 |
| TATDN1_chr8_−_125531058_{—} | LINC00339_chr1_+_22353906_{—} | UBE3A_chr15_−_25652213_{—} |
| 125531122_125528271_125535177 | 22354027_22352132_22356772 | 25652409_25650649_25654234 |
| TATDN1_chr8_−_125530982_{—} | CHTF8_chr16_−_69154955_{—} | ADGRL1_chr19_−_14275432_{—} |
| 125531122_125528271_125535177 | 69155073_69154552_69155338 | 14275517_14274218_14277827 |
| MRRF_chr9_+_125048317_{—} | EFS_chr14_−_23829763_{—} | GLYATL1_chr11_+_58714518_{—} |
| 125048445_125047566_125054027 | 23830042_23829490_23834216 | 58714541_58711139_58715330 |
| PSTK_chr10_+_124746849_{—} | LEMD1_chr1_−_205353415_{—} | RLIM_chrX_−_73832416_{—} |
| 124747020_124746478_124749689 | 205353492_205350984_205389582 | 73832478_73815835_73834256 |
| ANAPC5_chr12_−_121756079_{—} | ALKBH3_chr11_+_43923065_{—} | POLL_chr10_−_103345072_{—} |
| 121756207_121746494_121756303 | 43923275_43911378_43940587 | 103345235_103344676_103347002 |
| EAF2_chr3_+_121555491_{—} | SEC24D_chr4_−_119679566_{—} | WASF1_chr6_−_110481837_{—} |
| 121555641_121554238_121563299 | 119679782_119679000_119681545 | 110481935_110448832_110500641 |
| TIAL1_chr10_−_121339982_{—} | ARID1B_chr6_+_157471858_{—} | SLC2A8_chr9_+_130167096_{—} |
| 121340358_121339522_121341433 | 157471987_157470085_157488173 | 130167270_130159565_130169390 |
| LAMP2_chrX_−_119572930_{—} | C19orf57_chr19_−_13994120_{—} | LRMP_chr12_+_25214525_{—} |
| 119573148_119565317_119575584 | 13994213_13993765_13996773 | 25214587_25205890_25216653 |
| TMEM39A_chr3_−_119176864_{—} | DUSP22_chr6_+_335113_{—} | MYO15A_chr17_+_18062919_{—} |
| 119177031_119171377_119180808 | 335163_311962_345853 | 18062993_18062661_18063248 |
| TAMM41_chr3_−_11874476_{—} | TXNL1_chr18_−_54314679_{—} | RBCK1_chr20_+_401514_{—} |
| 11874625_11871338_11880695 | 54314766_54305736_54318247 | 401675_400375_402770 |
| REV3L_chr6_−_111793215_{—} | PPP3CB-AS1_chr10_+_75258490_{—} | ZNF17_chr19_+_57929279_{—} |
| 111793343_111737675_111803952 | 75258625_75256664_75259352 | 57929406_57925004_57931002 |
| RBM15_chr1_+_110888160_{—} | ADAMTS13_chr9_+_136301948_{—} | KIAA1191_chr5_−_175786813_{—} |
| 110888271_110884890_110888929 | 136302075_136298824_136302868 | 175786921_175775359_175788604 |
| DDO_chr6_−_110725976_{—} | NAXD_chr13_+_111279785_{—} | PCCA_chr13_+_101167680_{—} |
| 110726153_110714545_110729536 | 111279894_111276626_111286891 | 101167821_101101559_101179928 |
| C12orf76_chr12_−_110482224_{—} | CEP350_chr1_+_180021358_{—} | SLC44A3_chr1_+_95357836_{—} |
| 110482338_110480264_110486167 | 180021451_180017840_180022104 | 95358073_95356823_95360373 |
| MAGOHB_chr12_−_10761696_{—} | GPBP1L1_chr1_−_46151247_{—} | PHKB_chr16_+_47497809_{—} |
| 10761982_10760535_10762429 | 46151292_46126897_46152664 | 47497904_47495337_47531309 |
| TCP11L2_chr12_+_106706070_{—} | MAPKAPK5_chr12_+_112306556_{—} | GCNT2_chr6_+_10527706_{—} |
| 106706212_106705010_106708135 | 112306665_112305473112308074 | 10527893_10521827_10621583 |
| ATXN7L1_chr7_−_105289853_{—} | SDR39U1_chr14_−_24910879_{—} | SUCO_chr1_+_172554997_{—} |
| 105290007_105283568_105305512 | 24911001_24910132_24911551 | 172555088_172548407_172557899 |
| NT5C2_chr10_−_104871501_{—} | GUCD1_chr22_−_24950916_{—} | SMUG1_chr12_−_54582298_{—} |
| 104871562_104866463_104899162 | 24951104_24944969_24951582 | 54582380_54581689_54582734 |
| KLC1_chr14_+_104151322_{—} | BTBD2_chr19_−_2028706_{—} | PGAP2_chr11_+_3832479_{—} |
| 104151373_104145855_104153417 | 2028833_2017299_2034749 | 3832654_3829881_3845112 |
| KLC1_chr14_+_104151322_{—} | MAD2L2_chr1_−_11741095_{—} | TCF12_chr15_+_57544618_{—} |
| 104151373_104145882_104153417 | 11741495_11740670_11751469 | 57544690_57543621_57545459 |
| APOPT1_chr14_+_104029551_{—} | PRPF3_chr1_+_150300015_{—} | GUF1_chr4_+_44690023_{—} |
| 104029716_104029461_104037959 | 150300111_150298339_150300778 | 44690163_44688730_44691302 |
| SRRT_chr7_+_100480385_{—} | FLCN_chr17_−_17124850_{—} | MAMDC4_chr9_+_139752637_{—} |
| 100480711_100479862_100481690 | 17124942_17122523_17129489 | 139752728_139752549_139752851 |
| SRRT_chr7_+_100478316_{—} | PODNL1_chr19_−_14046792_{—} | TDP1_chr14_+_90422921_{—} |
| 100478390_100473333_100478905 | 14046857_14044806_14047179 | 90423144_90422314_90429451 |
| RP4-714D9.5_chr1_+_100464816_{—} | FGGY_chr1_+_59811918_{—} | TOM1L1_chr17_+_52982849_{—} |
| 100464971_100459297_100472589 | 59812070_59787422_59844420 | 52982928_52978284_52990026 |
| DLG3_chrX_+_69675636_{—} | QSER1_chr11_+_32948702_{—} | NCOR1_chr17_−_15990485_{—} |
| 69675952_69675113_69698999 | 32948815_32914880_32953288 | 15990659_15989756_15995176 |
| NMU_chr4_−_56482504_{—} | MAPK8_chr10_+_49533939_{—} | UBE3A_chr15_−_25652213_{—} |
| 56482552_56475346_56496568 | 49534008_49514864_49609654 | 25652284_25650649_25654234 |
| MOB4_chr2_+_198388347_{—} | ARMCX5-GPRASP2_chrX_+_101856391_{—} | MEGF8_chr19_+_42862938_{—} |
| 198388410_198380870_198400253 | 101856437_101854775_101860409 | 42863106_42862459_42863249 |
| DHX33_chr17_−_5354126_{—} | TKFC_chr11_+_61103311_{—} | PPP1R7_chr2_+_242092897_{—} |
| 5354254_5353726_5356899 | 61103409_61102203_61105412 | 242093019_242089962_242097221 |
| TPX2_chr20_+_30330343_{—} | NEK11_chr3_+_130746773_{—} | PACRGL_chr4_+_20709425_{—} |
| 30330450_30327424_30345209 | 130746846_130745850_130748456 | 20709493_20706437_20715054 |
| ATG5_chr6_−_106756238_{—} | RCC1_chr1_+_28857034_{—} | KPNA5_chr6_+_117013217_{—} |
| 106756366_106740981_106763975 | 28857085_28856451_28858314 | 117013319_117010616_117013455 |
| GALNS_chr16_−_88909113_{—} | CHTF18_chr16_+_841850_{—} | MCPH1-AS1_chr8_−_6527509_{—} |
| 88909237_88908379_88923165 | 841948_841370_842223 | 6527761_6492940_6565661 |
| AC005083.1_chr7_+_20257410_{—} | PROM1_chr4_−_15981503_{—} | PFAS_chr17_+_8168605_{—} |
| 20257481_20257245_20257878 | 15981527_15972705_15982044 | 8168786_8168443_8168873 |
| TTLL3_chr3_+_9876110_{—} | CCDC112_chr5_−_114605399_{—} | AC005154.6_chr7_−_30591715_{—} |
| 9876187_9874929_9876364 | 114605495_114604697_114606909 | 30591795_30590397_30603346 |
| ACSL4_chrX_−_108939372_{—} | TMEM161B_chr5_−_87493485_{—} | CANT1_chr17_−_76994229_{—} |
| 108939425_108926601_108976367 | 87493582_87492305_87494792 | 76994368_76994045_77001453 |
| HPS1_chr10_−_100190887_{—} | METTL4_chr18_−_2544193_{—} | BICDL1_chr12_+_120510314_{—} |
| 100191048_100190427_100193696 | 2544285_2539144_2547353 | 120510533_120509605_120518690 |
| KIF1A_chr2_−_241696735_{—} | SENP1_chr12_−_48482945_48483076_{—} | ANKRD36_chr2_+_97830133_{—} |
| 241697011_241689964_241697776 | 48482743_48491776 | 97830206_97830048_97833313 |
| SKA1_chr18_+_47911585_{—} | CRTC1_chr19_+_18882251_{—} | NFRKB_chr11_−_129763191_{—} |
| 47911723_47908596_47917493 | 18882356_18879603_18885709 | 129763280_129762765_129764253 |
| CTSC_chr11_−_88061279_{—} | COQ4_chr9_+_131087421_{—} | SNX5_chr20_−_17949017_{—} |
| 88061364_88059611_88068104 | 131087518_131085426_131088057 | 17949100_17937681_17949342 |
| PISD_chr22_−_32019669_{—} | SIGIRR_chr11_−_409867_{—} | EBP_chrX_+_48380580_{—} |
| 32019835_32017871_32034351 | 410027_408206_417293 | 48380640_48380295_48382086 |
| CTNND1_chr11_+_57561481_{—} | EFCAB6_chr22_−_44030981_{—} | ARMC6_chr19_+_19144939_{—} |
| 57561553_57529518_57563048 | 44031096_44028118_44062983 | 19145047_19144739_19153519 |
| ABHD5_chr3_+_43759257_{—} | YAF2_chr12_−_42604349_{—} | CEACAM19_chr19_+_45182124_{—} |
| 43759349_43756550_43759934 | 42604421_42555567_42631400 | 45182208_45179693_45185838 |
| RBM27_chr5_+_145631273_{—} | STAMBP_chr2_+_74056531_{—} | LPGAT1_chr1_−_212003524_{—} |
| 145631438_145616995_145634505 | 74056637_74056123_74057971 | 212003672_212002661_212003914 |
| ANKMY1_chr2_−_241465645_{—} | CCDC18-AS1_chr1_−_93790191_{—} | SYDE2_chr1_−_85643828_{—} |
| 241465862_241465266_241468453 | 93790286_93771027_93802933 | 85643955_85634908_85647780 |
| TMEM39B_chr1_+_32542764_{—} | CAMTA2_chr17_−_4885383_{—} | PLOD2_chr3_−_145795648_{—} |
| 32542919_32541423_32557275 | 4885522_4885126_4886051 | 145795711_145794682_145796902 |
| LSM14B_chr20_+_60702640_{—} | SNX22_chr15_+_64444441_{—} | SLTM_chr15_−_59191667_{—} |
| 60702757_60701495_60705274 | 64444525_64444049_64444836 | 59192136_59191051_59193458 |
| RP11-321G12.1_chr15_+_63723403_{—} | R3HDM2_chr12_−_57686354_{—} | ARHGEF38_chr4_+_106604078_{—} |
| 63723541_63690666_63724350 | 57686450_57682823_57689181 | 106604203_106603766_106616716 |
| KAT6A_chr8_−_41907093_{—} | ARHGEF12_chr11_+_120300420_{—} | GMDS-AS1_chr6_+_2263834_{—} |
| 41907225_41906820_41909418 | 120300540_120300226_120302479 | 2263918_2249160_2329516 |
| KLK11_chr19_−_51528847_{—} | CSPP1_chr8_+_68062017_{—} | ADGRD1_chr12_+_131484927_{—} |
| 51528922_51526484_51529793 | 68062170_68049838_68066258 | 131484987_131476937_131488730 |
| DICER1_chr14_−_95600697_{—} | RIMKLB_chr12_+_8932630_{—} | ZNF410_chr14_+_74388768_{—} |
| 95600840_95599840_95623566 | 8932708_8930398_8933170 | 74388909_74387808_74390097 |
| CCDC191_chr3_−_113737524_{—} | BTBD3_chr20_+_11899733_{—} | CRACR2A_chr12_−_3842107_{—} |
| 113737715_113729868_113750472 | 11899824_11899249_11900365 | 3842376_3822483_3862180 |
| ATG4D_chr19_+_10657514_{—} | SLC35B1_chr17_−_47784326_{—} | BLOC1S6_chr15_+_45884332_{—} |
| 10657747_10655806_10657864 | 47784430_47783671_47785091 | 45884474_45879723_45890077 |
| L3MBTL3_chr6_+_130370900_{—} | ALOX12B_chr17_−_7979492_{—} | ARHGAP23_chr17_+_36633819_{—} |
| 130370975_130370538_130372393 | 7979662_7976636_7979974 | 36633946_36628247_36634068 |
| ZNF611_chr19_−_53217267_{—} | ACACA_chr17_−_35567380_{—} | ATXN7L1_chr7_−_105279933_{—} |
| 53217394_53210795_53219084 | 35567404_35564714_35578644 | 105280016_105279056_105283284 |
| RFXANK_chr19_+_19307994_{—} | MAPKBP1_chr15_+_42109811_{—} | ERP29_chr12_+_112457559_{—} |
| 19308060_19307855_19308329 | 42109933_42109656_42110206 | 112457698_112451413_112459953 |
| PPP2R2A_chr8_+_26197418_{—} | ABI1_chr10_−_27044583_{—} | DDX5_chr17_−_62497174_{—} |
| 26197489_26196503_26211983 | 27044670_27040712_27047990 | 62497261_62496891_62497378 |
| KAT6A_chr8_−_41907137_{—} | PLD3_chr19_+_40871624_{—} | ITGB1BP1_chr2_−_9547577_{—} |
| 41907225_41906820_41909418 | 40871837_40854675_40872325 | 9547727_9547034_9548241 |
| AP000275.65_chr21_−_33974581_{—} | MPDZ_chr9_−_13143464_{—} | TCIRG1_chr11_+_67812513_{—} |
| 33974677_33954725_33975470 | 13143563_13140148_13147546 | 67812569_67811811_67814899 |
| RP11-96H17.1_chr16_−_63094197_{—} | UBA6_chr4_−_68562362_{—} | NRSN2_chr20_+_330281_{—} |
| 63094314_63092558_63124165 | 68562425_68547931_68566766 | 330476_330007_333853 |
| IFT80_chr3_−_160109333_{—} | KTN1_chr14_+_56130672_{—} | ALCAM_chr3_+_105270987_{—} |
| 160109431_160102434_160116933 | 56130759_56128330_56133958 | 105271026_105269103_105271311 |
| SENP1_chr12_−_48482945_{—} | PIP4K2C_chr12_+_57993182_{—} | LRBA_chr4_−_151842349_{—} |
| 48483076_48482743_48490125 | 57993221_57992994_57994106 | 151842445_151837890_151849667 |
| TBC1D32_chr6_−_121576473_{—} | EPN2_chr17_+_19183170_{—} | TNKS_chr8_+_9590788_{—} |
| 121576556_121563485_121600266 | 19183293_19152839_19185267 | 9590954_9584230_9592374 |
| IL17RB_chr3_+_53889320_{—} | ASCC1_chr10_−_73975539_{—} | LINC01748_chr1_−_61009148_{—} |
| 53889368_53886153_53890870 | 73975867_73973089_73975964 | 61009193_61008083_61053708 |
| SLC38A9_chr5_−_54945034_{—} | DSTYK_chr1_−_205117332_{—} | MINDY1_chr1_−_150974640_{—} |
| 54945142_54941723_54948356 | 205117467_205116873_205119807 | 150974876_150974258_150978787 |
| SYNE2_chr14_+_64682003_{—} | CTU2_chr16_+_88776630_{—} | BHLHB9_chrX_+_102002744_{—} |
| 64682072_64681188_64682965 | 88776690_88773006_88778042 | 102002839_101975955_102003626 |
| KIF9_chr3_−_47323629_{—} | MTHFSD_chr16_−_86585638_{—} | RGP1_chr9_+_35751952_{—} |
| 47323875_47318869_47323984 | 86585752_86582183_86588250 | 35752142_35751751_35752647 |
| ANKRD20A8P_chr2_−_95504557_{—} | EZH2_chr7_−_148533913_{—} | ERCC1_chr19_−_45917220_{—} |
| 95504588_95501874_95511124 | 148534092_148526940_148543561 | 45917292_45917003_45918118 |
| DYRK3_chr1_+_206810994_{—} | P4HA2_chr5_−_131562389_{—} | SIGIRR_chr11_−_408694_{—} |
| 206811106_206809125_206820732 | 131562914_131554337_131563482 | 408893_408206_417293 |
| PAPLN_chr14_+_73735264_{—} | FOXM1_chr12_−_2974520_{—} | C16orf95_chr16_−_87343902_{—} |
| 73735431_73732215_73739202 | 2974565_2973921_2975558 | 87343939_87339511_87348576 |
| FAT3_chr11_+_92622354_{—} | ITPA_chr20_+_3193814_{—} | ZNF185_chrX_+_152090716_{—} |
| 92622414_92620279_92623656 | 3193872_3190263_3194630 | 152090800_152089292_152097118 |
| PCGF3_chr4_+_726188_{—} | CEP44_chr4_+_175219116_{—} | MICAL1_chr6_−_109769077_{—} |
| 726287_724899_727460 | 175219213_175205291_175220222 | 109769166_109768925_109769405 |
| SLC25A48_chr5_+_135209709_{—} | SMAD2_chr18_−_45456731_{—} | KLHL8_chr4_−_88106402_{—} |
| 135209843_135207407_135215656 | 45456817_45423180_45457170 | 88106723_88104541_88116475 |
| PAX8-AS1_chr2_+_114012966_{—} | YARS_chr1_−_33263192_{—} | RHBG_chr1_+_156351596_{—} |
| 114013126_113995789_114020854 | 33263239_33252676_33263363 | 156351734_156351304_156351858 |
| PHF8_chrX_−_54020033_{—} | SLC29A2_chr11_−_66136530_{—} | HECTD1_chr14_−_31583082_{—} |
| 54020336_54019272_54022125 | 66136602_66136142_66136839 | 31583265_31582690_31583491 |
| ADGRL1_chr19_−_14271404_{—} | PALB2_chr16_−_23649779_{—} | ARHGAP27_chr17_−_43509581_{—} |
| 14271508_14271109_14272123 | 23649913_23649450_23652430 | 43509715_43509118_43510117 |
| ARMC10_chr7_+_102732923_{—} | EYA3_chr1_−_28354299_{—} | OTUD7B_chr1_−_149949360_{—} |
| 102733100_102727211_102737723 | 28354437_28343750_28362054 | 149949511_149943179_149982396 |
| STARD3NL_chr7_+_38256625_{—} | ASIC1_chr12_+_50470750_{—} | USMG5_chr10_−_105153955_{—} |
| 38256679_38254706_38256788 | 50470843_50453737_50470995 | 105154151_105152223_105155502 |
| LINC01572_chr16_−_72604333_{—} | CHD3_chr17_+_7813745_{—} | PTPN6_chr12_+_7060771_{—} |
| 72604414_72589225_72661433 | 7813909_7812656_7814164 | 7060894_7055902_7061145 |
| CEACAM19_chr19_+_45174575_{—} | PRPSAP2_chr17_+_18768781_{—} | CFAP69_chr7_+_89933282_{—} |
| 45174708_45165665_45175017 | 18768877_18761574_18769114 | 89933420_89929373_89934057 |
| SLC29A1_chr6_+_44193797_{—} | TROVE2_chr1_+_193038163_{—} | HEATR5A_chr14_−_31814292_{—} |
| 44193904_44191378_44194999 | 193038764_193028906_193044949 | 31814482_31813271_31816953 |
| RPL17_chr18_−_47018105_{—} | USP39_chr2_+_85872936_{—} | CANT1_chr17_−_76994229_{—} |
| 47018203_47017954_47018644 | 85873022_85872206_85875883 | 76994368_76994045_77005745 |
| TBL1XR1_chr3_−_176797614_{—} | SPIN1_chr9_+_91077410_{—} | LSM14B_chr20_+_60704840_{—} |
| 176797733_176782810_176914908 | 91077664_91063904_91083286 | 60705008_60701495_60705274 |
| ATXN7L1_chr7_−_105516257_{—} | GCNT1_chr9_+_79074975_{—} | SNHG17_chr20_−_37056322_{—} |
| 105516326_105429154_105516823 | 79075093_79074221_79115831 | 37056409_37055146_37059683 |
| IRF1_chr5_−_131825083_{—} | ZNF417_chr19_−_58426705_{—} | TMEM87B_chr2_+_112838911_{—} |
| 131825175_131822822_131826236 | 58426899_58423557_58427746 | 112839095_112838695_112843581 |
| ATP5SL_chr19_−_41939176_{—} | MCRS1_chr12_−_49961429_{—} | NQO2_chr6_+_3004728_{—} |
| 41939339_41938313_41939441 | 49961532_49959998_49961830 | 3004885_3000319_3006701 |
| NAA16_chr13_+_41936866_{—} | LRMP_chr12_+_25219298_{—} | CRYZ_chr1_−_75172786_{—} |
| 41937009_41936295_41941574 | 25219446_25216750_25222283 | 75172888_75172678_75175781 |
| MRI1_chr19_+_13876767_{—} | DNAJC2_chr7_−_102967778_{—} | SGSM3_chr22_+_40797596_{—} |
| 13876943_13875923_13879172 | 102967825_102967131_102968102 | 40797679_40796817_40798142 |
| GUSBP11_chr22_−_24042912_{—} | GAS5_chr1_−_173835898_{—} | GUCD1_chr22_−_24939809_{—} |
| 24043032_24026054_24047615 | 173835934_173835344_173836128 | 24940051_24939068_24942881 |
| DIS3L2_chr2_+_233063236_{—} | GORASP1_chr3_−_39144183_{—} | CCDC77_chr12_+_514676_{—} |
| 233063486_233028342_233075035 | 39144372_39142593_39148969 | 514730_498652_520912 |
| DNAH1_chr3_+_52409970_{—} | SYNE4_chr19_−_36498026_{—} | PGAP2_chr11_+_3844842_{—} |
| 52410009_52409429_52412617 | 36498170_36497573_36499455 | 3844970_3832654_3845112 |
| ENAH_chr1_−_225704897_{—} | KIAA0895_chr7_−_36396510_{—} | CRYZL1_chr21_−_34969585_{—} |
| 225705692_225702602_225706899 | 36397199_36375906_36406719 | 34969707_34968142_34971455 |
| NUTM2B-AS1_chr10_−_81568514_{—} | FAM204A_chr10_−_120101238_{—} | HSCB_chr22_+_29139869_{—} |
| 81568588_81565878_81574368 | 120101439_120095935_120101781 | 29139911_29138319_29147228 |
| BCS1L_chr2_+_219524759_{—} | D2HGDH_chr2_+_242688279_{—} | ATXN2_chr12_−_111953957_{—} |
| 219524968_219524466_219526128 | 242689344_242684292_242689565 | 111954167_111951343_111956052 |
| EZH2_chr7_−_148533913_{—} | UBXN2B_chr8_+_59325857_{—} | MAMDC4_chr9_+_139751616_{—} |
| 148534092_148529842_148543561 | 59325930_59324028_59329408 | 139751750_139751493_139751818 |
| KIN_chr10_−_7822226_{—} | SLC50A1_chr1_+_155109303_{—} | ZNF81_chrX_+_47702048_{—} |
| 7822270_7822141_7829782 | 155109427_155108852_155110454 | 47702167_47696406_47705503 |
| KYAT1_chr9_−_131609711_{—} | SYNE4_chr19_−_36498026_{—} | ANKRD36C_chr2_−_96604576_{—} |
| 131609922_131607690_131644175 | 36498170_36496339_36499455 | 96604649_96601392_96604734 |
| RAD1_chr5_−_34915520_{—} | HOTAIRM1_chr7_+_27138692_{—} | SLCO4A1-AS1_chr20_−_61297211_{—} |
| 34915731_34915066_34918156 | 27138985_27136007_27139397 | 61297362_61294839_61297736 |
| TPGS2_chr18_−_34385336_{—} | ZDHHC16_chr10_+_99213555_{—} | ECHDC1_chr6_−_127637594_{—} |
| 34385360_34380274_34387809 | 99213603_99213420_99214470 | 127637647_127636041_127648146 |
| SLC44A3-AS1_chr1_−_95144980_{—} | NBEAL2_chr3_+_47033964_{—} | UBL7_chr15_−_74740818_{—} |
| 95145163_95104114_95197813 | 47034045_47033436_47035426 | 74740899_74738568_74741526 |
| ACSF3_chr16_+_89178499_{—} | MKI67_chr10_−_129913191_{—} | SELENON_chr1_+_26128506_{—} |
| 89178654_89160404_89187208 | 129914271_129911866_129914754 | 26128608_26127651_26131632 |
| SSBP3_chr1_−_54723741_{—} | SLC37A4_chr11_−_118900941_{—} | ZFAND6_chr15_+_80390757_{—} |
| 54723822_54717533_54747110 | 118901445_118900274_118901558 | 80390920_80352151_80412669 |
| SHC1_chr1_−_154945768_{—} | AMMECR1L_chr2_−_128628387_{—} | LACE1_chr6_+_108831414_{—} |
| 154945984_154942675_154946723 | 128628502_128624570_128631401 | 108831555_108798495_108840899 |
| NSUN5P1_chr7_+_75040937_{—} | N4BP2L2_chr13_−_33052364_{—} | MRPS28_chr8_−_80940929_{—} |
| 75041042_75040009_75042066 | 33052412_33052185_33054726 | 80941031_80915415_80941995 |
| EXD2_chr14_+_69670633_{—} | AKIP1_chr11_+_8938833_{—} | YPEL5_chr2_+_30371110_{—} |
| 69670717_69658308_69695532 | 8938914_8936477_8940883 | 30371407_30369928_30379493 |
| RP11-286N22.8_chr11_+_61205475_{—} | SIRT2_chr19_−_39389018_{—} | PROM1_chr4_−_15981017_{—} |
| 61205585_61197654_61213412 | 39389065_39384611_39390145 | 15981086_15972705_15982044 |
| PACSIN2_chr22_−_43272893_{—} | SDR39U1_chr14_−_24911383_{—} | KDM4A_chr1_+_44119333_{—} |
| 43273016_43272339_43275053 | 24911466_24910132_24911551 | 44119470_44118984_44121261 |
| TSPAN15_chr10_+_71243446_{—} | TYW3_chr1_+_75204373_{—} | CROT_chr7_+_87022263_{—} |
| 71243632_71211446_71255349 | 75204472_75202306_75214434 | 87022383_87022139_87027839 |
| GGA1_chr22_+_38009125_{—} | ZNF480_chr19_+_52817405_{—} | NAB1_chr2_+_191520702_{—} |
| 38009198_38005161_38010196 | 52817532_52803737_52819086 | 191520879_191514086_191523883 |
| MYEOV_chr11_+_69181806_{—} | PMPCB_chr7_+_102948042_{—} | BAIAP2L2_chr22_−_38485528_{—} |
| 69181880_69181634_69182062 | 102948155_102944937_102949398 | 38485681_38483271_38493038 |
| PEX5_chr12_+_7342617_{—} | DPP9_chr19_−_4683201_{—} | CTTN_chr11_+_70267575_{—} |
| 7342812_7342346_7342957 | 4683336_4682850_4683488 | 70267686_70266616_70269045 |
| PRRC2A_chr6_+_31590466_{—} | NUMA1_chr11_−_71723446_{—} | ANAPC1_chr2_−_112631189_{—} |
| 31590678_31588635_31592037 | 71723488_71721900_71723940 | 112631241_112630985_112636386 |
| KCTD9_chr8_−_25301807_{—} | LSAMP_chr3_−_115553408_{—} | IRF7_chr11_−_614173_{—} |
| 25301851_25298189_25315714 | 115553444_115535493_115560691 | 614399_613865_614475 |
| ZNF195_chr11_−_3382972_{—} | VWDE_chr7_−_12406982_{—} | CDC42BPB_chr14_−_103420534_{—} |
| 3383119_3381795_3383775 | 12407119_12401147_12409170 | 103420612_103418940_103420938 |
| BLOC1S1_chr12_+_56110716_{—} | WDR45_chrX_−_48935301_{—} | TET2_chr4_+_106155053_{—} |
| 56110789_56110501_56112874 | 48935406_48934412_48935495 | 106158508_106068136_106180775 |
| EXD3_chr9_−_140246517_{—} | BCL3_chr19_+_45261502_{—} | LRRFIP2_chr3_−_37163125_{—} |
| 140246653_140245980_140247071 | 45261730_45260980_45261980 | 37163182_37163027_37170553 |
| TBX3_chr12_−_115117717_{—} | ARHGEF7_chr13_+_111862218_{—} | WNK1_chr12_+_980430_{—} |
| 115117777_115117456_115118683 | 111862349_111857720_111870025 | 980514_971436_987377 |
| TIA1_chr2_−_70456190_{—} | C19orf60_chr19_+_18700288_{—} | IL17RB_chr3_+_53889320_{—} |
| 70456223_70454954_70456395 | 18700493_18699887_18701663 | 53889368_53887024_53890870 |
| CHEK2_chr22_−_29133872_{—} | PREPL_chr2_−_44586635_{—} | TFAM_chr10_+_60150524_{—} |
| 29134026_29130715_29137756 | 44586889_44573529_44588662 | 60150620_60148579_60154117 |
| ZNF213-AS1_chr16_−_3182850_{—} | ATRX_chrX_−_76851181_{—} | ARL4A_chr7_+_12727259_{—} |
| 3182943_3182104_3184634 | 76851255_76849319_76854879 | 12727353_12726668_12727790 |
| IST1_chr16_+_71957190_{—} | SF1_chr11_−_64533422_{—} | ARL4A_chr7_+_12727259_{—} |
| 71957283_71956583_71958671 | 64533627_64532990_64534371 | 12727353_12726668_12727793 |
| ST18_chr8_−_53134199_{—} | KIAA1456_chr8_+_12848342_{—} | DQX1_chr2_−_74752135_{—} |
| 53134353_53126817_53142591 | 12848509_12809867_12863710 | 74752329_74751434_74752619 |
| BCORL1_chrX_+_129158964_{—} | RHOC_chr1_−_113247721_{—} | FNIP1_chr5_−_131046270_{—} |
| 129159354_129156952_129162609 | 113247745_113246428_113249699 | 131046354_131044965_131052257 |
| BIN1_chr2_−_127808729_{—} | PGAP3_chr17_−_37829766_{—} | NDRG2_chr14_−_21492188_{—} |
| 127808819_127808488_127816586 | 37829903_37829119_37830245 | 21492255_21491480_21493835 |
| GUSBP11_chr22_−_24032422_{—} | SHC1_chr1_−_154940679_{—} | RP1-234P15.4_chr6_+_75999969_{—} |
| 24032543_24026054_24036305 | 154940733_154940507_154940970 | 76000054_75995025_76000664 |
| HBP1_chr7_+_106814892_{—} | ZNF207_chr17_+_30693683_{—} | EZH2_chr7_−_148533913_{—} |
| 106815190_106810575_106820323 | 30693776_30692506_30694790 | 148534092_148529842_148581255 |
| DENNDIA_chr9_−_126531792_{—} | LRP8_chr1_−_53742363_{—} | STAP2_chr19_−_4324451_{—} |
| 126531842_126520101_126554865 | 53742750_53741425_53755238 | 4324661_4324194_4325212 |
| HIVEP1_chr6_+_12136023_{—} | DNAI1_chr9_+_34489997_{—} | RBMXL1_chr1_−_89453934_{—} |
| 12136125_12126103_12161671 | 34490122_34489447_34490366 | 89454034_89449749_89458267 |
| C16orf59_chr16_+_2510283_{—} | PTPN4_chr2_+_120639361_{—} | TRPV1_chr17_−_3491511_{—} |
| 2510382_2510171_2510623 | 120639406_120635118_120639672 | 3491661_3489220_3493100 |
| TPD52L1_chr6_+_125578243_{—} | KCTD17_chr22_+_37456862_{—} | ALDOA_chr16_+_30077196_{—} |
| 125578304_125569529_125583979 | 37456962_37455478_37458564 | 30077248_30075826_30078554 |
| HNRNPH1_chr5_−_179046269_{—} | ATG4B_chr2_+_242594685_{—} | TMUB2_chr17_+_42264984_{—} |
| 179046408_179045324_179047892 | 242594758_242594062_242606059 | 42265111_42264477_42266389 |
| FRMD8_chr11_+_65156831_{—} | ZNF195_chr11_−_3394806_{—} | BCS1L_chr2_+_219524759_{—} |
| 65156999_65154592_65161511 | 3394905_3392933_3400267 | 219524968_219524466_219525661 |
| TCIRG1_chr11_+_67817953_{—} | METTL26_chr16_−_685611_{—} | PEX2_chr8_−_77898422_{—} |
| 67818131_67817721_67818207 | 685774_685340_686093 | 77898532_77896431_77912225 |
| BDH1_chr3_−_197281432_{—} | PSAP_chr10_−_73583644_{—} | YIF1A_chr11_−_66052851_{—} |
| 197281584_197260432_197282651 | 73583653_73581764_73585593 | 66053007_66052437_66053171 |
| ITPA_chr20_+_3193964_{—} | SNRPA1_chr15_−_101833229_{—} | FAM49B_chr8_−_130916744_{—} |
| 3194029_3190263_3194630 | 101833377_101827907_101835301 | 130916831_130915596_130951853 |
| HNRNPH3_chr10_+_70097614_{—} | OXR1_chr8_+_107715133_{—} | SLAIN2_chr4_+_48396592_{—} |
| 70097753_70097090_70098259 | 107715318_107705105_107718609 | 48396670_48385801_48422141 |
| RPS6KL1_chr14_−_75385215_{—} | CASD1_chr7_+_94147514_{—} | PUF60_chr8_−_144906482_{—} |
| 75385308_75378544_75386547 | 94147635_94146968_94156473 | 144906566_144904083_144911449 |
| LINC01572_chr16_−_72651081_{—} | MYH10_chr17_−_8479960_{—} | ABHD14A_chr3_+_52014408_{—} |
| 72651133_72604414_72698790 | 8479990_8473130_8480553 | 52014644_52012390_52014851 |
| CRTC1_chr19_+_18885709_{—} | SDR39U1_chr14_−_24910879_{—} | PRRC2C_chr1_+_171556786_{—} |
| 18885796_18879603_18886450 | 24911001_24910132_24911303 | 171556890_171556360_171557517 |
| PPP1CB_chr2_+_29011523_{—} | GS1-124K5.12_chr7_−_66043515_{—} | TFAP2E_chr1_+_36039527_{—} |
| 29011675_29006844_29022064 | 66043714_66038537_66053498 | 36040010_36039205_36040549 |
| NME6_chr3_−_48342016_{—} | HPS1_chr10_−_100195028_{—} | CCDC92_chr12_−_124440830_{—} |
| 48342124_48340013_48342768 | 100195171_100193848_100195391 | 124440991_124428911_124457075 |
| HM13_chr20_+_30155880_{—} | CNTRL_chr9_+_123898083_{—} | C1orf52_chr1_−_85724617_{—} |
| 30156027_30149539_30156922 | 123898260_123886362_123900822 | 85724744_85724405_85725040 |
| TTC39C_chr18_+_21703797_{—} | NFE2L1_chr17_+_46134393_{—} | WNK2_chr9_+_96069058_{—} |
| 21703907_21698196_21708849 | 46134483_46133960_46134705 | 96069103_96060349_96070609 |
| ABCA7_chr19_+_1054568_{—} | C19orf60_chr19_+_18700222_{—} | DCAF8_chr1_−_160231074_{—} |
| 1054693_1054340_1054778 | 18700493_18699887_18701663 | 160231148_160213824_160232238 |
| SYNE2_chr14_+_64473760_{—} | SORBS2_chr4_−_186560030_{—} | BPTF_chr17_+_65925074_{—} |
| 64473940_64470048_64476683 | 186560189_186551752_186567821 | 65925236_65924717_65925451 |
| MFF_chr2_+_228217229_{—} | FANCL_chr2_−_58421363_{—} | HHAT_chr1_+_210522276_{—} |
| 228217289_228212100_228220392 | 58421408_58393009_58425728 | 210522410_210502448_210536195 |
| SH3BP2_chr4_+_2811505_{—} | SEC22C_chr3_−_42605256_{—} | RALY_chr20_+_32661624_{—} |
| 2811556_2794865_2822340 | 42605472_42605178_42610356 | 32661672_32661441_32663679 |
| NAXD_chr13_+_111277536_{—} | FER1L5_chr2_+_97313514_{—} | NDUFAF7_chr2_+_37469777_{—} |
| 111277625_111276626_111286891 | 97313623_97312190_97315359 | 37469836_37468780_37471005 |
| NSMF_chr9_−_140349690_{—} | ZNF211_chr19_+_58145390_{—} | ACBD4_chr17_+_43214385_{—} |
| 140349759_140348895_140350862 | 58145429_58144804_58145996 | 43214506_43214143_43214734 |
| CTD-2228K2.7_chr5_+_477358_{—} | LAS1L_chrX_−_64753489_{—} | SOX13_chr1_+_204082042_{—} |
| 477451_476678_477920 | 64753615_64752510_64754359 | 204082262_204042839_204083448 |
| KLHL8_chr4_−_88116475_{—} | MLLT10_chr10_+_21971153_{—} | INPP5K_chr17_−_1419181_{—} |
| 88116842_88104541_88141569 | 21971186_21970305_22002700 | 1419412_1418879_1419749 |
| NLGN2_chr17_+_7311330_{—} | HNRNPA2B1_chr7_−_26237450_{—} | NUMA1_chr11_−_71760464_{—} |
| 7312031_7308929_7315475 | 26237486_26237352_26240191 | 71760585_71747021_71780887 |
| HSD3B7_chr16_+_30996845_{—} | P4HA2_chr5_−_131562624_{—} | YTHDC2_chr5_+_112860677_{—} |
| 30997145_30996618_30997369 | 131562914_131554337_131563482 | 112860874_112851059_112868575 |
| C20orf96_chr20_−_270199_{—} | SNHG15_chr7_−_45025619_{—} | PAFAH2_chr1_−_26323938_{—} |
| 270317_264722_270899 | 45025696_45023645_45026123 | 26324112_26317354_26324515 |
| PPP1R35_chr7_−_100033253_{—} | RALGPS1_chr9_+_129975246_{—} | PIF1_chr15_−_65113565_{—} |
| 100033390_100033156_100033470 | 129975328_129974998_129977029 | 65113719_65113480_65114464 |
| FAXDC2_chr5_−_154218122_{—} | CLSPN_chr1_−_36212366_{—} | SLC43A1_chr11_−_57265225_{—} |
| 154218499_154217738_154230042 | 36212593_36211163_36213507 | 57265318_57263637_57268251 |
| AC159540.1_chr2_−_98089381_{—} | IL17RB_chr3_+_53889320_{—} | MTMR3_chr22_+_30366964_{—} |
| 98089511_98088865_98091456 | 53889368_53883822_53890870 | 30367051_30353077_30384468 |
| ZNF326_chr1_+_90472903_{—} | LDLRAD4_chr18_+_13484035_{—} | CPTP_chr1_+_1262215_{—} |
| 90473309_90470803_90475646 | 13484118_13465110_13621115 | 1262412_1260482_1262620 |
| GRIK2_chr6_+_102372475_{—} | RP11-252A24.2_chr16_−_74383639_{—} | EPSTI1_chr13_−_43493435_{—} |
| 102372594_102337738_102376289 | 74383757_74382913_74384514 | 43493468_43491760_43500471 |
| SMARCA4_chr19_+_11144442_{—} | ITM2C_chr2_+_231738131_{—} | LINC00174_chr7_−_65920645_{—} |
| 11144541_11144193_11144798 | 231738272_231729860_231740334 | 65920840_65915223_65943995 |
| MIB2_chr1_+_1560344_{—} | TXNRD3_chr3_−_126334197_{—} | CCDC88A_chr2_−_55530204_{—} |
| 1560565_1560281_1560665 | 126334305_126329975_126340584 | 55530288_55529208_55535944 |
| NACA_chr12_−_57113509_{—} | NAF1_chr4_−_164054305_{—} | STIM2_chr4_+_27023115_{—} |
| 57115243_57113380_57118235 | 164054408_164050500_164058350 | 27023234_27019606_27024140 |
| UGGT1_chr2_+_128913965_{—} | ZNF337-AS1_chr20_+_25612793_{—} | C1orf228_chr1_+_45154780_{—} |
| 128913989_128913161_128914825 | 25612924_25604771_25614684 | 45154896_45140545_45155347 |
| RP11-252A24.2_chr16_−_74384514_{—} | IL17RB_chr3_+_53886025_{—} | AC005154.6_chr7_−_30601081_{—} |
| 74384636_74382913_74385937 | 53886153_53882715_53889320 | 30601744_30591795_30608448 |
| SYN3_chr22_−_32934021_{—} | FAM234A_chr16_+_298242_{—} | RHOC_chr1_−_113247721_{—} |
| 32934096_32929881_32937556 | 298396_284856_299547 | 113248874_113246428_113249699 |
| TSPAN14_chr10_+_82228302_{—} | STAP2_chr19_−_4328671_{—} | SH2D6_chr2_+_85662535_{—} |
| 82228443_82214127_82248972 | 4328806_4327382_4329957 | 85662619_85662246_85663959 |
| EPB41L2_chr6_−_131199243_{—} | POLR2J3_chr7_−_102183971_{—} | DNAH10_chr12_+_124284762_{—} |
| 131199390_131191266_131206235 | 102184149_102182109_102185152 | 124284935_124283918_124288207 |
| ARHGAP18_chr6_−_129905132_{—} | SLCO4A1_chr20_+_61299509_{—} | HACE1_chr6_−_105291097_{—} |
| 129905257_129901276_129920360 | 61299536_61299262_61299828 | 105291173_105244900_105297016 |
| WASHC5_chr8_−_126095954_{—} | ERCC2_chr19_−_45872327_{—} | MDC1_chr6_−_30675342_{—} |
| 126096264_126095500_126103856 | 45872405_45872250_45873390 | 30676134_30675231_30679188 |
| ANKRD35_chr1_+_145560074_{—} | AAMDC_chr11_+_77552064_{—} | TSR1_chr17_−_2239330_{—} |
| 145560259_145558941_145560888 | 77552106_77532287_77553524 | 2239434_2239024_2239624 |
| SSPO_chr7_+_149519600_{—} | MFSD14C_chr9_−_99696076_{—} | AIM1L_chr1_−_26657979_{—} |
| 149519768_149519286_149520440 | 99696197_99681108_99711836 | 26658109_26655364_26662585 |
| TBL1XR1_chr3_−_176816253_{—} | MON2_chr12_+_62959793_{—} | SDR39U1_chr14_−_24911314_{—} |
| 176816329_176782810_176849150 | 62959811_62959160_62960101 | 24911466_24910132_24911551 |
| SLC2A4RG_chr20_+_62373221_{—} | YPEL5_chr2_+_30371110_{—} | PTBP1_chr19_+_805491_{—} |
| 62373409_62372832_62373482 | 30371407_30370957_30379493 | 805569_805187_806407 |
| CHD3_chr17_+_7813756_{—} | TMEM63B_chr6_+_44114583_{—} | RPL32P3_chr3_−_129115059_{—} |
| 7813909_7812656_7814164 | 44114664_44108835_44115113 | 129115263_129112638_129115882 |
| ESRRA_chr11_+_64082215_{—} | EEF1D_chr8_−_144672777_{—} | AKR1A1_chr1_+_46018107_{—} |
| 64082383_64081839_64083178 | 144672899_144672251_144679517 | 46018235_46016827_46027460 |
| SHARPIN_chr8_−_145153983_{—} | TCHP_chr12_+_110346390_{—} | PKP4_chr2_+_159535092_{—} |
| 145154108_145153897_145154179 | 110346503_110345504_110346675 | 159535166_159530512_159536940 |
| RP11-295P9.13_chr10_+_13655740_{—} | PPP6R3_chr11_+_68334466_{—} | CYLD_chr16_+_50778640_{—} |
| 13655881_13653683_13656014 | 68334634_68331900_68337215 | 50778777_50776752_50783486 |
| HTR3E_chr3_+_183819272_{—} | MINDY3_chr10_−_15863654_{—} | FAT3_chr11_+_92623021_{—} |
| 183819317_183818439_183823552 | 15863725_15838171_15902204 | 92623057_92620279_92623656 |
| FRMD8_chr11_+_65161043_{—} | PTP4A2_chr1_−_32381495_{—} | WAPL_chr10_−_88232368_{—} |
| 65161145_65154592_65161511 | 32381588_32377427_32384570 | 88232521_88232101_88233634 |
| ADAMTS6_chr5_−_64592934_{—} | TTLL3_chr3_+_9857757_{—} | RP11-235E17.2_chr17_−_3491481_{—} |
| 64593071_64587297_64595811 | 9857886_9855029_9867483 | 3491661_3489220_3493100 |
| DIAPH1_chr5_−_140967790_{—} | SPATA4_chr4_−_177114108_{—} | NSUN5P1_chr7_+_75042066_{—} |
| 140967817_140966764_140998364 | 177114227_177113998_177114603 | 75042210_75039743_75044162 |
| FGGY_chr1_+_59811918_{—} | SLAMF7_chr1_+_160720093_{—} | FUS_chr16_+_31200440_{—} |
| 59812070_59762822_59844420 | 160720213_160719883_160721134 | 31200547_31199678_31201360 |
| C1orf168_chr1_−_57233501_{—} | EBPL_chr13_−_50243912_{—} | DYRK2_chr12_+_68043576_{—} |
| 57233611_57221600_57252847 | 50243982_50237331_50265389 | 68043725_68042962_68050885 |
| C3orf67_chr3_−_59025282_{—} | NEURL1B_chr5_+_172110421_{—} | PQLC1_chr18_−_77679183_{—} |
| 59025355_58923488_59031954 | 172111141_172068440_172113173 | 77679400_77664183_77703328 |
| MGRN1_chr16_+_4730032_{—} | MTO1_chr6_+_74190015_{—} | C16orf87_chr16_−_46843514_{—} |
| 4730098_4727573_4731550 | 74190090_74189849_74190397 | 46843697_46836982_46858297 |
| MRPS28_chr8_−_80940929_{—} | IMPA1_chr8_−_82597997_{—} | MALT1_chr18_+_56378152_{—} |
| 80941031_80915415_80942288 | 82598198_82593819_82598486 | 56378185_56377304_56381314 |
| CCDC88B_chr11_+_64118631_{—} | PPP4R1L_chr20_−_56813266_{—} | NBPF10_chr1_+_145295422_{—} |
| 64118727_64117140_64118947 | 56813367_56811330_56814296 | 145295471_145293580_145296356 |
| DPP8_chr15_−_65746600_{—} | SPATS2_chr12_+_49780836_{—} | MTFMT_chr15_−_65316009_{—} |
| 65746753_65744440_65748562 | 49780920_49761370_49854552 | 65316132_65312610_65319168 |
| SPTBN5_chr15_−_42171931_{—} | WDR45_chrX_−_48935301_{—} | JPX_chrX_+_73219189_{—} |
| 42172062_42171697_42173231 | 48935406_48934409_48935495 | 73220331_73218679_73224104 |
| FER1L5_chr2_+_97312800_{—} | SH2D6_chr2_+_85661595_{—} | MRPL33_chr2_+_27997290_{—} |
| 97312892_97312190_97313514 | 85661620_85661493_85662187 | 27997397_27995559_28002299 |
| ZNF783_chr7_+_148985711_{—} | CNTNAP1_chr17_+_40840893_{—} | PFDN1_chr5_−_139680000_{—} |
| 148985782_148984867_148987028 | 40841065_40840629_40841538 | 139680167_139661118_139682625 |
| ACSS1_chr20_−_25002024_{—} | FAM208B_chr10_+_5777266_{—} | AARSD1_chr17_−_41105740_{—} |
| 25002172_24995866_25011394 | 5777509_5773166_5781580 | 41105795_41103911_41106892 |
| GRAMD1C_chr3_+_113594330_{—} | KCNQ1_chr11_+_2606441_{—} | PISD_chr22_−_32028168_{—} |
| 113594434_113588438_113601598 | 2606537_2604775_2608799 | 32028277_32022002_32034351 |
| EIF2D_chr1_−_206772816_{—} | BID_chr22_−_18226568_{—} | NGLY1_chr3_−_25773809_{—} |
| 206772966_206772446_206773086 | 18226779_18222254_18257146 | 25773974_25770809_25775362 |
| SSPO_chr7_+_149484949_{—} | SLC50A1_chr1_+_155109303_{—} | MEF2A_chr15_+_100138634_{—} |
| 149485059_149484882_149485408 | 155109427_155108852_155110036 | 100138716_100105874_100173182 |
| PSMC5_chr17_+_61905033_{—} | ZNF592_chr15_+_85321413_{—} | SERPINB8_chr18_+_61645532_{—} |
| 61905283_61904874_61905497 | 85321543_85308046_85325887 | 61645710_61637421_61647034 |
| LYRM1_chr16_+_20913807_{—} | VIPAS39_chr14_−_77923437_{—} | SBF2_chr11_−_9849672_{—} |
| 20914039_20912211_20926877 | 77923708_77920445_77923837 | 9849768_9838571_9850902 |
| ALGIL_chr3_−_125651492_{—} | AADACL2_chr3_+_151458433_{—} | MKS1_chr17_−_56283825_{—} |
| 125651552_125650595_125652417 | 151458656_151451961_151461880 | 56283908_56283741_56284445 |
| LSM14B_chr20_+_60705274_{—} | HCG18_chr6_−_30262629_{—} | CCDC15_chr11_+_124861356_{—} |
| 60705352_60705008_60705585 | 30262741_30260376_30263908 | 124861479_124858030_124862475 |
| ISOC2_chr19_−_55967002_{—} | EEF1D_chr8_−_144674816_{—} | GPT2_chr16_+_46919378_{—} |
| 55967212_55966697_55967715 | 144675063_144672251_144679517 | 46919489_46919243_46931559 |
| NEURL1B_chr5_+_172096787_{—} | GOLGA2P5_chr12_−_100552575_{—} | DRC3_chr17_+_17877165_{—} |
| 172097333_172068440_172113173 | 100552651_100551855_100552735 | 17877276_17876288_17880895 |
| ARHGAP22_chr10_−_49687630_{—} | KIAA1191_chr5_−_175786464_{—} | DRC3_chr17_+_17876978_{—} |
| 49687807_49667934_49763507 | 175786570_175782752_175786813 | 17877276_17876288_17880895 |
| SAR1B_chr5_−_133967766_{—} | CLMN_chr14_−_95659655_{—} | GSTT1_chr22_−_24381699_{—} |
| 133967885_133959727_133968417 | 95659752_95658065_95660185 | 24381787_24379511_24384119 |
| SNHG15_chr7_−_45023955_{—} | GUCY1B2_chr13_−_51622355_{—} | PICALM_chr11_−_85689112_{—} |
| 45024025_45023645_45026123 | 51622450_51608295_51636956 | 85689136_85685855_85692171 |
| RAP2C_chrX_−_131352259_{—} | CCNDBP1_chr15_+_43482252_{—} | RP11-252A24.2_chr16_−_74378676_{—} |
| 131352369_131351834_131353397 | 43482349_43481478_43482522 | 74378869_74376152_74382764 |
| USP37_chr2_−_219344372_{—} | MTMR2_chr11_−_95620775_{—} | PER2_chr2_−_239169468_{—} |
| 219344438_219341704_219346792 | 95620848_95598840_95621319 | 239169594_239168694_239170369 |
| TMEM131_chr2_−_98460607_{—} | PLOD2_chr3_−_145795648_{—} | PDE4DIP_chr1_−_144955215_{—} |
| 98460724_98458400_98475766 | 145795711_145791139_145796902 | 144955292_144952689_144994590 |
| POLL_chr10_−_103345618_{—} | PUS1_chr12_+_132425836_{—} | ING4_chr12_−_6762395_{—} |
| 103345913_103344676_103347002 | 132426528_132416857_132428083 | 6762562_6762216_6765892 |
| STAU2_chr8_−_74439925_{—} | STRBP_chr9_−_125901807_{—} | GSN_chr9_+_124062333_{—} |
| 74440039_74334937_74464246 | 125901966_125898794_125909133 | 124062404_124048463_124064240 |
| MICAL3_chr22_−_18310409_{—} | MYO5A_chr15_−_52635313_{—} | MOCS1_chr6_−_39876830_{—} |
| 18310547_18305826_18314619 | 52635394_52632591_52638557 | 39876878_39874893_39877578 |
| TMTC4_chr13_−_101315217_{—} | ARIH2_chr3_+_48982568_{—} | LINC00571_chr13_−_38636285_{—} |
| 101315434_101294565_101316474 | 48982614_48965246_48999044 | 38636371_38635590_38663709 |
| SEC22C_chr3_−_42605256_{—} | SH2D6_chr2_+_85657486_{—} | PLA2G4C_chr19_−_48581027_{—} |
| 42605372_42605178_42610356 | 85657557_85656735_85660693 | 48581081_48578131_48588315 |
| RHPN1_chr8_+_144461998_{—} | DCTD_chr4_−_183837571_{—} | PEX26_chr22_+_18561061_{—} |
| 144462155_144461678_144462758 | 183837692_183836728_183838463 | 18561372_18560813_18562639 |
| RBFOX2_chr22_−_36232366_{—} | PROX1-AS1_chr1_−_214049980_{—} | NRG2_chr5_−_139244699_{—} |
| 36232486_36206051_36236238 | 214050149_214005965_214068005 | 139244758_139239497_139245133 |
| UBE3A_chr15_−_25653718_{—} | WIPI2_chr7_+_5232748_{—} | PAM_chr5_+_102360834_{—} |
| 25653831_25652409_25654234 | 5232802_5230124_5239206 | 102361038_102355547_102363888 |
| GALNT14_chr2_−_31337608_{—} | XIAP_chrX_+_122994016_{—} | PAM_chr5_+_102360834_{—} |
| 31337693_31215873_31348019 | 122994143_122993755_123019480 | 102361038_102355547_102363885 |
| MTHFSD_chr16_−_86585784_{—} | SLTM_chr15_−_59191667_{—} | OFD1_chrX_+_13781863_{—} |
| 86585898_86582183_86588250 | 59192082_59191051_59193458 | 13781974_13780563_13785245 |
| CD44_chr11_+_35232792_{—} | CAMTA2_chr17_−_4885383_{—} | SLC25A3_chr12_+_98989210_{—} |
| 35232996_35211612_35236398 | 4885455_4885126_4886051 | 98989335_98987913_98991633 |
| DAG1_chr3_+_49514281_{—} | FAM208B_chr10_+_5754785_{—} | RP11-321G12.1_chr15_+_63723255_{—} |
| 49514333_49507866_49524660 | 5754881_5727138_5759621 | 63723541_63690666_63724350 |
| SYTL1_chr1_+_27677284_{—} | HNRNPC_chr14_−_21731469_{—} | CCDC57_chr17_−_80091948_{—} |
| 27677443_27676976_27677748 | 21731741_21702388_21737456 | 80092070_80086473_80115623 |
| CTBP1_chr4_−_1235112_{—} | WDPCP_chr2_−_63713675_{—} | GSTZ1_chr14_+_77793814_{—} |
| 1235307_1232125_1242703 | 63713720_63712121_63714580 | 77793895_77787526_77795465 |
| KB-1572G7.2_chr22_−_24032418_{—} | SH2D6_chr2_+_85662535_{—} | RP11-284E5.1_chr2_−_191505676_{—} |
| 24032543_24026054_24036305 | 85662619_85662246_85663588 | 191506188_191493441_191512945 |
| ABI1_chr10_−_27044583_{—} | HSD3B7_chr16_+_30996973_{—} | WIPI2_chr7_+_5232748_{—} |
| 27044670_27040712_27054146 | 30997145_30996618_30997369 | 5232976_5230124_5239206 |
| XXbac-BPG32J3.22_chr6_+_31637095_{—} | NME9_chr3_−_138023714_{—} | POLB_chr8_+_42196529_{—} |
| 31637285_31636949_31638932 | 138023869_138022485_138024845 | 42196587_42196203_42202470 |
| HTR3E_chr3_+_183819272_{—} | ZNF639_chr3_+_179042829_{—} | MITD1_chr2_−_99787805_{—} |
| 183819317_183818439_183821969 | 179043110_179041079_179045348 | 99787892_99787115_99787972 |
| AC004076.9_chr19_−_57987027_{—} | COL4A3BP_chr5_−_74695134_{—} | HDAC11_chr3_+_13542212_{—} |
| 57987154_57949460_57988001 | 74695212_74685512_74706835 | 13542289_13525064_13543370 |
| PEX5_chr12_+_7342617_{—} | MRPL47_chr3_−_179320439_{—} | CRCP_chr7_+_65592690_{—} |
| 7342812_7342127_7342957 | 179320525_179319578_179322314 | 65592727_65579957_65617194 |
| PTPN18_chr2_+_131116969_{—} | RPRDIA_chr18_−_33620769_{—} | ALOX12B_chr17_−_7979492_{—} |
| 131117065_131116548_131117180 | 33620828_33611060_33647216 | 7979662_7979034_7979974 |
| TTC13_chr1_−_231079551_{—} | GEMIN7_chr19_+_45583164_{—} | FAM221B_chr9_−_35819193_{—} |
| 231079644_231076302_231081134 | 45583287_45582635_45593364 | 35819391_35819006_35819886 |
| MAP4_chr3_−_47960208_{—} | FRMD8_chr11_+_65156831_{—} | FAM47E-STBD1_chr4_+_77199221_{—} |
| 47960331_47958664_47963254 | 65156999_65154592_65161043 | 77199377_77192921_77230296 |
| UROS_chr10_−_127477851_{—} | SEMA6C_chr1_−_151114979_{—} | ITPA_chr20_+_3193903_{—} |
| 127477932_127477574_127483448 | 151115151_151112566_151116860 | 3194029_3190263_3194630 |
| EFCAB14_chr1_−_47154024_{—} | PABPC4_chr1_−_40029507_{—} | DST_chr6_−_56394245_{—} |
| 47154216_47152542_47155258 | 40029594_40029413_40030142 | 56394572_56393723_56394771 |
| KAT2B_chr3_+_20189894_{—} | DICER1_chr14_−_95600697_{—} | NUTM2B-AS1_chr10_−_81568518_{—} |
| 20189979_20189798_20193823 | 95600840_95599840_95606995 | 81568719_81565878_81574368 |
| PCMTD1_chr8_−_52752006_{—} | NADSYN1_chr11_+_71191264_{—} | DGKH_chr13_+_42795399_{—} |
| 52752198_52746249_52758220 | 71191320_71189515_71191800 | 42795530_42793930_42803234 |
| SHMT1_chr17_−_18238872_{—} | RAD51C_chr17_+_56783238_{—} | PARPBP_chr12_+_102569260_{—} |
| 18238989_18233985_18243356 | 56783383_56780690_56787219 | 102569444_102559661_102589728 |
| MECP2_chrX_−_153357641_{—} | PLEKHJ1_chr19_−_2234148_{—} | SNRPA1_chr15_−_101826418_{—} |
| 153357765_153298008_153363060 | 2234239_2234060_2235760 | 101826498_101826006_101827112 |
| DNAH14_chr1_+_225394669_{—} | MAN2B1_chr19_−_12759949_{—} | HNRNPDL_chr4_−_83346715_{—} |
| 225394922_225393881_225418774 | 12760030_12759216_12760726 | 83346820_83346036_83347189 |
| FBXW11_chr5_−_171384600_{—} | VPS37A_chr8_+_17123415_{—} | POGZ_chr1_−_151384100_{—} |
| 171384702_171341409_171433461 | 17123490_17104878_17126364 | 151384274_151381304_151384771 |
| AC009120.6_chr16_−_74340152_{—} | ZMYND8_chr20_−_45984193_{—} | RIBC1_chrX_+_53455230_{—} |
| 74340319_74339478_74366464 | 45984264_45976670_45985400 | 53455575_53455032_53456801 |
| FBLN2_chr3_+_13663274_{—} | SEC31A_chr4_−_83782783_{—} | ELMO2_chr20_−_45015976_{—} |
| 13663415_13661331_13667944 | 83782861_83778917_83784470 | 45016070_45014914_45017677 |
| PKIB_chr6_+_122803043_{—} | PVT1_chr8_+_128808139_{—} | DDR1_chr6_+_30853401_{—} |
| 122803101_122799084_122907065 | 128808254_128806980_128867400 | 30853457_30852487_30856464 |
| STRADA_chr17_−_61784606_{—} | CAMTA2_chr17_−_4885383_{—} | CFAP70_chr10_−_75095187_{—} |
| 61784778_61784099_61787850 | 4885470_4885126_4886051 | 75095282_75091034_75101156 |
| SNAP47_chr1_+_227919285_{—} | NAXD_chr13_+_111289475_{—} | TMTC4_chr13_−_101308634_{—} |
| 227919413_227916487_227935392 | 111289596_111287935_111290724 | 101308722_101294565_101316474 |
| PARL_chr3_−_183551511_{—} | CEACAM19_chr19_+_45175161_{—} | RP11-216L13.19_chr9_+_139703706_{—} |
| 183551613_183551377_183558357 | 45175258_45165665_45175867 | 139703881_139702778_139704139 |
| TBL1XR1_chr3_−_176849150_{—} | GSN_chr9_+_124044716_{—} | COL6A6_chr3_+_130360495_{—} |
| 176849237_176816329_176914908 | 124044828_124043840_124060043 | 130360558_130354605_130361675 |
| HP1BP3_chr1_−_21106837_{—} | SERPINB8_chr18_+_61645532_{—} | TMPRSS13_chr11_−_117782482_{—} |
| 21107033_21106404_21113687 | 61645710_61637404_61647034 | 117782575_117781465_117784491 |
| DDR1_chr6_+_30852314_{—} | PYCR1_chr17_−_79892528_{—} | EPN3_chr17_+_48616244_{—} |
| 30852487_30851922_30856464 | 79892621_79892365_79892801 | 48616325_48615558_48616547 |
| SLTM_chr15_−_59205697_{—} | MACC1_chr7_−_20203878_{—} | PCGF5_chr10_+_93000240_{—} |
| 59205895_59204809_59209133 | 20204022_20201493_20256921 | 93000337_92982740_93008261 |
| ZNF852_chr3_−_44544456_{—} | CBR3-AS1_chr21_−_37509391_{—} | FAM228B_chr2_+_24384375_{—} |
| 44544548_44544160_44552044 | 37509606_37506059_37518553 | 24384483_24369956_24387067 |
| DFFB_chr1_+_3782847_{—} | MTFMT_chr15_−_65313851_{—} | SPTAN1_chr9_+_131371929_{—} |
| 3782962_3782564_3784537 | 65313954_65312610_65319168 | 131371944_131371563_131373992 |
| CEP164_chr11_+_117253511_{—} | LRP8_chr1_−_53715051_{—} | PRR5_chr22_+_45130906_{—} |
| 117253658_117252584_117261492 | 53715228_53712728_53716361 | 45131042_45128271_45132651 |
| METTL6_chr3_−_15457004_{—} | SORBS1_chr10_−_97082502_{—} | MYCBP2_chr13_−_77807290_{—} |
| 15457090_15456421_15457278 | 97082562_97081778_97098889 | 77807398_77799689_77817193 |
| TAMM41_chr3_−_11868191_{—} | ELFN2_chr22_−_37813807_{—} | RPL17_chr18_−_47018105_{—} |
| 11868279_11858811_11871187 | 37813958_37772036_37822910 | 47018203_47017954_47018627 |
| PRKAR2A_chr3_−_48789608_{—} | TRIM2_chr4_+_154215456_{—} | CORO6_chr17_−_27948241_{—} |
| 48789750_48789151_48793811 | 154215637_154197282_154216464 | 27948441_27943865_27949705 |
| APH1B_chr15_+_63579622_{—} | CSGALNACT2_chr10_+_43659313_{—} | POMGNT1_chr1_−_46662450_{—} |
| 63579745_63578827_63594543 | 43659492_4365604343671398 | 46662521_46661749_46662641 |
| YY1AP1_chr1_−_155657861_{—} | DENND3_chr8_+_142170730_{—} | CARF_chr2_+_203789019_{—} |
| 155657992_155650247_155658720 | 142170886_142166069_142173431 | 203789138_203782766_203799224 |
| HPS5_chr11_−_18339297_{—} | PROM2_chr2_+_95942341_{—} | CRYZ_chr1_−_75196065_{—} |
| 18339454_18333571_18343492 | 95942405_95942095_95942719 | 75196104_75190518_75198639 |
| RNF185_chr22_+_31591454_{—} | HNRNPC_chr14_−_21731469_{—} | GZF1_chr20_+_23350220_{—} |
| 31591567_31588688_31597483 | 21731495_21702388_21737456 | 23350378_23349566_23350727 |
| PI4KAP2_chr22_−_21842512_{—} | TNS1_chr2_−_218677937_{—} | IFT46_chr11_−_118428452_{—} |
| 21842670_21841961_21846282 | 218678030_218677168_218678407 | 118428605_118427760_118430499 |
| ZNF394_chr7_−_99096338_{—} | SS18L1_chr20_+_60729336_{—} | LRP8_chr1_−_53746258_{—} |
| 99096465_99092254_99097260 | 60729521_60718945_60733727 | 53746387_53737018_53755238 |
| KDM4C_chr9_+_7105401_{—} | MED23_chr6_−_131936463_{—} | FOXRED1_chr11_+_126144821_{—} |
| 7105500_7103870_7128065 | 131936481_131931386_131937046 | 126144916_126143349_126145221 |
| FLCN_chr17_−_17127235_{—} | TMPO_chr12_+_98938219_{—} | FGGY_chr1_+_59787207_{—} |
| 17127457_17122523_17129489 | 98938315_98931350_98938728 | 59787422_59762822_59844420 |
| ANKRD6_chr6_+_90321990_{—} | MFF_chr2_+_228211941_{—} | LINC00511_chr17_−_70416840_{—} |
| 90322089_90315824_90333128 | 228212100_228205096_228220392 | 70417105_70400957_70424376 |
| CEACAM1_chr19_−_43015727_{—} | RP13-507P19.2_chr12_+_131833872_{—} | KIAA1468_chr18_+_59947006_{—} |
| 43015780_43015072_43016501 | 131834089_131832206_131850500 | 59947089_59942706_59947592 |
| GMIP_chr19_−_19746452_{—} | SLC35B1_chr17_−_47784704_{—} | TSC2_chr16_+_2127598_{—} |
| 19746530_19746378_19747515 | 47784806_47783671_47785091 | 2127727_2126586_2129032 |
| SP100_chr2_+_231314886_{—} | PDXDC1_chr16_+_15095632_{—} | INTS11_chr1_−_1258526_{—} |
| 231314970_231314425_231325976 | 15095713_15092262_15098043 | 1258667_1250998_1259960 |
| RP11-696N14.1_chr4_+_100207951_{—} | UBTF_chr17_−_42289711_{—} | PATZ1_chr22_−_31724772_{—} |
| 100208190_100125588_100209822 | 42289822_42289374_42290186 | 31724845_31723295_31731677 |
| CRTC1_chr19_+_18882251_{—} | CNTRL_chr9_+_123887993_{—} | MTMR2_chr11_−_95615537_{—} |
| 18882356_18879603_18886450 | 123888214_123886362_123900822 | 95615660_95599650_95621319 |
| MARK2_chr11_+_63671457_{—} | AC005154.6_chr7_−_30591715_{—} | ELOC_chr8_−_74882813_{—} |
| 63671619_63670630_63672257 | 30591795_30590397_30601081 | 74882869_74872053_74884104 |
| PVT1_chr8_+_128867400_{—} | KMT2E_chr7_+_104678572_{—} | HAUS8_chr19_−_17173507_{—} |
| 128867565_128808254_128902834 | 104678646_104654982_104681285 | 17173589_17170902_17179840 |
| PPP3CB_chr10_−_75199629_{—} | HUWE1_chrX_−_53589791_{—} | LTB4R2_chr14_+_24779376_{—} |
| 75199659_75198178_75206249 | 53589890_53589205_53590706 | 24779461_24775073_24779860 |
| DAXX_chr6_−_33288512_{—} | JOSD2_chr19_−_51010830_{—} | RP11-110G21.1_chr8_+_52825616_{—} |
| 33289344_33288368_33290638 | 51010956_51009829_51013542 | 52825740_52812406_52858995 |
| LINC00511_chr17_−_70427268_{—} | SMCHD1_chr18_+_2722516_{—} | PCK2_chr14_+_24572368_{—} |
| 70427365_70424439_70588341 | 2722661_2718432_2724896 | 24572464_24572099_24572718 |
| CCDC159_chr19_+_11462732_{—} | TMC6_chr17_−_76117636_{—} | OASL_chr12_−_121465378_{—} |
| 11462809_11460875_11464126 | 76117792_76117245_76118685 | 121465620_121461940_121469244 |
| KIAA1191_chr5_−_175786483_{—} | CAMK2G_chr10_−_75579289_{—} | MFF_chr2_+_228217229_{—} |
| 175786570_175782752_175786813 | 75579403_75577312_75581439 | 228217289_228205096_228220392 |
| ABLIM1_chr10_−_116233637_{—} | PPIP5K2_chr5_+_102523014_{—} | MRI1_chr19_+_13876767_{—} |
| 116233715_116232891_116247716 | 102523077_102522140_102526542 | 13876879_13875923_13879172 |
| MB_chr22_−_36013209_{—} | PBRM1_chr3_−_52592264_{—} | CANT1_chr17_−_76996181_{—} |
| 36013312_36007153_36018524 | 52592429_52584833_52595782 | 76996352_76994045_77001453 |
| ZNF326_chr1_+_90473170_{—} | PHPT1_chr9_+_139744957_{—} | NRCAM_chr7_−_107866088_{—} |
| 90473309_90470803_90475646 | 139745012_139744589_139745206 | 107866145_107864280_107866651 |
| TCOF1_chr5_+_149771106_{—} | KIAA1468_chr18_+_59947592_{—} | ZMIZ2_chr7_+_44790570_{—} |
| 149771220_149769586_149771519 | 59947675_59942706_59947878 | 44790690_44789579_44795786 |
| NUCB2_chr11_+_17336932_{—} | ARIH2_chr3_+_48962150_{—} | SLC9B2_chr4_−_103978957_{—} |
| 17337022_17333667_17351673 | 48962404_48960244_48964894 | 103979128_103971539_103987483 |
| EML3_chr11_−_62373326_{—} | ZNF195_chr11_−_3394806_{—} | RTN3_chr11_+_63486173_{—} |
| 62373452_62373245_62373534 | 3394886_3392933_3400267 | 63488504_63472379_63517462 |
| GPER1_chr7_+_1127833_{—} | SFSWAP_chr12_+_132249000_{—} | CSNK1E_chr22_−_38691392_{—} |
| 1127957_1126880_1131042 | 132249231_132241189_132250662 | 38691453_38690540_38694790 |
| PKIB_chr6_+_122803043_{—} | CNOT10_chr3_+_32778901_{—} | NEK3_chr13_−_52715155_{—} |
| 122803101_122799084_122803291 | 32778982_32776468_32800949 | 52715205_52711038_52718050 |
| ATG4D_chr19_+_10657514_{—} | NEO1_chr15_+_73567032_{—} | ACSF3_chr16_+_89164998_{—} |
| 10657791_10655806_10657864 | 73567065_73566346_73570471 | 89165171_89160404_89169011 |
| C14orf80_chr14_+_105960171_{—} | ADGRL1_chr19_−_14275432_{—} | HDAC2_chr6_−_114283530_{—} |
| 105960270_105959071_105963693 | 14275517_14274218_14281493 | 114283558_114281182_114291643 |
| KIAA1958_chr9_+_115380150_{—} | CS_chr12_−_56692392_{—} | NAB1_chr2_+_191520702_{—} |
| 115380234_115337531_115407929 | 56692475_56680429_56693611 | 191520879_191514086_191548463 |
| PDPK1_chr16_+_2616356_{—} | NEURL1B_chr5_+_172110421_{—} | MORC4_chrX_−_106242906_{—} |
| 2616454_2588137_2627425 | 172111141_172097333_172113173 | 106242979_106236592_106243097 |
| ABL2_chr1_−_179102446_{—} | EPB41L1_chr20_+_34761685_{—} | ARHGAP22_chr10_−_49687678_{—} |
| 179102509_179100616_179112067 | 34761876_34742818_34763472 | 49687807_49667934_49732097 |
| DMTF1_chr7_+_86823999_{—} | ERBB4_chr2_−_212522674_{—} | CAMK2G_chr10_−_75579289_{—} |
| 86824144_86823418_86824346 | 212522719_212522553_212530047 | 75579375_75577312_75581439 |
| PAM16_chr16_−_4393207_{—} | RP11-23J9.4_chr9_+_100053652_{—} | EDC3_chr15_−_74969281_{—} |
| 4393292_4391673_4401232 | 100053769_100053184_100070017 | 74969363_74967483_74979431 |
| BECN1_chr17_−_40965965_{—} | EHMT2_chr6_−_31856745_{—} | KIF13A_chr6_−_17790102_{—} |
| 40966026_40962946_40966541 | 31856847_31856524_31857004 | 17790141_17788106_17794802 |
| TNS2_chr12_+_53448053_{—} | FAM13B_chr5_−_137354643_{—} | FLOT1_chr6_−_30709568_{—} |
| 53448225_53447798_53448969 | 137354835_137354203_137356719 | 30709644_30709110_30709923 |
| ASCC1_chr10_−_73975539_{—} | P3H4_chr17_−_39959538_{—} | TFDP1_chr13_+_114292132_{—} |
| 73975612_73973089_73975964 | 39959683_39959231_39963047 | 114292211_114291015_114294434 |
| KYAT1_chr9_−_131608958_{—} | CFAP70_chr10_−_75036997_{—} | LINC00893_chrX_−_148619164_{—} |
| 131609138_131607690_131644175 | 75037119_75035356_75037936 | 148619274_148617745_148619646 |
| WNK2_chr9_+_96018580_{—} | POSTN_chr13_−_38148708_{—} | DIS3_chr13_−_73355426_{—} |
| 96018736_96010116_96019229 | 38148789_38145595_38151889 | 73355494_73355141_73355742 |
| OGT_chrX_+_70775039_{—} | ATRX_chrX_−_76949312_{—} | ZCCHC7_chr9_+_37121596_{—} |
| 70775235_70774442_70775803 | 76949426_76944417_76952064 | 37121669_37120773_37126308 |
| SLC44A3_chr1_+_95286504_{—} | ARHGEF10_chr8_+_1828213_{—} | RPL5_chr1_+_93298945_{—} |
| 95286612_95286034_95290048 | 1828330_1824900_1830800 | 93299015_93297674_93301746 |
| AC005154.6_chr7_−_30601081_{—} | UBR3_chr2_+_170734003_{—} | STAG3L5P-PVRIG2P-PILRB_chr7_+_{—} |
| 30601744_30590397_30603346 | 170734147_170732459_170735034 | 99950995_99951635_99950893_99952765 |
| EPS15L1_chr19_−_16487932_{—} | MBD1_chr18_−_47800555_{—} | NUP62_chr19_−_50430950_{—} |
| 16488065_16466662_16495939 | 47800723_47800233_47801346 | 50431072_50413141_50432582 |
| ACYP1_chr14_−_75530172_{—} | BRD8_chr5_−_137495243_{—} | TNRC6C_chr17_+_76093825_{—} |
| 75530264_75520362_75535912 | 137495288_137492956_137495757 | 76093942_76089844_76094423 |
| PLSCR3_chr17_−_7296919_{—} | ZNF195_chr11_−_3394806_{—} | CCDC18-AS1_chr1_−_93791325_{—} |
| 7297155_7296828_7297422 | 3394886_3392933_3398846 | 93791452_93771027_93802933 |
| RGS14_chr5_+_176798475_{—} | RP11-242J7.1_chr4_−_185544575_{—} | PLSCR1_chr3_−_146254326_{—} |
| 176798590_176798397_176798873 | 185544612_185540471_185545892 | 146254352_146251337_146262253 |
| ARHGAP44_chr17_+_12877405_{—} | SH2D6_chr2_+_85662187_{—} | AGA_chr4_−_178357451_{—} |
| 12877627_12862214_12883374 | 85662246_85660754_85663959 | 178357505_178355635_178358558 |
| RP11-958N24.1_chr16_+_15013101_{—} | RGS12_chr4_+_3430284_{—} | TERF1_chr8_+_73942570_{—} |
| 15013285_15012983_15013719 | 3430438_3429896_3432133 | 73942630_73939287_73944276 |
| ADNP_chr20_−_49545187_{—} | UAP1L1_chr9_+_139972501_{—} | GEMIN7_chr19_+_45583164_{—} |
| 49545362_49520538_49547362 | 139972706_139972273_139972953 | 45583287_45582537_45593364 |
| FBXL19_chr16_+_30939787_{—} | TTC31_chr2_+_74717151_{—} | SDR39U1_chr14_−_24911303_{—} |
| 30939949_30939284_30941393 | 74717254_74710537_74717370 | 24911466_24910132_24911551 |
| SLC38A5_chrX_−_48324401_{—} | PRDX5_chr11_+_64087205_{—} | DYSF_chr2_+_71895883_{—} |
| 48324480_48321365_48325185 | 64087340_64085858_64088336 | 71895972_71894645_71896241 |
| SNHG11_chr20_+_37076572_{—} | PPM1N_chr19_+_46003907_{—} | IRF1_chr5_−_131823617_{—} |
| 37076736_37076266_37077304 | 46003968_46003819_46005274 | 131823717_131822822_131826236 |
| SETD4_chr21_−_37429681_{—} | TMEM181_chr6_+_159028275_{—} | PPM1K_chr4_−_89199687_{—} |
| 37429775_37429502_37431113 | 159028375_159026379_159029364 | 89199794_89198395_89205557 |
| SLCO4A1_chr20_+_61299506_{—} | PRKDC_chr8_−_48694722_{—} | SEPT3_chr22_+_42377648_{—} |
| 61299536_61299262_61299828 | 48694815_48691654_48694938 | 42377840_42373078_42381987 |
| ZC3H18_chr16_+_88694029_{—} | MTMR2_chr11_−_95615537_{—} | PROM2_chr2_+_95950716_{—} |
| 88694190_88691675_88695165 | 95615660_95598840_95621319 | 95950863_95947974_95951386 |
| ANKMY1_chr2_−_241496606_{—} | EIF2AK4_chr15_+_40308702_{—} | DIS3_chr13_−_73354983_{—} |
| 241496769_241494472_241500147 | 40308873_40303547_40309308 | 73355141_73352518_73355426 |
| PATL2_chr15_−_44967683_{—} | DCAF8_chr1_−_160190248_{—} | ALOX12B_chr17_−_7976974_{—} |
| 44967889_44966428_44968672 | 160190481_160188758_160192440 | 7977075_7976636_7979974 |
| NCAPG2_chr7_−_158494938_{—} | RNF38_chr9_−_36390463_{—} | SCNN1D_chr1_+_1220950_{—} |
| 158495039_158494629_158496340 | 36390613_36376124_36400093 | 1221044_1219470_1221305 |
| ATG13_chr11_+_46639874_{—} | SPTBN5_chr15_−_42172357_{—} | RICTOR_chr5_−_39074212_{—} |
| 46639925_46639440_46642609 | 42172510_42171697_42173231 | 39074260_39021238_39074430 |
| PTP4A2_chr1_−_32396533_{—} | ELP2_chr18_+_33719381_{—} | CCDC162P_chr6_+_109674041_{—} |
| 32396743_32385259_32403355 | 33719576_33718835_33721099 | 109674117_109669750_109676050 |
| HTR3C_chr3_+_183772508_{—} | C11orf1_chr11_+_111753859_{—} | USO1_chr4_+_76716488_{—} |
| 183772675_183770935_183773099 | 111753926_111753322_111754494 | 76716509_76715054_76720774 |
| DDO_chr6_−_110734493_{—} | PTPN6_chr12_+_7060667_{—} | CEACAM19_chr19_+_45182124_{—} |
| 110734669_110729645_110736669 | 7060894_7055902_7061145 | 45182208_45179693_45183559 |
| FAM208B_chr10_+_5751492_{—} | ATP6V0E2-AS1_chr7_−_149567330_{—} | MICAL2_chr11_+_12277189_{—} |
| 5751626_5727138_5754785 | 149567440_149566947_149568730 | 12277297_12265659_12278331 |
| CLTCL1_chr22_−_19175069_{—} | DPH7_chr9_−_140459344_{—} | SNRNP48_chr6_+_7599905_{—} |
| 19175240_19171124_19175492 | 140459410_140459058_140459536 | 7599913_7595334_7601568 |
| TSPAN14_chr10_+_82222843_{—} | CARD8_chr19_−_48753007_{—} | ZNF83_chr19_−_53119970_{—} |
| 82222883_82214127_82228302 | 48753104_48752890_48759048 | 53120094_53118050_53122188 |
| XPO1_chr2_−_61752404_{—} | SH2D6_chr2_+_85662788_{—} | RABGEF1_chr7_+_66240213_{—} |
| 61752468_61749818_61752626 | 85662947_85662246_85663959 | 66240380_66205779_66260497 |
| ATP13A2_chr1_−_17322880_{—} | MFSD14C_chr9_−_99704884_{—} | PLCB2_chr15_−_40582225_{—} |
| 17322973_17322795_17323514 | 99705034_99696197_99711836 | 40582283_40581559_40582777 |
| SMG7_chr1_+_183516237_{—} | ARIH2_chr3_+_48962150_{—} | FGGY_chr1_+_59805629_{—} |
| 183516387_183515472_183518342 | 48962272_48960244_48964894 | 59805741_59762822_59811918 |
| ACSF3_chr16_+_89167069_{—} | CHID1_chr11_−_893426_{—} | TOP3B_chr22_−_22336810_{—} |
| 89167755_89160404_89169011 | 893519_884169_899339 | 22336846_22330559_22337126 |
| RC3H2_chr9_−_125613601_{—} | KIF1C_chr17_+_4902307_{—} | PGAP2_chr11_+_3844842_{—} |
| 125613715_125613508_125616230 | 4902341_4901451_4903144 | 3844970_3829545_3845112 |
| CCDC189_chr16_−_30770974_{—} | TXNL4A_chr18_−_77737597_{—} | FIS1_chr7_−_100887287_{—} |
| 30771045_30770779_30771604 | 77737701_77733856_77748239 | 100887420_100884187_100888240 |
| DNM1L_chr12_+_32890798_{—} | ADGRV1_chr5_+_90445846_{—} | TBL1XR1_chr3_−_176878658_{—} |
| 32890876_32890095_32891197 | 90446038_90398157_90449037 | 176878776_176782810_176914908 |
| KCNH3_chr12_+_49934681_{—} | SEC31A_chr4_−_83783686_{—} | SYCP2_chr20_−_58452439_{—} |
| 49934915_49933275_49935412 | 83783725_83782861_83784470 | 58452610_58450524_58453062 |
| SLC12A9_chr7_+_100454489_{—} | SEC31A_chr4_−_83783686_{—} | PRDX5_chr11_+_64088132_{—} |
| 100454798_100452000_100456456 | 83783725_83778917_83784470 | 64088264_64085858_64088336 |
| RFT1_chr3_−_53159924_{—} | FLAD1_chr1_+_154962814_{—} | PPIA_chr7_+_44838845_{—} |
| 53160010_53157856_53164353 | 154963004_154962733_154965188 | 44838876_44836392_44840885 |
| MTA1_chr14_+_105915695_{—} | BODIL1_chr4_−_13574325_{—} | ARAP2_chr4_−_36020765_{—} |
| 105915746_105905076_105916394 | 13574466_13571752_13578461 | 36020908_36014453_36047600 |
| CCDC43_chr17_−_42757952_{—} | DHRS3_chr1_−_12638745_{—} | RASGEF1B_chr4_−_82378652_{—} |
| 42758020_42756411_42759370 | 12638984_12632881_12640550 | 82378775_82377942_82380485 |
| LRP8_chr1_−_53741302_{—} | GALNT14_chr2_−_31348019_{—} | L3MBTL3_chr6_+_130370795_{—} |
| 53741425_53737018_53755238 | 31348119_31215873_31360823 | 130370975_130370538_130372393 |
| FAM207A_chr21_+_46393111_{—} | EPB41L1_chr20_+_34802278_{—} | KMT2D_chr12_−_49417835_{—} |
| 46393180_46387096_46396594 | 34802362_34800298_34806797 | 49417883_49416658_49418360 |
| ZFAND5_chr9_−_74978385_{—} | KCNQ4_chr1_+_41289768_{—} | MAPK9_chr5_−_179713974_{—} |
| 74978522_74975703_74979611 | 41289930_41288074_41296755 | 179714067_179707608_179718847 |
| PTAR1_chr9_−_72365696_{—} | DNAI1_chr9_+_34517282_{—} | TNS2_chr12_+_53453950_{—} |
| 72365866_72356774_72374768 | 34517465_34514737_34520655 | 53454042_53453755_53454188 |
| MECR_chr1_−_29527885_{—} | RPRDIA_chr18_−_33620769_{—} | CRACR2B_chr11_+_830865_{—} |
| 29528030_29527101_29528454 | 33620828_33613800_33647216 | 831032_830713_831223 |
| SAMD11_chr1_+_876523_{—} | LINC01572_chr16_−_72604349_{—} | SSBP2_chr5_−_80946085_{—} |
| 876686_874840_877515 | 72604414_72589225_72661433 | 80946158_80911376_81047486 |
| GOLGA8N_chr15_+_32892899_{—} | ANKS6_chr9_−_101552385_{—} | AMPD2_chr1_+_110163535_{—} |
| 32892968_32892644_32894213 | 101552888_101547158_101558414 | 110163888_110162900_110167924 |
| DAG1_chr3_+_49530255_{—} | SP110_chr2_−_231036411_{—} | FAM189B_chr1_−_155220066_{—} |
| 49530406_49507866_49547851 | 231036483_231035477_231036781 | 155220720_155218264_155220885 |
| LUC7L_chr16_−_258599_{—} | PHKB_chr16_+_47497792_{—} | RALGPS2_chr1_+_178861364_{—} |
| 258663_258187_270647 | 47497904_47495337_47531309 | 178861442_178858831_178863053 |
| GSN_chr9_+_124045596_{—} | ARHGEF39_chr9_−_35664752_{—} | CCDC125_chr5_−_68588014_{—} |
| 124045670_124043840_124060043 | 35664847_35664489_35673807 | 68588189_68581294_68595838 |
| CASP6_chr4_−_110617565_{—} | SH2D6_chr2_+_85662788_{—} | NLRC5_chr16_+_57080463_{—} |
| 110617642_110615856_110618777 | 85662939_85661493_85663959 | 57080553_57079404_57081456 |
| TNRC18_chr7_−_5401224_{—} | SNX14_chr6_−_86248555_{—} | CSGALNACT2_chr10_+_43662451_{—} |
| 5401353_5399200_5401527 | 86248582_86246642_86251702 | 43662546_4365949243671398 |
| LPIN2_chr18_−_2928588_{—} | ZNF76_chr6_+_35261527_{—} | PIK3C3_chr18_+_39637846_{—} |
| 2928658_2927809_2929062 | 35261692_35260821_35262232 | 39638015_39629569_39644703 |
| EVA1B_chr1_−_36788571_{—} | C1orf61_chr1_−_156376871_{—} | AC005154.6_chr7_−_30618621_{—} |
| 36788668_36788326_36789059 | 156376989_156374393_156389962 | 30618744_30617707_30618846 |
| BCAM_chr19_+_45317408_{—} | PAM_chr5_+_102360837_{—} | HMGN3_chr6_−_79911779_{—} |
| 45317545_45316877_45317860 | 102361038_102355547_102363888 | 79911872_79911443_79911992 |
| SMUG1_chr12_−_54580630_{—} | FLNB_chr3_+_58117653_{—} | TMPRSS5_chr11_−_113563792_{—} |
| 54580795_54577743_54582298 | 58117746_58116635_58118534 | 113563971_113558996_113565199 |
| FXR1_chr3_+_180693100_{—} | TPST1_chr7_+_65817491_{—} | PLA2G6_chr22_−_38524275_{—} |
| 180693192_180688146_180693909 | 65817542_65751696_65824881 | 38524437_38522456_38525460 |
| PDCD10_chr3_−_167443188_{—} | NUP62_chr19_−_50430950_{—} | MYL6_chr12_+_56553370_{—} |
| 167443261_167438061_167452246 | 50431105_50413141_50432582 | 56553406_56552495_56553758 |
| TM2D1_chr1_−_62152463_{—} | FAM136A_chr2_−_70528539_{—} | EFHD2_chr1_+_15753645_{—} |
| 62152567_62149218_62160368 | 70528735_70528112_70529056 | 15753780_15752514_15755088 |
| MICAL2_chr11_+_12270730_{—} | USPL1_chr13_+_31195209_{—} | AKNA_chr9_−_117143339_{—} |
| 12270793_12265659_12278331 | 31195376_31192193_31195900 | 117143726_117139812_117156636 |
| GUCD1_chr22_−_24944884_{—} | PAQR5_chr15_+_69681992_{—} | UBXN11_chr1_−_26627416_{—} |
| 24944969_24944041_24951582 | 69682119_69672349_69689806 | 26627515_26624553_26628184 |
| PLSCR3_chr17_−_7296462_{—} | ASPH_chr8_−_62546241_{—} | SPTBN5_chr15_−_42162643_{—} |
| 7296683_7296271_7296785 | 62546286_62538839_62550505 | 42162758_42162551_42163572 |
| PLD3_chr19_+_40871459_{—} | ZKSCAN5_chr7_+_99117449_{—} | ATXN7L1_chr7_−_105258238_{—} |
| 40871837_40854675_40872325 | 99117532_99110214_99123435 | 105258544_105255263_105260646 |
| C17orf58_chr17_−_65989159_{—} | NMU_chr4_−_56471441_{—} | TMEM9B_chr11_−_8983638_{—} |
| 65989351_65988219_65989539 | 56471516_56466742_56473464 | 8983730_8977845_8985755 |
| FMR1_chrX_+_147019617_{—} | MEF2D_chr1_−_156446285_{—} | EEF1D_chr8_−_144672777_{—} |
| 147019680_147019119_147022094 | 156446306_156445029_156446803 | 144672905_144672251_144679517 |
| EMILIN1_chr2_+_27308027_{—} | PVT1_chr8_+_128867400_{—} | SOS1_chr2_−_39216410_{—} |
| 27308165_27307876_27308545 | 128867565_128806980_128902834 | 39216455_39214732_39222263 |
| ATG4D_chr19_+_10657514_{—} | NTRK1_chr1_+_156843424_{—} | HKR1_chr19_+_37815697_{—} |
| 10657758_10655806_10657864 | 156843751_156838439_156844362 | 37815838_37815167_37838091 |
| DNAH14_chr1_+_225533663_{—} | NOP14-AS1_chr4_+_2939701_{—} | LZTFL1_chr3_−_45868847_{—} |
| 225534082_225528403_225534157 | 2939869_2939571_2948599 | 45868951_45867824_45869930 |
| AMMECR1L_chr2_−_128628822_{—} | DCAF8_chr1_−_160190248_{—} | LLOXNC01-237H1.2_chrX_+_102155950_{—} |
| 128628933_128624570_128631401 | 160190322_160188758_160192440 | 102156061_102155760_102156558 |
| TMEM51_chr1_+_15541390_{—} | PAK4_chr19_+_39658869_{—} | MROH6_chr8_−_144649780_{—} |
| 15541927_15480450_15545821 | 39658952_39647490_39660171 | 144649846_144649635_144649955 |
| ATXN7L1_chr7_−_105260646_{—} | MYL6_chr12_+_56553406_{—} | ANO7_chr2_+_242135117_{—} |
| 105260768_105255263_105264521 | 56553514_56552495_56553758 | 242135260_242130666_242138730 |
| METTL8_chr2_−_172182551_{—} | APOBEC3C_chr22_+_39411599_{—} | YLPM1_chr14_+_75264282_{—} |
| 172182658_172182416_172187068 | 39411756_39410384_39413770 | 75266400_75249028_75269258 |
| YY1AP1_chr1_−_155657861_{—} | CRAMP1_chr16_+_1716073_{—} | GOLGA2P5_chr12_−_100552456_{—} |
| 155657992_155650247_155658449 | 1716178_1715139_1716422 | 100552651_100551855_100552735 |
| RNPEPL1_chr2_+_241513191_{—} | APP_chr21_−_27369674_{—} | SPTBN5_chr15_−_42166042_{—} |
| 241513308_241512678_241513529 | 27369731_27354790_27394155 | 42166243_42165806_42166489 |
| GNMT_chr6_+_42930809_{—} | ZNF83_chr19_−_53119970_{—} | PLA2G12A_chr4_−_110639844_{—} |
| 42930952_42930616_42931065 | 53120128_53118050_53122188 | 110639915_110638869_110650757 |
| PAX8-AS1_chr2_+_114016840_{—} | ASIC1_chr12_+_50470750_{—} | PAQR6_chr1_−_156216471_{—} |
| 114017161_113995789_114020854 | 50470832_50453737_50470995 | 156216547_156216041_156217747 |
| MTCL1_chr18_+_8809445_{—} | CCDC125_chr5_−_68590619_{—} | ACIN1_chr14_−_23540333_{—} |
| 8809559_8807058_8812976 | 68590727_68581294_68595838 | 23540395_23538826_23540635 |
| DIAPH2_chrX_+_95990756_{—} | MLH1_chr3_+_37090007_{—} | MYNN_chr3_+_169491818_{—} |
| 95990789_95940189_95993584 | 37090100_37089174_37090394 | 169491885_169491250_169492052 |
| TPRA1_chr3_−_127295663_{—} | PALM3_chr19_−_14168162_{—} | RRP8_chr11_−_6622155_{—} |
| 127295736_127295539_127295832 | 14168243_14167616_14169926 | 6622285_6622005_6622378 |
| FES_chr15_+_91434211_{—} | PPP6R1_chr19_−_55752606_{—} | BCL3_chr19_+_45261502_{—} |
| 91434421_91433714_91434783 | 55752759_55752447_55752844 | 45261670_45260980_45261980 |
| MBD1_chr18_−_47800555_{—} | KIAA1191_chr5_−_175777615_{—} | ERBIN_chr5_+_65367996_{—} |
| 47800720_47800233_47801346 | 175777740_175775359_175788604 | 65368119_65350779_65370851 |
| USP21_chr1_+_161130155_{—} | KCNH3_chr12_+_49943862_{—} | ELOC_chr8_−_74876721_{—} |
| 161130296_161129468_161130409 | 49944112_49943423_49948119 | 74876860_74872053_74884104 |
| ALOX5_chr10_+_45939563_{—} | WARS_chr14_−_100841619_{—} | CCDC189_chr16_−_30770974_{—} |
| 45939734_45939276_45940955 | 100841740_100835595_100842596 | 30771130_30770779_30771604 |
| MAPKAPK5_chr12_+_112308074_{—} | ZDHHC3_chr3_−_44974398_{—} | PILRB_chr7_+_99951517_{—} |
| 112308164_112306665112308888 | 44974482_44970928_44974609 | 99951635_99951106_99952765 |
| IFI44_chr1_+_79126238_{—} | SCOC_chr4_+_141300722_{—} | BAIAP2_chr17_+_79010662_{—} |
| 79126376_79125168_79128388 | 141300806_141300346_141302115 | 79010761_79009108_79027467 |
| EEF1D_chr8_−_144672777_{—} | CBLC_chr19_+_45287520_{—} | TRIP12_chr2_−_230725121_{—} |
| 144672908_144672251_144679517 | 45287658_45285748_45293260 | 230725247_230724290_230744697 |
| MYO9B_chr19_+_17321141_{—} | ATAD2B_chr2_−_24050667_{—} | ARVCF_chr22_−_19978107_{—} |
| 17321189_17320518_17321523 | 24050730_24046439_24051718 | 19978335_19969614_19997977 |
| TRAPPC12_chr2_+_3481462_{—} | BOLA3_chr2_−_74369398_{—} | PPP1R12B_chr1_+_202414173_{—} |
| 3481566_3469466_3482616 | 74369487_74362785_74372315 | 202414356_202411700_202418116 |
| R3HDM1_chr2_+_136374237_{—} | AGTPBP1_chr9_−_88293249_{—} | MIS18BP1_chr14_−_45719233_{—} |
| 136374327_136289203_136379063 | 88293313_88292497_88307603 | 45719341_45716580_45722237 |
| TTYH3_chr7_+_2687592_{—} | PRSS22_chr16_−_2905574_{—} | HNRNPA1_chr12_+_54676862_{—} |
| 2687688_2687272_2689200 | 2905852_2904023_2906082 | 54677018_54676449_54677595 |
| SLC29A2_chr11_−_66136530_{—} | EFCAB6_chr22_−_44204874_{—} | TSPAN14_chr10_+_82222843_{—} |
| 66136670_66136142_66136839 | 44205011_44178205_44208047 | 82222883_82219263_82264483 |
| TET2_chr4_+_106111516_{—} | ZNF271P_chr18_+_32870973_{—} | MTA1_chr14_+_105911754_{—} |
| 106111662_106068136_106180775 | 32871196_32870355_32885939 | 105911848_105905076_105916394 |
| ARPC4-TTLL3_chr3_+_9862229_{—} | CANT1_chr17_−_76994229_{—} | HPF1_chr4_−_170669894_{—} |
| 9862425_9855029_9867483 | 76994368_76994045_76996181 | 170669993_170663258_170671686 |
| TMUB2_chr17_+_42265274_{—} | ZNF300_chr5_−_150282702_{—} | CDC42BPA_chr1_−_227239581_{—} |
| 42265377_42265111_42266302 | 150282744_150278116_150283425 | 227239686_227235711_227257477 |
| HNRNPH1_chr5_−_179046269_{—} | C12orf49_chr12_−_117157567_{—} | OGDHL_chr10_−_50945831_{—} |
| 179046361_179045324_179047892 | 117157681_117155698_117175594 | 50945904_50944566_50945992 |
| FAM208B_chr10_+_5754356_{—} | MFF_chr2_+_228211941_{—} | DGKI_chr7_−_137178528_{—} |
| 5754502_5727138_5754785 | 228212100_228205096_228217229 | 137178582_137154365_137206611 |
| ABCA1_chr9_−_107665894_{—} | PLD3_chr19_+_40871568_{—} | MFSD14C_chr9_−_99704884_{—} |
| 107666052_107651476_107690215 | 40871837_40854675_40872325 | 99705034_99681108_99711836 |
| ZNF706_chr8_−_102214560_{—} | TMPO_chr12_+_98938007_{—} | FNBP1_chr9_−_132678244_{—} |
| 102214675_102213971_102217662 | 98938127_98931350_98938728 | 132678259_132671278_132687238 |
| METTL4_chr18_−_2544651_{—} | INPP5J_chr22_+_31521818_{—} | ARRB1_chr11_−_74982744_{—} |
| 2544758_2539144_2547353 | 31521996_31520892_31522361 | 74982768_74980003_74983938 |
| OGDHL_chr10_−_50948755_{—} | STAMBP_chr2_+_74097269_{—} | TANGO2_chr22_+_20049052_{—} |
| 50948883_50946308_50950873 | 74097479_74089431_74100478 | 20049206_20043536_20050860 |
| FAM189B_chr1_−_155220885_{—} | NSUN5P2_chr7_−_72422690_{—} | RUSC1-AS1_chr1_−_155292033_{—} |
| 155220955_155218264_155223415 | 72422834_72420735_72423864 | 155292228_155291486_155293868 |
| KLHL12_chr1_−_202862366_{—} | LINC01993_chr17_−_76267332_{—} | CADPS2_chr7_−_122033249_{—} |
| 202862553_202861787_202863311 | 76268048_76260303_76274524 | 122033369_122028792_122033494 |
| SLC25A22_chr11_−_796042_{—} | BPNT1_chr1_−_220246191_{—} | MTMR2_chr11_−_95620775_{—} |
| 796367_795169_798216 | 220246299_220242774_220247308 | 95620923_95598840_95621319 |
| CHN1_chr2_−_175869621_{—} | ALOX12B_chr17_−_7982713_{—} | NASP_chr1_+_46072992_{—} |
| 175869808_175816930_175869933 | 7982857_7980511_7983086 | 46074009_46072263_46078840 |
| KLHL8_chr4_−_88106402_{—} | SLC43A1_chr11_−_57263503_{—} | SPTBN5_chr15_−_42172357_{—} |
| 88106951_88104541_88116475 | 57263637_57261644_57268251 | 42172510_42172062_42173231 |
| DYRK3_chr1_+_206810219_{—} | CD47_chr3_−_107768465_{—} | DNAH1_chr3_+_52386567_{—} |
| 206810313_206809125_206810994 | 107768498_107766139_107776323 | 52386723_52386119_52387118 |
| EPB41L1_chr20_+_34802281_{—} | U2SURP_chr3_+_142773784_{—} | TARBP1_chr1_−_234536598_{—} |
| 34802362_34800298_34806797 | 142773961_142772636_142775153 | 234536694_234534299_234536926 |
| PQLC1_chr18_−_77693968_{—} | MKNK1_chr1_−_47040618_{—} | SLC29A1_chr6_+_44193709_{—} |
| 77694022_77664183_77703328 | 47040692_47037839_47042246 | 44193904_44191378_44194999 |
| COPS7B_chr2_+_232656442_{—} | FGFR1OP_chr6_+_167416680_{—} | HM13_chr20_+_30154012_{—} |
| 232656518_232646593_232658972 | 167416735_167413601_167424300 | 30154098_30149539_30156922 |
| C1orf61_chr1_−_156384445_{—} | LRMP_chr12_+_25215753_{—} | C6orf52_chr6_−_10672779_{—} |
| 156384545_156374393_156391346 | 25215834_25205890_25216653 | 10672869_10671831_10683419 |
| MCCC2_chr5_+_70922466_{—} | TROVE2_chr1_+_193038163_{—} | MTMR2_chr11_−_95620775_{—} |
| 70922580_70900295_70927947 | 193038764_193028906_193045630 | 95620848_95599650_95621319 |
| BDH1_chr3_−_197281432_{—} | NACA_chr12_−_57109654_{—} | MPRIP_chr17_+_17046869_{—} |
| 197281584_197273357_197282651 | 57109990_57108471_57113320 | 17046983_17046079_17049349 |
| TBL1XR1_chr3_−_176816253_{—} | TRA2A_chr7_−_23561739_{—} | HUWE1_chrX_−_53713214_{—} |
| 176816329_176782810_176914908 | 23562051_23561459_23571407 | 53713313_53707139_53713532 |
| FAM3B_chr21_+_42694849_{—} | RAD52_chr12_−_1023596_{—} | FAM208B_chr10_+_5751492_{—} |
| 42694993_42688825_42710304 | 1023698_1023287_1025509 | 5751626_5734881_5754356 |
| SF1_chr11_−_64533422_{—} | JMJD1C_chr10_−_64979637_{—} | UBA2_chr19_+_34921480_{—} |
| 64533627_64532990_64534663 | 64979743_64977091_65024410 | 34921564_34919475_34922765 |
| SGO1_chr3_−_20215740_{—} | IQCE_chr7_+_2608587_{—} | TMEM144_chr4_+_159136342_{—} |
| 20216547_20212724_20218092 | 2608633_2598851_2611159 | 159136465_159133928_159140461 |
| SSH1_chr12_−_109205035_{—} | ACAD10_chr12_+_112148076_{—} | CCDC159_chr19_+_11462581_{—} |
| 109205104_109203534_109210813 | 112148169_112147488_112150301 | 11462649_11461679_11462732 |
| CTPS1_chr1_+_41473120_{—} | HCG18_chr6_−_30262247_{—} | PPP2R3C_chr14_−_35565763_{—} |
| 41473217_41471766_41474330 | 30262741_30260376_30263908 | 35565839_35564390_35568457 |
| ATP6V1C2_chr2_+_10917710_{—} | PRPF4B_chr6_+_4061888_{—} | ANKRD19P_chr9_+_95591219_{—} |
| 10917848_10915197_10918697 | 4061934_4061381_4062446 | 95591445_95588860_95599058 |
| DNM1L_chr12_+_32890798_{—} | G3BP2_chr4_−_76579166_{—} | SCAF1_chr19_+_50148277_{—} |
| 32890876_32890095_32892997 | 76579265_76573925_76580249 | 50148391_50145499_50149364 |
| RAC1_chr7_+_6438292_{—} | WDR17_chr4_+_177036946_{—} | IFI44_chr1_+_79126238_{—} |
| 6438349_6431672_6439756 | 177037130_177032854_177041017 | 79126339_79125168_79128388 |
| FIZ1_chr19_−_56106985_{—} | MPP2_chr17_−_41981797_{—} | KDM5D_chrY_−_21901413_{—} |
| 56107100_56105012_56108937 | 41981861_41975748_41984841 | 21901548_21897636_21903203 |
| PPP1CB_chr2_+_29016728_{—} | SNU13_chr22_−_42078359_{—} | SSPO_chr7_+_149476116_{—} |
| 29016863_29006844_29022064 | 42078591_42076368_42084797 | 149476196_149476028_149479269 |
| HPS1_chr10_−_100189547_{—} | FOXJ3_chr1_−_42671432_{—} | MTO1_chr6_+_74190040_{—} |
| 100189646_100189399_100190327 | 42671534_42664901_42693553 | 74190090_74189849_74190397 |
| PIEZO1_chr16_−_88791416_{—} | KCTD9_chr8_−_25303644_{—} | KLHL8_chr4_−_88106402_{—} |
| 88791488_88790379_88791823 | 25303766_25298189_25315714 | 88106723_88104541_88141569 |
| NSD2_chr4_+_1895456_{—} | ENTPD4_chr8_−_23261466_{—} | DTNB_chr2_−_25818954_{—} |
| 1895636_1873269_1902352 | 23261586_23243558_23291829 | 25819109_25803695_25851039 |
| NAA25_chr12_−_112487286_{—} | ZNF789_chr7_+_99075896_{—} | CHCHD7_chr8_+_57125320_{—} |
| 112487415_112486247_112491361 | 99075979_99074103_99077283 | 57125448_57124396_57127156 |
| EHBP1L1_chr11_+_65349009_{—} | FBXW11_chr5_−_171341346_{—} | CNN2_chr19_+_1036065_{—} |
| 65349487_65348844_65351711 | 171341409_171337801_171384600 | 1036245_1032695_1036414 |
| EGFL7_chr9_+_139564057_{—} | KITLG_chr12_−_88909310_{—} | ARHGAP28_chr18_+_6876129_{—} |
| 139564173_139563125_139564365 | 88909394_88900914_88910110 | 6876207_6873774_6882135 |
| ENTPD6_chr20_+_25201867_{—} | AP2M1_chr3_+_183898432_{—} | RAB25_chr1_+_156035701_{—} |
| 25201969_25198217_25203473 | 183898438_183898039_183898636 | 156035897_156031234_156038060 |
| HPGD_chr4_−_175414301_{—} | TAF1D_chr11_−_93464315_{—} | MAP2K5_chr15_+_68040568_{—} |
| 175414465_175413245_175416698 | 93464382_93463878_93467790 | 68040595_67995746_68040906 |
| PUM1_chr1_−_31452908_{—} | GTSE1_chr22_+_46704046_{—} | CFAP157_chr9_+_130472879_{—} |
| 31453013_31447649_31454158 | 46704104_46693376_46704215 | 130473033_130471972_130473507 |
| SULT1C3_chr2_+_108869800_{—} | FAM175A_chr4_−_84393374_{—} | TMEM62_chr15_+_43430771_{—} |
| 108869898_108868949_108872027 | 84393441_84390304_84397795 | 43430817_43427847_43438690 |
| EXTL3_chr8_+_28494818_{—} | ZNF654_chr3_+_88177958_{—} | ING4_chr12_−_6761436_{—} |
| 28494946_28480327_28570973 | 88178161_88175419_88178836 | 6761475_6760551_6761827 |
| TOGARAM1_chr14_+_45492112_{—} | SSPO_chr7_+_149490397_{—} | SH2D6_chr2_+_85662187_{—} |
| 45492176_45481278_45494985 | 149490551_149489817_149490653 | 85662246_85661493_85663959 |
| ITGB4_chr17_+_73735752_{—} | NETO2_chr16_−_47120136_{—} | SNRPN_chr15_+_25165619_{—} |
| 73735786_73733525_73735960 | 47120250_47117712_47143393 | 25165694_25165271_25207260 |
| SYNE4_chr19_−_36499118_{—} | GOLIM4_chr3_−_167758573_{—} | RP13-794C1.1_chr3_+_48502759_{—} |
| 36499269_36497846_36499455 | 167758657_167754782_167759179 | 48502844_48502198_48505143 |
| PRKCZ_chr1_+_1990979_{—} | MFSD8_chr4_−_128851837_{—} | SSFA2_chr2_+_182785323_{—} |
| 1991030_1987001_2066700 | 128851972_128843118_128854139 | 182785389_182784173_182786674 |
| MRPS28_chr8_−_80940929_{—} | RP1-27K12.2_chr6_−_53434277_{—} | PREPL_chr2_−_44586635_{—} |
| 80941031_80915415_80942139 | 53434403_53429687_53481683 | 44587177_44573529_44588518 |
| LYRM1_chr16_+_20913807_{—} | SSPO_chr7_+_149479934_{—} | NBPF15_chr1_+_148566781_{—} |
| 20914039_20911857_20926877 | 149480110_149479387_149480194 | 148566880_148561115_148568735 |
| SUGP2_chr19_−_19102148_{—} | SMC5_chr9_+_72961520_{—} | PXN_chr12_−_120653362_{—} |
| 19102362_19101958_19105174 | 72961565_72959185_72961981 | 120653464_120653220_120657009 |
| ZNF185_chrX_+_152088871_{—} | DAG1_chr3_+_49524686_{—} | DCAF6_chr1_+_167992225_{—} |
| 152088955_152087625_152089253 | 49524848_49514338_49547851 | 167992285_167974031_168007608 |
| BBC3_chr19_−_47731414_{—} | PDDC1_chr11_−_770992_{—} | CCDC57_chr17_−_80109435_{—} |
| 47731703_47725175_47735771 | 771104_767373_771332 | 80109649_80086473_80115623 |
| PCBP1-AS1_chr2_−_70286774_{—} | IFT122_chr3_+_129183477_{—} | |
| 70286812_70283042_70310669 | 129183624_129182469_129188184 | |

TABLE 1c
Myc AS Tissue

| AS Events | AS Events | AS Events |
|---|---|---|
| AARSD1_chr17_−_41105740_{—} | MAN2B1_chr19_−_12769241_{—} | TRMU_chr22_+_46739158_{—} |
| 41105795_41103911_41106892 | 12769324_12769158_12772073 | 46739265_46733841_46742318 |
| AASS_chr7_−_121741424_{—} | MANBAL_chr20_+_35925698_{—} | TRNAU1AP_chr1_+_28887624_{—} |
| 121741492_121738920_121741674 | 35925937_35918089_35927165 | 28887772_28887244_28887857 |
| ABCA5_chr17_−_67247887_{—} | MANBAL_chr20_+_35927165_{—} | TSC2_chr16_+_2132436_{—} |
| 67248007_67246762_67249713 | 35927282_35918089_35929610 | 2132505_2131799_2133695 |
| ABCD4_chr14_−_74756729_{—} | MAP2K7_chr19_+_7970692_{—} | TSFM_chr12_+_58186768_{—} |
| 74756821_74756222_74756993 | 7970740_7968953_7974639 | 58186856_58180945_58189959 |
| ABHD16A_chr6_−_31669050_{—} | MAP3K6_chr1_−_27691151_{—} | TSNAX_chr1_+_231699210_{—} |
| 31669117_31668805_31670926 | 27691175_27690885_27691263 | 231699375_231697001_231700273 |
| ABHD4_chr14_+_23074692_{—} | MAP3K7_chr6_−_91254270_{—} | TSPAN4_chr11_+_862549_{—} |
| 23074772_23072984_23075327 | 91254351_91246120_91256976 | 862741_850367_864436 |
| ABI1_chr10_−_27044583_{—} | MAP4_chr3_−_47910703_{—} | TTC27_chr2_+_33036090_{—} |
| 27044670_27040712_27047990 | 47910817_47899002_47912302 | 33036288_33012216_33042523 |
| ABI1_chr10_−_27044583_{—} | MAPK12_chr22_−_50686120_{—} | TTC31_chr2_+_74717151_{—} |
| 27044670_27040712_27047990 | 50686205_50685395_50686318 | 74717254_74710537_74717370 |
| ABLIM3_chr5_+_148622053_{—} | MAPK3_chr16_−_30126915_{—} | TTC7A_chr2_+_47185633_{—} |
| 148622101_148619451_148624443 | 30126966_30126029_30127956 | 47185691_47184146_47202111 |
| AC005154.6_chr7_−_30590933_{—} | MAPT_chr17_+_44060543_{—} | TTC7B_chr14_−_91069596_{—} |
| 30591095_30590397_30603346 | 44061296_44055806_44064405 | 91069647_91044652_91077085 |
| AC005154.6_chr7_−_30618621_{—} | MARK3_chr14_+_103964838_{—} | TTC7B_chr14_−_91069596_{—} |
| 30618744_30617707_30618846 | 103964865_103958371_103966492 | 91069647_91059970_91077085 |
| ACAA1_chr3_−_38173086_{—} | MARK3_chr14_+_103964838_{—} | TTLL7_chr1_−_84414310_{—} |
| 38173129_38170879_38173416 | 103964865_103958371_103969218 | 84414378_84412965_84415547 |
| ACLY_chr17_−_40052872_{—} | MARK3_chr14_+_103966492_{—} | U2AF1_chr21_−_44521475_{—} |
| 40052902_40049427_40054001 | 103966537_103946827_103969218 | 44521542_44520629_44524424 |
| ACOT9_chrX_−_23752457_{—} | MARK3_chr14_+_103966492_{—} | U2SURP_chr3_+_142742816_{—} |
| 23752484_23751334_23754035 | 103966537_103958371_103969218 | 142742860_142741906_142745990 |
| ACSF3_chr16_+_89180746_{—} | MATR3_chr5_+_138642927_{—} | UBAP2L_chr1_+_154241837_{—} |
| 89180895_89178654_89187208 | 138644016_138629494_138650363 | 154241888_154241430_154243356 |
| ACTN1_chr14_−_69345174_{—} | MBNL1_chr3_+_152164492_{—} | UPP1_chr7_+_48139266_48139384_{—} |
| 69345240_69343957_69345705 | 152164546_152163328_152165408 | 48134424_48141420 |
| ACTN1_chr14_−_69345705_{—} | MBNL1_chr3_+_152173330_{—} | USP15_chr12_+_62768170_{—} |
| 69345786_69345240_69346678 | 152173366_152165562_152177059 | 62768316_62749256_62775270 |
| ACTR3B_chr7_+_152508781_{—} | MBNL1_chr3_+_152174055_{—} | USP28_chr11_−_113702641_{—} |
| 152508818_152498816_152511634 | 152174150_152165562_152177059 | 113702715_113700067_113704141 |
| ACY1_chr3_+_52019222_{—} | MCAT_chr22_−_43533086_{—} | USP54_chr10_−_75279554_{—} |
| 52019287_52018174_52019376 | 43533304_43529492_43537167 | 75279750_75277505_75283340 |
| ADA_chr20_−_43251228_{—} | MCCC1_chr3_−_182746880_{—} | USP54_chr10_−_75280665_{—} |
| 43251293_43249788_43251469 | 182746977_182743592_182751778 | 75280785_75277505_75283340 |
| ADAM15_chr1_+_155034051_{—} | MCCC1_chr3_−_182810196_{—} | UTP6_chr17_−_30205720_{—} |
| 155034122_155033965_155034720 | 182810333_182804576_182812346 | 30205800_30205346_30207591 |
| ADAM15_chr1_+_155034379_{—} | MCF2L_chr13_+_113745434_{—} | VMP1_chr17_+_57911372_{—} |
| 155034451_155033308_155034720 | 113745509_113744042_113748827 | 57911411_57895134_57915655 |
| ADAM33_chr20_−_3652780_{—} | ME3_chr11_−_86161342_{—} | VRK3_chr19_−_50500760_{—} |
| 3652976_3652632_3653183 | 86161440_86159297_86176132 | 50500827_50498532_50504046 |
| ADAM33_chr20_−_3652780_{—} | MEMO1_chr2_−_32118310_{—} | WBP1_chr2_+_74686604_{—} |
| 3652976_3652632_3653367 | 32118390_32117203_32142994 | 74686689_74685798_74686769 |
| ADSL_chr22_+_40749076_{—} | METTL6_chr3_−_15457004_{—} | WBP1_chr2_+_74686604_{—} |
| 40749121_40746039_40754867 | 15457090_15455669_15457278 | 74686689_74686225_74686769 |
| AFMID_chr17_+_76201173_{—} | MGEA5_chr10_−_103553669_{—} | WDFY3_chr4_−_85648013_{—} |
| 76201271_76200981_76202026 | 103553755_103552700_103557736 | 85648064_85645747_85654534 |
| AFMID_chr17_+_76201683_{—} | MICAL3_chr22_−_18286576_{—} | WDSUB1_chr2_−_160114455_{—} |
| 76201819_76198832_76202026 | 18286627_18274067_18291609 | 160114496_160112886_160116320 |
| AFMID_chr17_+_76201683_{—} | MICAL3_chr22_−_18295272_{—} | WNK1_chr12_+_988738_{—} |
| 76201819_76200981_76202026 | 18295323_18293579_18299455 | 989197_987527_989886 |
| AFMID_chr17_+_76201683_{—} | MICAL3_chr22_−_18309219_{—} | WNK2_chr9_+_96062332_{—} |
| 76201834_76198832_76202026 | 18309282_18305826_18314619 | 96062431_96061543_96070609 |
| AFMID_chr17_+_76201683_{—} | MICAL3_chr22_−_18310409_{—} | WNK2_chr9_+_96069058_{—} |
| 76201834_76200981_76202026 | 18310547_18305826_18314619 | 96069103_96060349_96070609 |
| AGA_chr4_−_178357429_{—} | MICAL3_chr22_−_18355512_{—} | WNK2_chr9_+_96069058_{—} |
| 178357505_178355643_178358558 | 18355620_18354789_18368643 | 96069103_96061543_96070609 |
| AHI1_chr6_−_135622545_{—} | MINDY3_chr10_−_15880226_{—} | WRNIP1_chr6_+_2770353_{—} |
| 135622677_135621696_135639656 | 15880278_15879317_15883424 | 2770595_2766678_2779496 |
| AKAP8L_chr19_−_15509440_{—} | MINK1_chr17_+_4762507_{—} | WRNIP1_chr6_+_2770428_{—} |
| 15509514_15508666_15510112 | 4762623_4736935_4781611 | 2770595_2766678_2779496 |
| AKAP8L_chr19_−_15509440_{—} | MINK1_chr17_+_4795696_{—} | WSB1_chr17_+_25624212_{—} |
| 15509577_15508666_15510112 | 4795807_4795529_4795950 | 25624334_25621461_25630392 |
| AKAP9_chr7_+_91671359_{—} | MIS12_chr17_+_5391515_{—} | YBX3_chr12_−_10862506_{—} |
| 91671500_91670212_91671981 | 5391909_5390004_5392142 | 10862713_10856747_10865809 |
| ALKBH3_chr11_+_43913590_{—} | MMS19_chr10_−_99221252_{—} | YLPM1_chr14_+_75290958_{—} |
| 43913679_43911378_43923065 | 99221379_99220764_99221584 | 75291010_75287840_75295915 |
| AMDHD2_chr16_+_2577573_{—} | MMS19_chr10_−_99228722_{—} | YPEL5_chr2_+_30371110_{—} |
| 2577616_2571124_2577773 | 99228861_99228163_99229402 | 30371407_30369928_30379493 |
| ANK3_chr10_−_61905725_{—} | MPND_chr19_+_4355093_{—} | YTHDC2_chr5_+_112860677_{—} |
| 61905779_61898845_61926348 | 4355170_4354417_4357249 | 112860874_112851059_112868575 |
| ANKMY1_chr2_−_241468453_{—} | MPRIP_chr17_+_17078606_{—} | ZBTB1_chr14_+_64983317_{—} |
| 241468926_241465266_241492330 | 17078726_17077389_17079739 | 64983469_64971664_64988204 |
| ANO8_chr19_−_17444230_{—} | MRPL1_chr4_+_78828099_{—} | ZC3H7A_chr16_−_11862886_{—} |
| 17444363_17444048_17444498 | 78828364_78815404_78830419 | 11862951_11862357_11864638 |
| AP1B1_chr22_−_29725700_{—} | MRPL10_chr17_−_45906504_{—} | ZCCHC6_chr9_−_88933872_{—} |
| 29725709_29724884_29726366 | 45906602_45906036_45908825 | 88933973_88932191_88934498 |
| AP1G2_chr14_−_24035024_{—} | MRPL22_chr5_+_154330362_{—} | ZCCHC6_chr9_−_88941285_{—} |
| 24035101_24034910_24035272 | 154330498_154320825_154335930 | 88941406_88940429_88943254 |
| AP2A2_chr11_+_1000431_{—} | MRPL22_chr5_+_154330380_{—} | ZCCHC8_chr12_−_122967825_{—} |
| 1000598_994245_1003721 | 154330498_154320825_154335930 | 122967891_122967242_122973932 |
| APLP2_chr11_+_129993506_{—} | MRPL33_chr2_+_27997290_{—} | ZCCHC8_chr12_−_122967825_{—} |
| 129993674_129992408_129996594 | 27997397_27995559_28002299 | 122968110_122967242_122973932 |
| APLP2_chr11_+_130007150_{—} | MRPL47_chr3_−_179320439_{—} | ZFR_chr5_−_32380180_{—} |
| 130007186_130003623_130010292 | 179320585_179319578_179322314 | 32380278_32379316_32385613 |
| APP_chr21_−_27369674_{—} | MRPL48_chr11_+_73554291_{—} | ZMIZ1_chr10_+_81070680_{—} |
| 27369731_27354790_27372329 | 73554412_73536841_73555851 | 81070941_81067328_81072398 |
| ARF1_chr1_+_228284151_{—} | MRPL55_chr1_−_228296137_{—} | ZMYND8_chr20_−_45841286_{—} |
| 228284205_228270551_228284778 | 228296175_228296019_228296655 | 45841370_45839542_45848908 |
| ARFGAP2_chr11_−_47194260_{—} | MRPL55_chr1_−_228296137_{—} | ZNF160_chr19_−_53594665_{—} |
| 47194302_47193884_47196565 | 228296175_228296019_228296849 | 53594750_53589574_53594889 |
| ARHGEF11_chr1_−_156908209_{—} | MRPL55_chr1_−_228296137_{—} | ZNF160_chr19_−_53594665_{—} |
| 156908305_156907288_156909339 | 228296175_228296019_228296961 | 53594782_53589574_53594889 |
| ARHGEF9_chrX_−_62947070_{—} | MRPL55_chr1_−_228296137_{—} | ZNF160_chr19_−_53594665_{—} |
| 62947299_62944591_62974194 | 228296175_228296022_228296655 | 53594806_53589574_53594889 |
| ARIH2_chr3_+_48982414_{—} | MRPL55_chr1_−_228296137_{—} | ZNF207_chr17_+_30693683_{—} |
| 48982614_48956431_48999044 | 228296209_228296019_228296655 | 30693776_30692506_30694790 |
| ARIH2_chr3_+_48982568_{—} | MRPS33_chr7_−_140710218_{—} | ZNF213-AS1_chr16_−_3182850_{—} |
| 48982614_48965246_48999044 | 140710460_140706335_140714710 | 3182946_3182104_3184630 |
| ARL4A_chr7_+_12727259_{—} | MTIF3_chr13_−_28015153_{—} | ZNF213-AS1_chr16_−_3182850_{—} |
| 12727353_12726668_12727790 | 28015214_28014586_28019224 | 3183195_3182104_3184630 |
| ARMCX2_chrX_−_100913446_{—} | MTMR10_chr15_−_31260122_{—} | ZNF263_chr16_+_3335058_{—} |
| 100913511_100913128_100914742 | 31260213_31253276_31266516 | 3335239_3334205_3336022 |
| ARMCX2_chrX_−_100913446_{—} | MTMR3_chr22_+_30419445_{—} | ZNF271P_chr18_+_32870973_{—} |
| 100913555_100913128_100914742 | 30419472_30418686_30421618 | 32871196_32870355_32885939 |
| ARMCX2_chrX_−_100914404_{—} | MVK_chr12_+_110016969_{—} | ZNF273_chr7_+_64377407_{—} |
| 100914487_100913128_100914742 | 110017087_110013950_110017606 | 64377496_64363797_64377958 |
| ARNT_chr1_−_150814899_{—} | MYEF2_chr15_−_48459545_{—} | ZNF326_chr1_+_90472903_{—} |
| 150814944_150812130_150818738 | 48459598_48458325_48460827 | 90473309_90470803_90475646 |
| ATAD3B_chr1_+_1425071_{—} | MYL6_chr12_+_56553370_{—} | ZNF326_chr1_+_90473170_{—} |
| 1425191_1424654_1425636 | 56553406_56552495_56553758 | 90473309_90470803_90475646 |
| ATG16L1_chr2_+_234182366_{—} | MYO18A_chr17_−_27412621_{—} | ZNF528-AS1_chr19_−_52900447_{—} |
| 234182423_234181698_234183321 | 27412666_27409456_27413455 | 52900520_52899003_52900714 |
| ATG2A_chr11_−_64671061_{—} | MYO18A_chr17_−_27443461_{—} | ZNF605_chr12_−_133524733_{—} |
| 64671132_64670836_64673034 | 27443473_27442858_27445062 | 133524856_133522383_133532828 |
| ATP6V0B_chr1_+_44441471_{—} | NAA16_chr13_+_41936866_{—} | ZNF638_chr2_+_71649943_{—} |
| 44441520_44440779_44441761 | 41937009_41936295_41941574 | 71651168_71645769_71651779 |
| ATP9B_chr18_+_76973961_{—} | NAA25_chr12_−_112487286_{—} | ZNF7_chr8_+_146054427_{—} |
| 76974038_76967012_77013380 | 112487415_112486247_112491361 | 146054475_146052994_146062778 |
| ATP9B_chr18_+_77108131_{—} | NACA_chr12_−_57109654_{—} | ZNF706_chr8_−_102214560_{—} |
| 77108196_77107925_77119353 | 57109990_57108471_57113320 | 102214675_102213971_102217662 |
| B3GALNT2_chr1_−_235652472_{—} | NADSYN1_chr11_+_71191264_{—} | ZNF761_chr19_+_53948253_{—} |
| 235652573_235647831_235657990 | 71191320_71189515_71191800 | 53948376_53935281_53949479 |
| BAZ2B_chr2_−_160253584_{—} | NAGLU_chr17_+_40689415_{—} | ZNF83_chr19_−_53119970_{—} |
| 160253611_160252345_160253855 | 40689563_40688673_40690356 | 53120068_53118050_53122188 |
| BCAR1_chr16_−_75270779_{—} | NAP1L1_chr12_−_76461150_{—} | ZNF83_chr19_−_53119970_{—} |
| 75270896_75269884_75276367 | 76461253_76454059_76467982 | 53120094_53118050_53122188 |
| BCAS3_chr17_+_59465978_{—} | NBEA_chr13_+_36220005_{—} | ZNF83_chr19_−_53119970_{—} |
| 59466069_59457932_59469337 | 36220068_36202385_36220395 | 53120128_53118050_53122188 |
| BET1_chr7_−_93623663_{—} | NCOA5_chr20_−_44708025_{—} | ZNHIT3_chr17_+_34842778_{—} |
| 93623697_93605412_93625576 | 44708092_44699175_44718455 | 34842810_34842629_34848656 |
| BIRC6_chr2_+_32815872_{—} | NCOR2_chr12_−_124811954_{—} | ZSWIM7_chr17_−_15880892_{—} |
| 32816045_32800433_32818981 | 124812179_124810916_124815390 | 15881014_15880406_15881357 |
| BPTF_chr17_+_65871671_{—} | NCOR2_chr12_−_124812092_{—} | SRRT_chr7_+_100478316_{—} |
| 65871860_65871136_65882243 | 124812179_124810916_124815390 | 100478390_100473333_100478905 |
| BPTF_chr17_+_65871671_{—} | NDEL1_chr17_+_8366637_{—} | SRRT_chr7_+_100480385_{—} |
| 65871860_65871136_65887959 | 8366672_8363478_8370247 | 100480711_100479862_100481690 |
| BPTF_chr17_+_65959448_{—} | NDRG2_chr14_−_21492188_{—} | TARSL2_chr15_−_102239533_{—} |
| 65959622_65955991_65960327 | 21492255_21491480_21493835 | 102239638_102226265_102241288 |
| BRD8_chr5_−_137495243_{—} | NDUFAF7_chr2_+_37469777_{—} | WASHC3_chr12_−_102443966_{—} |
| 137495288_137492956_137495757 | 37469836_37468780_37471005 | 102444069_102439897_102455025 |
| BTBD1_chr15_−_83718824_{—} | NECAP1_chr12_+_8244435_{—} | RAD52_chr12_−_1035961_{—} |
| 83718930_83710677_83725140 | 8244446_8242895_8245271 | 1036112_1034691_1036310 |
| BYSL_chr6_+_41897869_{—} | NFIB_chr9_−_14116179_{—} | KLC1_chr14_+_104158695_{—} |
| 41898008_41895274_41898372 | 14116345_14088325_14120438 | 104158762_104153548_104166991 |
| C12orf4_chr12_−_4627939_{—} | NFIB_chr9_−_14116206_{—} | C12orf73_chr12_−_104347191_{—} |
| 4628046_4614546_4634419 | 14116345_14088325_14120438 | 104347312_104345408_104350408 |
| C17orf62_chr17_−_80407045_{—} | NFIX_chr19_+_13189426_{—} | C12orf73_chr12_−_104348652_{—} |
| 80407168_80404572_80408575 | 13189549_13186485_13192493 | 104348746_104345408_104350408 |
| C17orf62_chr17_−_80407049_{—} | NFU1_chr2_−_69650713_{—} | C12orf73_chr12_−_104348652_{—} |
| 80407168_80403836_80408575 | 69650849_69646736_69659033 | 104348746_104347312_104350408 |
| C17orf62_chr17_−_80407049_{—} | NFYA_chr6_+_41048549_{—} | NT5C2_chr10_−_104860419_{—} |
| 80407168_80404572_80408575 | 41048636_41046903_41051784 | 104860700_104859776_104860801 |
| C1orf43_chr1_−_154192311_{—} | NGLY1_chr3_−_25761504_{—} | NT5C2_chr10_−_104860508_{—} |
| 154192413_154187050_154192817 | 25761682_25761126_25770623 | 104860700_104859776_104860801 |
| C2CD5_chr12_−_22611417_{—} | NHLRC3_chr13_+_39618226_{—} | NT5C2_chr10_−_104871501_{—} |
| 22611489_22610095_22612425 | 39618318_39613848_39621176 | 104871562_104866463_104899162 |
| C2CD5_chr12_−_22611417_{—} | NIN_chr14_−_51223209_{—} | TBCK_chr4_−_107158647_{—} |
| 22611489_22610095_22622642 | 51225348_51221585_51226574 | 107158695_107157965_107163626 |
| C2CD5_chr12_−_22611417_{—} | NKTR_chr3_+_42661507_{—} | MAGOHB_chr12_−_10761696_{—} |
| 22611519_22610095_22612425 | 42661535_42661200_42662920 | 10761982_10760535_10762429 |
| C2CD5_chr12_−_22611417_{—} | NOC4L_chr12_+_132631825_{—} | MAGOHB_chr12_−_10765238_{—} |
| 22611519_22610095_22622642 | 132631933_132630210_132635525 | 10765577_10763279_10766037 |
| C9orf3_chr9_+_97848211_{—} | NOD1_chr7_−_30477188_{—} | TCTN1_chr12_+_111072474_{—} |
| 97848401_97843062_97848963 | 30477272_30469073_30485756 | 111072584_111070364_111074285 |
| CACNAID_chr3_+_53752708_{—} | NPHP3_chr3_−_132415574_{—} | CARS2_chr13_−_111296411_{—} |
| 53752768_53752415_53753781 | 132415657_132413809_132416103 | 111296529_111294868_111296731 |
| CAD_chr2_+_27463434_{—} | NRDC_chr1_−_52302040_{—} | MAGI3_chr1_+_114224833_{—} |
| 27463485_27463229_27463778 | 52302110_52301884_52305897 | 114224924_114223958_114225518 |
| CALD1_chr7_+_134620438_{—} | NSFL1C_chr20_−_1436358_{—} | DNAJC25_chr9_+_114405136_{—} |
| 134620516_134618141_134625842 | 1436364_1435777_1438844 | 114405374_114394023_114409386 |
| CAMK2D_chr4_−_114424091_{—} | NSUN5P1_chr7_+_75043917_{—} | ATG12_chr5_−_115176193_{—} |
| 114424133_114421667_114430793 | 75044076_75042210_75044162 | 115176309_115173461_115177086 |
| CAMK2G_chr10_−_75577966_{—} | NTAN1_chr16_−_15141853_{—} | AGTRAP_chr1_+_11805986_{—} |
| 75578653_75577312_75579289 | 15141956_15141407_15149747 | 11806280_11805894_11807496 |
| CAMK2G_chr10_−_75577966_{—} | NUDT2_chr9_+_34336226_{—} | TAMM41_chr3_−_11874476_{—} |
| 75578653_75577312_75581439 | 34336339_34329597_34338710 | 11874625_11871338_11880695 |
| CAMK2G_chr10_−_75599296_{—} | NUDT2_chr9_+_34336297_{—} | SEPT6_chrX_−_118759297_{—} |
| 75599359_75597269_75601926 | 34336339_34329597_34338710 | 118759342_118750705_118763280 |
| CAMKK2_chr12_−_121682375_{—} | NUDT9_chr4_+_88366473_{—} | SEPT6_chrX_−_118759297_{—} |
| 121682418_121678672_121682942 | 88366659_88363067_88370293 | 118759342_118754014_118763280 |
| CAPN3_chr15_+_42698123_{—} | NUMB_chr14_−_73745988_{—} | SEPT6_chrX_−_118759297_{—} |
| 42698141_42695975_42700408 | 73746132_73744001_73749066 | 118759359_118754014_118763280 |
| CAPN7_chr3_+_15282959_{—} | NUP35_chr2_+_184016235_{—} | LAMP2_chrX_−_119572930_{—} |
| 15283095_15282360_15283684 | 184016377_183998351_184022171 | 119573148_119565317_119575584 |
| CARD19_chr9_+_95874160_{—} | NUP88_chr17_−_5307418_{—} | RPLP0_chr12_−_120636356_{—} |
| 95874220_95873003_95874499 | 5307566_5302970_5308376 | 120636573_120635265_120636656 |
| CASK_chrX_−_41414852_{—} | NUTF2_chr16_+_67881180_{—} | PXN_chr12_−_120654075_{—} |
| 41414888_41413168_41416284 | 67881359_67880888_67899004 | 120654919_120653464_120659425 |
| CASP6_chr4_−_110617565_{—} | OFD1_chrX_+_13771486_{—} | TIAL1_chr10_−_121339982_{—} |
| 110617642_110615856_110618777 | 13771560_13769487_13774696 | 121340358_121339522_121341433 |
| CAST_chr5_+_96062457_{—} | OGDH_chr7_+_44695916_{—} | AASS_chr7_−_121722841_{—} |
| 96062563_96058402_96063192 | 44695961_44687133_44706334 | 121722945_121721649_121726065 |
| CAST_chr5_+_96062497_{—} | ORMDL1_chr2_−_190647739_{—} | RSRC2_chr12_−_123005050_{—} |
| 96062563_96058402_96063192 | 190647849_190647328_190648994 | 123005128_123003598_123005931 |
| CBWD1_chr9_−_151304_{—} | OSBPL9_chr1_+_52135105_{—} | PLEKHA1_chr10_+_124187791_{—} |
| 151427_146158_152033 | 52135184_52117713_52179674 | 124187936_124186547_124189139 |
| CCDC130_chr19_+_13872304_{—} | OSGEP_chr14_−_20919415_{—} | MRRF_chr9_+_125048317_{—} |
| 13872382_13870086_13873112 | 20919611_20917425_20920132 | 125048445_125047566_125054027 |
| CCDC25_chr8_−_27606011_{—} | OSR2_chr8_+_99960400_{—} | SCARB1_chr12_−_125267228_{—} |
| 27606115_27605796_27610028 | 99960649_99957051_99961066 | 125267357_125263132_125270902 |
| CCNDBP1_chr15_+_43482252_{—} | PAAF1_chr11_+_73597973_{—} | TRMT11_chr6_+_126327987_{—} |
| 43482349_43481478_43482522 | 73598144_73589864_73598398 | 126328100_126320759_126329537 |
| CCSER2_chr10_+_86259630_{—} | PAAF1_chr11_+_73598075_{—} | STX2_chr12_−_131280539_{—} |
| 86259715_86237420_86273204 | 73598144_73589864_73598398 | 131280665_131276522_131283069 |
| CD151_chr11_+_834457_{—} | PAAF1_chr11_+_73598084_{—} | CRAT_chr9_−_131871457_{—} |
| 834591_833026_836062 | 73598144_73589864_73598398 | 131871556_131870356_131872761 |
| CD151_chr11_+_834529_{—} | PAOX_chr10_+_135197463_{—} | CCNL2_chr1_−_1326676_{—} |
| 834591_833022_836062 | 135197716_135195163_135202459 | 1326955_1326245_1328058 |
| CDC25B_chr20_+_3783376_{—} | PARD3_chr10_−_34625126_{—} | PPP2R2D_chr10_+_133748397_{—} |
| 3783455_3783023_3783556 | 34625171_34620272_34626202 | 133748510_133748059_133753534 |
| CEP95_chr17_+_62512840_{—} | PASK_chr2_−_242047881_{—} | DDX46_chr5_+_134125795_{—} |
| 62512946_62510476_62515438 | 242048222_242047715_242051654 | 134125912_134124281_134126159 |
| CHTOP_chr1_+_153614721_{—} | PBRM1_chr3_−_52588739_{—} | BCLAF1_chr6_−_136590278_{—} |
| 153614905_153610924_153617539 | 52588895_52584833_52595782 | 136590441_136589477_136590574 |
| CLASP2_chr3_−_33600616_{—} | PBRM1_chr3_−_52592264_{—} | BCLAF1_chr6_−_136595100_{—} |
| 33600667_33592887_33600798 | 52592429_52584833_52595782 | 136595327_136594325_136596669 |
| CLCC1_chr1_−_109504930_{—} | PCBP2_chr12_+_53861588_{—} | UVSSA_chr4_+_1374428_{—} |
| 109505091_109493070_109505982 | 53861627_53861077_53862560 | 1374535_1374018_1374667 |
| CLSTN1_chr1_−_9797555_{—} | PCBP2_chr12_+_53861588_{—} | NDUFAF5_chr20_+_13795063_{—} |
| 9797612_9796100_9801151 | 53861627_53861077_53862563 | 13795161_13789548_13797108 |
| CLSTN1_chr1_−_9816538_{—} | PCBP4_chr3_−_51995956_{—} | HNMT_chr2_+_138724666_{—} |
| 9816568_9815367_9833329 | 51996104_51995320_51996825 | 138724956_138722198_138727734 |
| CLTC_chr17_+_57764361_{—} | PCBP4_chr3_−_51996825_{—} | ATP11C_chrX_−_138813809_{—} |
| 57764382_57763169_57767996 | 51996908_51995320_52001341 | 138813914_138811121_138820074 |
| CMTM7_chr3_+_32490945_{—} | PCBP4_chr3_−_51996825_{—} | LUC7L2_chr7_+_139060254_{—} |
| 32491044_32483505_32493883 | 51996908_51996104_52001341 | 139060329_139045068_139060807 |
| CNNM2_chr10_+_104831530_{—} | PCSK7_chr11_−_117078369_{—} | APBB3_chr5_−_139941428_{—} |
| 104831596_104828479_104835842 | 117078451_117077876_117078685 | 139941580_139941286_139941684 |
| COL16A1_chr1_−_32136156_{—} | PDCD2_chr6_−_170889089_{—} | GK5_chr3_−_141903552_{—} |
| 32136247_32134456_32137215 | 170889193_170888058_170892144 | 141904635_141901891_141904770 |
| COL16A1_chr1_−_32136202_{—} | PDCD4_chr10_+_112636427_{—} | ATR_chr3_−_142169307_{—} |
| 32136247_32134456_32137215 | 112636511_112635828_112640990 | 142169444_142168444_142171969 |
| COPS7A_chr12_+_6837091_{—} | PDDC1_chr11_−_773521_{—} | DDX39A_chr19_−_14521232_{—} |
| 6837167_6833984_6837388 | 773629_772521_774007 | 14521417_14521146_14521800 |
| COQ5_chr12_−_120964259_{—} | PDIA5_chr3_+_122849326_{—} | DDX39A_chr19_−_14521359_{—} |
| 120964349_120960166_120966742 | 122849463_122843190_122864368 | 14521417_14521146_14521800 |
| CPNE1_chr20_−_34218662_{—} | PDK1_chr2_+_173433468_{—} | TCERG1_chr5_+_145889629_{—} |
| 34218717_34218412_34218822 | 173433545_173431661_173435453 | 145889723_145888808_145890003 |
| CPNE4_chr3_−_131753410_{—} | PDLIM7_chr5_−_176918404_{—} | EZH2_chr7_−_148516069_{—} |
| 131753603_131624288_131756400 | 176918421_176918147_176918807 | 148516151_148515209_148516687 |
| CPNE4_chr3_−_131753410_{—} | PELO_chr5_+_52095718_{—} | IDS_chrX_−_148583604_{—} |
| 131754286_131624288_131756400 | 52096954_52084248_52097242 | 148583707_148582568_148584841 |
| CRAT_chr9_−_131870686_{—} | PEX1_chr7_−_92138642_{—} | TARS2_chr1_+_150464073_{—} |
| 131870757_131870356_131871457 | 92138725_92136440_92140257 | 150464138_150463987_150464886 |
| CRAT_chr9_−_131870686_{—} | PEX19_chr1_−_160253319_{—} | AGAP3_chr7_+_150817606_{—} |
| 131870757_131870356_131872761 | 160253429_160252899_160254844 | 150817832_150817232_150819811 |
| CRELD1_chr3_+_9979698_{—} | PHLDB2_chr3_+_111667778_{—} | AGAP3_chr7_+_150817606_{—} |
| 9979790_9976601_9982533 | 111667922_111664204_111672776 | 150817832_150817232_150820880 |
| CROCCP2_chr1_−_16969261_{—} | PIGB_chr15_+_55642896_{—} | THEM4_chr1_−_151862458_{—} |
| 16969345_16961663_16971140 | 55643001_55634000_55646995 | 151862690_151861849_151867483 |
| CRTC1_chr19_+_18854917_{—} | PIGB_chr15_+_55642896_{—} | INTS3_chr1_+_153733495_{—} |
| 18854965_18853836_18856632 | 55643110_55634000_55646995 | 153733585_153733394_153734045 |
| CSDE1_chr1_−_115284147_{—} | PIGP_chr21_−_38444444_{—} | AKAP8_chr19_−_15479877_{—} |
| 115284294_115282511_115300545 | 38444610_38441924_38444733 | 15480035_15479133_15480956 |
| CSNK1G3_chr5_+_122941032_{—} | PIGQ_chr16_+_631198_{—} | CC2D2A_chr4_+_15480842_{—} |
| 122941056_122940524_122950035 | 631341_630972_632247 | 15480952_15480430_15482327 |
| CTBS_chr1_−_85035613_{—} | PILRB_chr7_+_99950995_{—} | CC2D2A_chr4_+_15480846_{—} |
| 85035822_85031695_85036264 | 99951106_99950746_99951517 | 15480952_15480430_15482327 |
| CTNND1_chr11_+_57558856_{—} | PKD1_chr16_−_2141423_{—} | HACL1_chr3_−_15628031_{—} |
| 57559145_57529518_57561481 | 2141598_2141175_2141781 | 15628109_15624496_15631046 |
| CTNND1_chr11_+_57558856_{—} | PLA2G5_chr1_+_20415317_{—} | MYH11_chr16_−_15802659_{—} |
| 57559145_57529591_57561481 | 20415394_20412720_20417060 | 15802698_15797980_15808765 |
| CTNND1_chr11_+_57558963_{—} | PLA2G5_chr1_+_20416281_{—} | MAP3K4_chr6_+_161529982_{—} |
| 57559145_57529518_57561481 | 20416388_20412720_20417060 | 161530073_161529891_161530786 |
| CTNND1_chr11_+_57558965_{—} | PLA2G6_chr22_−_38523413_{—} | TCF3_chr19_−_1615283_{—} |
| 57559145_57529518_57561481 | 38523465_38522456_38524275 | 1615484_1612432_1615684 |
| CTPS1_chr1_+_41473120_{—} | PLCB4_chr20_+_9353693_{—} | TCF3_chr19_−_1615283_{—} |
| 41473217_41471766_41474330 | 9353751_9353050_9360700 | 1615514_1612432_1615684 |
| CTTN_chr11_+_70268614_{—} | PLEKHA1_chr10_+_124187791_{—} | OXNAD1_chr3_+_16313651_{—} |
| 70268737_70266616_70269045 | 124187832_124186547_124189139 | 16313828_16313229_16327848 |
| CXorf40B_chrX_−_149102594_{—} | PLEKHA7_chr11_−_16828690_{—} | BZW2_chr7_+_16725548_{—} |
| 149102791_149102355_149105650 | 16828771_16824842_16834659 | 16725665_16722470_16729421 |
| CXXC1_chr18_−_47813792_{—} | PLEKHM2_chr1_+_16047823_{—} | DCAF6_chr1_+_167988782_{—} |
| 47813878_47813228_47813956 | 16047883_16046415_16051811 | 167988905_167974031_167992225 |
| CYP20A1_chr2_+_204137368_{—} | PLPP1_chr5_−_54786787_{—} | NADK_chr1_−_1688177_{—} |
| 204137471_204131404_204143295 | 54786942_54771278_54830399 | 1688321_1688047_1688619 |
| CYP20A1_chr2_+_204137392_{—} | PMF1_chr1_+_156195347_{—} | NOSTRIN_chr2_+_169712395_{—} |
| 204137471_204131404_204143295 | 156195459_156182967_156202110 | 169712720_169711970_169713199 |
| CYP2J2_chr1_−_60370037_{—} | PML_chr15_+_74324912_{—} | CBR4_chr4_−_169930102_{—} |
| 60370188_60366775_60370542 | 74325056_74317268_74325496 | 169930205_169928907_169931098 |
| D2HGDH_chr2_+_242688279_{—} | PMM2_chr16_+_8898623_{—} | RANBP17_chr5_+_170455875_{—} |
| 242689344_242684292_242689565 | 8898700_8895767_8900172 | 170455993_170395381_170597133 |
| DAZAP1_chr19_+_1425876_{—} | PMPCB_chr7_+_102948042_{—} | GORAB_chr1_+_170505450_{—} |
| 1425959_1422395_1428840 | 102948155_102944937_102949398 | 170505562_170501425_170508350 |
| DBN1_chr5_−_176886603_{—} | POFUT2_chr21_−_46697147_{—} | MPRIP_chr17_+_17083920_{—} |
| 176886741_176886269_176887432 | 46697688_46697057_46698017 | 17083983_17083402_17088136 |
| DCAF6_chr1_+_168007608_{—} | POLR3C_chr1_−_145597513_{—} | TBP_chr6_+_170876313_{—} |
| 168007726_167974031_168012262 | 145597574_145597054_145598535 | 170876593_170876097_170878699 |
| DCAF8_chr1_−_160231074_{—} | POT1_chr7_−_124469307_{—} | SLC27A1_chr19_+_17599656_{—} |
| 160231140_160213824_160232238 | 124469396_124467359_124475332 | 17599748_17598338_17599816 |
| DCAF8_chr1_−_160231074_{—} | PPHLN1_chr12_+_42745686_{—} | TSPAN17_chr5_+_176078754_{—} |
| 160231148_160213824_160232238 | 42745851_42729776_42748962 | 176078901_176074703_176079743 |
| DCN_chr12_−_91573138_{—} | PPOX_chr1_+_161137160_{—} | UIMC1_chr5_−_176396053_{—} |
| 91573463_91572362_91576431 | 161137276_161137024_161140409 | 176396292_176385155_176396601 |
| DCUN1D5_chr11_−_102953476_{—} | PPP1R12A_chr12_−_80173100_{—} | FAM193B_chr5_−_176958953_{—} |
| 102953568_102937296_102953984 | 80173131_80172380_80175594 | 176959132_176952206_176959443 |
| DDR1_chr6_+_30853401_{—} | PPP1R12A_chr12_−_80173118_{—} | FAM193B_chr5_−_176958953_{—} |
| 30853457_30850942_30856464 | 80173131_80172380_80175594 | 176959132_176958522_176959443 |
| DECR2_chr16_+_456889_{—} | PPP3CB_chr10_−_75199629_{—} | FAM193B_chr5_−_176958953_{—} |
| 456952_456833_457424 | 75199659_75198178_75204482 | 176959201_176952206_176959443 |
| DENNDIA_chr9_−_126150008_{—} | PPP4R1_chr18_−_9562919_{—} | FAM193B_chr5_−_176958953_{—} |
| 126150137_126146192_126165680 | 9563044_9562073_9563375 | 176959201_176958522_176959443 |
| DFFB_chr1_+_3782847_{—} | PPT2_EGFL8_chr6_+_32132355_{—} | FAM193B_chr5_−_176963358_{—} |
| 3782962_3782564_3784537 | 32132434_32130399_32134274 | 176963487_176959642_176964873 |
| DGUOK_chr2_+_74184251_{—} | PQLC1_chr18_−_77693968_{—} | SREBF1_chr17_−_17720861_{—} |
| 74184367_74177859_74185272 | 77694022_77679400_77703328 | 17720905_17720771_17721009 |
| DHODH_chr16_+_72055022_{—} | PRKAG1_chr12_−_49399525_{—} | PHYKPL_chr5_−_177658669_{—} |
| 72055210_72051005_72057063 | 49399664_49399326_49406844 | 177658867_177658524_177659492 |
| DHX9_chr1_+_182824711_{—} | PRMT1_chr19_+_50183128_{—} | HNRNPH1_chr5_−_179042546_{—} |
| 182824848_182823313_182825666 | 50183182_50180573_50185166 | 179042596_179041960_179043126 |
| DIAPH2_chrX_+_95990756_{—} | PRMT2_chr21_+_48056350_{—} | PRKRA_chr2_−_179310152_{—} |
| 95990789_95940189_95993584 | 48056459_48055675_48056807 | 179310262_179309227_179312231 |
| DMPK_chr19_−_46274228_{—} | PRUNE2_chr9_−_79239938_{—} | DNAJC10_chr2_+_183604271_{—} |
| 46274314_46273898_46274825 | 79239974_79229516_79244107 | 183604436_183601113_183605025 |
| DMPK_chr19_−_46274607_{—} | PRUNE2_chr9_−_79239938_{—} | TRA2B_chr3_−_185649364_{—} |
| 46274654_46273898_46274825 | 79239974_79234303_79244107 | 185649640_185643414_185655612 |
| DMTN_chr8_+_21924595_{—} | PSAP_chr10_−_73583644_{—} | TRA2B_chr3_−_185649364_{—} |
| 21924670_21924404_21925037 | 73583653_73581764_73585593 | 185649640_185644522_185655612 |
| DMWD_chr19_−_46287898_{—} | PSMC5_chr17_+_61905033_{—} | PIGX_chr3_+_196453525_{—} |
| 46287973_46287548_46288851 | 61905283_61904874_61905497 | 196453642_196449427_196454793 |
| DNAJC14_chr12_−_56223272_{—} | PSMC5_chr17_+_61905188_{—} | MINOS1_chr1_+_19927280_{—} |
| 56223420_56222498_56224479 | 61905283_61904874_61905497 | 19927465_19923603_19949967 |
| DNAJC19_chr3_−_180704730_{—} | PSMC5_chr17_+_61905210_{—} | ARVCF_chr22_−_19958738_{—} |
| 180704810_180703784_180705810 | 61905283_61904874_61905497 | 19958858_19958266_19959408 |
| DNAJC2_chr7_−_102967778_{—} | PTCD3_chr2_+_86352917_{—} | NOP58_chr2_+_203142934_{—} |
| 102967825_102967131_102968102 | 86353003_86352638_86354283 | 203143052_203142725_203147073 |
| DNAJC8_chr1_−_28556641_{—} | PTDSS2_chr11_+_473894_{—} | CD46_chr1_+_207963597_{—} |
| 28556763_28555534_28559431 | 473977_460288_479084 | 207963690_207959027_207966863 |
| DNM1L_chr12_+_32890798_{—} | PTGES2_chr9_−_130887057_{—} | HS1BP3_chr2_−_20840732_{—} |
| 32890876_32890095_32891197 | 130887150_130886829_130887522 | 20840940_20838412_20845099 |
| DNM2_chr19_+_10919244_{—} | PTPA_chr9_+_131890242_{—} | RCOR3_chr1_+_211485696_{—} |
| 10919256_10916643_10922939 | 131890347_131885417_131891263 | 211485829_211477482_211486061 |
| DPH7_chr9_−_140470160_{—} | PTPRS_chr19_−_5216730_{—} | STK36_chr2_+_219553113_{—} |
| 140470344_140469295_140470531 | 5216778_5215606_5218430 | 219553216_219549951_219553419 |
| DPP9_chr19_−_4683201_{—} | PTRH2_chr17_−_57776231_{—} | ACIN1_chr14_−_23536522_{—} |
| 4683336_4682850_4683488 | 57777550_57775339_57784731 | 23537880_23535217_23538684 |
| DPY30_chr2_−_32117060_{—} | PTS_chr11_+_112100930_{—} | ATG4B_chr2_+_242592721_{—} |
| 32117203_32095021_32142994 | 112100953_112099396_112101348 | 242592784_242590750_242592926 |
| DPY30_chr2_−_32248745_{—} | PVT1_chr8_+_129010445_{—} | SRRM1_chr1_+_24973569_{—} |
| 32248877_32248543_32254662 | 129010605_129001537_129021835 | 24973699_24973280_24975349 |
| DST_chr6_−_56333779_{—} | PXN_chr12_−_120653362_{—} | TMEM214_chr2_+_27260657_{—} |
| 56333797_56330993_56334680 | 120653464_120653076_120659425 | 27260760_27260570_27261013 |
| DYRK4_chr12_+_4716492_{—} | PXN_chr12_−_120653362_{—} | TMEM214_chr2_+_27260657_{—} |
| 4716553_4714252_4719320 | 120653464_120653220_120659425 | 27260760_27260570_27261569 |
| EBPL_chr13_−_50243912_{—} | PXN_chr12_−_120654075_{—} | IL4R_chr16_+_27352390_{—} |
| 50243982_50237331_50265389 | 120654384_120653464_120659425 | 27352634_27351594_27353441 |
| ECHDC2_chr1_−_53370705_{—} | PYROXD1_chr12_+_21605014_{—} | TMEM222_chr1_+_27657475_{—} |
| 53372283_53364896_53373539 | 21605088_21602625_21608065 | 27657628_27657295_27658560 |
| EDRF1_chr10_+_127417571_{—} | QTRT2_chr3_+_113786833_{—} | SRRM2_chr16_+_2818505_{—} |
| 127417673_127414407_127417926 | 113786910_113785130_113789472 | 2818600_2818262_2818997 |
| EED_chr11_+_85979497_{—} | RAB11FIP3_chr16_+_541133_{—} | NSRP1_chr17_+_28483557_{—} |
| 85979603_85977258_85988021 | 541268_539000_546823 | 28483680_28445191_28499559 |
| EHBP1_chr2_+_63215065_{—} | RABGEF1_chr7_+_66204982_{—} | BRE_chr2_+_28550140_{—} |
| 63215173_63206470_63217850 | 66205779_66177211_66236869 | 28550314_28521358_28561316 |
| EIF2D_chr1_−_206773086_{—} | RABGGTB_chr1_+_76255836_{—} | SRSF4_chr1_−_29486375_{—} |
| 206773190_206772446_206773616 | 76255959_76255742_76256969 | 29486570_29485998_29486886 |
| ELK1_chrX_−_47509319_{—} | RBM10_chrX_+_47034417_{—} | NAP1L4_chr11_−_2970456_{—} |
| 47509425_47500874_47509821 | 47034491_47032596_47035898 | 2970494_2966876_2972488 |
| EMC8_chr16_−_85813984_{—} | RBM26_chr13_−_79927287_{—} | MAZ_chr16_+_29820860_{—} |
| 85814079_85813473_85814816 | 79927359_79918929_79928573 | 29821085_29820062_29821397 |
| ENAH_chr1_−_225692692_{—} | RBM3_chrX_+_48434202_{—} | GATSL3_chr22_−_30684694_{—} |
| 225692755_225688772_225695652 | 48434471_48434055_48434701 | 30684765_30683549_30685373 |
| ENOSF1_chr18_−_678695_{—} | RBM3_chrX_+_48434309_{—} | ASXL1_chr20_+_30959580_{—} |
| 678737_677872_683245 | 48434471_48434055_48434701 | 30959677_30956926_30959966 |
| ENTPD6_chr20_+_25187711_{—} | RBM41_chrX_−_106356626_{—} | SMTN_chr22_+_31496870_{—} |
| 25188033_25176503_25190484 | 106356698_106332069_106358581 | 31497035_31495882_31500301 |
| ENTPD6_chr20_+_25187714_{—} | RBM4B_chr11_−_66433742_{—} | DTNA_chr18_+_32464691_{—} |
| 25188033_25176503_25190484 | 66433964_66433049_66436085 | 32464740_32462165_32470263 |
| EP400NL_chr12_+_132588541_{—} | RBM5_chr3_+_50137964_{—} | NT5C3A_chr7_−_33075545_{—} |
| 132588715_132575488_132593116 | 50138038_50137484_50140515 | 33075600_33066527_33102179 |
| EPB41L1_chr20_+_34783250_{—} | RBM5_chr3_+_50147811_{—} | ZNF263_chr16_+_3335680_{—} |
| 34783286_34782282_34785780 | 50147896_50147121_50148111 | 3335754_3334205_3336022 |
| EPB41L1_chr20_+_34797409_{—} | RBM6_chr3_+_50012755_{—} | AK2_chr1_−_33497144_{—} |
| 34797820_34785963_34800193 | 50012825_50000118_50085677 | 33497262_33490168_33502336 |
| EPB41L2_chr6_−_131201283_{—} | RCC1_chr1_+_28834639_{—} | EVA1C_chr21_+_33873724_{—} |
| 131201346_131191266_131206235 | 28834672_28832596_28843236 | 33873848_33867480_33887123 |
| ERBIN_chr5_+_65370851_{—} | REPS1_chr6_−_139247537_{—} | CPNE1_chr20_−_34243123_{—} |
| 65371058_65350779_65372143 | 139247618_139242261_139251113 | 34243266_34220845_34252681 |
| ESYT2_chr7_−_158545471_{—} | RFC5_chr12_+_118455494_{—} | RBM39_chr20_−_34328446_{—} |
| 158545534_158542414_158552176 | 118455858_118454697_118456876 | 34328519_34326939_34328745 |
| ETHE1_chr19_−_44030352_{—} | RFC5_chr12_+_118455799_{—} | DONSON_chr21_−_34954470_{—} |
| 44030501_44015718_44030666 | 118455858_118454697_118456876 | 34954552_34954361_34956895 |
| EVI5L_chr19_+_7921977_{—} | RHBDF1_chr16_−_109296_{—} | DONSON_chr21_−_34955793_{—} |
| 7922010_7920954_7923076 | 109347_109091_109416 | 34955972_34954361_34956895 |
| EWSR1_chr22_+_29687550_{—} | RHOC_chr1_−_113247721_{—} | C20orf24_chr20_+_35236292_{—} |
| 29687588_29684775_29688125 | 113247823_113246428_113249699 | 35236403_35236221_35238003 |
| EWSR1_chr22_+_29687553_{—} | RHOC_chr1_−_113247721_{—} | SRSF3_chr6_+_36567597_{—} |
| 29687588_29684775_29688125 | 113248874_113246428_113249699 | 36568053_36566760_36568928 |
| EXD3_chr9_−_140269063_{—} | RHOT1_chr17_+_30536368_{—} | CWC25_chr17_−_36966528_{—} |
| 140269215_140268051_140277764 | 30536464_30535328_30551634 | 36966653_36966018_36966720 |
| EXOC1_chr4_+_56755053_{—} | RIC8B_chr12_+_107273552_{—} | GOLGA4_chr3_+_37402733_{—} |
| 56755098_56750094_56756388 | 107273725_107254190_107279681 | 37402796_37396678_37407570 |
| EXOC7_chr17_−_74086409_{—} | RILP_chr17_−_1550199_{—} | PLA2G6_chr22_−_38521645_{—} |
| 74086478_74085401_74090494 | 1550283_1549913_1551128 | 38521698_38519265_38522377 |
| EXOSC1_chr10_−_99198419_{—} | RIOK3_chr18_+_21047362_{—} | TMEM184B_chr22_−_38642790_{—} |
| 99198460_99197507_99200927 | 21047490_21046240_21053392 | 38642891_38642106_38643775 |
| EZH1_chr17_−_40871122_{—} | RNF146_chr6_+_127606352_{—} | TMEM184B_chr22_−_38642811_{—} |
| 40871225_40865407_40872290 | 127606493_127601485_127607194 | 38642891_38642106_38643775 |
| F3_chr1_−_95006127_{—} | RNF19A_chr8_−_101312755_{—} | HNRNPLL_chr2_−_38805061_{—} |
| 95006622_95005924_95007092 | 101312879_101300495_101322094 | 38805144_38804674_38809054 |
| FAAH_chr1_+_46871709_{—} | RNFT1_chr17_−_58037428_{—} | SRSF7_chr2_−_38976039_{—} |
| 46871750_46871466_46874130 | 58037529_58035805_58039900 | 38976315_38975795_38976670 |
| FAM111A_chr11_+_58910969_{—} | RNPEP_chr1_+_201958510_{—} | SRSF7_chr2_−_38976039_{—} |
| 58913329_58910777_58919222 | 201958659_201958172_201965274 | 38976488_38975795_38976670 |
| FAM114A2_chr5_−_153390800_{—} | ROGDI_chr16_−_4851267_{—} | THUMPD2_chr2_−_39984387_{—} |
| 153390880_153382529_153405958 | 4851322_4850579_4851503 | 39984508_39983100_39988470 |
| FAM136A_chr2_−_70528539_{—} | RP11-20I23.3_chr16_+_2577561_{—} | NT5C3B_chr17_−_39991839_{—} |
| 70528735_70528112_70529056 | 2577616_2571124_2577773 | 39991894_39991524_39992110 |
| FAM13A_chr4_−_89658622_{—} | RP11-216L13.19_chr9_+_139703706_{—} | NT5C3B_chr17_−_39991839_{—} |
| 89658706_89653349_89660180 | 139703881_139702778_139704139 | 39991934_39991524_39992110 |
| FAM189B_chr1_−_155221523_{—} | RP11-529K1.3_chr16_+_70346511_{—} | HMGN1_chr21_−_40717755_{—} |
| 155221699_155220955_155223415 | 70346560_70333257_70348804 | 40717884_40717200_40719304 |
| FAM192A_chr16_−_57208080_{—} | RP11-848P1.9_chr17_+_29360312_{—} | HMGN1_chr21_−_40717755_{—} |
| 57208198_57207781_57219732 | 29360383_29359524_29360941 | 40717884_40717200_40720217 |
| FAM192A_chr16_−_57208531_{—} | RP4-800G7.2_chr7_+_148985521_{—} | HMGN1_chr21_−_40717755_{—} |
| 57208600_57207781_57219732 | 148986548_148984867_148987028 | 40719218_40717200_40719304 |
| FAM192A_chr16_−_57209450_{—} | RPL17_chr18_−_47018115_{—} | XPNPEP3_chr22_+_41266051_{—} |
| 57209612_57207781_57219732 | 47018203_47017954_47018644 | 41266143_41265119_41277773 |
| FAM192A_chr16_−_57212413_{—} | RPLP0_chr12_−_120636356_{—} | NDUFAF1_chr15_−_41686302_{—} |
| 57212764_57207781_57219732 | 120636434_120635265_120636656 | 41686556_41680720_41687056 |
| FAM192A_chr16_−_57212413_{—} | RPS24_chr10_+_79797722_{—} | SRSF6_chr20_+_42087792_{—} |
| 57212764_57207781_57219942 | 79797740_79797062_79800372 | 42088060_42087149_42088410 |
| FAM192A_chr16_−_57212621_{—} | RPS3A_chr4_+_152022126_{—} | GPATCH8_chr17_−_42544464_{—} |
| 57212764_57207781_57219732 | 152022314_152020866_152024022 | 42544482_42541912_42552196 |
| FAM193B_chr5_−_176959156_{—} | RSRC2_chr12_−_122998313_{—} | TCF20_chr22_−_42564614_{—} |
| 176959201_176958522_176959443 | 122998421_122995735_122999651 | 42564742_42557364_42565852 |
| FAM193B_chr5_−_176974168_{—} | RUBCN_chr3_−_197417944_{—} | HAUS2_chr15_+_42852979_{—} |
| 176974229_176966148_176981249 | 197418019_197411088_197420585 | 42853068_42851606_42853467 |
| FAM193B_chr5_−_176980168_{—} | RUFY1_chr5_+_179013331_{—} | TMEM62_chr15_+_43438690_{—} |
| 176980351_176966148_176981249 | 179013476_179012866_179016546 | 43438832_43427847_43440952 |
| FAM206A_chr9_+_111698587_{—} | S100A13_chr1_−_153600596_{—} | TP53BP1_chr15_−_43738963_{—} |
| 111698717_111697969_111712786 | 153600713_153600074_153603486 | 43739108_43738788_43739563 |
| FAM221A_chr7_+_23731801_{—} | S100A4_chr1_−_153517945_{—} | TP53BP1_chr15_−_43738963_{—} |
| 23734532_23731215_23737810 | 153517994_153517285_153518228 | 43739112_43738788_43739563 |
| FAM221A_chr7_+_23731801_{—} | SBDSP1_chr7_+_72301271_{—} | CORO7_chr16_−_4411749_{—} |
| 23734532_23731215_23740404 | 72301393_72300362_72302181 | 4411804_4411470_4411985 |
| FANCG_chr9_−_35078137_{—} | SBF1_chr22_−_50895462_{—} | ST3GAL3_chr1_+_44386453_{—} |
| 35078340_35077396_35078601 | 50895540_50895102_50897683 | 44386600_44365399_44395803 |
| FANCL_chr2_−_58390163_{—} | SCRIB_chr8_−_144886737_{—} | ATP6V0B_chr1_+_44441336_{—} |
| 58390209_58388773_58390568 | 144886995_144886326_144887103 | 44441520_44440779_44441761 |
| FAR2P2_chr2_−_131183738_{—} | SCRIB_chr8_−_144889721_{—} | ZNF223_chr19_+_44567417_{—} |
| 131183836_131182731_131185276 | 144889784_144889183_144890778 | 44567719_44564994_44570216 |
| FAT1_chr4_−_187511521_{—} | SEC11A_chr15_−_85223162_{—} | NMRALI_chr16_−_4521314_{—} |
| 187511557_187510374_187516842 | 85223200_85214013_85230855 | 4521450_4519466_4524093 |
| FBXL6_chr8_−_145581098_{—} | SEC16A_chr9_−_139339503_{—} | PRPF39_chr14_+_45565626_{—} |
| 145581162_145580781_145581287 | 139339563_139338352_139340096 | 45565961_45565431_45566089 |
| FBXL6_chr8_−_145581116_{—} | SEC16A_chr9_−_139339503_{—} | PRPF39_chr14_+_45565798_{—} |
| 145581162_145580781_145581287 | 139339563_139338352_139341306 | 45565961_45565431_45566089 |
| FBXO31_chr16_−_87392016_{—} | SEC16A_chr9_−_139340096_{—} | SPATA5L1_chr15_+_45699226_{—} |
| 87392103_87380856_87393900 | 139340171_139338352_139341306 | 45699421_45697703_45702569 |
| FCGRT_chr19_+_50024951_{—} | SEC31A_chr4_−_83763292_{—} | TTC38_chr22_+_46665038_{—} |
| 50025070_50016730_50027763 | 83763634_83750211_83765538 | 46665186_46664488_46668231 |
| FLAD1_chr1_+_154962035_{—} | SEC31A_chr4_−_83763337_{—} | POFUT2_chr21_−_46685936_{—} |
| 154962183_154961325_154962634 | 83763634_83750211_83765538 | 46686142_46685550_46687504 |
| FLOT2_chr17_−_27211242_{—} | SEC31A_chr4_−_83782783_{—} | TTC38_chr22_+_46688099_{—} |
| 27211333_27210249_27212874 | 83782861_83778917_83784470 | 46688225_46685796_46688687 |
| FLOT2_chr17_−_27211242_{—} | SEC61A2_chr10_+_12197776_{—} | MKNK1_chr1_−_47051545_{—} |
| 27211333_27210249_27215962 | 12197930_12191960_12198905 | 47051646_47049001_47059784 |
| FLOT2_chr17_−_27212874_{—} | SERHL2_chr22_+_42959394_{—} | DHX34_chr19_+_47880171_{—} |
| 27212965_27211333_27215962 | 42959487_42956271_42967126 | 47880246_47879817_47880356 |
| FNIP1_chr5_−_131046270_{—} | SETD3_chr14_−_99927528_{—} | HDAC7_chr12_−_48186320_{—} |
| 131046354_131044965_131052257 | 99927677_99925522_99929822 | 48186452_48185787_48187151 |
| FOLH1_chr11_−_49179503_{—} | SETD4_chr21_−_37429681_{—} | ROGDI_chr16_−_4851050_{—} |
| 49179595_49178359_49186256 | 37429775_37429502_37431113 | 4851322_4850579_4851503 |
| FOLH1_chr11_−_49228329_{—} | SF1_chr11_−_64540901_{—} | SMAD4_chr18_+_48603676_{—} |
| 49228426_49227724_49229843 | 64540977_64537880_64543969 | 48603789_48603146_48604625 |
| FOPNL_chr16_−_15976805_{—} | SHMT2_chr12_+_57625263_{—} | UBE2V1_chr20_−_48713019_{—} |
| 15976877_15973745_15977864 | 57625343_57624783_57625495 | 48713357_48700791_48729643 |
| FOXRED1_chr11_+_126144821_{—} | SHROOM1_chr5_−_132161968_{—} | KANSL2_chr12_−_49056342_{—} |
| 126144916_126143349_126145221 | 132162279_132161874_132166285 | 49056437_49054402_49061475 |
| FOXRED1_chr11_+_126144825_{—} | SLC12A7_chr5_−_1056696_{—} | LMBRIL_chr12_−_49500293_{—} |
| 126144916_126143349_126145221 | 1056711_1053597_1057585 | 49500529_49499740_49500743 |
| FRG1_chr4_+_190874222_{—} | SLC20A1_chr2_+_113410465_{—} | LMBRIL_chr12_−_49500327_{—} |
| 190874280_190873442_190878552 | 113410524_113410375_113414698 | 49500529_49499740_49500743 |
| FRRS1_chr1_−_100176910_{—} | SLC25A13_chr7_−_95864113_{—} | LMBRIL_chr12_−_49500436_{—} |
| 100177088_100176505_100177660 | 95864229_95838289_95906507 | 49500529_49499740_49500743 |
| FUS_chr16_+_31198122_{—} | SLC25A45_chr11_−_65144803_{—} | SNRNP70_chr19_+_49605370_{—} |
| 31198157_31196500_31199645 | 65144894_65144547_65146846 | 49605430_49604728_49607890 |
| FXR1_chr3_+_180688862_{—} | SLC27A3_chr1_+_153750236_{—} | SNRNP70_chr19_+_49605370_{—} |
| 180688943_180688146_180693100 | 153750361_153749696_153750636 | 49605442_49604728_49607890 |
| FXYD3_chr19_+_35610071_{—} | SLC29A2_chr11_−_66133901_{—} | SNRNP70_chr19_+_49605370_{—} |
| 35610155_35607263_35610266 | 66134035_66133702_66134934 | 49606844_49604728_49607890 |
| G6PC3_chr17_+_42152336_{—} | SLC38A10_chr17_−_79223869_{—} | TBC1D17_chr19_+_50382579_{—} |
| 42152455_42152138_42152677 | 79223893_79220861_79225292 | 50382761_50381829_50383535 |
| GALK2_chr15_+_49611168_{—} | SLC38A5_chrX_−_48324401_{—} | CERS5_chr12_−_50538551_{—} |
| 49611313_49584734_49611800 | 48324480_48321365_48325185 | 50538752_50537840_50560883 |
| GATAD2A_chr19_+_19612777_{—} | SLC4A7_chr3_−_27472788_{—} | LETMD1_chr12_+_51445874_{—} |
| 19612852_19612225_19613139 | 27473160_27465643_27475406 | 51445990_51442968_51450132 |
| GBP3_chr1_−_89476586_{—} | SLK_chr10_+_105770573_{—} | MBD2_chr18_−_51714083_{—} |
| 89476799_89475177_89478867 | 105770666_105768114_105777917 | 51714207_51692536_51715243 |
| GCFC2_chr2_−_75919102_{—} | SLTM_chr15_−_59182479_{—} | DCUN1D4_chr4_+_52753288_{—} |
| 75919226_75917845_75921366 | 59182660_59181753_59185095 | 52753410_52752804_52757925 |
| GEMIN4_chr17_−_653053_{—} | SLTM_chr15_−_59191667_{—} | NUP88_chr17_−_5321334_{—} |
| 653149_651272_655372 | 59192082_59191051_59193458 | 5321461_5320002_5322673 |
| GFM2_chr5_−_74026084_{—} | SLTM_chr15_−_59191667_{—} | HRAS_chr11_−_533276_{—} |
| 74026223_74021951_74028846 | 59192136_59191051_59193458 | 533358_532755_533452 |
| GGT1_chr22_+_25004956_{—} | SMARCA2_chr9_+_2170418_{—} | RDH13_chr19_−_55559413_{—} |
| 25005198_25003990_25005931 | 2170472_2161903_2181570 | 55559625_55558856_55559696 |
| GGT1_chr22_+_25004956_{—} | SMARCA4_chr19_+_11144442_{—} | NPEPL1_chr20_+_57280553_{—} |
| 25005198_25003990_25006324 | 11144541_11144193_11144798 | 57280625_57276214_57282178 |
| GGT5_chr22_−_24628018_{—} | SMARCC2_chr12_−_56558086_{—} | TMX2_chr11_+_57505307_{—} |
| 24628176_24627498_24640520 | 56558152_56557549_56558431 | 57505498_57505140_57505825 |
| GIT2_chr12_−_110383064_{—} | SMIM7_chr19_−_16764845_{—} | CTNND1_chr11_+_57556508_{—} |
| 110383154_110377052_110385060 | 16764936_16758072_16770205 | 57556627_57529518_57558963 |
| GLT8D1_chr3_−_52738739_{—} | SMPD4_chr2_−_130917085_{—} | CTNND1_chr11_+_57556508_{—} |
| 52738968_52734512_52739462 | 130917207_130914969_130918758 | 57556627_57529518_57561481 |
| GMFB_chr14_−_54948839_{—} | SMPDL3A_chr6_+_123116821_{—} | CTNND1_chr11_+_57556508_{—} |
| 54948920_54948176_54950388 | 123117035_123110603_123117968 | 57556627_57529518_57563048 |
| GOLGA2_chr9_−_131035063_{—} | SMPDL3A_chr6_+_123122454_{—} | SLMAP_chr3_+_57911571_{—} |
| 131035144_131030803_131036128 | 123122551_123118113_123124808 | 57911661_57908750_57913022 |
| GOLGA2P7_chr15_−_84873982_{—} | SMPDL3A_chr6_+_123122454_{—} | FLNB_chr3_+_58138434_{—} |
| 84874071_84873727_84898653 | 123122551_123118113_123126053 | 58138581_58135729_58139101 |
| GOLGA8A_chr15_−_34727583_{—} | SMTN_chr22_+_31489769_{—} | STX3_chr11_+_59564755_{—} |
| 34727672_34699936_34729598 | 31489862_31487468_31491288 | 59564869_59562955_59568327 |
| GPATCH2_chr1_−_217668356_{—} | SMTN_chr22_+_31496870_{—} | AHSA2_chr2_+_61410681_{—} |
| 217668417_217665092_217671697 | 31496939_31495882_31500301 | 61410797_61408540_61411846 |
| GTPBP1_chr22_+_39104849_{—} | SNHG16_chr17_+_74555026_{—} | SLC38A6_chr14_+_61514868_{—} |
| 39104961_39102152_39111911 | 74555125_74553939_74557369 | 61515015_61512885_61517229 |
| GUF1_chr4_+_44690023_{—} | SNHG5_chr6_−_86387508_{—} | TPM1_chr15_+_63353396_{—} |
| 44690163_44688730_44691302 | 86387593_86387210_86387671 | 63353472_63353138_63353911 |
| GUK1_chr1_+_228329326_{—} | SNHG5_chr6_−_86387512_{—} | TPM1_chr15_+_63353911_{—} |
| 228329530_228328989_228333211 | 86387593_86387210_86387671 | 63353987_63353472_63354413 |
| GUSB_chr7_−_65435268_{—} | SNRPA1_chr15_−_101826418_{—} | TPM1_chr15_+_63356262_{—} |
| 65435353_65432894_65439281 | 101826498_101826006_101827112 | 63356389_63354844_63358094 |
| GUSB_chr7_−_65444713_{—} | SNRPA1_chr15_−_101833229_{—} | USP3_chr15_+_63821212_{—} |
| 65444898_65441189_65445210 | 101833377_101827907_101835301 | 63821365_63797029_63824845 |
| GUSB_chr7_−_65444713_{—} | SNRPG_chr2_−_70516481_{—} | USP3_chr15_+_63826001_{—} |
| 65444898_65444528_65445210 | 70516504_70515324_70520749 | 63826117_63824906_63829223 |
| GUSB_chr7_−_65444820_{—} | SON_chr21_+_34944856_{—} | NUDT22_chr11_+_63997321_{—} |
| 65444898_65441189_65445210 | 34944936_34941393_34945613 | 63997542_63997043_63997654 |
| GUSB_chr7_−_65444820_{—} | SORBS2_chr4_−_186605907_{—} | VPS54_chr2_−_64140958_{—} |
| 65444898_65444528_65445210 | 186605996_186599976_186696380 | 64141059_64140444_64141315 |
| HACL1_chr3_−_15626754_{—} | SORBS2_chr4_−_186605907_{—} | TMEM5_chr12_+_64176320_{—} |
| 15626849_15624496_15631046 | 186606000_186599976_186696380 | 64176484_64174954_64178749 |
| HAPLN3_chr15_−_89422648_{—} | SPAG9_chr17_−_49053223_{—} | RAB40C_chr16_+_646316_{—} |
| 89422683_89422500_89424587 | 49053262_49052308_49054468 | 646448_640433_667207 |
| HDAC6_chrX_+_48676626_{—} | SPIN1_chr9_+_91033692_{—} | TM7SF2_chr11_+_64883246_{—} |
| 48676819_48676516_48681029 | 91033866_91003453_91041296 | 64883294_64882866_64883364 |
| HDAC6_chrX_+_48678512_{—} | SPIN1_chr9_+_91033764_{—} | UHRF2_chr9_+_6492302_{—} |
| 48678662_48676516_48681029 | 91033866_91003453_91041296 | 6492401_6486925_6493825 |
| HDDC2_chr6_−_125619859_{—} | SPOP_chr17_−_47714120_{—} | ARFIP2_chr11_−_6499808_{—} |
| 125619962_125614055_125621683 | 47714171_47700238_47723477 | 6499882_6499428_6499967 |
| HDHD2_chr18_−_44663605_{—} | SPTAN1_chr9_+_131355261_{—} | GUSB_chr7_−_65444385_{—} |
| 44663667_44662820_44676748 | 131355321_131353904_131356453 | 65444528_65441189_65445210 |
| HDHD2_chr18_−_44663605_{—} | SPTAN1_chr9_+_131391403_{—} | SREK1_chr5_+_65451892_{—} |
| 44663709_44662820_44676748 | 131391466_131390221_131392599 | 65454760_65449424_65455046 |
| HEATR5B_chr2_−_37217790_{—} | SRPK2_chr7_−_104909252_{—} | SREK1_chr5_+_65454636_{—} |
| 37217942_37216002_37227728 | 104909316_104844232_105029094 | 65454760_65449424_65455046 |
| HEXA_chr15_−_72646031_{—} | SRSF2_chr17_−_74731853_{—} | CRCP_chr7_+_65595113_{—} |
| 72646078_72643575_72647899 | 74731957_74731240_74732235 | 65595235_65592727_65595730 |
| HEXA_chr15_−_72646031_{—} | SSBP3_chr1_−_54723741_{—} | DPP8_chr15_−_65748049_{—} |
| 72646078_72645519_72647899 | 54723822_54722859_54747110 | 65748179_65746753_65748562 |
| HINFP_chr11_+_119002952_{—} | ST7_chr7_+_116738666_{—} | XAF1_chr17_+_6662574_{—} |
| 119003007_119002692_119003205 | 116738869_116593745_116739815 | 6662838_6661543_6662980 |
| HMGN1_chr21_−_40719172_{—} | ST7_chr7_+_116774177_{—} | RPS6KB2_chr11_+_67199823_{—} |
| 40719218_40717200_40719304 | 116774246_116772014_116776134 | 67199963_67198986_67200070 |
| HMGN3_chr6_−_79911779_{—} | ST7_chr7_+_116830186_{—} | RPS6KB2_chr11_+_67199826_{—} |
| 79911872_79911443_79911992 | 116830322_116829447_116830887 | 67199963_67198986_67200070 |
| HNRNPA1_chr12_+_54676862_{—} | ST7L_chr1_−_113159434_{—} | CHKA_chr11_−_67828959_{—} |
| 54677018_54676658_54677595 | 113159517_113153625_113161530 | 67829297_67821514_67829419 |
| HNRNPA2B1_chr7_−_26230612_{—} | STAU1_chr20_−_47795658_{—} | MCRIP2_chr16_+_696471_{—} |
| 26230748_26230080_26232114 | 47795781_47770608_47804652 | 696608_692249_697416 |
| HNRNPAB_chr5_+_177637132_{—} | STIM1_chr11_+_4105902_{—} | PPFIA1_chr11_+_70228193_{—} |
| 177637273_177636448_177637553 | 4105995_4104728_4107706 | 70228264_70224301_70229108 |
| HNRNPH1_chr5_−_179046269_{—} | STRN3_chr14_−_31398406_{—} | SLC25A16_chr10_−_70276840_{—} |
| 179046361_179045324_179047892 | 31398517_31381388_31404368 | 70276942_70276600_70287002 |
| HNRNPH1_chr5_−_179046269_{—} | STX8_chr17_−_9460750_{—} | SLC25A16_chr10_−_70276840_{—} |
| 179046408_179045324_179047892 | 9460845_9408479_9471687 | 70277022_70276600_70287002 |
| HNRNPH3_chr10_+_70097614_{—} | STYXL1_chr7_−_75643059_{—} | COG4_chr16_−_70549770_{—} |
| 70097753_70097090_70098259 | 75643205_75634722_75651168 | 70549943_70548412_70551528 |
| HNRNPK_chr9_−_86588816_{—} | SUPT20H_chr13_−_37599456_{—} | SF3B3_chr16_+_70584552_{—} |
| 86588888_86588314_86589431 | 37599574_37598579_37600340 | 70584581_70582345_70588348 |
| HNRNPM_chr19_+_8527412_{—} | SVIL_chr10_−_29797270_{—} | SRSF11_chr1_+_70697541_{—} |
| 8527465_8520458_8528380 | 29797318_29788192_29801663 | 70697658_70694238_70697950 |
| IDE_chr10_−_94240549_{—} | SVIL_chr10_−_29820930_{—} | NADSYN1_chr11_+_71187078_{—} |
| 94240673_94239178_94243011 | 29821101_29820217_29839525 | 71188484_71185572_71189440 |
| IFI44_chr1_+_79126238_{—} | SVIL_chr10_−_29821457_{—} | NAGK_chr2_+_71302691_{—} |
| 79126339_79125168_79128388 | 29822387_29820217_29839525 | 71302772_71300724_71304687 |
| IFI44_chr1_+_79126238_{—} | SVIL_chr10_−_29824917_{—} | ZRANB2_chr1_−_71531360_{—} |
| 79126376_79125168_79128388 | 29824998_29820217_29839525 | 71531435_71530820_71532458 |
| IFT172_chr2_−_27688277_{—} | SYNE2_chr14_+_64682003_{—} | TBL2_chr7_−_72990870_{—} |
| 27688385_27686048_27693794 | 64682072_64681188_64682965 | 72991031_72988843_72992749 |
| IFT172_chr2_−_27688612_{—} | SYNE4_chr19_−_36497503_{—} | ABHD11_chr7_−_73152205_{—} |
| 27688749_27686048_27693794 | 36497573_36496339_36497651 | 73152402_73152065_73152657 |
| IFT43_chr14_+_76524984_{—} | SYNE4_chr19_−_36497503_{—} | ABHD11_chr7_−_73152205_{—} |
| 76525017_76488737_76548637 | 36497573_36496339_36499455 | 73152472_73152065_73152657 |
| IFT81_chr12_+_110581186_{—} | SYNE4_chr19_−_36498026_{—} | NUP85_chr17_+_73225092_{—} |
| 110581350_110574665_110584757 | 36498170_36496339_36499455 | 73225226_73222252_73227434 |
| IFT88_chr13_+_21237401_{—} | SZRD1_chr1_+_16717869_{—} | TMEM94_chr17_+_73488319_{—} |
| 21237525_21230569_21237636 | 16717919_16693803_16721532 | 73488418_73487981_73488554 |
| IGHMBP2_chr11_+_68697790_{—} | TAMM41_chr3_−_11868191_{—} | MTO1_chr6_+_74181182_{—} |
| 68697902_68696825_68700766 | 11868279_11858811_11871187 | 74181280_74176329_74183087 |
| ILF3_chr19_+_10791021_{—} | TARBP1_chr1_−_234536598_{—} | COQ6_chr14_+_74422153_{—} |
| 10791119_10790603_10791694 | 234536694_234534299_234536926 | 74422212_74420272_74422507 |
| IMMP1L_chr11_−_31477806_{—} | TAX1BP1_chr7_+_27856508_{—} | JMJD6_chr17_−_74717344_{—} |
| 31477933_31455117_31531065 | 27856657_27839709_27867356 | 74717433_74716580_74717879 |
| IMMP1L_chr11_−_31484718_{—} | TBC1D1_chr4_+_38054726_{—} | JMJD6_chr17_−_74717344_{—} |
| 31484852_31455117_31531065 | 38054846_38051519_38055819 | 74717438_74716580_74717879 |
| IMMP2L_chr7_−_111172573_{—} | TBC1D22B_chr6_+_37259018_{—} | ABCD4_chr14_−_74764632_{—} |
| 111172641_111161505_111201906 | 37259133_37254848_37280693 | 74764772_74763152_74766250 |
| ING3_chr7_+_120604795_{—} | TBC1D23_chr3_+_100030676_{—} | TMEM70_chr8_+_74891313_{—} |
| 120604892_120595678_120606679 | 100030721_100029386_100034942 | 74891406_74891096_74893389 |
| INO80C_chr18_−_33067349_{—} | TBCE_chr1_+_235564817_{—} | FAM219B_chr15_−_75197322_{—} |
| 33067403_33060527_33077682 | 235564902_235543464_235577747 | 75197380_75197053_75197494 |
| INO80E_chr16_+_30012734_{—} | TBCE_chr1_+_235596260_{—} | FAM219B_chr15_−_75197322_{—} |
| 30012851_30012361_30016541 | 235596413_235594119_235597518 | 75197400_75197053_75197494 |
| INO80E_chr16_+_30012785_{—} | TBRG4_chr7_−_45143697_{—} | RHBDD2_chr7_+_75510682_{—} |
| 30012851_30012361_30016541 | 45143855_45143042_45144136 | 75510804_75508578_75511146 |
| INO80E_chr16_+_30014756_{—} | TBRG4_chr7_−_45144136_{—} | AFMID_chr17_+_76201520_{—} |
| 30014847_30012851_30015888 | 45144308_45143042_45145039 | 76201599_76200981_76202026 |
| INO80E_chr16_+_30014970_{—} | TBX3_chr12_−_115117717_{—} | BIRC5_chr17_+_76218908_{—} |
| 30014991_30012851_30015888 | 115117777_115117456_115118683 | 76219073_76210870_76219545 |
| INO80E_chr16_+_30015875_{—} | TCOF1_chr5_+_149771106_{—} | BIRC5_chr17_+_76218908_{—} |
| 30015978_30012361_30016541 | 149771358_149769586_149771519 | 76219073_76212862_76219545 |
| INO80E_chr16_+_30015888_{—} | TDG_chr12_+_104376913_{—} | PDDC1_chr11_−_773427_{—} |
| 30015978_30012851_30016541 | 104376996_104376712_104377072 | 773629_772521_774007 |
| INTS4_chr11_−_77622015_{—} | TEAD1_chr11_+_12900435_{—} | PQLC1_chr18_−_77690227_{—} |
| 77622198_77618856_77629866 | 12900447_12886447_12901254 | 77690311_77679400_77693968 |
| IPO9_chr1_+_201804081_{—} | TEP1_chr14_−_20840891_{—} | PQLC1_chr18_−_77690227_{—} |
| 201804192_201798500_201816406 | 20841016_20839791_20841169 | 77690311_77679400_77703328 |
| IQCB1_chr3_−_121507130_{—} | TEX264_chr3_+_51708286_{—} | TXNL4A_chr18_−_77736717_{—} |
| 121507279_121500721_121508919 | 51708578_51705304_51718428 | 77736787_77733856_77748239 |
| IQSEC1_chr3_−_12944272_{—} | THAP9-AS1_chr4_−_83816844_{—} | IDH3A_chr15_+_78449249_{—} |
| 12944322_12943022_12949848 | 83816927_83816000_83821229 | 78449504_78447617_78449889 |
| KCTD6_chr3_+_58479879_{—} | THOC5_chr22_−_29927066_{—} | ATP5C1_chr10_+_7848936_{—} |
| 58480135_58477896_58484439 | 29927099_29925228_29927819 | 7848973_7844817_7849621 |
| KDM6A_chrX_+_44921891_{—} | TIAL1_chr10_−_121336358_{—} | BAIAP2_chr17_+_79084713_{—} |
| 44921993_44920664_44922666 | 121336417_121336262_121336591 | 79084759_79082309_79089569 |
| KIAA1191_chr5_−_175786464_{—} | TIAL1_chr10_−_121339982_{—} | RPS24_chr10_+_79799958_{—} |
| 175786570_175782752_175786813 | 121340050_121339522_121341433 | 79799983_79797062_79800372 |
| KIAA1468_chr18_+_59947006_{—} | TKFC_chr11_+_61103311_{—} | RPS24_chr10_+_79799961_{—} |
| 59947089_59942706_59947592 | 61103409_61102203_61105412 | 79799983_79797062_79800372 |
| KIF13A_chr6_−_17771344_{—} | TMED5_chr1_−_93624371_{—} | HEXDC_chr17_+_80398348_{—} |
| 17771449_17765177_17772138 | 93624442_93622040_93625685 | 80398489_80397589_80398872 |
| KIF21A_chr12_−_39724043_{—} | TMEM136_chr11_+_120198149_{—} | WDR73_chr15_−_85189203_{—} |
| 39724064_39720126_39724547 | 120198349_120196077_120200685 | 85189316_85189067_85189414 |
| KLHL29_chr2_+_23907268_{—} | TMEM14B_chr6_+_10751365_{—} | ME3_chr11_−_86168407_{—} |
| 23907407_23865720_23914543 | 10751467_10749931_10770309 | 86168801_86161440_86176132 |
| KLK2_chr19_+_51380959_{—} | TMEM168_chr7_−_112424909_{—} | METTL22_chr16_+_8738385_{—} |
| 51381012_51380264_51381659 | 112425008_112415373_112430214 | 8738582_8736422_8739964 |
| KLK2_chr19_+_51380962_{—} | TMEM183A_chr1_+_202986883_{—} | METTL22_chr16_+_8738413_{—} |
| 51381012_51380264_51381659 | 202987047_202985268_202987608 | 8738582_8736422_8739964 |
| KTN1_chr14_+_56130672_{—} | TMEM184A_chr7_−_1589182_{—} | C12orf29_chr12_+_88433924_{—} |
| 56130759_56128330_56133958 | 1589240_1588324_1589489 | 88434042_88429515_88436601 |
| KXD1_chr19_+_18671229_{—} | TMEM260_chr14_+_57088248_{—} | THNSL2_chr2_+_88485069_{—} |
| 18671360_18668724_18672845 | 57088420_57085481_57092099 | 88485243_88478532_88485416 |
| LASIL_chrX_−_64744443_{—} | TMEM62_chr15_+_43430771_{—} | GALNS_chr16_−_88909437_{—} |
| 64744494_64744142_64744844 | 43430817_43427847_43440952 | 88909607_88909237_88923165 |
| LAS1L_chrX_−_64748139_{—} | TMEM62_chr15_+_43470804_{—} | HAPLN3_chr15_−_89422648_{—} |
| 64748249_64744930_64749091 | 43470909_43461875_43473378 | 89423841_89422500_89424587 |
| LIMCH1_chr4_+_41640948_{—} | TNFAIP2_chr14_+_103590711_{—} | POLG_chr15_−_89875448_{—} |
| 41640984_41621457_41646516 | 103590843_103589820_103592646 | 89875605_89873507_89876326 |
| LMAN2L_chr2_−_97403685_{—} | TNFSF10_chr3_−_172229406_{—} | TCF25_chr16_+_89944868_{—} |
| 97403804_97400263_97405590 | 172229449_172227111_172232650 | 89945035_89940267_89949758 |
| LPAR2_chr19_−_19738562_{—} | TOM1L2_chr17_−_17761082_{—} | MBOAT2_chr2_−_9002400_{—} |
| 19738778_19738093_19738900 | 17761169_17754266_17764789 | 9002452_9000894_9002719 |
| LRRC23_chr12_+_7015008_{—} | TOMM34_chr20_−_43577370_{—} | C14orf159_chr14_+_91690022_{—} |
| 7015118_7014923_7015572 | 43577518_43572220_43580473 | 91690139_91681900_91691027 |
| LRRC27_chr10_+_134155716_{—} | TPCN2_chr11_+_68847301_{—} | PPP4R3A_chr14_−_91932604_{—} |
| 134155775_134151199_134158001 | 68847351_68846488_68848867 | 91932760_91931763_91937180 |
| LRRC37B_chr17_+_30372718_{—} | TPM1_chr15_+_63335904_{—} | MED17_chr11_+_93527415_{—} |
| 30372837_30362658_30374779 | 63336030_63335142_63336225 | 93527605_93527201_93528073 |
| LRRC75A-AS1_chr17_+_16342841_{—} | TPTEP1_chr22_+_17092547_{—} | TMEM175_chr4_+_942198_{—} |
| 16343017_16342728_16343498 | 17092783_17083105_17119468 | 942403_941942_944208 |
| LRRC75A-AS1_chr17_+_16342973_{—} | TRA2A_chr7_−_23561739_{—} | ANKRD49_chr11_+_94231067_{—} |
| 16343017_16342728_16343424 | 23562051_23561459_23571407 | 94231130_94230117_94231236 |
| LRRFIP1_chr2_+_238626404_{—} | TRA2A_chr7_−_23561750_{—} | TMEM175_chr4_+_942298_{—} |
| 238626452_238617273_238628165 | 23562051_23561459_23570799 | 942403_941942_944208 |
| LRRFIP1_chr2_+_238647874_{—} | TRA2A_chr7_−_23561750_{—} | NOL8_chr9_−_95082224_{—} |
| 238647952_238617273_238657006 | 23562051_23561459_23571407 | 95082419_95081638_95083949 |
| LRRFIP1_chr2_+_238647874_{—} | TRA2A_chr7_−_23561972_{—} | NOL8_chr9_−_95082224_{—} |
| 238647952_238629465_238657006 | 23562051_23561459_23571407 | 95082661_95081638_95083949 |
| LRRFIP1_chr2_+_238659842_{—} | TRAK2_chr2_−_202265740_{—} | FAM76B_chr11_−_95512241_{—} |
| 238659914_238657967_238661951 | 202265817_202264216_202272125 | 95512299_95512118_95512770 |
| LRRFIP2_chr3_−_37132957_{—} | TRDN_chr6_−_123696749_{—} | FAM76B_chr11_−_95512241_{—} |
| 37133029_37125297_37136282 | 123696776_123687327_123698860 | 95512299_95512121_95512770 |
| LRRFIP2_chr3_−_37146945_{—} | TRIM14_chr9_−_100872350_{—} | LMAN2L_chr2_−_97399255_{—} |
| 37147014_37138151_37162982 | 100872516_100872266_100881263 | 97399338_97377762_97400145 |
| LTBP3_chr11_−_65307483_{—} | TRIP10_chr19_+_6746039_{—} | MMS19_chr10_−_99236591_{—} |
| 65307624_65307352_65307715 | 6746207_6745005_6746462 | 99236676_99236501_99237103 |
| LUC7L_chr16_−_258599_{—} | TRIP4_chr15_+_64706283_{—} | MMS19_chr10_−_99236591_{—} |
| 258663_258187_270647 | 64706410_64702027_64710739 | 99236720_99236501_99237103 |
| LUC7L_chr16_−_278331_{—} | TRIT1_chr1_−_40318402_{—} | AP4M1_chr7_+_99699868_{—} |
| 278401_277335_279277 | 40318548_40315933_40319641 | 99700016_99699591_99700297 |
| MADD_chr11_+_47310518_{—} | TRMT2B_chrX_−_100306239_{—} | |
| 47310578_47308085_47310941 | 100306722_100297301_100306805 | |
| MAN2B1_chr19_−_12769241_{—} | TRMT2B_chrX_−_100306632_{—} | |
| 12769321_12769158_12772073 | 100306722_100297301_100306805 | |

TABLE 1d
Oversap AS CL&Myc

| AS Events | AS Events | AS Events |
|---|---|---|
| SRSF3_chr6_+_36567597_{—} | OXNAD1_chr3_+_16313651_{—} | ABI1_chr10_−_27044583_{—} |
| 36568053_36566760_36568928 | 16313828_16313229_16327848 | 27044670_27040712_27054146 |
| FAM76B_chr11_−_95512241_{—} | TRA2A_chr7_−_23561739_{—} | SETD4_chr21_−_37429681_{—} |
| 95512299_95512121_95512770 | 23562051_23561459_23571407 | 37429775_37429502_37431113 |
| PPP4R3A_chr14_−_91932604_{—} | TRA2A_chr7_−_23561750_{—} | PMPCB_chr7_+_102948042_{—} |
| 91932760_91931763_91937180 | 23562051_23561459_23571407 | 102948155_102944937_102949398 |
| METTL22_chr16_+_8738413_{—} | SLTM_chr15_−_59191667_{—} | MYL6_chr12_+_56553370_{—} |
| 8738582_8736422_8739964 | 59192136_59191051_59193458 | 56553406_56552495_56553758 |
| ME3_chr11_−_86168407_{—} | SLTM_chr15_−_59191667_{—} | TTC31_chr2_+_74717151_{—} |
| 86168801_86161440_86176132 | 59192082_59191051_59193458 | 74717254_74710537_74717370 |
| RPS24_chr10_+_79799961_{—} | MYO18A_chr17_−_27412621_{—} | DNAJC2_chr7_−_102967778_{—} |
| 79799983_79797062_79800372 | 27412666_27409456_27413455 | 102967825_102967131_102968102 |
| JMJD6_chr17_−_74717344_{—} | HNRNPH1_chr5_−_179046269_{—} | TBX3_chr12_−_115117717_{—} |
| 74717433_74716580_74717879 | 179046408_179045324_179047892 | 115117777_115117456_115118683 |
| JMJD6_chr17_−_74717344_{—} | ZNF706_chr8_−_102214560_{—} | TAMM41_chr3_−_11868191_{—} |
| 74717438_74716580_74717879 | 102214675_102213971_102217662 | 11868279_11858811_11871187 |
| ZRANB2_chr1_−_{——}71531360_{—} | DAZAP1_chr19_+_1425876_{—} | ABI1_chr10_−_27044583_{—} |
| 71531435_71530820_71532458 | 1425959_1422395_1428840 | 27044670_27040712_27047990 |
| SRSF11_chr1_+_70697541_{—} | DPP9_chr19_−_4683201_{—} | NAA16_chr13_+_41936866_{—} |
| 70697658_70694238_70697950 | 4683336_4682850_4683488 | 41937009_41936295_41941574 |
| MCRIP2_chr16_+_696471_{—} | SNRPA1_chr15_−_101826418_{—} | SNRPA1_chr15_−_101833229_{—} |
| 696608_692249_697416 | 101826498_101826006_101827112 | 101833377_101827907_101835301 |
| SREK1_chr5_+_65454636_{—} | HNRNPH1_chr5_−_179046269_{—} | ARIH2_chr3_+_48982568_{—} |
| 65454760_65449424_65455046 | 179046361_179045324_179047892 | 48982614_48965246_48999044 |
| SREK1_chr5_+_65451892_{—} | NADSYN1_chr11_+_71191264_{—} | HMGN3_chr6_−_79911779_{—} |
| 65454760_65449424_65455046 | 71191320_71189515_71191800 | 79911872_79911443_79911992 |
| USP3_chr15_+_63826001_{—} | SYNE2_chr14_+_64682003_{—} | MRPL22_chr5_+_154330362_{—} |
| 63826117_63824906_63829223 | 64682072_64681188_64682965 | 154330498_154320825_154335930 |
| USP3_chr15_+_63821212_{—} | AC005154.6_chr7_−_30618621_{—} | ZNF207_chr17_+_30693683_{—} |
| 63821365_63797029_63824845 | 30618744_30617707_30618846 | 30693776_30692506_30694790 |
| HRAS_chr11_−_533276_{—} | FAM136A_chr2_−_70528539_{—} | MMS19_chr10_−_99228722_{—} |
| 533358_532755_533452 | 70528735_70528112_70529056 | 99228861_99228163_99229402 |
| DCUN1D4_chr4_+_52753288_{—} | PSMC5_chr17_+_61905033_{—} | RHOC_chr1_−_113247721_{—} |
| 52753410_52752804_52757925 | 61905283_61904874_61905497 | 113248874_113246428_113249699 |
| SNRNP70_chr19_+_49605370_{—} | PLEKHM2_chr1_+_16047823_{—} | KIAA1191_chr5_−_175786464_{—} |
| 49605430_49604728_49607890 | 16047883_16046415_16051811 | 175786570_175782752_175786813 |
| SNRNP70_chr19_+_49605370_{—} | NACA_chr12_−_57109654_{—} | GUF1_chr4_+_44690023_{—} |
| 49606844_49604728_49607890 | 57109990_57108471_57113320 | 44690163_44688730_44691302 |
| SNRNP70_chr19_+_49605370_{—} | MRPL33_chr2_+_27997290_{—} | SEC31A_chr4_−_83782783_{—} |
| 49605442_49604728_49607890 | 27997397_27995559_28002299 | 83782861_83778917_83784470 |
| HMGN1_chr21_−_40717755_{—} | KIAA1468_chr18_+_59947006_{—} | NDRG2_chr14_−_21492188_{—} |
| 40719218_40717200_40719304 | 59947089_59942706_59947592 | 21492255_21491480_21493835 |
| HMGN1_chr21_−_40717755_{—} | MAP2K7_chr19_+_7970692_{—} | RHOC_chr1_−_113247721_{—} |
| 40717884_40717200_40719304 | 7970740_7968953_7974639 | 113247823_113246428_113249699 |
| RBM39_chr20_−_34328446_{—} | PBRM1_chr3_−_52588739_{—} | DNAJC14_chr12_−_56223272_{—} |
| 34328519_34326939_34328745 | 52588895_52584833_52595782 | 56223420_56222498_56224479 |
| NT5C3A_chr7_−_33075545_{—} | LUC7L_chr16_−_258599_{—} | RFC5_chr12_+_118455494_{—} |
| 33075600_33066527_33102179 | 258663_258187_270647 | 118455858_118454697_118456876 |
| ASXL1_chr20_+_30959580_{—} | ZNF83_chr19_−_53119970_{—} | YTHDC2_chr5_+_112860677_{—} |
| 30959677_30956926_30959966 | 53120128_53118050_53122188 | 112860874_112851059_112868575 |
| SRRM1_chr1_+_24973569_{—} | ZNF83_chr19_−_53119970_{—} | ZNF271P_chr18_+_32870973_{—} |
| 24973699_24973280_24975349 | 53120068_53118050_53122188 | 32871196_32870355_32885939 |
| MINOS1_chr1_+_19927280_{—} | KIF13A_chr6_−_17771344_{—} | ZNF326_chr1_+_90472903_{—} |
| 19927465_19923603_19949967 | 17771449_17765177_17772138 | 90473309_90470803_90475646 |
| TRA2B_chr3_−_185649364_{—} | D2HGDH_chr2_+_242688279_{—} | EDRF1_chr10_+_127417571_{—} |
| 185649640_185644522_185655612 | 242689344_242684292_242689565 | 127417673_127414407_127417926 |
| TRA2B_chr3_−_185649364_{—} | ZNF83_chr19_−_53119970_{—} | LTBP3_chr11_−_65307483_{—} |
| 185649640_185643414_185655612 | 53120094_53118050_53122188 | 65307624_65307352_65307715 |
| MPRIP_chr17_+_17083920_{—} | IFI44_chr1_+_79126238_{—} | DFFB_chr1_+_3782847_{—} |
| 17083983_17083402_17088136 | 79126339_79125168_79128388 | 3782962_3782564_3784537 |
| OXNAD1_chr3_+_16313651_{—} | WNK2_chr9_+_96069058_{—} | DNM1L_chr12_+_32890798_{—} |
| 16313828_16313229_16327848 | 96069103_96060349_96070609 | 32890876_32890095_32891197 |
| MAP3K4_chr6_+_161529982_{—} | HNRNPH3_chr10_+_70097614_{—} | AARSD1_chr17_−_41105740_{—} |
| 161530073_161529891_161530786 | 70097753_70097090_70098259 | 41105795_41103911_41106892 |
| INTS3_chr1_+_153733495_{—} | ESYT2_chr7_−_158545471_{—} | MICAL3_chr22_−_18310409_{—} |
| 153733585_153733394_15373445 | 158545534_158542414_158552176 | 18310547_18305826_18314619 |
| THEM4_chr1_−_151862458_{—} | PBRM1_chr3_−_52592264_{—} | CALD1_chr7_+_134620438_{—} |
| 151862690_151861849_151867483 | 52592429_52584833_52595782 | 134620516_134618141_134625842 |
| TCERG1_chr5_+_145889629_{—} | SMARCA4_chr19_+_11144442_{—} | SLC38A5_chrX_−_48324401_{—} |
| 145889723_145888808_145890003 | 11144541_11144193_11144798 | 48324480_48321365_48325185 |
| DDX39A_chr19_−_14521232_{—} | PAOX_chr10_+_135197463_{—} | EBPL_chr13_−_50243912_{—} |
| 14521417_14521146_14521800 | 135197716_135195163_135202459 | 50243982_50237331_50265389 |
| DDX39A_chr19_−_14521359_{—} | CASP6_chr4_−_110617565_{—} | YPEL5_chr2_+_30371110_{—} |
| 14521417_14521146_14521800 | 110617642_110615856_110618777 | 30371407_30369928_30379493 |
| GK5_chr3_−_141903552_{—} | DCAF8_chr1_−_160231074_{—} | DIAPH2_chrX_+_95990756_{—} |
| 141904635_141901891_141904770 | 160231148_160213824_160232238 | 95990789_95940189_95993584 |
| NDUFAF5_chr20_+_13795063_{—} | FOXRED1_chr11_+_126144821_{—} | KTN1_chr14_+_56130672_{—} |
| 13795161_13789548_13797108 | 126144916_126143349_126145221 | 56130759_56128330_56133958 |
| BCLAF1_chr6_−_136590278_{—} | EPB41L1_chr20_+_34797409_{—} | BRD8_chr5_−_137495243_{—} |
| 136590441_136589477_136590574 | 34797820_34785963_34800193 | 137495288_137492956_137495757 |
| PPP2R2D_chr10_+_133748397_{—} | TKFC_chr11_+_61103311_{—} | RP11-216L13.19_chr9_+_139703706_{—} |
| 133748510_133748059_133753534 | 61103409_61102203_61105412 | 139703881_139702778_139704139 |
| TRMT11_chr6_+_126327987_{—} | LUC7L_chr16_−_278331_{—} | NDUFAF7_chr2_+_37469777_{—} |
| 126328100_126320759_126329537 | 278401_277335_279277 | 37469836_37468780_374715 |
| MRRF_chr9_+_125048317_{—} | IFI44_chr1_+_79126238_{—} | FNIP1_chr5_−_131046270_{—} |
| 125048445_125047566_125054027 | 79126376_79125168_79128388 | 131046354_131044965_131052257 |
| TIAL1_chr10_−_121339982_{—} | PSAP_chr10_−_73583644_{—} | SYNE4_chr19_−_36498026_{—} |
| 121340358_121339522_121341433 | 73583653_73581764_73585593 | 36498170_36496339_36499455 |
| LAMP2_chrX_−_119572930_{—} | NAA25_chr12_−_112487286_{—} | CCNDBP1_chr15_+_43482252_{—} |
| 119573148_119565317_119575584 | 112487415_112486247_112491361 | 43482349_43481478_43482522 |
| TAMM41_chr3_−_11874476_{—} | TIAL1_chr10_−_121339982_{—} | TCOF1_chr5_+_149771106_{—} |
| 11874625_11871338_11880695 | 121340050_121339522_121341433 | 149771358_149769586_149771519 |
| MAGOHB_chr12_−_10761696_{—} | ARL4A_chr7_+_12727259_{—} | ZNF326_chr1_+_90473170_{—} |
| 10761982_10760535_10762429 | 12727353_12726668_12727790 | 90473309_90470803_90475646 |
| NT5C2_chr10_−_104871501_{—} | TARBP1_chr1_−_234536598_{—} | CMTM7_chr3_+_32490945_{—} |
| 104871562_104866463_104899162 | 234536694_234534299_234536926 | 32491044_32483505_32493883 |
| SRRT_chr7_+_100480385_{—} | CTPS1_chr1_+_41473120_{—} | FLOT2_chr17_−_27211242_{—} |
| 100480711_100479862_100481690 | 41473217_41471766_41474330 | 27211333_27210249_27212874 |

### C. Methods

### 1. RNA-Seq Data Processing Framework

A comprehensive RNA-Seq dataset was compiled from published prostate cancer and normal prostate datasets that reflect the full progression of prostate cancer. In total, 876 samples were downloaded from different sources. RNA-Seq Fastq files of normal prostate samples (GTEx consortium (31)) and prostate cancer samples (Beltran study (10), Robinson study (11) and Stand-Up-To-Cancer study (12)) were downloaded from dbGAP (85, 86) via fastq-dump in SRA toolkit. RNA-Seq Fastq files from TCGA primary prostate cancer and adjacent benign samples were downloaded from GDC via gdc-client (87).

A unified RNA-Seq processing framework was constructed to perform read mapping as well as gene and isoform quantification on the collected multi-phenotypic prostate RNA-Seq samples. Specifically, read mapping was done by STAR 2.5.3a (88) with a STAR 2-pass function enabled to improve the detection of splicing junctions. The STAR genome index was built with—sjdbOverhang 100 as a generic parameter to handle differences in read length of RNA-Seq samples from various sources. The genome annotation file was downloaded from GENCODE V26 (89) under human genome version hg19 (GRCh37). The subsequent gene/isoform expression quantification is performed by Cufflinks (90) with default parameters.

RNA-Seq alternative splicing quantification is conducted uniformly with a newly engineered version (v4.0.2) of the rMATS-turbo software package (29, 30). An exon-based ratio metric, commonly defined as Percent-Spliced-In (PSI) ratio, was employed to measure the alternative splicing events. The PSI ratio is calculated as follows:

$$ψ = \frac{I / \text{?}}{S / {l}_{s} + I / \text{?}}$$

$$\text{?} \text{indicates text missing or illegible when filed}$$

where S and I are the number of read mapped to the junction supporting skipping and inclusion form. Effective length l is used for normalization.

Customized scripts were applied to calculate PSI value for each individual alternative splicing event from the rMATS-turbo junction count output. To build a confident set of exon events, the splice junction of each event was required to be covered by no less than 10 splice junction reads. Additionally, each event was required to have a PSI range greater than 5% across the entire dataset (|maxPSI−minPSI|>5%), with a mean skipping or inclusion value over 5%. Events with missing values in the majority (over 99%) of samples were removed.

### 2. Analysis and Evaluation of Alternative Splicing Profile of Prostate Cancer Meta-Dataset

Principal component analysis (PCA) was applied to inspect the RNA-Seq derived gene expression/alternative splicing profiles of the multi-phenotypic prostate cancer dataset. First, the matrix of sample vs. FPKM/PSI value was produced by customized scripts. Then, the matrix was completed and imputed by KNN method (knnImputation in DMwR package) (91) for missing values. Lastly, the matrix was mean centered and scaled (PSI matrix is not scaled). PCA was conducted via prcomp function in R. The top five PCs were inspected but only the first two that describe the highest percentage of the variance are shown.

In addition, silhouette width was applied to assess the fitness of the PCA clustering results derived from either alternative splicing or gene/isoform expression measurements (92). Specifically, disease conditions were used as sample labels to compute the silhouette width of each cluster. Average silhouette widths were compared between different metrics based PCA clustering results (93). The R package cluster (94) was used for Silhouette calculation based on PCA results and disease phenotype labels.

3. Gene Ontology (GO) Analysis with Background Correction for Expressed Genes

The GO annotation was queried via the EnrichR (95) API in R. A customized background gene list is required for the proper calculation of over- and under-representation of a GO term (96). For the alternative splicing analysis in this study, the background genes were selected from a set with sufficient sequence coverage at splice junctions to pass the filtering criteria described above. With the customized background list, a corrected p-value can be computed using the hypergeometric test. The Benjamini-Hochberg procedure was used to control for false discovery rate (FDR) at 5%. To reduce complexity, the resulting GO terms were required to contain at least 10 genes. For GO terms displayed in FIG. 2B, the inventors increased the minimum GO term size to 100 to display the most representative GO terms.

To visualize the GO result, the REVIGO (97) web server was employed with customizations in the R plotting scripts for FIGS. 3 and 6. For the REVIGO web settings, default settings were kept except that ‘Homo sapiens’ was selected for the ‘database with GO term size’. In the R script for plotting, log transformed p-value was used for the y-axis, and the ‘semantic distance X’ was used for the x-axis, which was computed based on Multidimensional scaling (MDS) according to the original publication (97). Only Molecular Function (MF) terms are displayed. The displayed terms were selected based on the following rules: first, find the term with dispensability<0.15 (calculated by REVIGO), then from these terms choose the term for the largest term size in the same cluster.

### 4. Pathway Enrichment-Guided Activity Study of Alternative Splicing (PEGASAS)

In order to identify exon incorporation shifts that could correspond to oncogenic pathway alternations during tumor progression, a correlation-based analysis was developed to define signaling pathway correlated alternative splicing events. It involves two major steps:

The first step is to define signaling pathway activity and alternative splicing levels. The quantification of gene expression and alternative splicing is detailed in the RNA-Seq data processing section. Signaling pathway activity can be characterized by assessing the expression level of its target genes as a set relative to other genes (42). The Molecular Signatures Database (MSigDB) (98) has compiled gene sets (42) for the use with gene set enrichment analysis (GSEA) (99) software or similar applications. Here, a group of well-defined gene sets, known as Hallmarks (42), was selected to assess a wide range of pathways in prostate cancers. To measure the activity of a given signaling pathway gene set, all genes (both genes within the gene set as well as those not in the gene set) were ranked according to their gene expression values, then a weight is assigned to each gene based on the number of genes in the set (pathway or non-pathway) they belonged to. This was used to construct empirical distributions for both sets, and a two-sample Kolmogorov-Smirnov (K-S) test statistic, which is the supremum of the differences between the two distributions, was computed as a measure of the activity of the signaling pathway, i.e. an “activity score”. Given the same pathway gene set and gene annotation file, the higher the score, the higher the collective activity of a signaling pathway in a sample. Note that the score should not be used to compare across signaling pathways as each gene set has distinct number of genes, which influences the score.

The second step is to identify pathway activity-correlated alternatively spliced exons. For each pathway, the pathway activity score defined above was correlated with all the AS events identified by rMATS-turbo. The Pearson correlation coefficient was computed for each pathway-exon pair across samples in the tables. A Pearson's correlation coefficient with an absolute value>0.3 was considered as correlated. Data points for each pathway-exon pair were permutated 5,000 times locally to produce empirical p-values to filter out faulty correlations caused by data structure or missing data points. A stringent empirical p-value<2×10⁻⁴ was required for this analysis. The analytical framework performs streamlined analysis of multiple gene sets (e.g. 50 Hallmark sets). Customized scripts were implemented to generate the summary plot.

### 5. Overlap Enrichment Assessment

Hypergeometric test p-value is used to measure the significance of the overlap between two groups of AS events. The triple intersection p-value is calculated by R package ‘SuperExactTest’ based on hypergeometric test (100).

### 6. Breast Cancer and Lung Cancer Myc-Correlated Alternative Splicing Analysis

The same RNA-Seq processing framework described in ‘RNA-Seq data processing’ section was applied to quantify gene expression and alternative splicing of GTEx normal breast and lung samples, and TCGA BRCA and LUAD tumor-adjacent normal samples and tumor samples that are matched to tumor-adjacent normal samples. The Myc pathway-dependent alternative splicing analysis is performed as described in the above section.

### 7. Lentiviral Constructs

The myrAKT1 lentiviral vector has been described previously (101). The inducible Myc lentiviral vector was cloned by inserting MYC into the BamHI site of the PSTV lentiviral backbone. Lentiviruses were prepared and titered as described (101).

### 8. Organotypic Human Prostate Transformation Assay

This assay was conducted as previously described (65, 102). Briefly, benign portions of de-identified, IRB-exempt, human prostatectomy specimens were procured by the UCLA Translational Pathology Core Laboratory. Tissue processing and isolation of the Trop2⁺/CD49f^{hi} basal cells were as described previously. These cells were subjected to lentiviral transformation and placed into 3D organoid culture in a growth factor-reduced Matrigel droplet (Corning, 356230) for 10-14 days. Doxycycline (1 ug/mL, Calbiochem 324385) was added to all culture media and renewed every 3 days.

### 9. Xenograft Outgrowth of Transformed Cells

The xenograft protocol has been previously described (65, 102). 10-14 day old prostate organoids were harvested by centrifugation after Dispase II (Life Technologies 17105041) dissociation of the growth factor-reduced Matrigel (Corning 356231), washed in PBS, placed in standard Matrigel (Corning 356234), and implanted subcutaneously in NSG mice (Jackson Laboratories 005557). Mice were fed sterile doxycycline chow (Bio-Serv S3888) continuously starting three days prior to implantation. Animals were sacrificed and the tumors harvested after 6-8 weeks of outgrowth.

### 10. Cell Line Derivation

Cell line initiation was performed as previously described (102) with the addition of 1 μg/mL doxycycline to all tissue culture media. Harvested tumors were digested to single cell suspension by mincing followed by trypsin and placed on ultra-low attachment plates (Corning 3262) in stem cell media. Stem cell media is composed of advanced DMEM/F12K (Gibco 12634028) base media with addition of B27 (Gibco 17504044), EGF (10 ng/ml, Peprotech 100-47), and FGF2 (10 ng/mL, Peprotech 100-18B) as well as Glutamax (Gibco 35050061). Normocin antibiotic (1:500, InvivoGen ANT-NR-1) was added to the culture for the first two weeks to prevent contamination from the non-sterile preparation and then withdrawn. After two weeks in ultra-low attachment plates, cells were transferred to standard tissue culture plates and remained suspended.

### 11. Myc Withdrawal Experiments

Cells were collected by centrifugation and washed with media three times to remove doxycycline. 1 million cells were plated for each condition. Doxycycline was added back to the appropriate wells and then harvested at the appropriate time point (0-24 h).

### 12. Histology

Portions of xenograft outgrowths were fixed in formalin overnight and transferred to 70% ethanol solution before submission for further processing by the Tissue Procurement Core Laboratory at UCLA (TPCL). Organoids were collected by dispase dissociation from matrigel, washed three times with PBS, and then formalin-fixed for 30 minutes at room temperature. The fixed organoids were again collected by centrifugation and resuspended in Histogel and submitted to TPCL. All samples were paraffin-embedded, sectioned at 4 μm, and mounted on glass slides. Hematoxylin and cosin staining was conducted according to standard protocols.

### 13. Immunohistochemistry

Immunohistochemical studies were conducted as previously described. Briefly, unstained slides were subjected to deparaffinization, rehydration, and heat-activated citric acid antigen retrieval. Rehydrated slides were blocked with 1% horse serum in PBS before overnight incubation with primary antibodies also diluted in 1% horse serum/PBS. Primary and secondary antibodies and their dilutions are listed below. Antibody binding was detected using an HRP-conjugated secondary antibody and a chromogenic substrate.

### 14. Immunoblotting

Portions of tumor xenografts or 10 million cultured cells were placed in 8M urea lysis buffer with protease inhibitors (Sigma-Aldrich 4693159001) and homogenized with a Dounce apparatus. The lysate was cleared by ultracentrifugation at 30,000×g for 30 minutes. Samples were denatured by boiling in SDS loading buffer under reducing conditions for one minute and subjected by polyacrylamide gel electrophoresis. Wet transfer to nitrocellulose membrane was followed by blocking in 1% milk/0.1% Tween/PBS and overnight primary antibody incubation at 5C in the same buffer. HRP-conjugated secondary antibodies were applied after washing and the blot visualized with a pro-luminescent substrate. Semi-quantitative blots of SRSF3 protein levels used PVDF membrane. Fluorescence levels were measured by Typhoon scanner and normalized to GAPDH levels. Antibody sources and dilutions are described below.

### 15. Antibodies for Flow Cytometry, Immunohistochemistry and Immunoblotting

Antibodies used for flow cytometry were the fluorochrome conjugates CD49f-PE (12-0495-82; eBiosciences) and Trop2-APC (FAB650A; R&D Systems). Primary antibodies used for immunohistochemistry include CK8 (1:1,000, Covance MMS-162P), AR (1:250, Santa Cruz sc-816), PSA (KLK3) (1:2000, Dako A0562), CK5 (1:1000, Covance PRB-160P), and p63 (1:250, Santa Cruz sc-8431). Secondary antibodies used were ImmPRESS Anti-Rabbit Ig Peroxidase and Anti-Mouse Ig Peroxidase (Vector Labs). Liquid DAB+substrate reagent (Dako) was used to perform direct chromogenic visualization. Primary antibodies used for immunoblotting include the following, all used at 1:1000 dilution unless otherwise noted: Myc (Abcam ab32072), pan-AKT (Cell Signaling 4691), p53 (Cell Signaling Technology 2527), PARP1 (AbCam ab32138), Cleaved PARP1 antibody (AbCam, ab32064), Anti-Cdk2 (AbCam ab32147), Anti-Cdk2 (phospho Y15) (AbCam ab76146), p21 Anti-p21 antibody [EPR3993] (ab109199), GAPDH (1:5,000, GeneTex GT239). Secondary antibodies used were Goat Anti-Rabbit-HRP Conjugate and Goat Anti-Mouse-HRP Conjugate (BioRad) for luminescent detection. Semi-quantitative Western blots used goat anti-mouse-cy5 (1:5000, Sigma-Aldrich GEPA45009).

### 16. Cell Cycle Analysis

One million cells were withdrawn from doxycycline as described above and harvested by centrifugation at the appropriate timepoint. Cell pellets were washed three times with PBS and then singly dissociated with trypsin prior to fixation in 10% cold ethanol. After overnight fixation at 5° C., cells were pelleted and rehydrated in PBS. RNAse was added and the suspension incubated at RT for 4 h before staining with 20 ng/mL 7AAD and analysis by flow cytometry.

### 17. Cell Growth Assay

Cells were washed with PBS and withdrawn from doxycycline then plated at a density of 100,000 cells per well. Cells were lysed with CellTiterGlo luciferase reagent at the appropriate time and submitted for luminometry.

### 18. Whole Transcriptome Sequencing Analysis

Total RNA was isolated by guanidinium thiocyanate-phenol-chloroform extraction followed by column clean-up. Isolated RNA was submitted for RNA integrity number (RIN) analysis and only samples with RIN>9 were carried forward. cDNA libraries were prepared from isolated RNA after poly-A selection using the TruSeq RNA Sample Prep Kit v2 (Illumina). High-throughput sequencing with 150 bp paired-end reads was performed using an Illumina HiSeq 2500. At least 100M reads were collected for each sample.

### 19. Cell Line Gene Expression and Alternative Splicing Differential Analysis

The same RNA-Seq processing framework described above was applied to quantify gene expression and alternative splicing of Myc cell line samples. Differentially expressed (DE) genes were identified and visualized by the Cuffdiff and cummerbund pipeline with a threshold of q-value<0.05 Skipped exon (SE) events quantified by rMATS-turbo were analyzed by the PAIRADISE statistical model for conducting paired tests of between Myc+/−conditions (90). PAIRADISE with equal·variance=TRUE was used to perform the test (70). The resulting events were first filtered by the coverage and deltaPSI requirements (≥10 splice junction reads per event, |deltaPSI|>0.05). Then, an FDR 5% cutoff was applied to identify significant differential alternative splicing events between the on and off states of the engineered Myc cell line.

### 20. Cell Line Exon Annotations

Exon annotations of known stop codons and the middle exon length were generated based on the same GENCODE gene annotation file used for alignment. Potential frameshift annotation is determined if the middle exon length cannot be divided by three. Potential RNA binding proteins were labeled according to the GO annotation term ‘RNA binding’.

### D. Supplementary Methods

1. Gene Ontology (GO) Analysis with Background Correction for Expressed Genes

The GO annotation was queried via the EnrichR API in R (M. V. Kuleshov et al., Nucleic Acids Res 44, W90-97 (2016)). A customized background gene list is required for the proper calculation of over- and under-representation of a GO term (P. Khatri, S. Draghici, Ontological analysis of gene expression data: current tools, limitations, and open problems. Bioinformatics 21, 3587-3595 (2005)). For the alternative splicing analysis in this study, the background genes were selected by having sufficient coverage at splice junctions to meet the filtering criteria described above. With this customized background list, a corrected p-value can be computed using the hypergeometric test. The Benjamini-Hochberg procedure was used to control for the false discovery rate (FDR) at 5%. To reduce complexity, the resulting GO terms were required to contain at least 10 genes, with an exception for FIG. 2B, where the minimum term size was increased to 100 to display the most representative terms. To visualize GO results, the REVIGO web server was employed with customized R plotting scripts for FIGS. 3 and 6 (F. Supek, et al., PLOS One 6, e21800 (2011)).

### 2. Overlap Enrichment Assessment

Hypergeometric test p-value is used to measure the significance of the overlap between two groups of alternative splicing events. The triple intersection p-value is calculated by R package “SuperExactTest” based on hypergeometric test (M. Wang, et al., Sci Rep 5, 16923 (2015)).

### 3. Breast Cancer and Lung Cancer Myc-Correlated Alternative Splicing Analysis

The RNA-Seq processing framework described above was applied to quantify gene expression and alternative splicing of GTEx normal breast and lung samples, and TCGA BRCA and LUAD tumor-adjacent normal samples and tumor samples that are matched to tumor-adjacent normal samples. These datasets are de-identified. The Myc pathway-dependent splicing analysis was performed as described above.

### 4. Lentiviral Constructs

The myrAKT1 lentiviral vector has been described previously (L. Xin, et al., Proc Natl Acad Sci USA 102, 6942-6947 (2005)). The inducible Myc lentiviral vector was cloned by inserting MYC into the BamHI site of the PSTV lentiviral backbone. Lentiviruses were prepared and titered as described (L. Xin, et al., Proc Natl Acad Sci USA 102, 6942-6947 (2005)).

### 5. Organotypic Human Prostate Transformation Assay

This assay was conducted as previously described (J. W. Park et al., Proc Natl Acad Sci USA 113, 4482-4487 (2016); J. W. Park et al., Science 362, 91-95 (2018)) with de-identified human prostate samples. Doxycycline (1 μg/mL, Calbiochem 324385) was added to all culture media and renewed every 3 days.

### 6. Xenograft Outgrowth of Transformed Cells and Cell Line Derivation

The xenograft and cell line derivation protocols have been previously described and were modified only to accommodate the doxycyline-inducible vector (J. W. Park et al., Proc Natl Acad Sci USA 113, 4482-4487 (2016); J. W. Park et al., Science 362, 91-95 (2018)). Mice were fed sterile doxycycline chow (Bio-Serv S3888) continuously starting 3 days before xenograft implantation. Cell line initiation was performed on harvested tumors with the addition of 1 μg/mL doxycycline to all media.

### 7. Cell Line Exon Annotations

Exon annotations of known stop codons and the middle exon length were generated based on the same GENCODE gene annotation file used for alignment. Potential frameshift annotation is determined if the middle exon length cannot be divided by three. Potential RNA binding proteins were labeled according to the GO annotation term ‘RNA binding’.

### 8. Cell Line Propagation

The engineered cell lines were grown in stem cell media, composed of advanced DMEM/F12K (Gibco 12634028) base media with addition of B27 (Gibco 17504044), EGF (10 ng/mL, Peprotech 100-47), and FGF2 (10 ng/mL, Peprotech 100-18B) as well as Glutamax (Gibco 35050061). Doxycycline (1 μg/mL) was added to cultures to maintain MYC expression. Media was renewed every 3 days.

### 9. Myc Withdrawal Experiments

Cells were collected by centrifugation and washed with media three times to remove doxycycline. 1 million cells were plated for each condition. Doxycycline was added back to the appropriate wells and then harvested at the appropriate time point (0-24 h).

### 10. Histology

Portions of xenograft outgrowths were fixed in formalin overnight and transferred to 70% ethanol solution before submission for further processing by the Tissue Procurement Core Laboratory at UCLA (TPCL). Organoids were collected by dispase dissociation from Matrigel, washed three times with PBS, and then formalin-fixed for 30 min at room temperature. The fixed organoids were again collected by centrifugation and resuspended in HistoGel and submitted to TPCL. All samples were paraffin-embedded, sectioned at 4 μm, and mounted on glass slides. Hematoxylin and cosin staining was conducted according to standard protocols.

### 11. Immunohistochemistry

Immunohistochemical studies were conducted as previously described (J. W. Park et al., Science 362, 91-95 (2018)). Briefly, unstained slides were subjected to deparaffinization, rehydration, and heat-activated citric acid antigen retrieval. Rehydrated slides were blocked with 1% horse serum in PBS before overnight incubation with primary antibodies also diluted in 1% horse serum/PBS. Primary and secondary antibodies and their dilutions are listed below. Antibody binding was detected using an HRP-conjugated secondary antibody and a chromogenic substrate.

### 12. Immunoblotting

Portions of tumor xenografts or 10 million cultured cells were placed in 8M urea lysis buffer with protease inhibitors (Sigma-Aldrich 4693159001) and homogenized with a Dounce apparatus. The lysate was cleared by ultracentrifugation at 30,000×g for 30 min. Samples were denatured by boiling in SDS loading buffer under reducing conditions for 1 min and subjected to polyacrylamide gel electrophoresis. Wet transfer to nitrocellulose membrane was followed by blocking in 1% milk/0.1% Tween/PBS and overnight primary antibody incubation at 5° C. in the same buffer. HRP-conjugated secondary antibodies were applied after washing and the blot visualized with a pro-luminescent substrate. Semi-quantitative blots of SRSF3 protein levels used PVDF membrane. Fluorescence levels were measured by Typhoon scanner and normalized to GAPDH levels. Antibody sources and dilutions are described below.

### 13. Antibodies for Flow Cytometry, Immunohistochemistry and Immunoblotting

Antibodies used for flow cytometry were the fluorochrome conjugates CD49f-PE (12-0495-82; eBiosciences) and Trop2-APC (FAB650A; R&D Systems). Primary antibodies used for immunohistochemistry included CK8 (1:1,000, Covance MMS-162P), AR (1:250, Santa Cruz sc-816), PSA (KLK3) (1:2000, Dako A0562), CK5 (1:1000, Covance PRB-160P), and p63 (1:250, Santa Cruz sc-8431). Secondary antibodies used were ImmPRESS anti-rabbit Ig peroxidase and anti-mouse Ig peroxidase (Vector Labs). Liquid DAB+substrate reagent (Dako) was used to perform direct chromogenic visualization. The following primary antibodies were used for immunoblotting (all at 1:1000 dilution, unless otherwise noted): Myc (Abcam ab32072), pan-AKT (Cell Signaling 4691), p53 (Cell Signaling Technology 2527), PARP1 (AbCam ab32138), cleaved PARP1 (AbCam, ab32064), anti-Cdk2 (AbCam ab32147), anti-Cdk2 (phospho Y15) (AbCam ab76146), p21 anti-p21 [EPR3993] (ab109199), and GAPDH (1:5,000, GeneTex GT239). HRP-conjugated goat anti-rabbit and goat-anti-mouse secondary antibodies (BioRad) were used for luminescent detection. For semi-quantitative Western blots, goat anti-mouse-cy5 (1:5000, Sigma-Aldrich GEPA45009) was used.

### 14. Cell Cycle Analysis

One million cells were withdrawn from doxycycline as described above and harvested by centrifugation at the appropriate time-point. Cell pellets were washed three times with PBS and then singly dissociated with trypsin prior to fixation in 10% cold ethanol. After overnight fixation at 5° C., cells were pelleted and rehydrated in PBS. RNAse was added and the suspension incubated at room temperature for 4 h before staining with 20 ng/ml 7AAD and analysis by flow cytometry.

### 15. Cell Growth Assay

Cells were washed with PBS, withdrawn from doxycycline, and plated at a density of 100,000 cells per well. Cells were lysed with CellTiterGlo luciferase reagent at the appropriate time and submitted for luminometry.

### 16. Whole Transcriptome Sequencing Analysis

Total RNA was isolated by guanidinium thiocyanate-phenol-chloroform extraction, followed by column clean-up. Isolated RNA was submitted for RNA integrity number (RIN) analysis. Only samples with RIN>9 were carried forward. cDNA libraries were prepared from isolated RNA after poly-A selection using the TruSeq RNA Sample Prep Kit v2 (Illumina). High-throughput sequencing with 150 bp paired-end reads was performed using an Illumina HiSeq 2500. At least 100 million reads were collected for each sample.

### 17. Cell Line Exon Annotations

Exon annotations of known stop codons and the middle exon length were generated based on the same GENCODE gene annotation file used for alignment. Potential frameshift annotation is determined if the middle exon length cannot be divided by three. Potential RNA binding proteins were labeled according to the GO annotation term ‘RNA binding’.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the references cited throughout the disclosure of the application, to the extent that they provide exemplary procedural or other details supplementary to those set forth herein, are specifically incorporated herein by reference in their entirety.
1. F. E. Baralle, J. Giudice, Alternative splicing as a regulator of development and tissue identity. Nat Rev Mol Cell Biol 18, 437-451 (2017).
2. S. Liu, C. Cheng, Alternative RNA splicing and cancer. Wiley Interdiscip Rev RNA 4, 547-566 (2013).
3. Y. Ho, S. M. Dehm, Androgen Receptor Rearrangement and Splicing Variants in Resistance to Endocrine Therapies in Prostate Cancer. Endocrinology 158, 1533-1542 (2017).
4. R. Catena et al., Increased expression of VEGF121/VEGF165-189 ratio results in a significant enhancement of human prostate tumor angiogenesis. Int J Cancer 120, 2096-2109 (2007).
5. G. Narla et al., KLF6-SV1 overexpression accelerates human and mouse prostate cancer progression and metastasis. J Clin Invest 118, 2711-2721 (2008).
6. R. M. Hagen et al., Quantitative analysis of ERG expression and its splice isoforms in formalin-fixed, paraffin-embedded prostate cancer samples: association with seminal vesicle invasion and biochemical recurrence. Am J Clin Pathol 142, 533-540 (2014).
7. D. R. Mercatante, C. D. Bortner, J. A. Cidlowski, R. Kole, Modification of alternative splicing of Bcl-x pre-mRNA in prostate and breast cancer cells. analysis of apoptosis and cell death. J Biol Chem 276, 16411-16417 (2001).
8. E. Antonopoulou, M. Ladomery, Targeting Splicing in Prostate Cancer. Int J Mol Sci 19
   (2018).
9. K. Arora, C. E. Barbieri, Molecular Subtypes of Prostate Cancer. Curr Oncol Rep 20, 58 (2018).
10. H. Beltran et al., Divergent clonal evolution of castration-resistant neuroendocrine prostate cancer. Nat Med 22, 298-305 (2016).
11. D. Robinson et al., Integrative clinical genomics of advanced prostate cancer. Cell 161, 1215-1228 (2015).
12. D. R. Robinson et al., Integrative clinical genomics of metastatic cancer. Nature 548, 297-303 (2017).
13. N. Cancer Genome Atlas Research, The Molecular Taxonomy of Primary Prostate Cancer. Cell 163, 1011-1025 (2015).
14. S. C. Baca et al., Punctuated evolution of prostate cancer genomes. Cell 153, 666-677 (2013).
15. R. B. Jenkins, J. Qian, M. M. Lieber, D. G. Bostwick, Detection of c-myc oncogene amplification and chromosomal anomalies in metastatic prostatic carcinoma by fluorescence in situ hybridization. Cancer Res 57, 524-531 (1997).
16. M. J. Linja et al., Amplification and overexpression of androgen receptor gene in hormone-refractory prostate cancer. Cancer Res 61, 3550-3555 (2001).
17. H. Chen et al., Pathogenesis of prostatic small cell carcinoma involves the inactivation of the P53 pathway. Endocr Relat Cancer 19, 321-331 (2012).
18. C. Tran et al., Development of a second-generation antiandrogen for treatment of advanced prostate cancer. Science 324, 787-790 (2009).
19. J. Mateo et al., DNA-Repair Defects and Olaparib in Metastatic Prostate Cancer. N Engl J Med 373, 1697-1708 (2015).
20. A. Paschalis et al., Alternative splicing in prostate cancer. Nat Rev Clin Oncol (2018).
21. K. Thorsen et al., Alternative splicing in colon, bladder, and prostate cancer identified by exon array analysis. Mol Cell Proteomics 7, 1214-1224 (2008).
22. S. Ren et al., RNA-seq analysis of prostate cancer in the Chinese population identifies recurrent gene fusions, cancer-associated long noncoding RNAs and aberrant alternative splicings. Cell Res 22, 806-821 (2012).
23. B. D. Wang et al., Alternative splicing promotes tumour aggressiveness and drug resistance in African American prostate cancer. Nat Commun 8, 15921 (2017).
24. H. R. Li et al., Two-dimensional transcriptome profiling: identification of messenger RNA isoform signatures in prostate cancer from archived paraffin-embedded cancer specimens. Cancer Res 66, 4079-4088 (2006).
25. C. Zhang et al., Profiling alternatively spliced mRNA isoforms for prostate cancer classification. BMC Bioinformatics 7, 202 (2006).
26. Y. Gan et al., Roles of Alternative RNA Splicing of the Bif-1 Gene by SRRM4 During the Development of Treatment-induced Neuroendocrine Prostate Cancer. EBioMedicine 31, 267-275 (2018).
27. A. R. Lee et al., Alternative RNA splicing of the MEAF6 gene facilitates neuroendocrine prostate cancer progression. Oncotarget 8, 27966-27975 (2017).
28. Y. Li et al., SRRM4 Drives Neuroendocrine Transdifferentiation of Prostate Adenocarcinoma Under Androgen Receptor Pathway Inhibition. Eur Urol 71, 68-78 (2017).
29. S. Shen et al., rMATS: robust and flexible detection of differential alternative splicing from replicate RNA-Seq data. Proc Natl Acad Sci USA 111, E5593-5601 (2014).
30. Z. Xie, Y. Xing, rMATS-turbo. http://rnaseq-rmats.sourceforge.net/rmats4.0.2 (2019).
31. G. T. Consortium, The Genotype-Tissue Expression (GTEx) project. Nat Genet 45, 580-585 (2013).
32. N. Cancer Genome Atlas Research et al., The Cancer Genome Atlas Pan-Cancer analysis project. Nat Genet 45, 1113-1120 (2013).
33. J. T. Leek et al., Tackling the widespread and critical impact of batch effects in high-throughput data. Nat Rev Genet 11, 733-739 (2010).
34. S. Anders, A. Reyes, W. Huber, Detecting differential usage of exons from RNA-seq data. Genome Res 22, 2008-2017 (2012).
35. S. Shen, Y. Wang, C. Wang, Y. N. Wu, Y. Xing, SURVIV for survival analysis of mRNA isoform variation. Nat Commun 7, 11548 (2016).
36. E. Park, Z. Pan, Z. Zhang, L. Lin, Y. Xing, The Expanding Landscape of Alternative Splicing Variation in Human Populations. Am J Hum Genet 102, 11-26 (2018).
37. N. T. Johnson, A. Dhroso, K. J. Hughes, D. Korkin, Biological classification with RNA-seq data: Can alternatively spliced transcript expression enhance machine learning classifiers? RNA 24, 1119-1132 (2018).
38. S. Djebali et al., Landscape of transcription in human cells. Nature 489, 101-108 (2012).
39. S. Frank, P. Nelson, V. Vasioukhin, Recent advances in prostate cancer research: large-scale genomic analyses reveal novel driver mutations and DNA repair defects. F1000Res 7 (2018).
40. V. K. Mootha et al., PGC-1alpha-responsive genes involved in oxidative phosphorylation are coordinately downregulated in human diabetes. Nat Genet 34, 267-273 (2003).
41. X. Qiu, H. Wu, R. Hu, The impact of quantile and rank normalization procedures on the testing power of gene differential expression analysis. BMC Bioinformatics 14, 124 (2013).
42. A. Liberzon et al., The Molecular Signatures Database (MSigDB) hallmark gene set collection. Cell Syst 1, 417-425 (2015).
43. S. R. Setlur et al., Estrogen-dependent signaling in a molecularly distinct subclass of aggressive prostate cancer. J Natl Cancer Inst 100, 815-825 (2008).
44. M. Krzywinski, I. Birol, S. J. Jones, M. A. Marra, Hive plots—rational approach to visualizing networks. Brief Bioinform 13, 627-644 (2012).
45. T. I. Zack et al., Pan-cancer patterns of somatic copy number alteration. Nat Genet 45. 45, 1134-1140 (2013).
46. C. V. Dang. MYC on the path to cancer. Cell 149, 22-35 (2012).
47. B. Gurel et al., Nuclear MYC protein overexpression is an early alteration in human prostate carcinogenesis. Mod Pathol 21, 1156-1167 (2008).
48. C. M. Koh et al., MYC and Prostate Cancer. Genes Cancer 1, 617-628 (2010).
49. D. Aran et al., Comprehensive analysis of normal adjacent to tumor transcriptomes. Nat Commun 8, 1077 (2017).
50. L. M. Urbanski, N. Leclair, O. Anczukow, Alternative-splicing defects in cancer: Splicing regulators and their downstream targets, guiding the way to novel cancer therapeutics. Wiley Interdiscip Rev RNA 9, e1476 (2018).
51. M. Cui et al., Genes involved in pre-mRNA 3′-end formation and transcription termination revealed by a lin-15 operon Muv suppressor screen. Proc Natl Acad Sci USA 105, 16665-16670 (2008).
52. X. He, P. Zhang. Serine/arginine-rich splicing factor 3 (SRSF3) regulates homologous recombination-mediated DNA repair. Mol Cancer 14, 158 (2015).
53. R. Jia, C. Li, J. P. McCoy, C. X. Deng. Z. M. Zheng, SRp20 is a proto-oncogene critical for cell proliferation and tumor induction and maintenance. Int J Biol Sci 6, 806-826 (2010).
54. C. Corbo, S. Orru, F. Salvatore, SRp20: an overview of its role in human diseases. Biochem Biophys Res Commun 436, 1-5 (2013).
55. H. Jumaa, P. J. Nielsen, The splicing factor SRp20 modifies splicing of its own mRNA and ASF/SF2 antagonizes this regulation. EMBO J 16, 5077-5085 (1997).
56. H. Land. L. F. Parada, R. A. Weinberg, Tumorigenic conversion of primary embryo fibroblasts requires at least two cooperating oncogenes. Nature 304, 596-602 (1983).
57. C. Wang. M. P. Lisanti, D. J. Liao, Reviewing once more the c-myc and Ras collaboration: converging at the cyclin D1-CDK4 complex and challenging basic concepts of cancer biology. Cell Cycle 10, 57-67 (2011).
58. J. B. Cohen, S. D. Broz, A. D. Levinson, Expression of the H-ras proto-oncogene is controlled by alternative splicing. Cell 58, 461-472 (1989).
59. M. Camats, M. Kokolo, K. J. Heesom, M. Ladomery, M. Bach-Elias, P19 H-ras induces G1/S phase delay maintaining cells in a reversible quiescence state. PLOS One 4, e8513 (2009).
60. B. Pereira et al., The somatic mutation profiles of 2,433 breast cancers refines their genomic and transcriptomic landscapes. Nat Commun 7, 11479 (2016).
61. N. Cancer Genome Atlas Research, Comprehensive molecular profiling of lung adenocarcinoma. Nature 511, 543-550 (2014).
62. H. Ji et al., Cell-type independent MYC target genes reveal a primordial signature involved in biomass accumulation. PLOS One 6, e26057 (2011).
63. K. I. Zeller, A. G. Jegga, B. J. Aronow, K. A. O'Donnell, C. V. Dang, An integrated database of genes responsive to the Myc oncogenic transcription factor: identification of direct genomic targets. Genome Biol 4, R69 (2003).
64. S. Chandriani et al., A core MYC gene expression signature is prominent in basal-like breast cancer but only partially overlaps the core serum response. PLOS One 4, e6693 (2009).
65. J. W. Park et al., Prostate epithelial cell of origin determines cancer differentiation state in an organoid transformation assay. Proc Natl Acad Sci USA 113, 4482-4487 (2016).
66. T. Stoyanova et al., Prostate cancer originating in basal cells progresses to adenocarcinoma propagated by luminal-like cells. Proc Natl Acad Sci USA 110, 20111-20116 (2013).
67. E. G. Bluemn et al., Androgen Receptor Pathway-Independent Prostate Cancer Is Sustained through FGF Signaling. Cancer Cell 32, 474-489 e476 (2017).
68. C. Dani et al., Extreme instability of myc mRNA in normal and transformed human cells. Proc Natl Acad Sci USA 81, 7046-7050 (1984).
69. A. L. Gartel et al., Myc represses the p21(WAF1/CIP1) promoter and interacts with Sp1/Sp3. Proc Natl Acad Sci USA 98, 4510-4515 (2001).
70. L. Demirdjian, Y. N. Wu, Y. Xing, PAIRADISE: Paired analysis of differential isoform expression. https://bioconductor.org/packages/release/bioc/html/PAIRADISE.html (2019).
71. A. Zehir et al., Mutational landscape of metastatic cancer revealed from prospective clinical sequencing of 10,000 patients. Nat Med 23, 703-713 (2017).
72. C. Hodges, J. G. Kirkland, G. R. Crabtree, The Many Roles of BAF (mSWI/SNF) and PBAF Complexes in Cancer. Cold Spring Harb Perspect Med 6 (2016).
73. S. F. Khan et al., The roles and regulation of TBX3 in development and disease. Gene, 144223 (2019).
74. B. P. Lewis, R. E. Green, S. E. Brenner, Evidence for the widespread coupling of alternative splicing and nonsense-mediated mRNA decay in humans. Proc Natl Acad Sci USA 100, 189-192 (2003).
75. C. M. Koh et al., MYC regulates the core pre-mRNA splicing machinery as an essential step in lymphomagenesis. Nature 523, 96-100 (2015).
76. T. Y. Hsu et al., The spliceosome is a therapeutic vulnerability in MYC-driven cancer. Nature 525, 384-388 (2015).
77. S. Das, O. Anczukow, M. Akerman, A. R. Krainer, Oncogenic splicing factor SRSF1 is a critical transcriptional target of MYC. Cell Rep 1, 110-117 (2012).
78. M. Ratnadiwakara et al., SRSF3 promotes pluripotency through Nanog mRNA export and coordination of the pluripotency gene expression program. Elife 7 (2018).
79. B. A. Smith et al., A Human Adult Stem Cell Signature Marks Aggressive Variants across Epithelial Cancers. Cell Rep 24, 3353-3366 e3355 (2018).
80. R. Sridharan et al., Role of the murine reprogramming factors in the induction of pluripotency. Cell 136, 364-377 (2009).
81. S. Nasif, L. Contu, O. Muhlemann, Beyond quality control: The role of nonsense-mediated mRNA decay (NMD) in regulating gene expression. Semin Cell Dev Biol 75, 78-87 (2018).
82. Z. Zhou, X. D. Fu, Regulation of splicing by SR proteins and SR protein-specific kinases. Chromosoma 122, 191-207 (2013).
83. Y. Liu, A. Beyer, R. Aebersold, On the Dependency of Cellular Protein Levels on mRNA Abundance. Cell 165, 535-550 (2016).
84. N. Martinez-Montiel, N. H. Rosas-Murrieta, M. Anaya Ruiz, E. Monjaraz-Guzman, R. Martinez-Contreras, Alternative Splicing as a Target for Cancer Treatment. Int J Mol Sci 19 (2018).
85. M. D. Mailman et al., The NCBI dbGaP database of genotypes and phenotypes. Nat Genet 39, 1181-1186 (2007).
86. K. A. Tryka et al., NCBI's Database of Genotypes and Phenotypes: dbGaP. Nucleic Acids Res 42, D975-979 (2014).
87. R. L. Grossman et al., Toward a Shared Vision for Cancer Genomic Data. N Engl J Med 375, 1109-1112 (2016).
88. A. Dobin et al., STAR: ultrafast universal RNA-seq aligner. Bioinformatics 29, 15-21 (2013).
89. J. Harrow et al., GENCODE: the reference human genome annotation for The ENCODE Project. Genome Res 22, 1760-1774 (2012).
90. C. Trapnell et al., Differential gene and transcript expression analysis of RNA-seq experiments with TopHat and Cufflinks. Nat Protoc 7, 562-578 (2012).
91. L. s. Torgo, Data mining with R: learning with case studies, Chapman & Hall/CRC data mining and knowledge discovery series (CRC Press, Taylor & Francis Group, Boca Raton, ed. Second edition., 2017), pp. xix, 405 pages.
92. D. Risso, F. Perraudeau, S. Gribkova, S. Dudoit, J. P. Vert, A general and flexible method for signal extraction from single-cell RNA-seq data. Nat Commun 9, 284 (2018).
93. P. J. Rousseeuw, Silhouettes: A graphical aid to the interpretation and validation of cluster analysis. Journal of Computational and Applied Mathematics 20, 53-65 (1987).
94. M. Mächler, P. Rousseeuw, A. Struyf, M. Hubert, K. Hornik, cluster: Cluster Analysis Basics and Extensions (R package version 2.0.7-1, 2018).
95. M. V. Kuleshov et al., Enrichr: a comprehensive gene set enrichment analysis web server 2016 update. Nucleic Acids Res 44, W90-97 (2016).
96. P. Khatri, S. Draghici, Ontological analysis of gene expression data: current tools, limitations, and open problems. Bioinformatics 21, 3587-3595 (2005).
97. F. Supek, M. Bosnjak, N. Skunca, T. Smuc, REVIGO summarizes and visualizes long lists of gene ontology terms. PLOS One 6, e21800 (2011).
98. A. Liberzon et al., Molecular signatures database (MSigDB) 3.0. Bioinformatics 27, 1739-1740 (2011).
99. A. Subramanian et al., Gene set enrichment analysis: a knowledge-based approach for interpreting genome-wide expression profiles. Proc Natl Acad Sci USA 102, 15545-15550 (2005).
100. M. Wang, Y. Zhao, B. Zhang, Efficient Test and Visualization of Multi-Set Intersections. Sci Rep 5, 16923 (2015).
101. L. Xin, D. A. Lawson, O. N. Witte, The Sca-1 cell surface marker enriches for a prostate-regenerating cell subpopulation that can initiate prostate tumorigenesis. Proc Natl Acad Sci USA 102, 6942-6947 (2005).
102. J. W. Park et al., Reprogramming normal human epithelial tissues to a common, lethal neuroendocrine cancer lineage. Science 362. 91-95 (2018).

1. A peptide comprising at least 70% sequence identity to a peptide of SEQ ID NO:1-19.

2. The peptide of claim 1, wherein the peptide comprises one of SEQ ID NOS: 1-19.

3. The peptide of claim 1, wherein the peptide comprises at least 6 contiguous amino acids of one of SEQ ID NOS:1-19.

4. The peptide of claim 1, wherein the peptide is 13 amino acids or fewer in length.

5. The peptide of claim 4, wherein the peptide consists of 9 amino acids.

6. The peptide of claim 1, wherein the peptide is immunogenic.

7. The peptide of claim 1, wherein the peptide is modified.

8. The peptide of claim 7, wherein the modification comprises conjugation to a molecule.

9. The peptide of claim 7, wherein the molecule comprises an antibody, a lipid, an adjuvant, or a detection moiety.

10. The peptide of claim 1, wherein the peptide has 1, 2 or 3 substitutions relative to a peptide of one of SEQ ID NOS:1-19.

11. A peptide comprising at least 6 contiguous amino acids from an alternatively spliced polypeptide,
wherein the at least 6 contiguous amino acids comprises an alternative splice site junction of the polypeptide or wherein the peptide comprises at least 6 contiguous amino acids from an alternatively spliced exon; and
wherein the alternatively spliced peptide, exon, or junction is one that is derived from an alternative splice event (AS event) identified in Table 1a, 1b, 1c, or 1d.

12. The peptide of claim 11, wherein the AS event is selected from an AS event in Table 1a.

13. The peptide of claim 11, wherein the AS event is selected from an AS event in Table 1b.

14. The peptide of claim 11, wherein the AS event is selected from an AS event in Table 1c.

15. The peptide of claim 11, wherein the AS event is selected from an AS event in Table 1d.

16. The peptide of claim 11, wherein the peptide comprises at least 10 amino acids.

17. The peptide of claim 11, wherein the peptide consists of 10 amino acids.

18. The peptide of claim 11, wherein the peptide is less than 20 amino acids in length.

19. The peptide of claim 11, wherein the peptide is conjugated to a molecule selected from an antibody, a lipid, an adjuvant or a detection moiety.

20-21. (canceled)

22. A molecular complex comprising the peptide of claim 1 and a MHC polypeptide.

23-137. (canceled)


SEQUENCE LISTING (19 sequences)

SEQ ID NO: 1 - protein, 10 aa, Artificial Sequence
  Description of Artificial Sequence Synthetic peptide
SLAIGGVTEA

SEQ ID NO: 2 - protein, 11 aa, Artificial Sequence
  Description of Artificial Sequence Synthetic peptide
LLLGIAKLLKV

SEQ ID NO: 3 - protein, 11 aa, Artificial Sequence
  Description of Artificial Sequence Synthetic peptide
HLMEENMIVYV

SEQ ID NO: 4 - protein, 9 aa, Artificial Sequence
  Description of Artificial Sequence Synthetic peptide
LLAEQPDQV

SEQ ID NO: 5 - protein, 10 aa, Artificial Sequence
  Description of Artificial Sequence Synthetic peptide
LVLISIVICV

SEQ ID NO: 6 - protein, 9 aa, Artificial Sequence
  Description of Artificial Sequence Synthetic peptide
LLAEQPDQV

SEQ ID NO: 7 - protein, 9 aa, Artificial Sequence
  Description of Artificial Sequence Synthetic peptide
ALVANLPLL

SEQ ID NO: 8 - protein, 10 aa, Artificial Sequence
  Description of Artificial Sequence Synthetic peptide
FTVTVTEPPV

SEQ ID NO: 9 - protein, 9 aa, Artificial Sequence
  Description of Artificial Sequence Synthetic peptide
ALAGKPLSL

SEQ ID NO: 10 - protein, 9 aa, Artificial Sequence
  Description of Artificial Sequence Synthetic peptide
FILTYWTPS

SEQ ID NO: 11 - protein, 9 aa, Artificial Sequence
  Description of Artificial Sequence Synthetic peptide
STMYYLWML

SEQ ID NO: 12 - protein, 10 aa, Artificial Sequence
  Description of Artificial Sequence Synthetic peptide
VSTMYYLWML

SEQ ID NO: 13 - protein, 10 aa, Artificial Sequence
  Description of Artificial Sequence Synthetic peptide
FLSELEPPAP

SEQ ID NO: 14 - protein, 10 aa, Artificial Sequence
  Description of Artificial Sequence Synthetic peptide
TLVLALVANL

SEQ ID NO: 15 - protein, 9 aa, Artificial Sequence
  Description of Artificial Sequence Synthetic peptide
STMYYLWML

SEQ ID NO: 16 - protein, 9 aa, Artificial Sequence
  Description of Artificial Sequence Synthetic peptide
FLSELEPPA

SEQ ID NO: 17 - protein, 11 aa, Artificial Sequence
  Description of Artificial Sequence Synthetic peptide
FLAIVFFASIV

SEQ ID NO: 18 - protein, 9 aa, Artificial Sequence
  Description of Artificial Sequence Synthetic peptide
STMYYLWML

SEQ ID NO: 19 - protein, 10 aa, Artificial Sequence
  Description of Artificial Sequence Synthetic peptide
KVWTETLIEA